(19) United States
(12) Patent Application Publication
(10) Pub. No.: US 2026/0325661 A1
(43) Pub. Date: Oct. 22, 2026

(54) ANTIBODY-CONJUGATED IMMUNO-ACTIVATING SMALL MOLECULE COMPOUND, AND USE

(71) Applicant: SHENZHEN CONJUGENIX PHARMACEUTICAL TECHNOLOGY CO., LTD, Shenzhen, Guangdong (CN)

(72) Inventors: Guangyi JIN, Shenzhen, Guangdong (CN); Zhulin WANG, Shenzhen, Guangdong (CN)

(73) Assignee: SHENZHEN CONJUGENIX PHARMACEUTICAL TECHNOLOGY CO., LTD, Shenzhen, Guangdong (CN)

(21) Appl. No.: 19/479,037

(22) PCT Filed: Apr. 12, 2024
(86) PCT No.: PCT/CN2024/087459

§ 371 Date: Oct. 27, 2025

(30) Foreign Application Priority Data
Apr. 27, 2023 (CN) 202310470735.X

(51) Int. Cl. A61K 47/68; A61P 35/00

(52) U.S. Cl. CPC A61K 47/68031; A61K 47/6803; A61K 47/68037; A61K 47/6849; A61P 35/00

(57) ABSTRACT

An antibody-conjugated immune-activating small molecule compound, and a use. The general formula of the conjugation structure of the antibody and the immune activation small molecule compound is as shown in the following formula: a linking functional group is a maleimide group, a carboxyl group, an isothiocyanate group, an azide group, or an alkynyl group; a linking chain is an alkoxy group or an alkyl chain; the antibody is a PD-L1 antibody; and a KX-body is a main body portion of an immuno-activating small molecule KX. While maintaining the anti-tumor function of the original antibody, the invention adds an immune priming and activation effect, thereby greatly improving the anti-tumor effect.

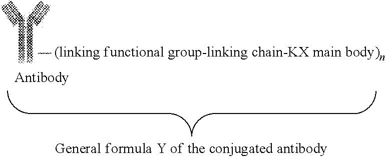

### FIELD OF THE INVENTION

The present invention relates to the field of antibody-drug conjugate (ADC), and more particularly, relates to an antibody-conjugated immune-activating small molecule compound and use.

### BACKGROUND OF THE INVENTION

Although antibody-drug conjugates have improved the efficacy of targeted anti-tumor therapy, drug resistance still develops with the prolonged duration of medication; in many cases, although tumors are inhibited for a certain period of time, recurrence or metastasis still occurs. This is because targeted elimination of tumors does not have the effect of stimulating immune function.

### SUMMARY OF THE INVENTION

The technical problem solved by the present invention is to provide an antibody-conjugated immune-activating small molecule compound and use, for addressing the problem that the aforementioned antibody-drug conjugates fail to stimulate immune function.

In one embodiment, the technical solution of the present invention to solve the above technical problem is to provide an antibody-conjugated immune-activating small molecule compound: the general formula formed by the conjugation of the antibody and the immune-activating small molecule compound is as shown in the following formula:

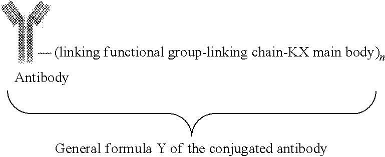

wherein the linking functional group is maleimide group, carboxyl, isothiocyano, azido or alkynyl, the linking chain is alkoxy or alkyl chain, the KX-main body is the main part of the immune-activating small molecule KX, the antibody is PD-L1 antibody, and the antibody-conjugated immune-activating small molecule compound is one of the following compounds:

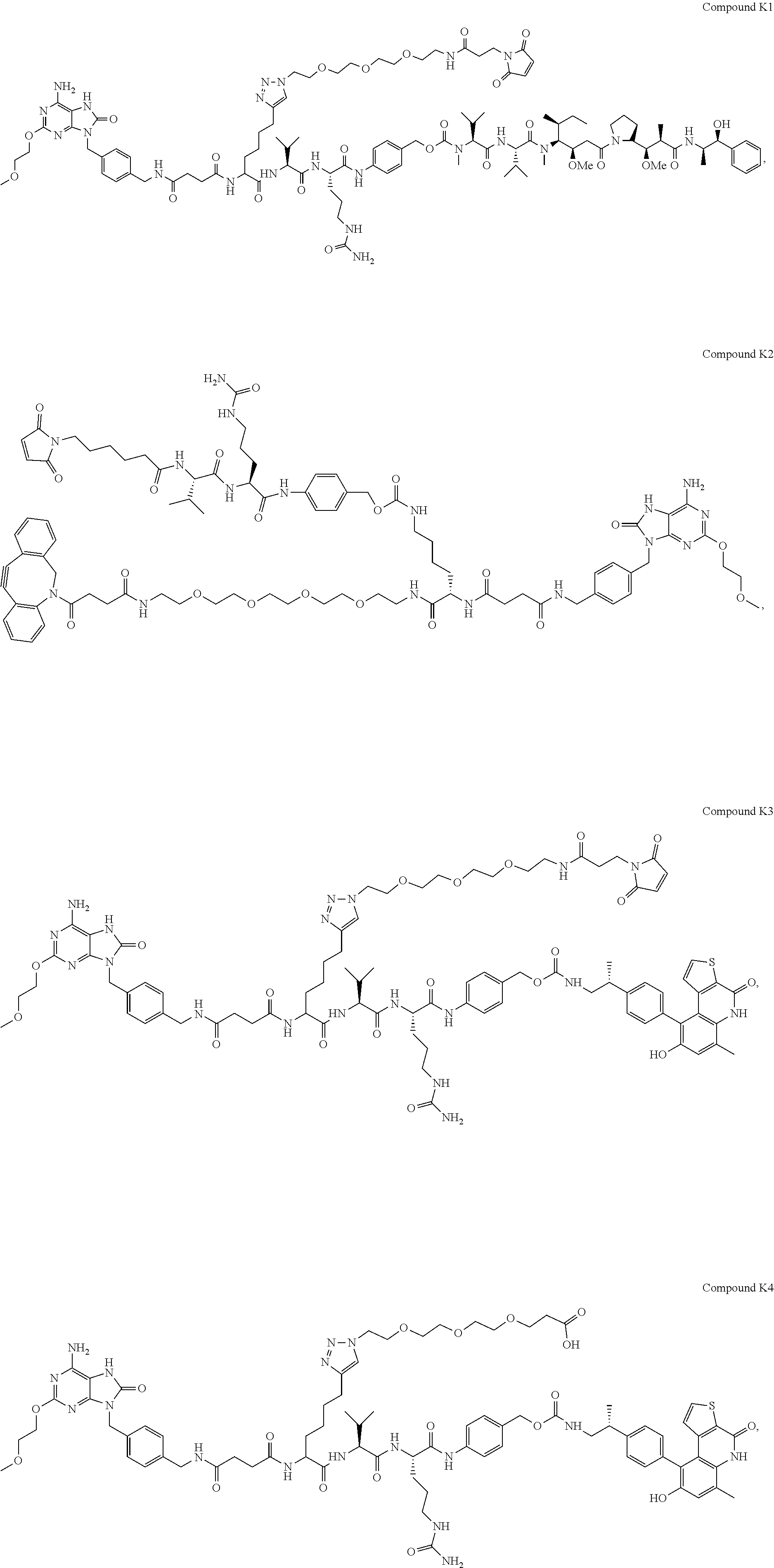

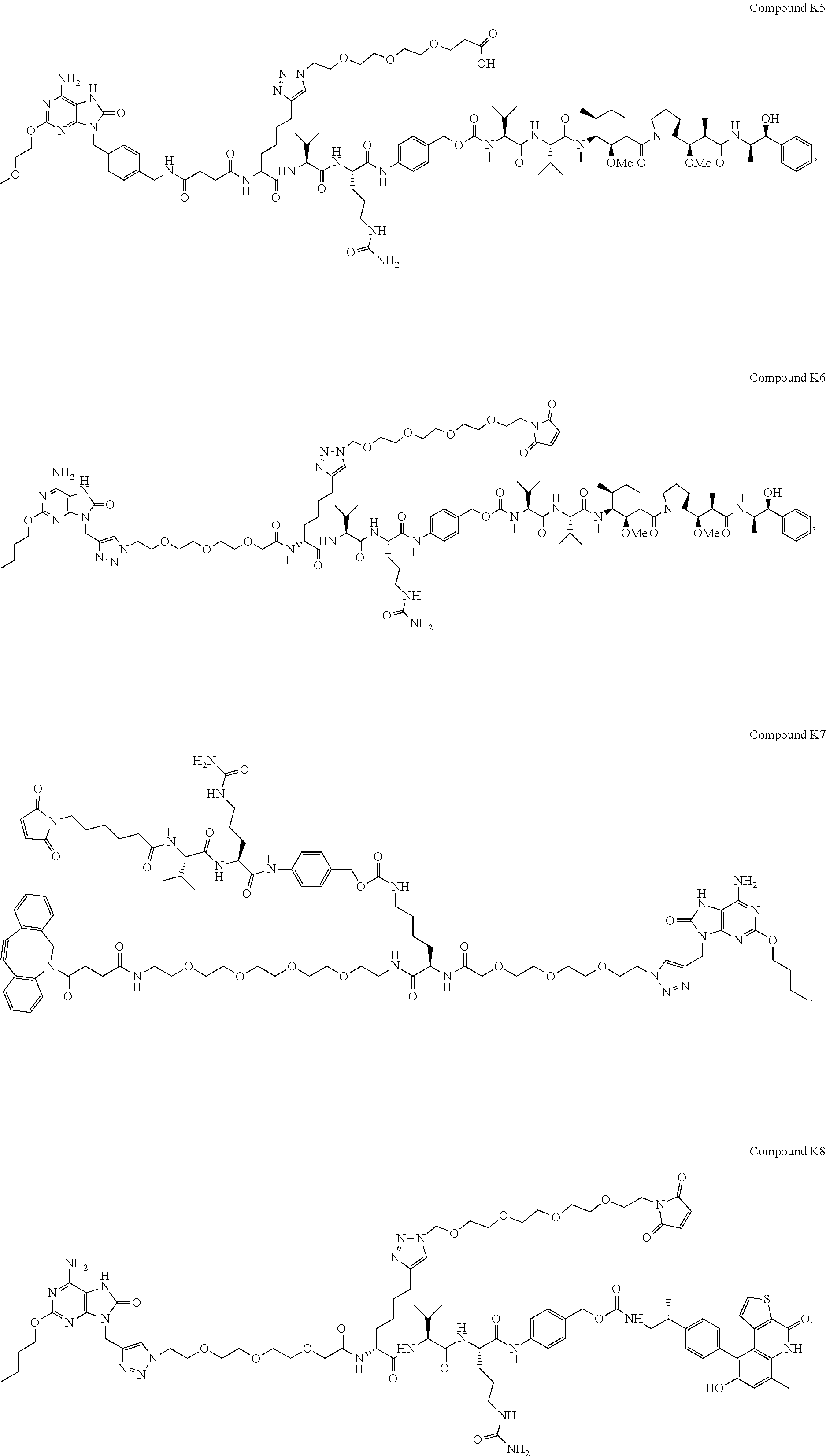

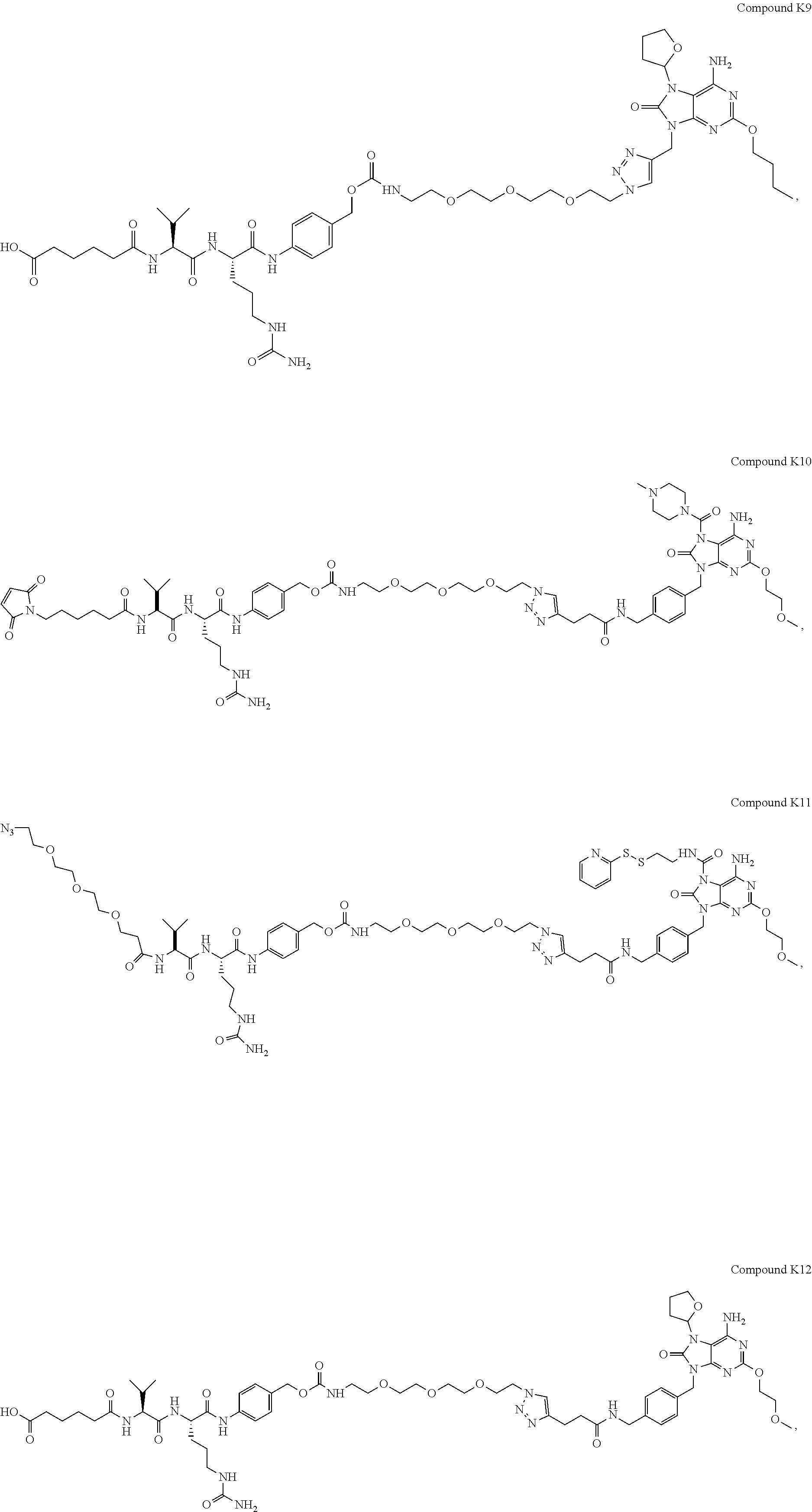

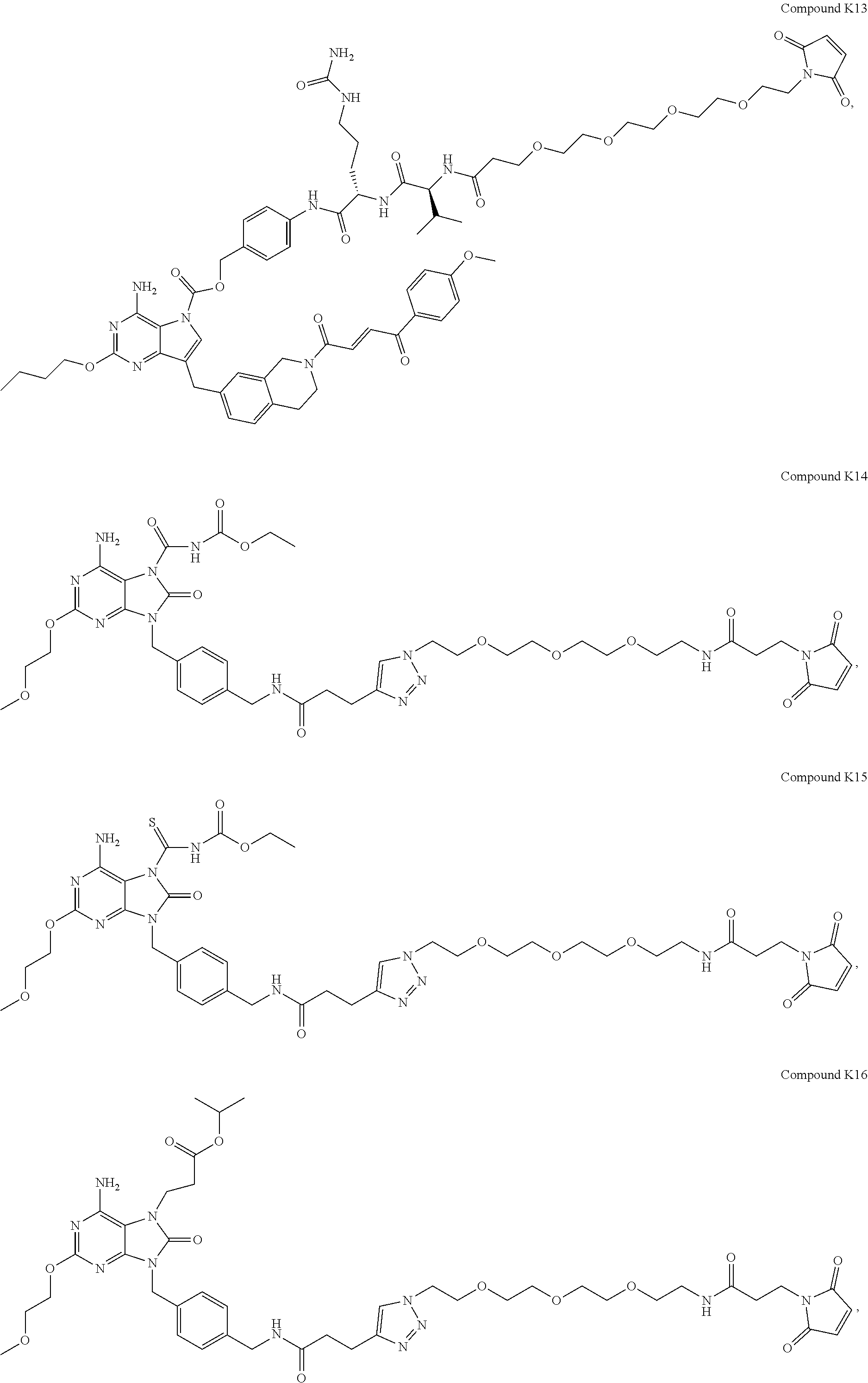 [SMILES: CCCCOc1nc(N)c2c(n1)c(Cc1ccc3c(c1)CN(C(=O)/C=C/C(=O)c1ccc(OC)cc1)CC3)cn2C(=O)OCc1ccc(NC(=O)[C@H](CCCNC(N)=O)NC(=O)[C@@H](NC(=O)CCOCCOCCOCCOCCN2C(=O)C=CC2=O)C(C)C)cc1 ; CCOC(=O)NC(=O)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21 ; CCOC(=O)NC(=S)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21 ; COCCOc1nc(N)c2c(n1)n(Cc1ccc(CNC(=O)CCc3cn(CCOCCOCCOCCNC(=O)CCN4C(=O)C=CC4=O)nn3)cc1)c(=O)n2CCC(=O)OC(C)C]

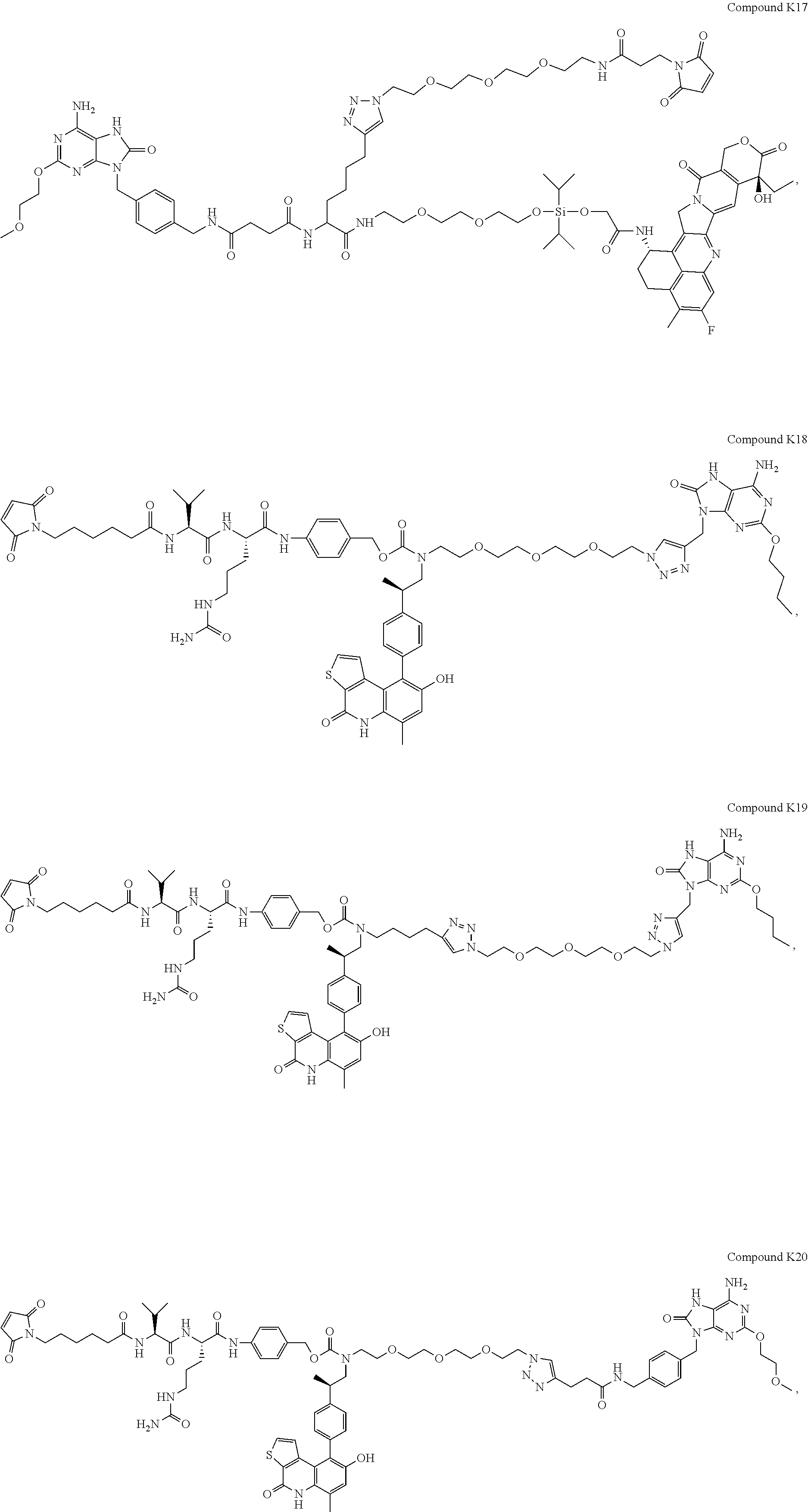

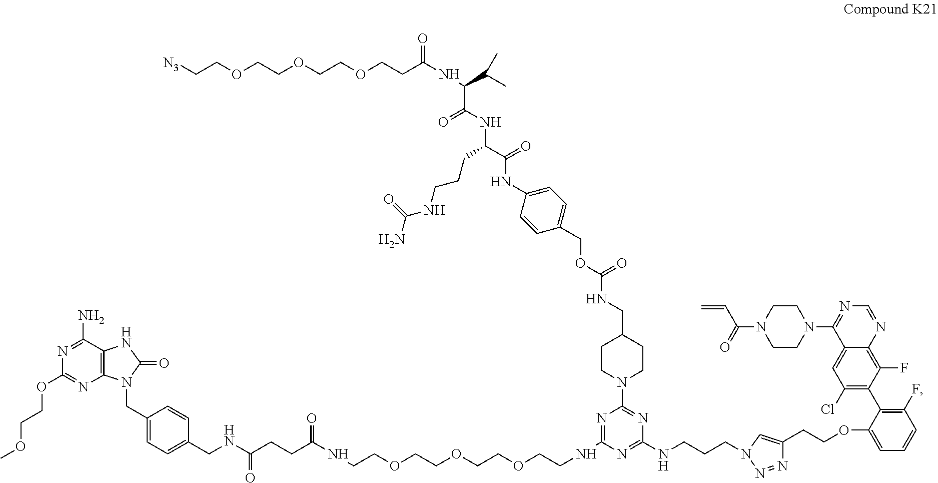 [SMILES: C=CC(=O)N1CCN(c2ncnc3c(F)c(-c4c(F)cccc4OCCc4cn(CCCNc5nc(NCCOCCOCCOCCNC(=O)CCC(=O)NCc6ccc(Cn7c(=O)[nH]c8c(N)nc(OCCOC)nc87)cc6)nc(N6CCC(CNC(=O)OCc7ccc(NC(=O)[C@H](CCCNC(N)=O)NC(=O)[C@@H](NC(=O)CCOCCOCCOCCN=[N+]=[N-])C(C)C)cc7)CC6)n5)nn4)c(Cl)cc23)CC1]

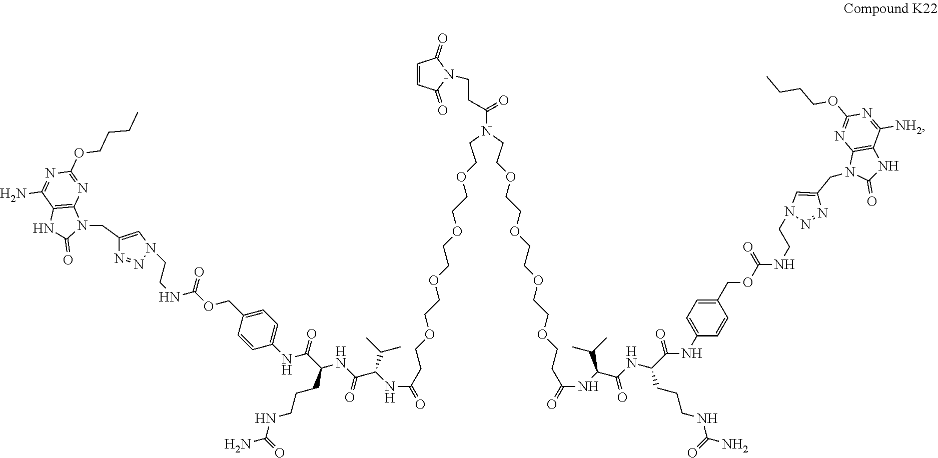 [SMILES: CCCCOc1nc(N)c2[nH]c(=O)n(Cc3cn(CCNC(=O)OCc4ccc(NC(=O)[C@H](CCCNC(N)=O)NC(=O)[C@@H](NC(=O)CCOCCOCCOCCOCCN(CCOCCOCCOCCOCCC(=O)N[C@H](C(=O)N[C@@H](CCCNC(N)=O)C(=O)Nc5ccc(COC(=O)NCCn6cc(Cn7c(=O)[nH]c8c(N)nc(OCCCC)nc87)nn6)cc5)C(C)C)C(=O)CCN5C(=O)C=CC5=O)C(C)C)cc4)nn3)c2n1]

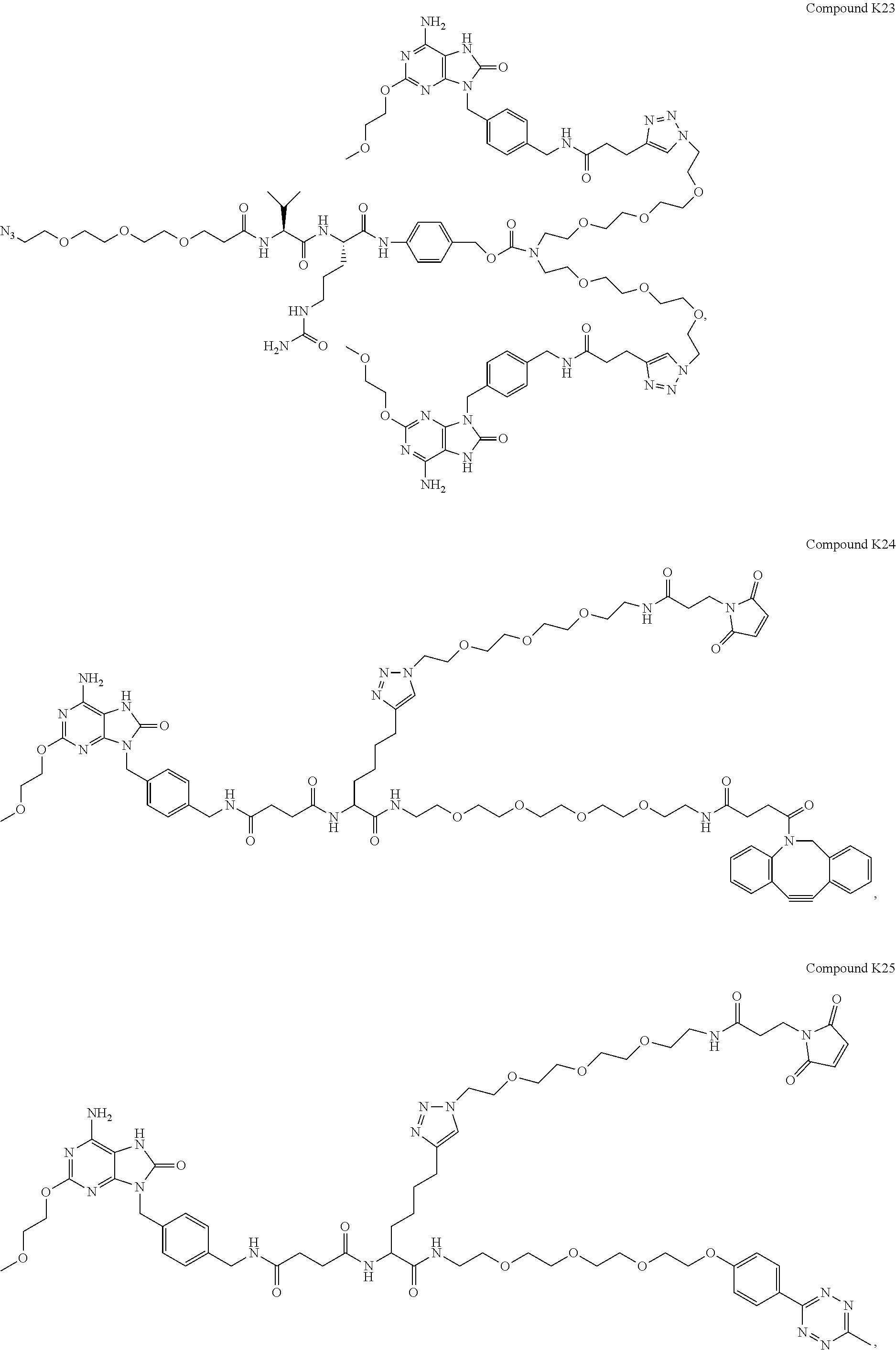

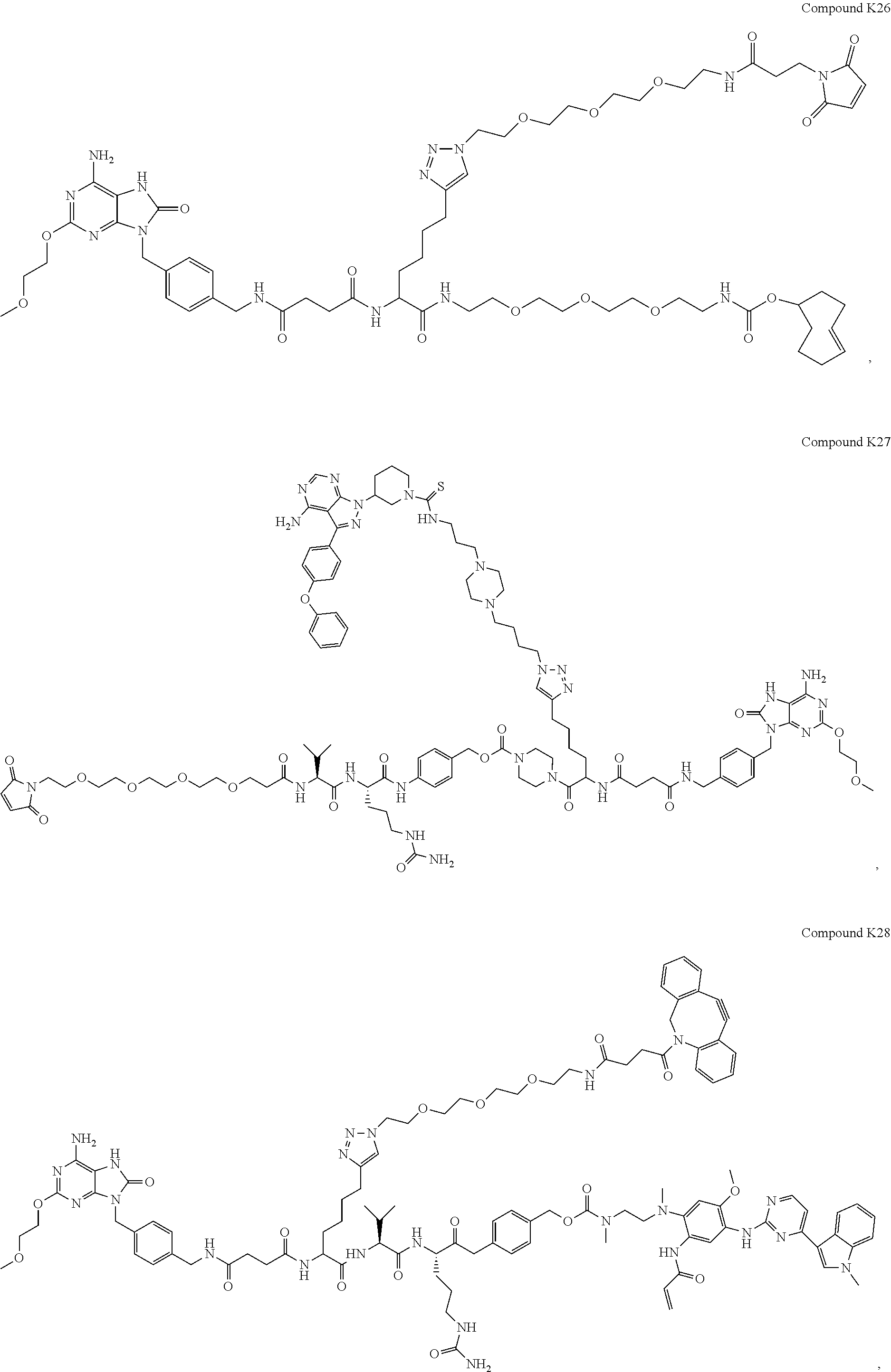

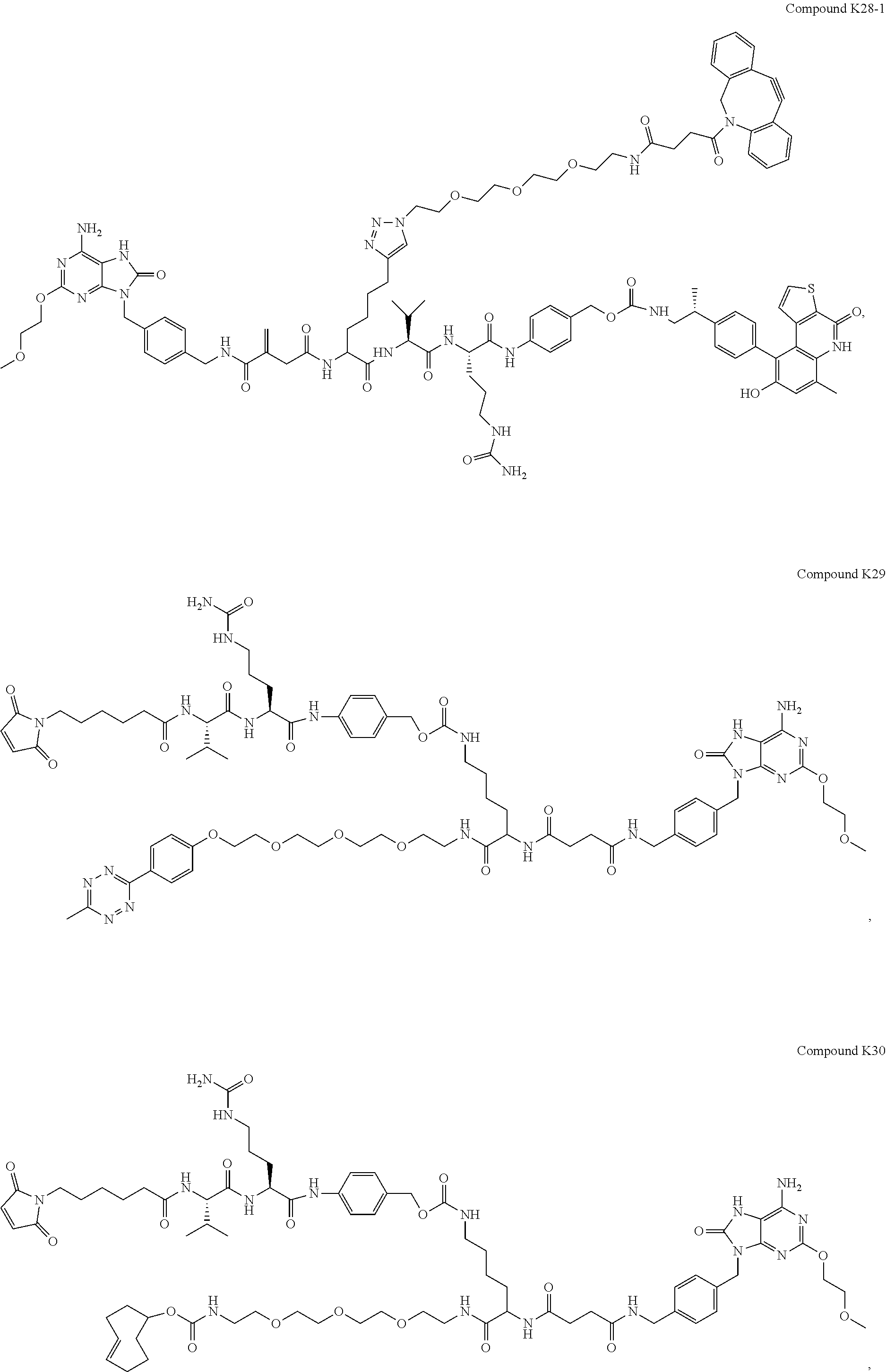

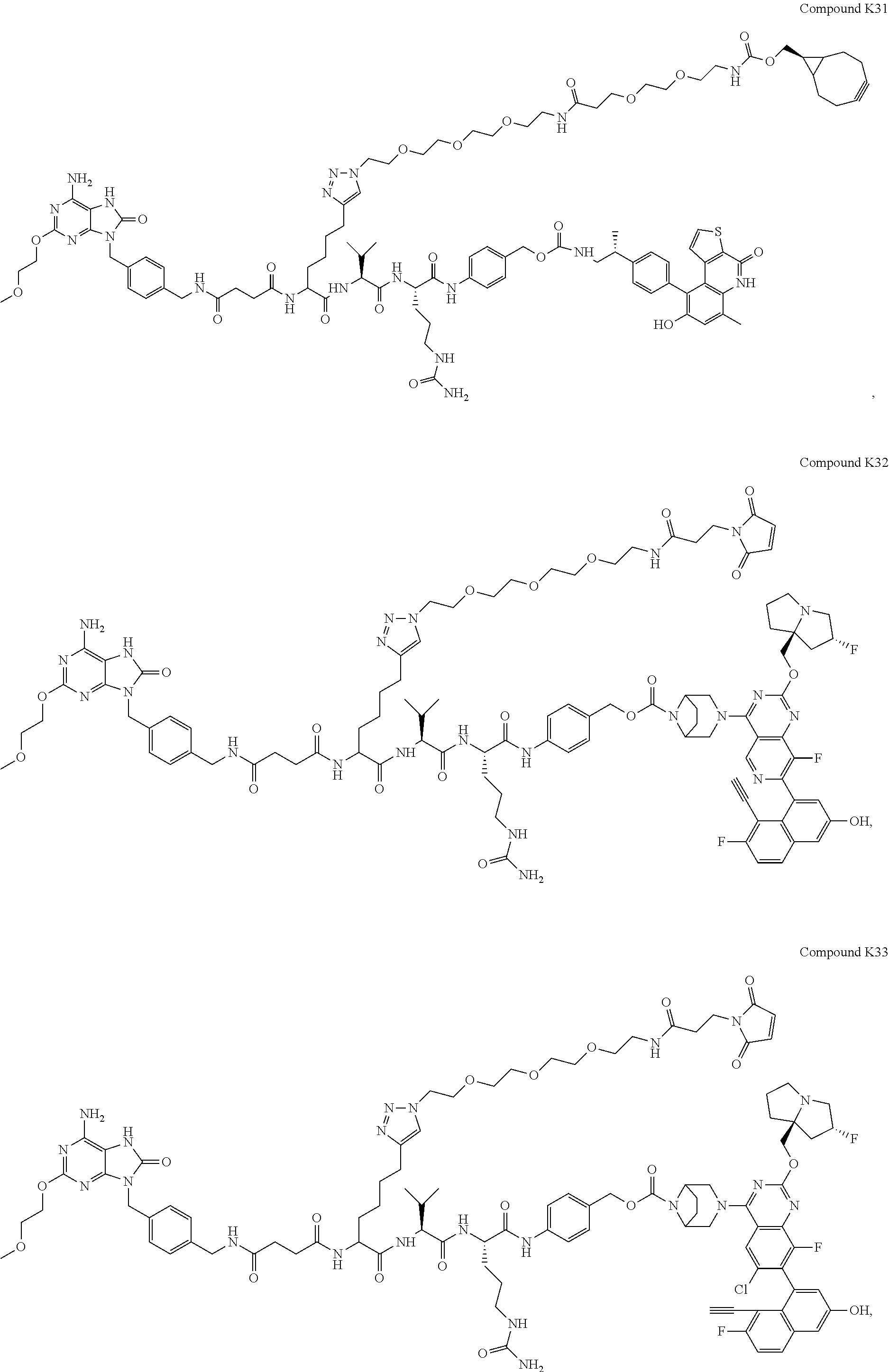

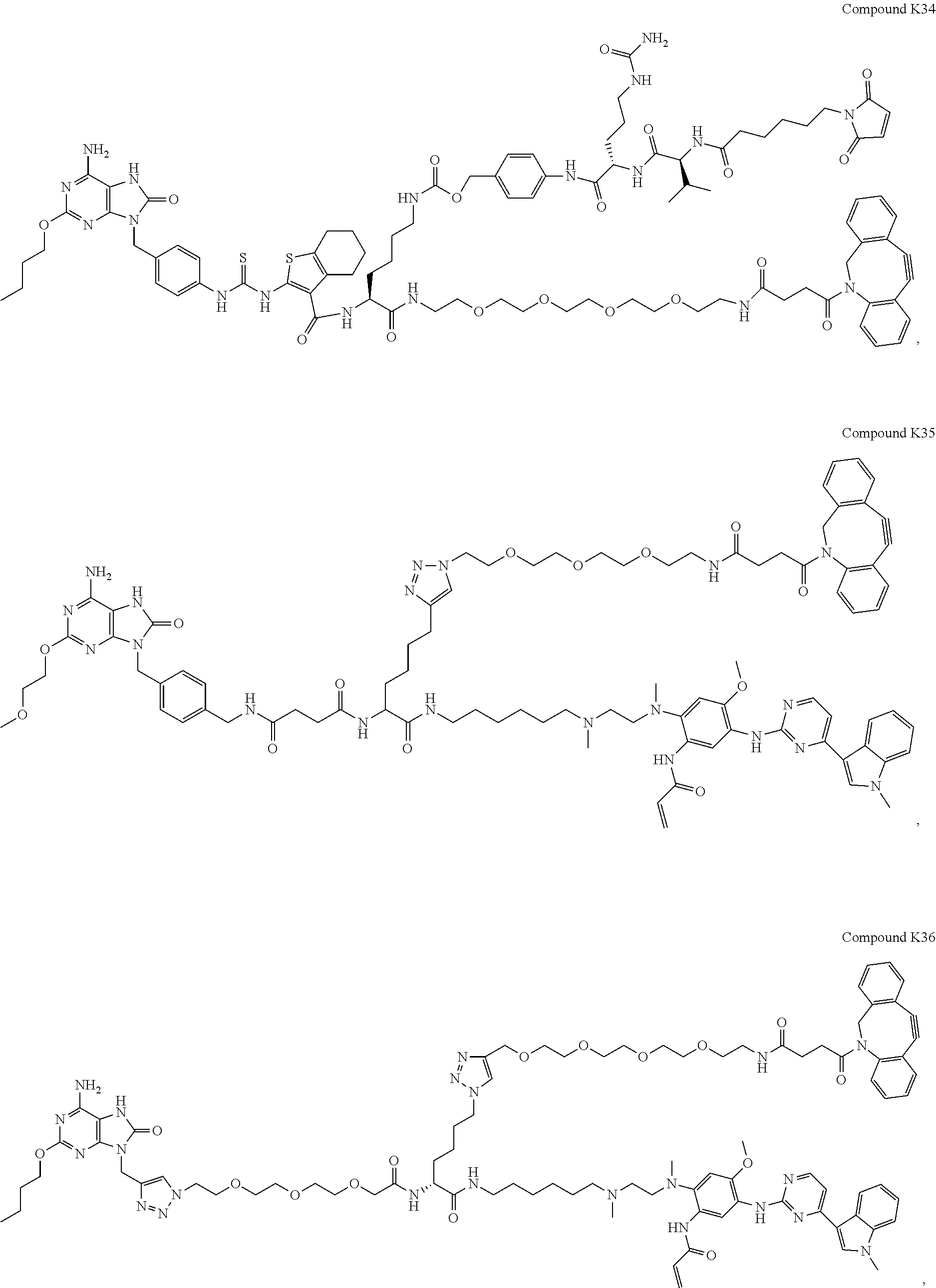

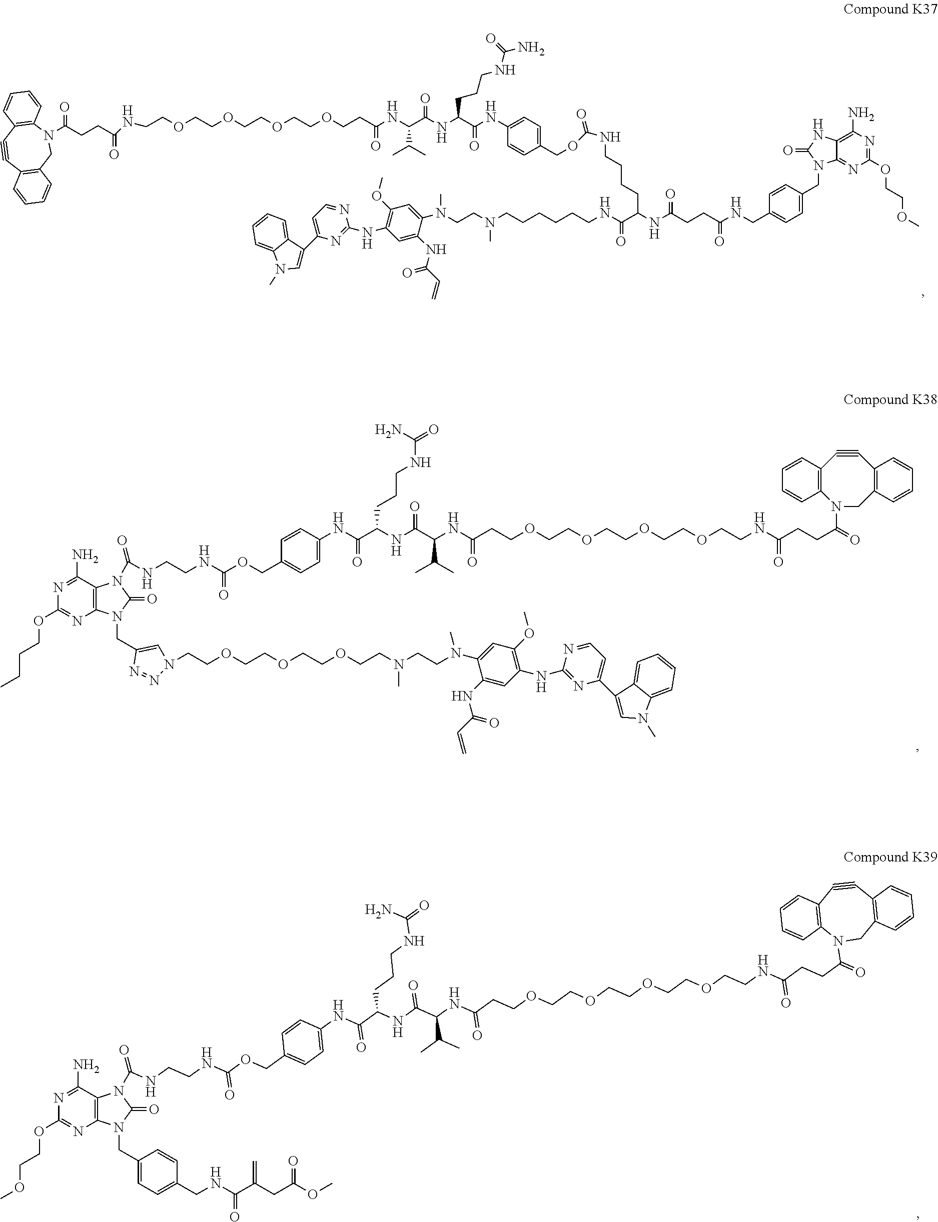

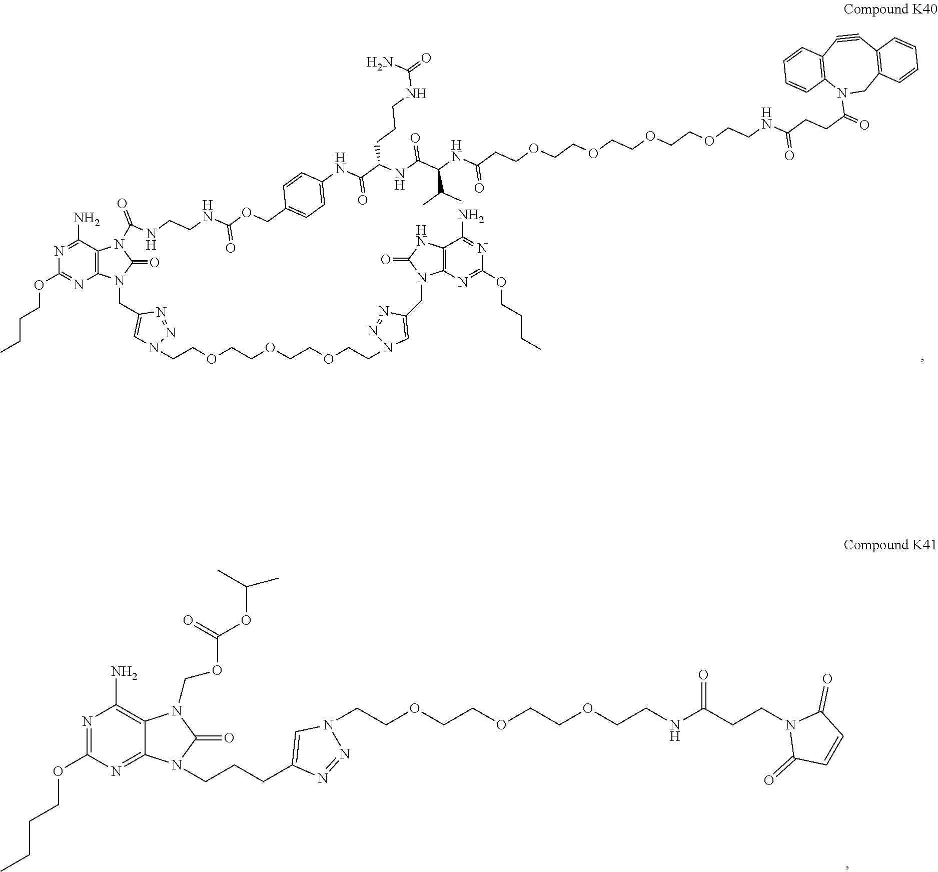 [SMILES: CCCCOc1nc(N)c2[nH]c(=O)n(Cc3cn(CCOCCOCCOCCn4cc(Cn5c(=O)n(C(=O)NCCNC(=O)OCc6ccc(NC(=O)[C@H](CCCNC(N)=O)NC(=O)[C@@H](NC(=O)CCOCCOCCOCCOCCNC(=O)CCC(=O)N7Cc8ccccc8C#Cc8ccccc87)C(C)C)cc6)c6c(N)nc(OCCCC)nc65)nn4)nn3)c2n1 ; CCCCOc1nc(N)c2c(n1)n(CCCc1cn(CCOCCOCCOCCNC(=O)CCN3C(=O)C=CC3=O)nn1)c(=O)n2COC(=O)OC(C)C]

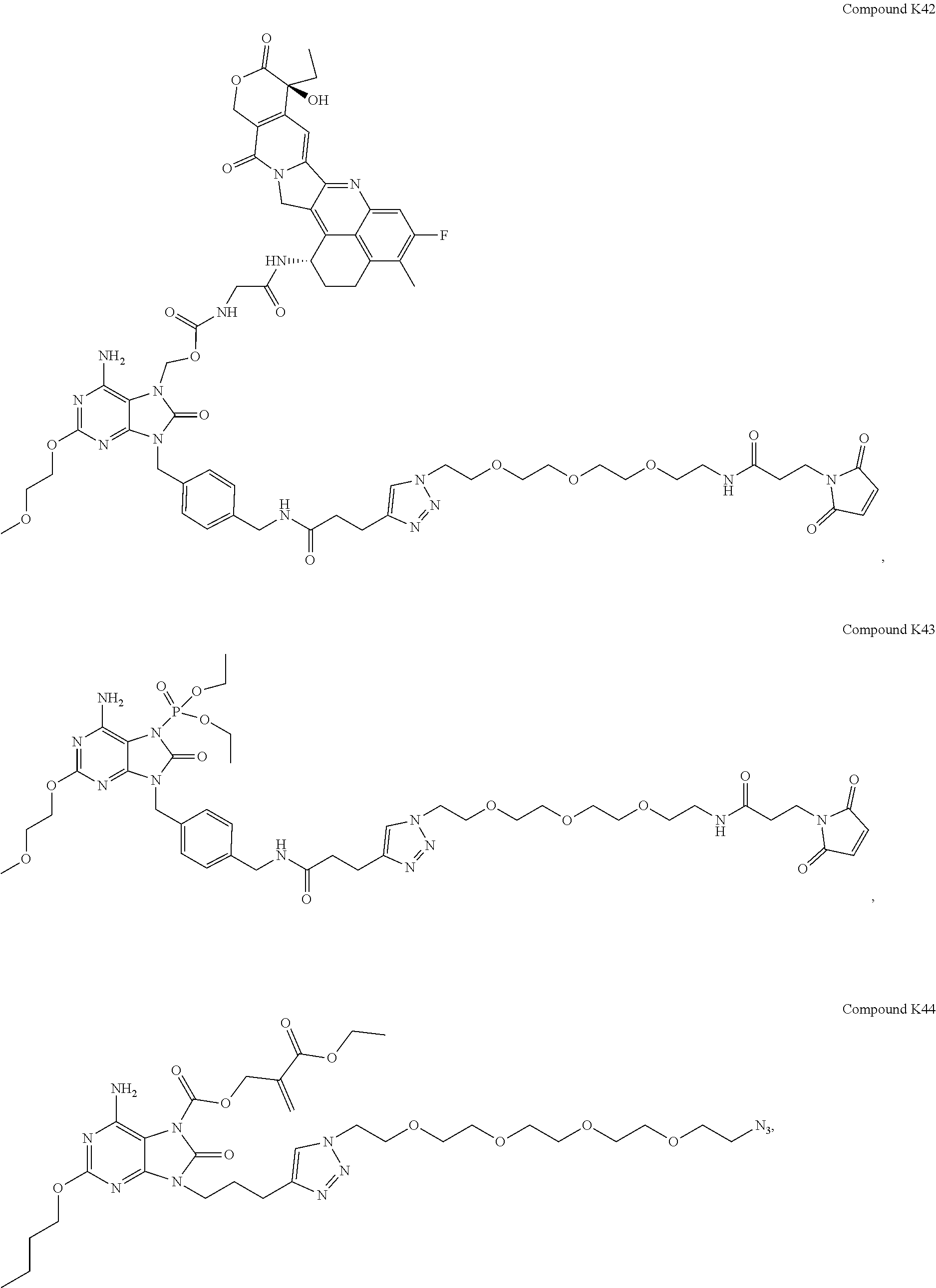 [SMILES: CC[C@@]1(O)C(=O)OCc2c1cc1n(c2=O)Cc2c-1nc1cc(F)c(C)c3c1c2[C@@H](NC(=O)CNC(=O)OCn1c(=O)n(Cc2ccc(CNC(=O)CCc4cn(CCOCCOCCOCCNC(=O)CCN5C(=O)C=CC5=O)nn4)cc2)c2nc(OCCOC)nc(N)c21)CC3 ; CCOP(=O)(OCC)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21 ; C=C(COC(=O)n1c(=O)n(CCCc2cn(CCOCCOCCOCCOCCN=[N+]=[N-])nn2)c2nc(OCCCC)nc(N)c21)C(=O)OCC]

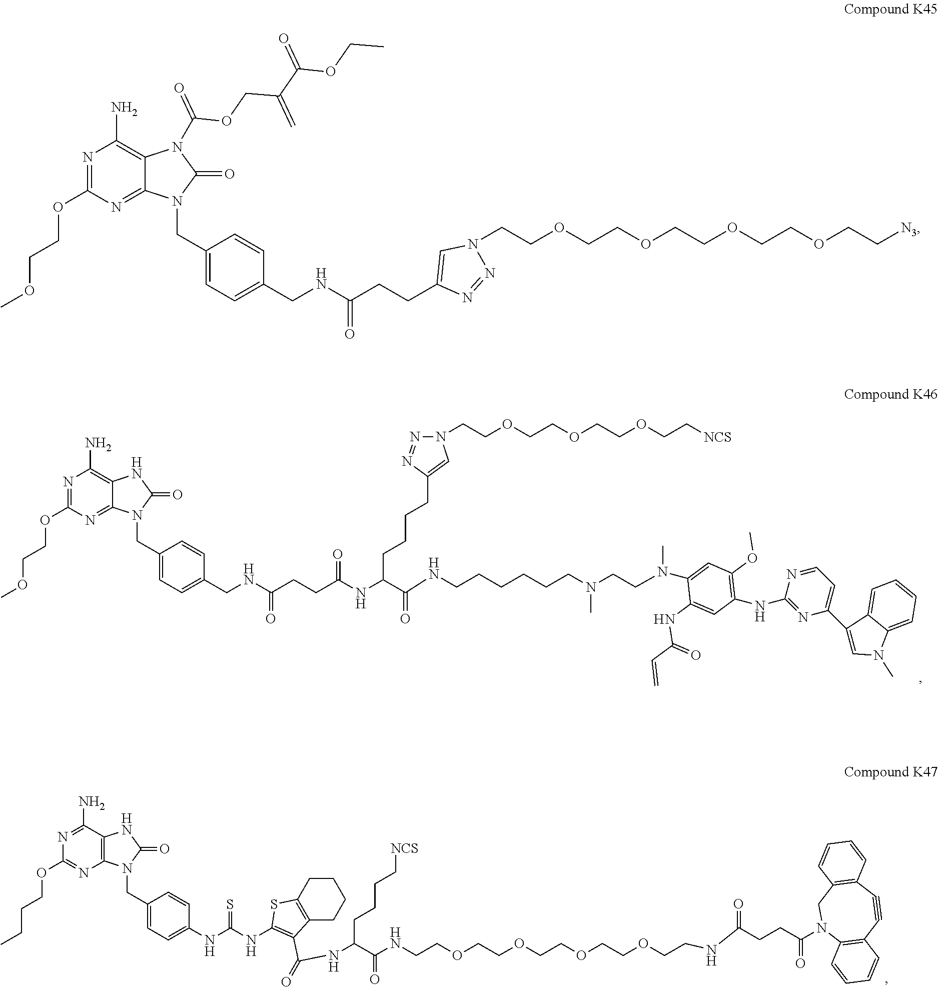

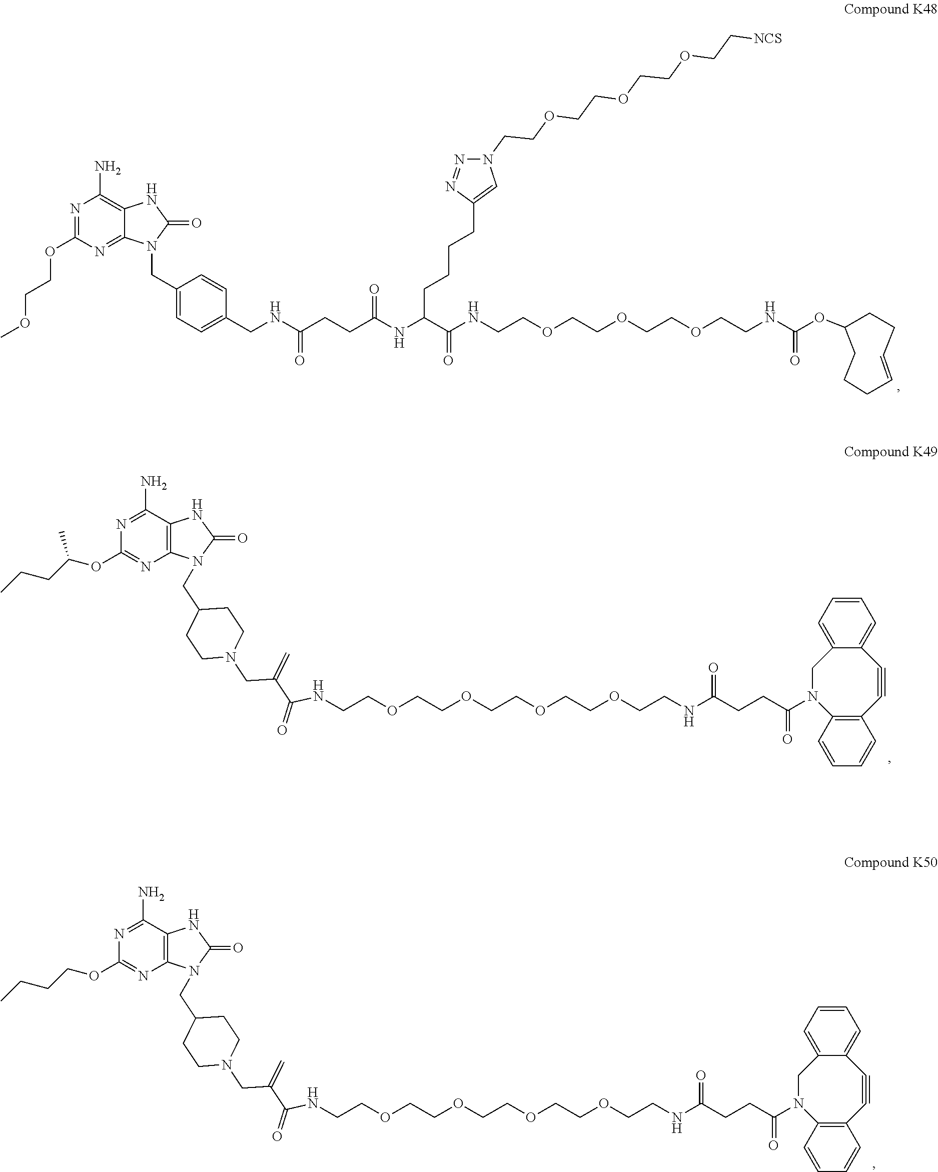

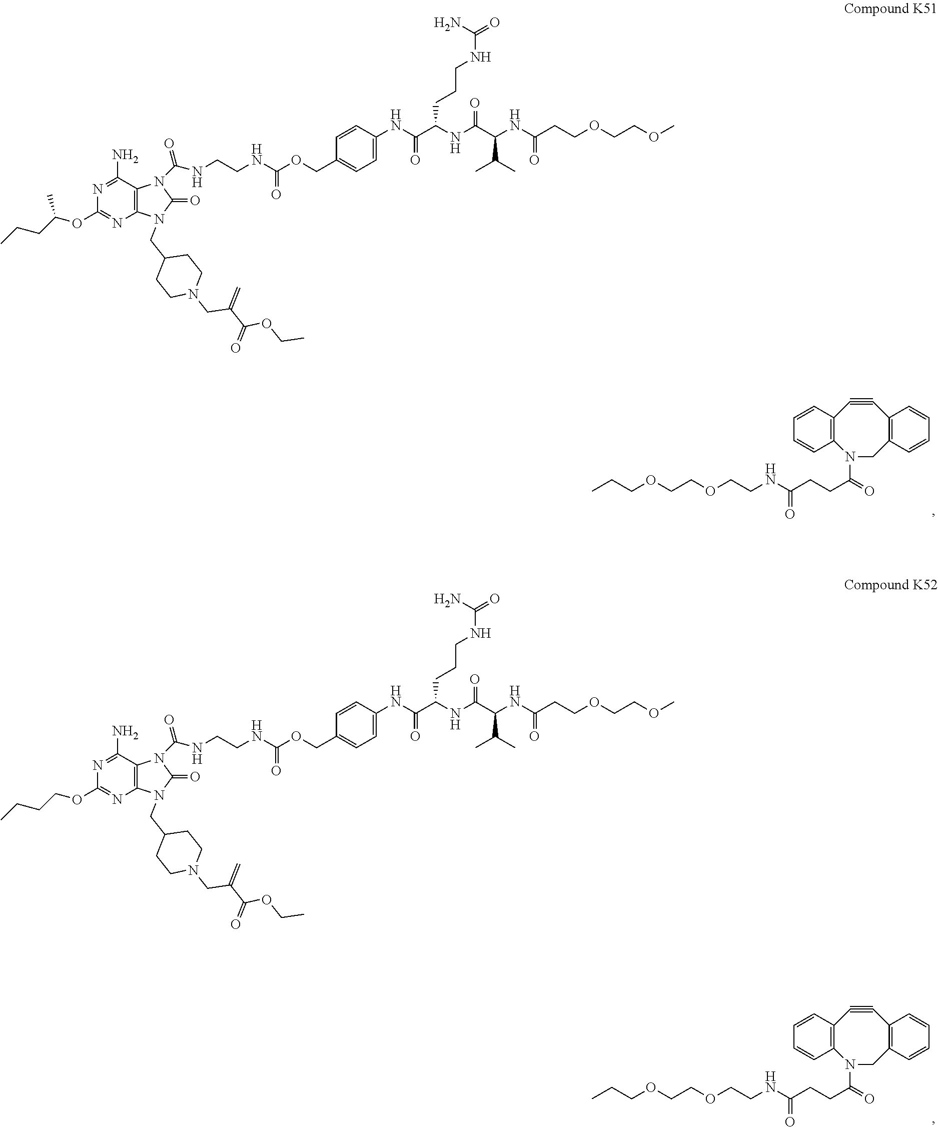

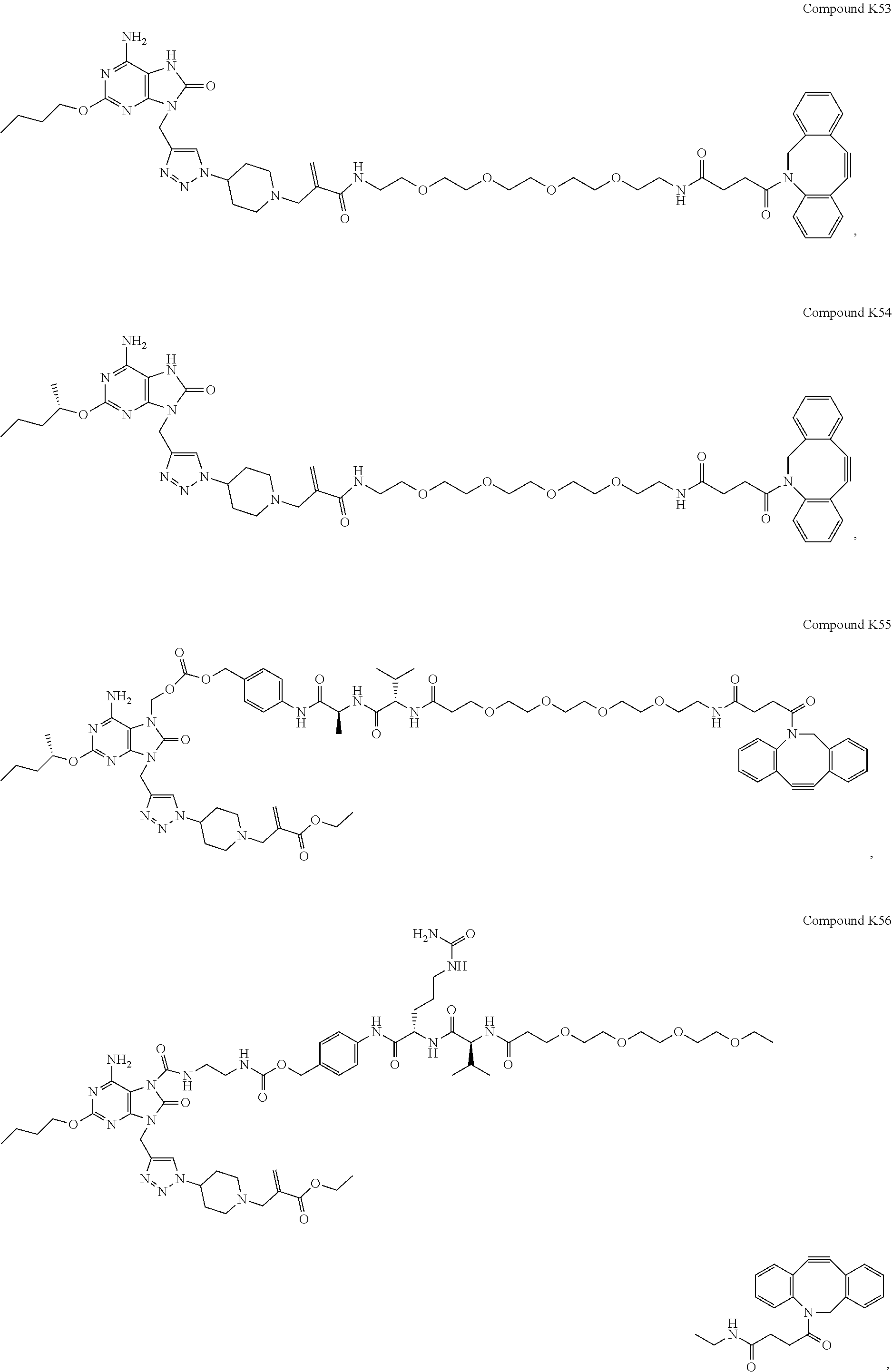

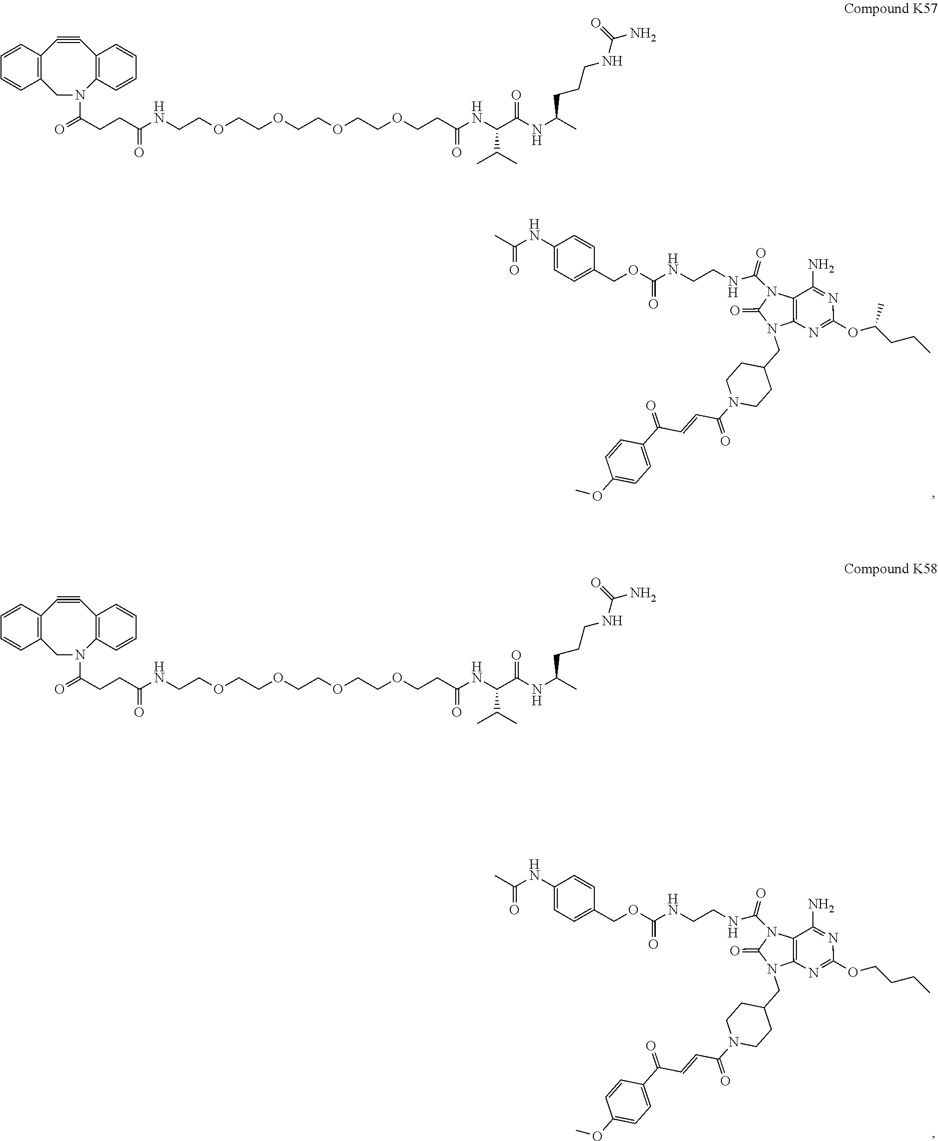 [SMILES: CC(C)[C@H](NC(=O)CCOCCOCCOCCOCCNC(=O)CCC(=O)N1Cc2ccccc2C#Cc2ccccc21)C(=O)N[C@H](C)CCCNC(N)=O ; CCC[C@@H](C)Oc1nc(N)c2c(n1)n(CC1CCN(C(=O)/C=C/C(=O)c3ccc(OC)cc3)CC1)c(=O)n2C(=O)NCCNC(=O)OCc1ccc(NC(C)=O)cc1 ; CC(C)[C@H](NC(=O)CCOCCOCCOCCOCCNC(=O)CCC(=O)N1Cc2ccccc2C#Cc2ccccc21)C(=O)N[C@H](C)CCCNC(N)=O ; CCCCOc1nc(N)c2c(n1)n(CC1CCN(C(=O)/C=C/C(=O)c3ccc(OC)cc3)CC1)c(=O)n2C(=O)NCCNC(=O)OCc1ccc(NC(C)=O)cc1]

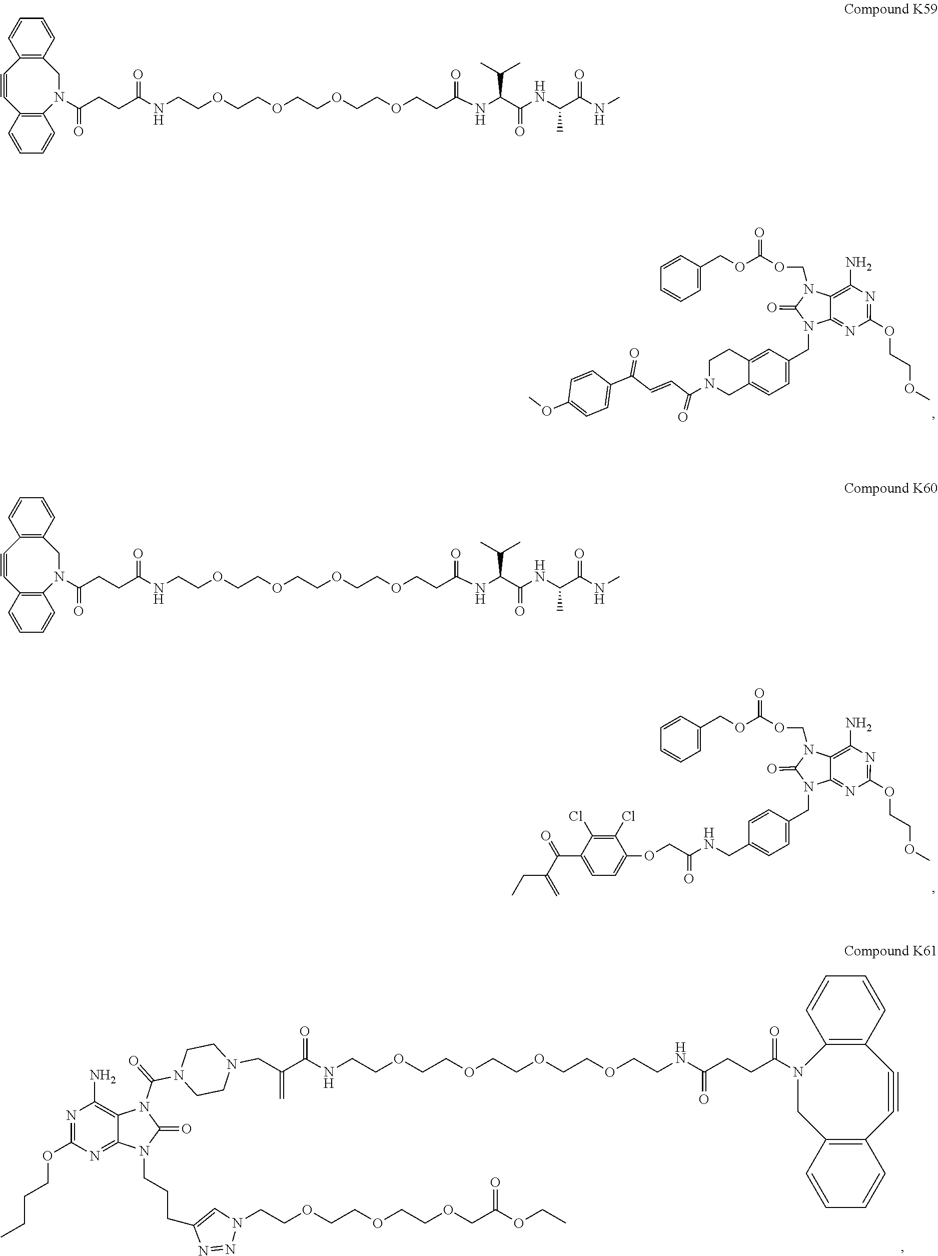

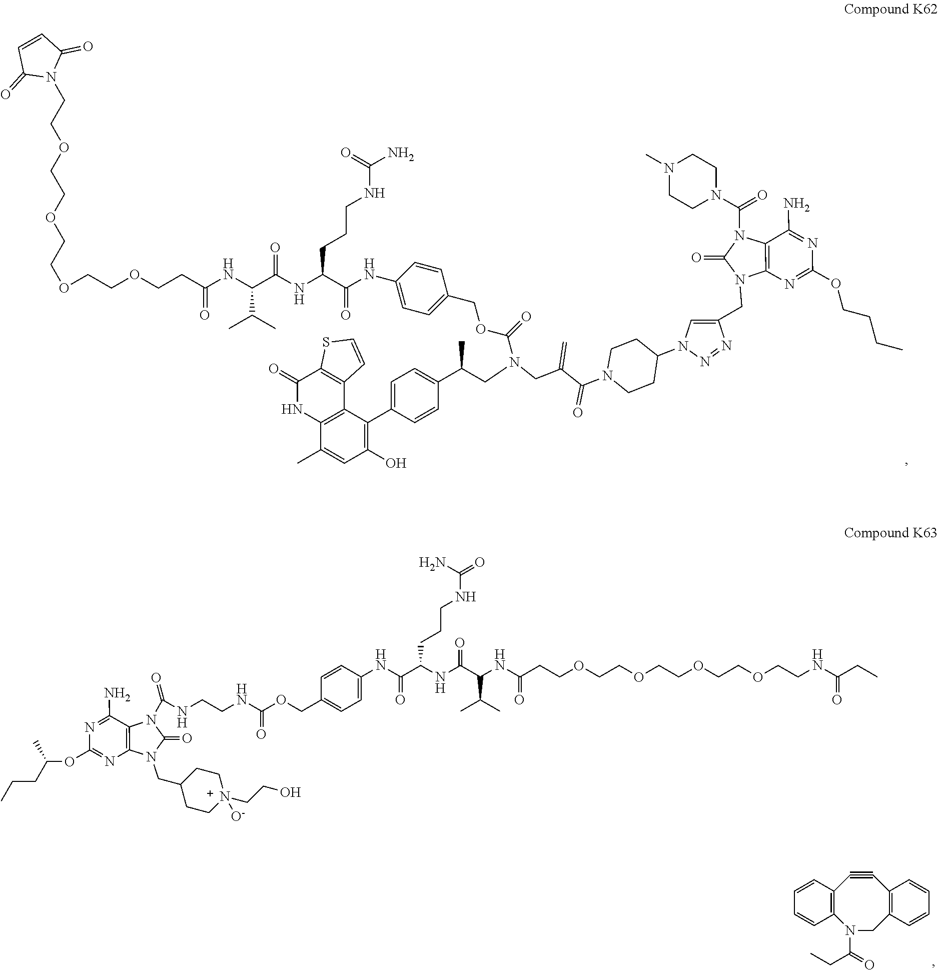

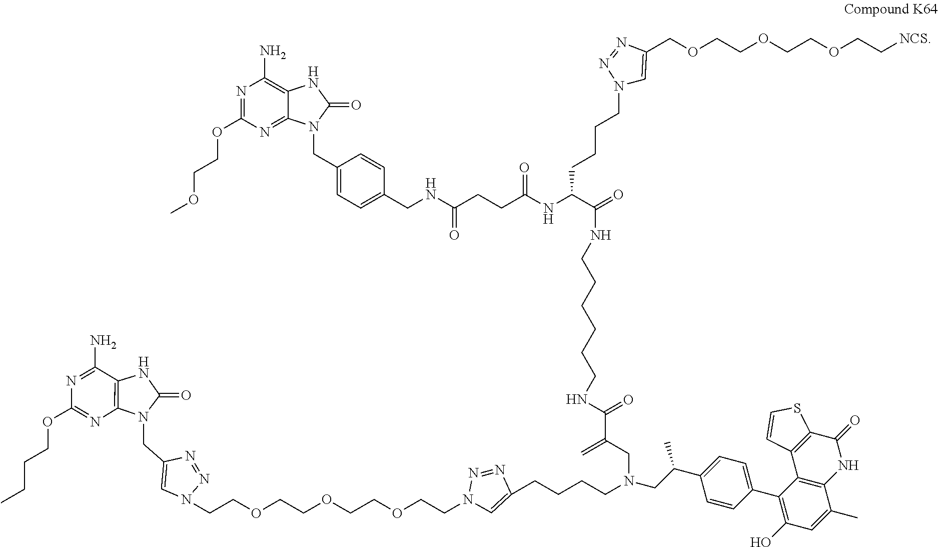

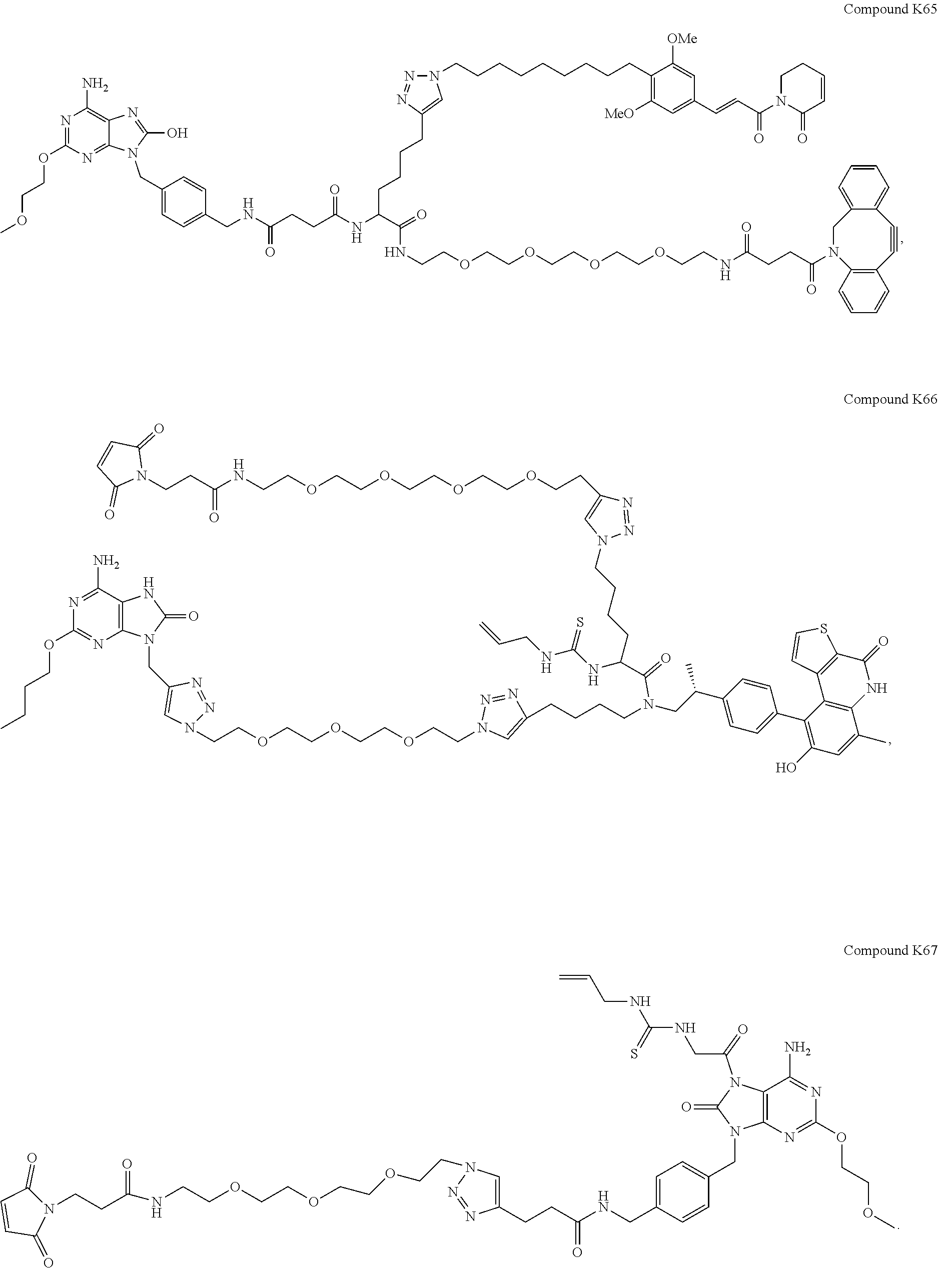 [SMILES: COCCOc1nc(N)c2nc(O)n(Cc3ccc(CNC(=O)CCC(=O)NC(CCCCc4cn(CCCCCCCCCc5c(OC)cc(/C=C/C(=O)N6CCC=CC6=O)cc5OC)nn4)C(=O)NCCOCCOCCOCCOCCNC(=O)CCC(=O)N4Cc5ccccc5C#Cc5ccccc54)cc3)c2n1 ; C=CCNC(=S)NC(CCCCn1cc(CCOCCOCCOCCOCCNC(=O)CCN2C(=O)C=CC2=O)nn1)C(=O)N(CCCCc1cn(CCOCCOCCOCCn2cc(Cn3c(=O)[nH]c4c(N)nc(OCCCC)nc43)nn2)nn1)C[C@H](C)c1ccc(-c2c(O)cc(C)c3[nH]c(=O)c4sccc4c23)cc1 ; C=CCNC(=S)NCC(=O)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21]

In a preferred embodiment, the PD-L1 antibody is Atezolizumab, and the linking functional group is maleimide group, forming the general formula I:

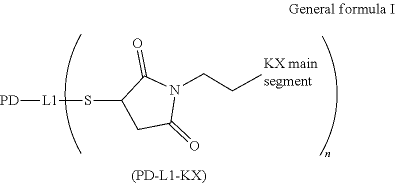

KX is an immune-activating small molecule compound, n is the DAR value, and the DAR value is 3.5-4.5.

In another preferred embodiment, the conjugation group is an azide group, forming the general formula II:

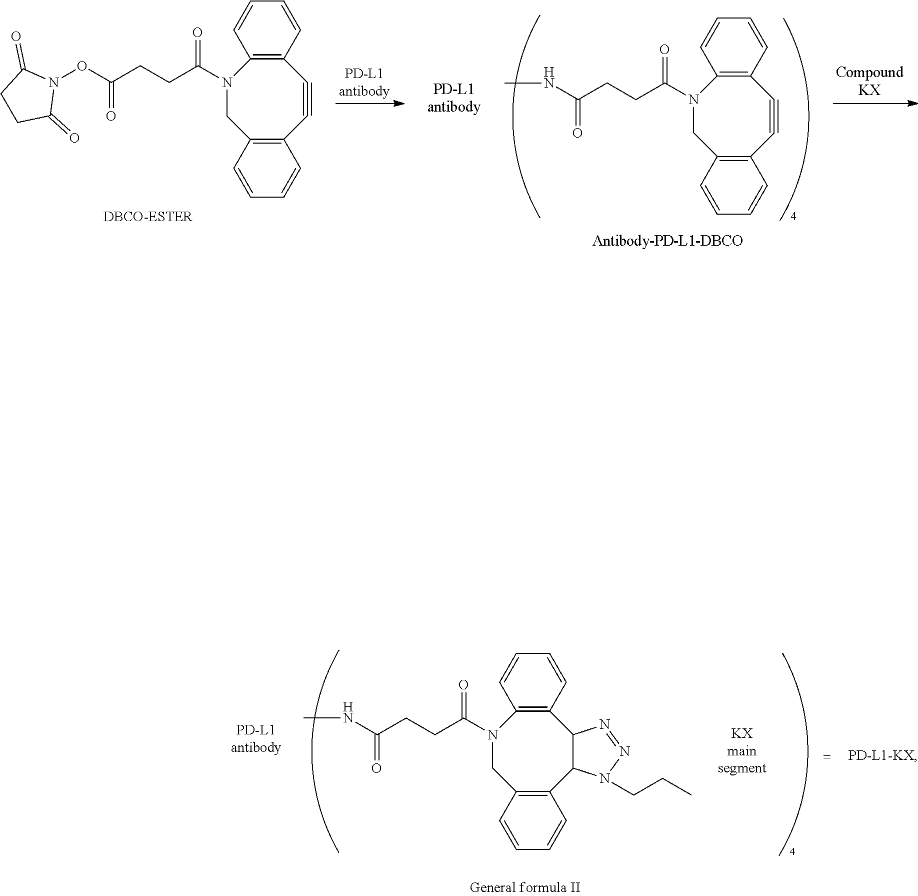

the general formula II represents PD-L1-K11, PD-L1-K21, PD-L1-K23, PD-L1-K44, and PD-L11-K45.

In another preferred embodiment, the conjugation group is DBCO group, forming the general formula III:

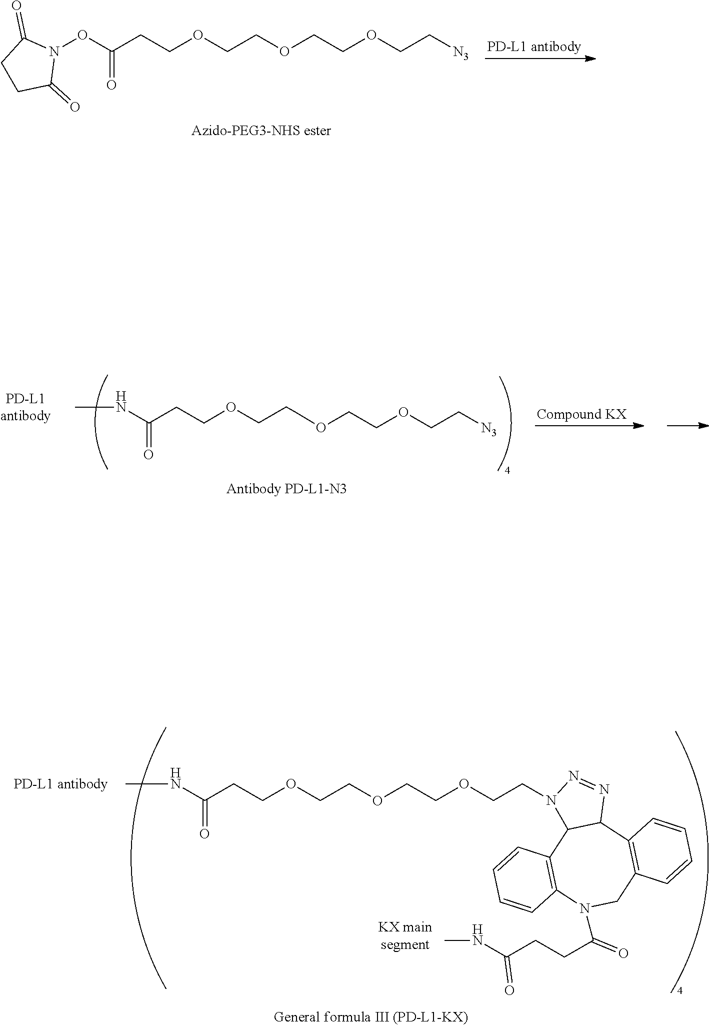

wherein the general formula III represents the following conjugated antibodies: PD-L1-K28, PD-L1-K28-1, PD-L1-K31, PD-L1-K35, PD-L1-K36, PD-L1-K37, PD-L1-K38, PD-L1-K39, PD-L1-K49, PD-L1-K50, PD-L1-K51, PD-L1-K52, PD-L1-K53, PD-L1-K54, PD-L1-K55, PD-L1-K56, PD-L1-K57, PD-L1-K58, PD-L1-K59, PD-L1-K60, PD-L1-K61, PD-L1-K63, PD-L1-K65.

In another preferred embodiment, the conjugation group is carboxyl, forming the general formula IV:

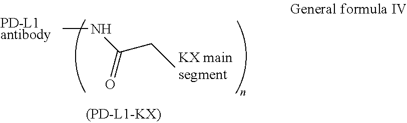

wherein the general formula IV represents the following conjugated antibodies: PD-L1-K4, PD-L1-K5, PD-L1-K9, PD-L1-K12.

In another preferred embodiment, the conjugation group is isothiocyano, forming the general formula V:

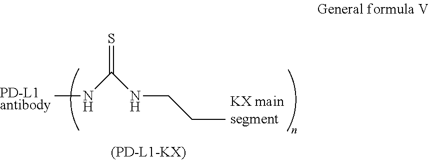

wherein the general formula V represents the following conjugated antibodies: PD-L1-K40, PD-L1-K46, PD-L1-K47, PD-L1-K48, PD-L1-K64.

In another embodiment, the present invention also provides use of the antibody-conjugated immune-activating small molecule compound as described above in the manufacture of an immunomodulatory drug.

In another embodiment, the present invention also provides use of the antibody-conjugated immune-activating small molecule compound as described above in the manufacture of a conjugated antibody.

In another embodiment, the present invention also provides use of the antibody-conjugated immune-activating small molecule compound as described above in the manufacture of an antitumor drug.

The present invention can superimpose the effect of immune initiation and activation on the basis of maintaining the antitumor function of the original antibody, thereby greatly improving the antitumor effect.

### DETAILED DESCRIPTION OF THE INVENTION

To make the objectives, technical solutions, and advantages of the present invention clearer, the present invention will be further described in detail below in view of the figures and Examples. It should be understood that the specific Examples described herein are only used to illustrate the present invention and are not intended to limit the present invention.

The present invention provides an antibody-conjugated immune-activating small molecule compound KX, and the general formula formed by the conjugation of the antibody and the immune-activating small molecule compound is as shown in the following formula:

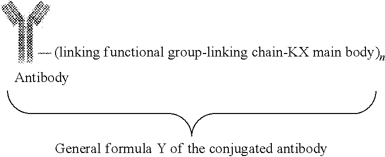

wherein the linking functional group is maleimide group, carboxyl, isothiocyano, azido or alkynyl, the linking chain is alkoxy or alkyl chain, the antibody is IgG1, IgG2, IgG4, IgA, IgM, IgY or nanobody, and the KX-main body is the main part of the immune-activating small molecule KX.

The following Examples are directed to the synthesis methods and application effects of the bifunctional small molecule compounds.

### Example 1

HPLC conditions (the purity of the compounds purified by HPLC or silica gel column chromatography reaches 98.5% or higher)

Mobile phase: C18 silica, acetonitrile/water with 0.05% trifluoroacetic acid gradient: isocratic elution with 45% acetonitrile/55% water and 0.05% trifluoroacetic acid at a flow rate of 5 mL/min.

UV: 254 nm; instrument model: Agilent 1260 infinity II; column model: YMC-Pack ODS-A, 250×20 mm, S-5 μm, 12 nm;

### Mass Spectrometric Conditions Mobile Phase

| Time | Acetonitrile | Water (1‰ | Flow Rate |
|---|---|---|---|
| (min) | concentration (%) | formic acid) (%) | (mL/min) |
| 0 | 5 | 95 | 0.5 |
| 5 | 90 | 10 | 0.5 |
| 7 | 90 | 10 | 0.5 |
| 10 | 5 | 5 | 0.5 |
| 12 | 5 | 5 | 0.5 |

UV: 254 un; mass spectrometer for substance identification: Angilent; liquid phase: Angilent 1260 Infinity 11; mass spectrometer: Angilent G6125B; column model: YMC-Pack ODS-A, 150×4.6 mm, S-5 μm, 12 nm; antibody and polypeptide detection instrument model: XevoG2XSQTOF Mass Spectrometer (manufacturer: Waters Corporation).

### Synthesis of Compound K1

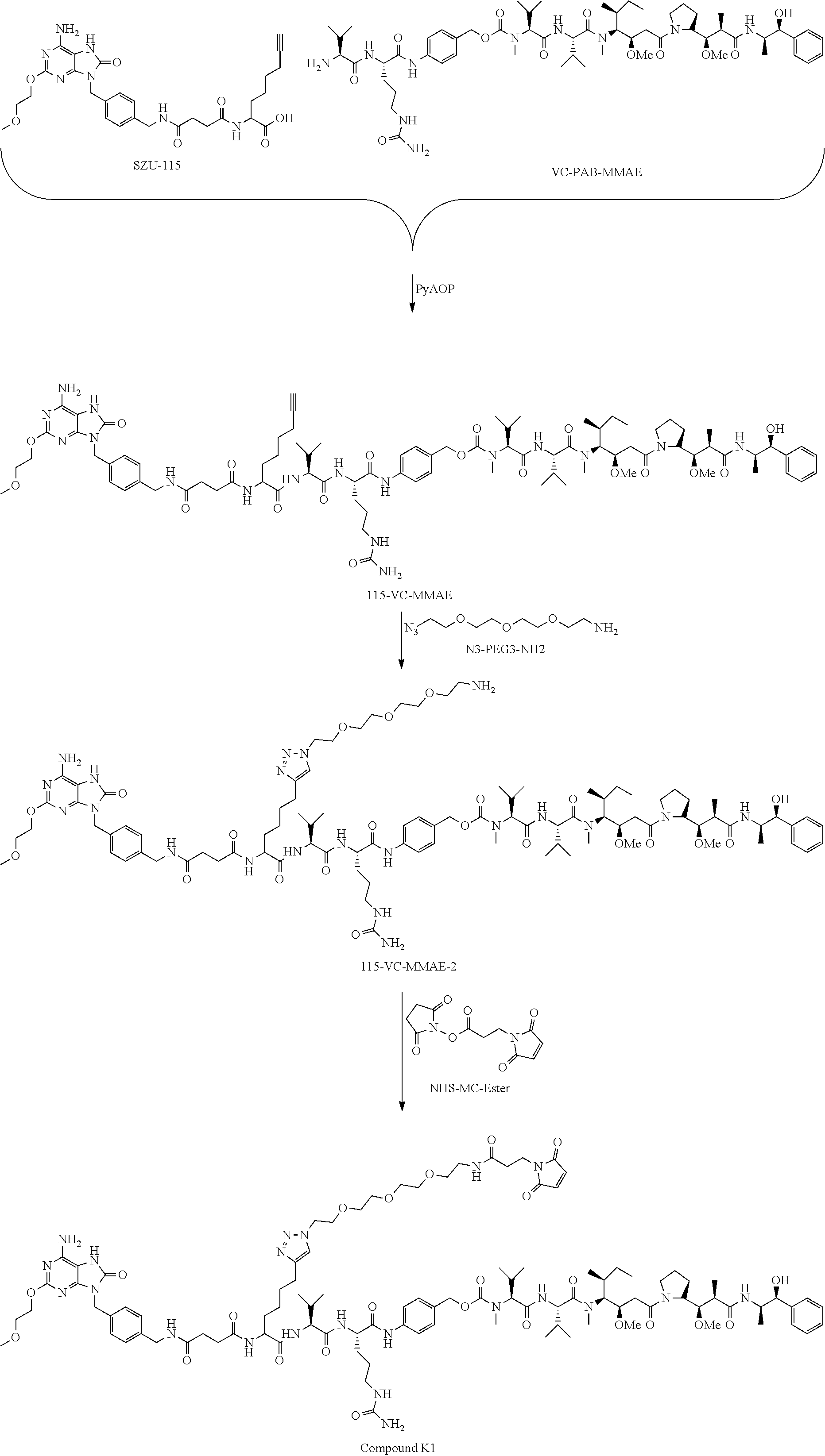

First, SZU-115 (52 mg, 0.089 mmol) and VC-PAB-MMAE (100 mg, 0.089 mmol) were dissolved In 2 mL of DM F, DIPE A (46 μL) was added and stirred uniformly, then Py AOP (48 mg) was added. The resultant mixture was stirred and reacted at room temperature for 3 hours, the product was separated and purified by preparative HPLC, and the purified solution was lyophilized to give compound 115S-VC-MMAE (119 mg, yield 79%), ESI-MS: m/z=844.4[(M/2)+H]+.

Then, 115-VC-MMAE (100 mg, 0.059 mmol) and N3-PEG3-NH2 (13 mg, 0.060 mmol) were dissolved in 2 mL of DMF-H2O mixture solvent (volume ratio: 3:1) mixed solvent, and then copper sulfate pentahydrate (37 mg) and sodium L-ascorbate (20 mg) were added. The resultant mixture was stirred and reacted at room temperature. The reaction was monitored by LC-MS until completion, and the product was separated and purified by preparative HPLC to give compound 115-VC-MMAE-2 (70 mg, yield 62%). ESI-MS: m/z=953.6[(M/2)+H]+.

115-MC-MMAE-2 (60 mg, 0.03 mmol) and NHS-MC-Ester (12 mg, 0.045 mmol) were taken and dissolved in 2 mL of DMF. The resultant mixture was stirred and reacted at room temperature overnight, and the product was separated and purified by preparative HPLC, and the purified solution was lyophilized to give compound K1 (49 mg, yield 79%), ESI-MS: m/z=686.4[(M/3)+H]+.

### Synthesis of Compound K2

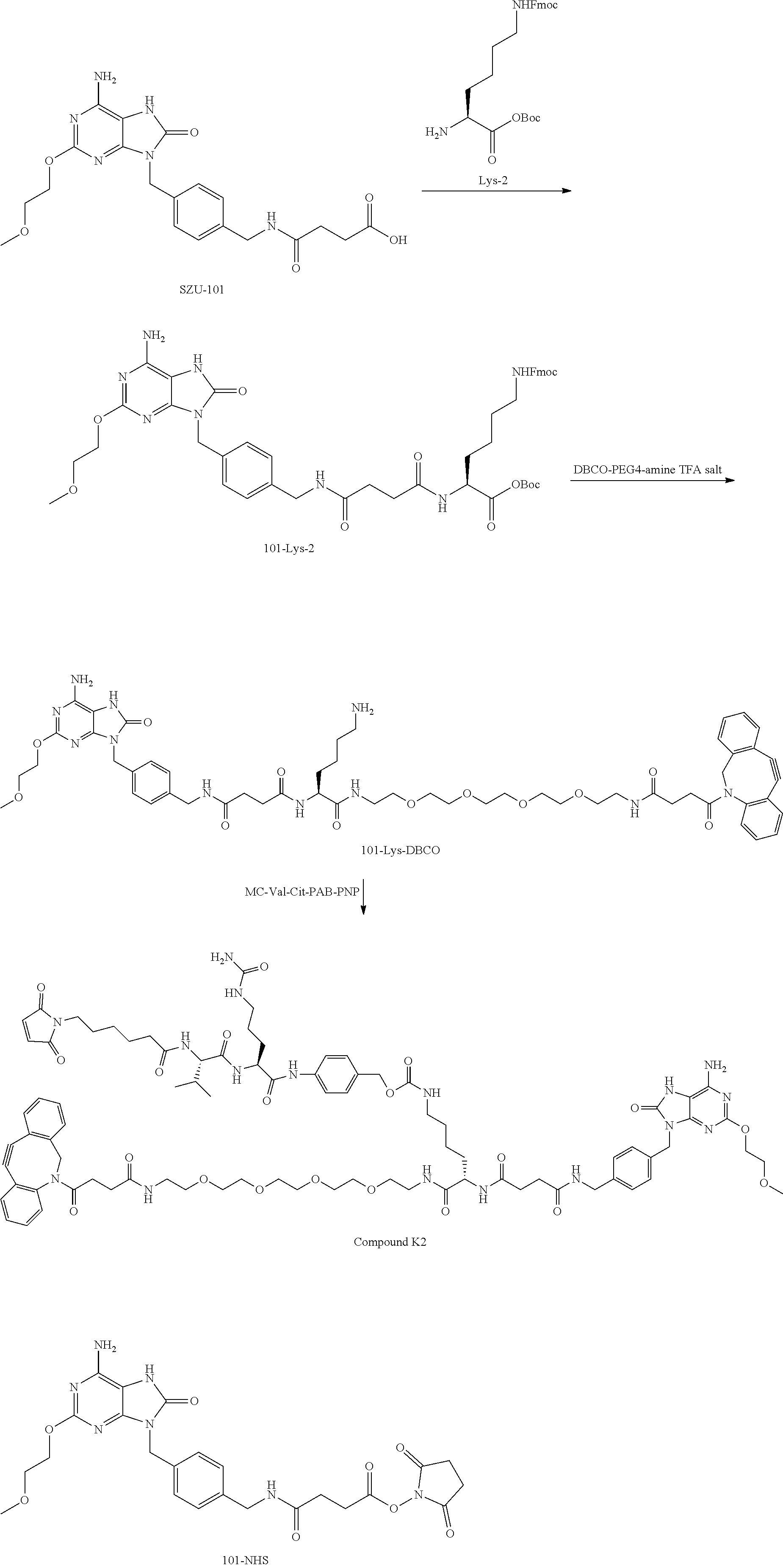

First, SZU-101 (100 mg, 0.225 mmol) and NIS (26 mg, 0.225 mmol) were dissolved in 2 mL of DMF, DIPEA (40 μL) and EDCI (45 mg) were added, and the resultant mixture was stirred at room temperature for 6 hours. After lyophilization, the lyophilizate was poured into cold water to precipitate a solid, and the solid was filtrated and dried under vacuum to give compound 101-NI-IS (104 mg, yield 85%). Lys-2 (71 mg, 0.15 mmol) and DMAP (10 mg) were dissolved in 2 mL of DMF, and 101-NHS (82 mg, 0.15 mmol) was added, and the mixture was stirred and reacted at room temperature for 6 hours; the solvent was removed by lyophilization, and the residue was added with TFA/DCM (mixed volume: 1:3, 4 mL) under cooling, and was stirred and reacted at room temperature for 1 hour. The solvent was removed by vacuum distillation, and the residue was separated and purified by preparative HPLC to give compound 101-Lys-2 (85 mg, yield 71%), ESI-MS: m/z=795.3 [M+H]+. Compound 101-Lys-2 (80 mg, 0.1 mmol) and triethylamine (28 μL) were taken and dissolved in 1.5 mL of dry DMF, PyAOP (52 mg, 0.1 mmol) was added and stirred at room temperature for 30 minutes, and then DBCO-PEG4-amine TFA salt (52 mg, 0.1 mmol) was added. The resultant mixture was stirred and reacted at room temperature for 3 hours, and then piperidine (30 μL) was added, and the mixture was stirred and reacted for another 3 hours. The reaction liquid was lyophilized and the lyophilizate was purified by preparative HPLC to give compound 101-Lys-DBCO (49 mg, yield 45%), ESI-MS: m/z=1078.5[M+H]+.

MC-Val-Cit-PAB-PNP (30 mg) was dissolved in 0.5 mL of dry DMF; 101-Lys-DBCO (44 mg, 0.04 mmol) and DIPEA (10 μL) were taken and mixed in 0.5 mL of DMF, and the solution was slowly added to the solution of MC-Val-Cit-PAB-PNP under cooling in an external bath at 0° C.; after the addition, the mixture was shaken and reacted at room temperature for 3 hours. The solvent was removed by lyophilization, and the residue was dissolved in a small amount of methanol and purified by preparative HPLC to give compound K2 (53 mg, yield 79%), ESI-MS: m/z=839.0[(M/2)+H]+.

### Synthesis of Compound K3

According to the synthesis method of compound K1 except replacing VC-PAB-MMAE with VC-PAB-514, compound K3 can be obtained, ESI-MS: m/z=852.4[(M/2)+H]+.

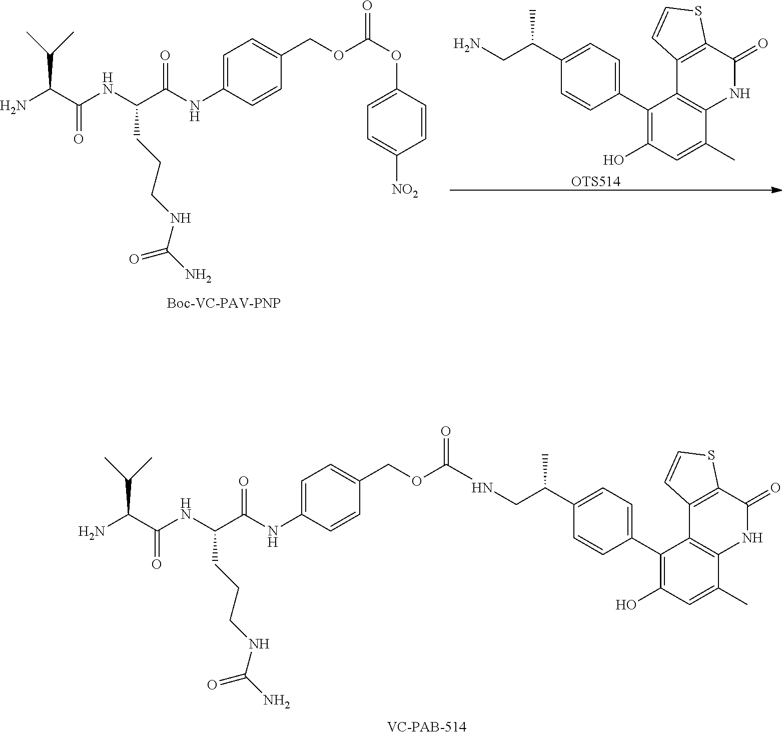 [SMILES: CC(C)[C@H](N)C(=O)N[C@@H](CCCNC(N)=O)C(=O)Nc1ccc(COC(=O)Oc2ccc([N+](=O)[O-])cc2)cc1 ; Cc1cc(O)c(-c2ccc([C@@H](C)CN)cc2)c2c1[nH]c(=O)c1sccc12 ; "OTS514" ; Cc1cc(O)c(-c2ccc([C@@H](C)CNC(=O)OCc3ccc(NC(=O)[C@H](CCCNC(N)=O)NC(=O)[C@@H](N)C(C)C)cc3)cc2)c2c1[nH]c(=O)c1sccc12]

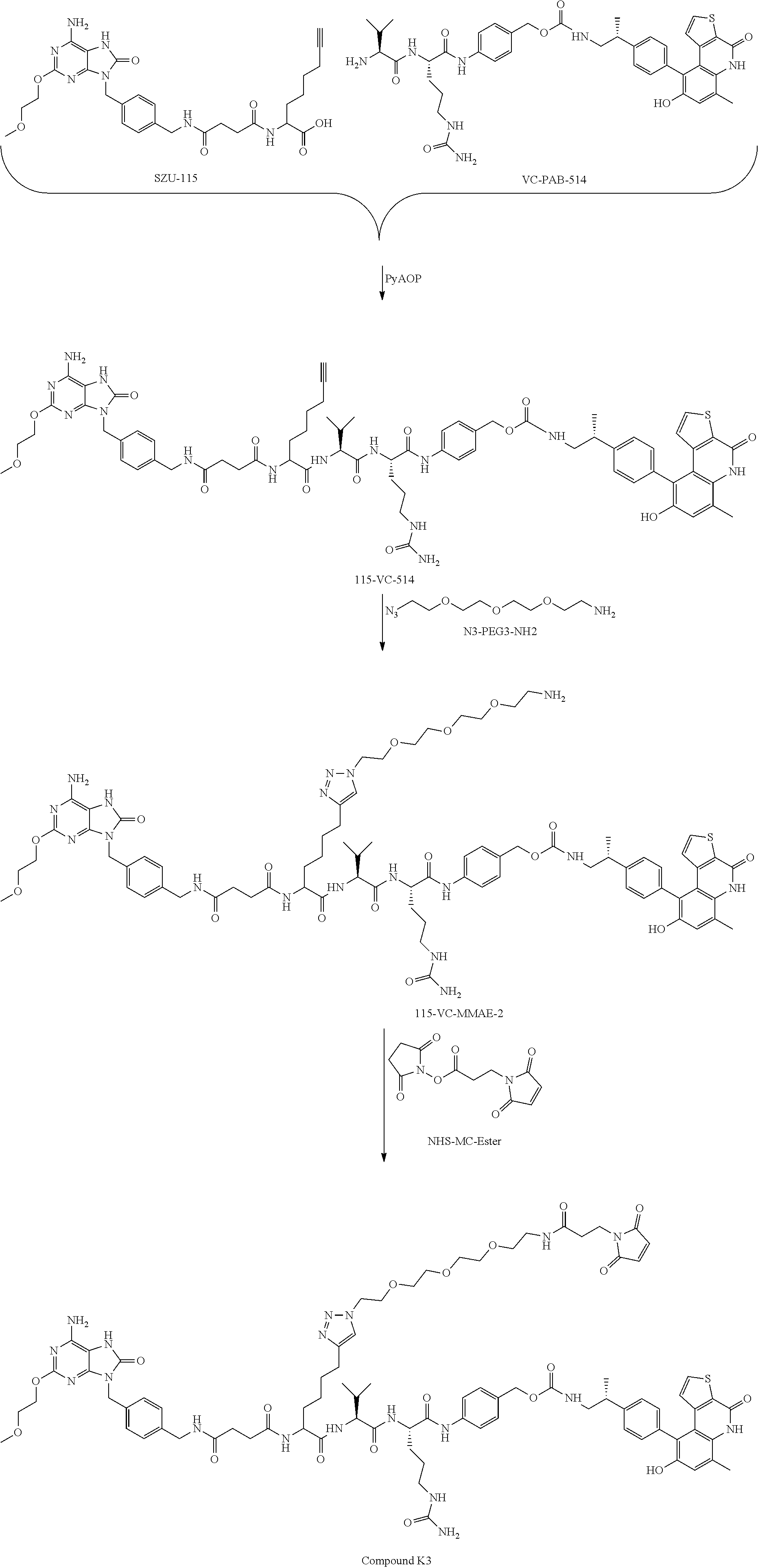

The synthesis procedures of VC-PAB-514 are as follows: Boc-VC-PAB-PNP (65 mg) and OTS514 (37 mg) were taken and dissolved in 1 mL of DMF, and DIPEA (52 μL) was added, and the mixture was shaken and reacted at room temperature for 1 hour. 1 mL of DCM-TFA mixed solvent (volume ratio 3:1) was added to the reaction mixture. After stirring at room temperature for 30 minutes, DCM-TFA was removed by vacuum distillation at 30° C. under reduced pressure, the residue was lyophilized, and the lyophilizate was dissolved in a small amount of 95% methanol, separated and purified by preparative HPLC to give VC-PAB-514 (51 mg), ESI-MS: m/z=771.1 [M+H]+.

### Synthesis of Compound K4

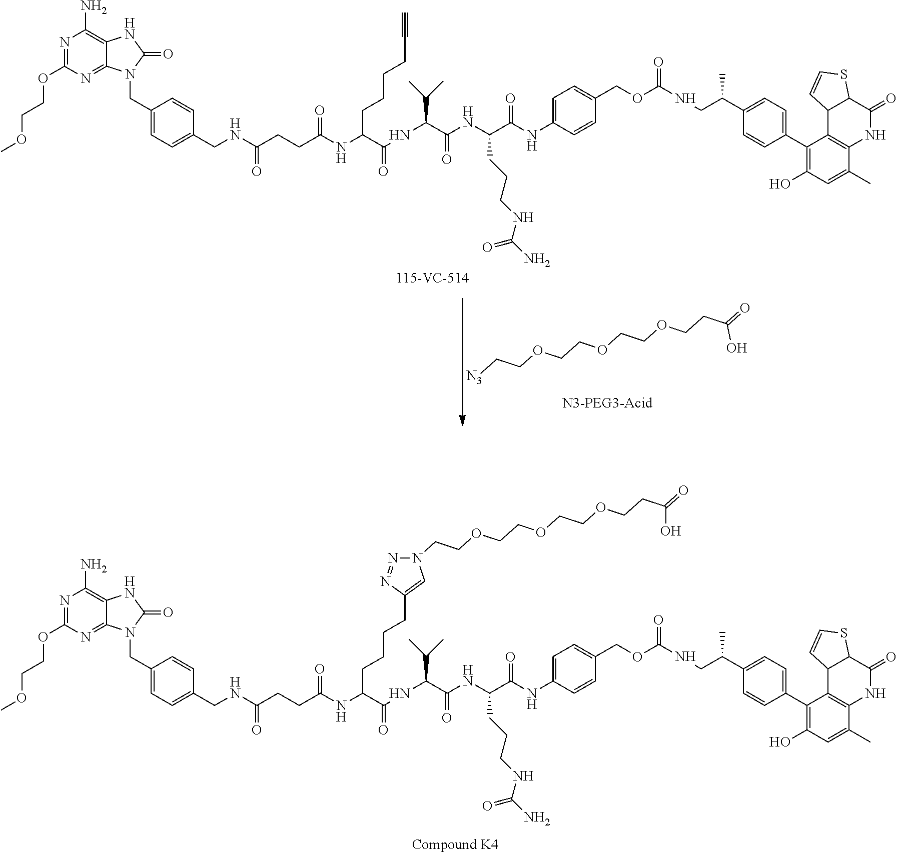 [SMILES: C#CCCCCC(NC(=O)CCC(=O)NCc1ccc(Cn2c(=O)[nH]c3c(N)nc(OCCOC)nc32)cc1)C(=O)N[C@H](C(=O)N[C@@H](CCCNC(N)=O)C(=O)Nc1ccc(COC(=O)NC[C@H](C)c2ccc(-c3c(O)cc(C)c4c3C3C=CSC3C(=O)N4)cc2)cc1)C(C)C ; [N-]=[N+]=NCCOCCOCCOCCC(=O)O ; COCCOc1nc(N)c2[nH]c(=O)n(Cc3ccc(CNC(=O)CCC(=O)NC(CCCCc4cn(CCOCCOCCOCCC(=O)O)nn4)C(=O)N[C@H](C(=O)N[C@@H](CCCNC(N)=O)C(=O)Nc4ccc(COC(=O)NC[C@H](C)c5ccc(-c6c(O)cc(C)c7c6C6C=CSC6C(=O)N7)cc5)cc4)C(C)C)cc3)c2n1]

First, 115-VC-514 (80 mg, 0.06 mmol) and N3-PEG3-Acid (15 mg, 0.06 mmol) were dissolved in 2 mL of DMF-H2O mixed solvent (volume ratio 3:1), and copper sulfate pentahydrate (37 mg) and sodium L-ascorbate (20 mg) were added. The resultant mixture was stirred and reacted at room temperature. The reaction was monitored by LC-MS until completion, and the product was separated and purified by preparative HPLC to give compound K4 (73 mg, yield 77%), ESI-MS: m/z=791.4[(M/2)+H]+.

According to the synthesis method of compound K4 except replacing 115-VC-514 with 115-VC-MMAE, compound K5 can be obtained, ESI-MS: m/z=968.2[(M/2)+H]+.

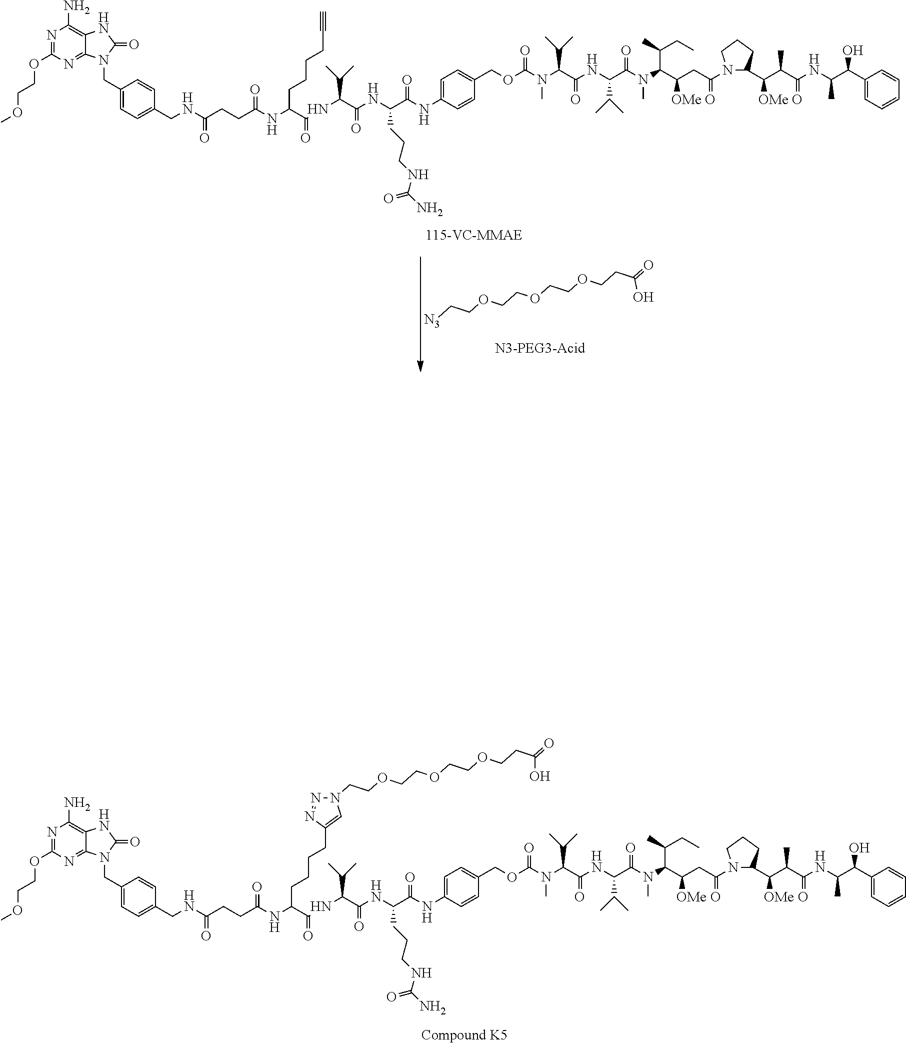

### Synthesis Route of Compound K6

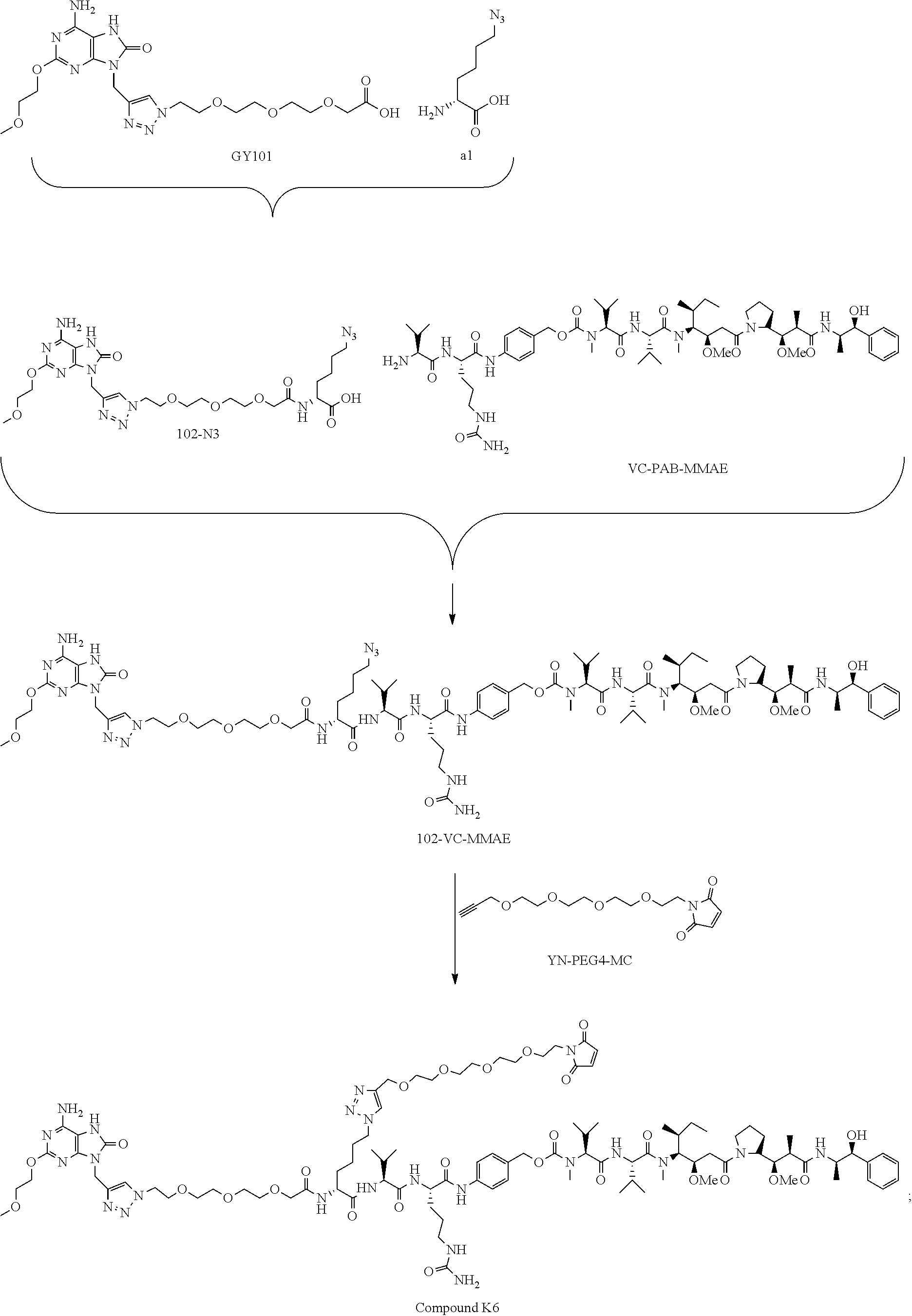

Into compound GY102 (500 mg, 1 mmol), N-hydroxysuccinimide (NHS, 120 mg) and 10 mL of DMF solution, EDC-HCl (210 mg) was added and stirred at room temperature for 3 hours. Compound a1 (175 mg, 1 mmol) and 2 ml, of solution of DIPEA (350 μL) in DMF were then slowly added. The resultant mixed solution was stirred at room temperature overnight. The reaction solution was lyophilized, and the lyophilizate was dissolved in 20 mL of saturated Na₂CO₃ Solution, stirred at room temperature for 30 minutes, and neutralized to pH 4 with acetic acid; the neutralized solution was extracted with ethyl acetate (3×20 mL); the extraction solution was dried over anhydrous sodium sulfate; the desiccant was filtered out; and the filtrate was concentrated under reduced pressure to give compound 102-N3 (337 mg, yield 52%), ESI-MS: m/z=649.3[M+H]+.

Compounds 102-N3 (130 mg, 0.2 mmol) and VC-PAP-MMAE (225 mg, 0.2 mmol) were dissolved in 5 mL of DMF, and DIPEA (40 μL) and PyAOP (115 mg) were added; the resultant mixture was stirred at room temperature for 3 hours. The reactant was lyophilized, and the lyophilizate was dissolved in a small amount of methanol, separated and purified by preparative HPLC to give compound 102-VC-MMAE (168 mg, yield 49%). ESI-MS: m/z=878.1 [(M/2)+H]+.

Compounds 102-VC-MMAE (88 mg, 0.05 mmol) and YN-PEG4-MC (16 mg, 0.05 mmol) were taken and dissolved in 4 mL of H₂O-DMF solvent (volume ratio 3:1), sodium L-ascorbate (11 mg) and copper sulfate pentahydrate (14 mg) were added; the resultant mixture was stirred and reacted at room temperature for 6 hours. The reactant was lyophilized, and the lyophilizate was dissolved in a small amount of methanol and filtered; and the filtrate was separated and purified by preparative HPLC to give compound K6 (57 mg, yield 55%), ESI-MS: nlz=689.5[(M/3)+H]+.

According to the synthesis method of compound K2 except replacing compound SZU-101 with GY101, compound K7 can be obtained, ESI-MS: m/z=863.9[(M/2)+H]+.

According to the synthesis method of compound K6 except replacing VC-P AB-MMAE with VC-PAB-514, compound K8 can be obtained, ESI-MS: m/z=856.4[(M/2)+H]+.

### Synthesis of Compound K9

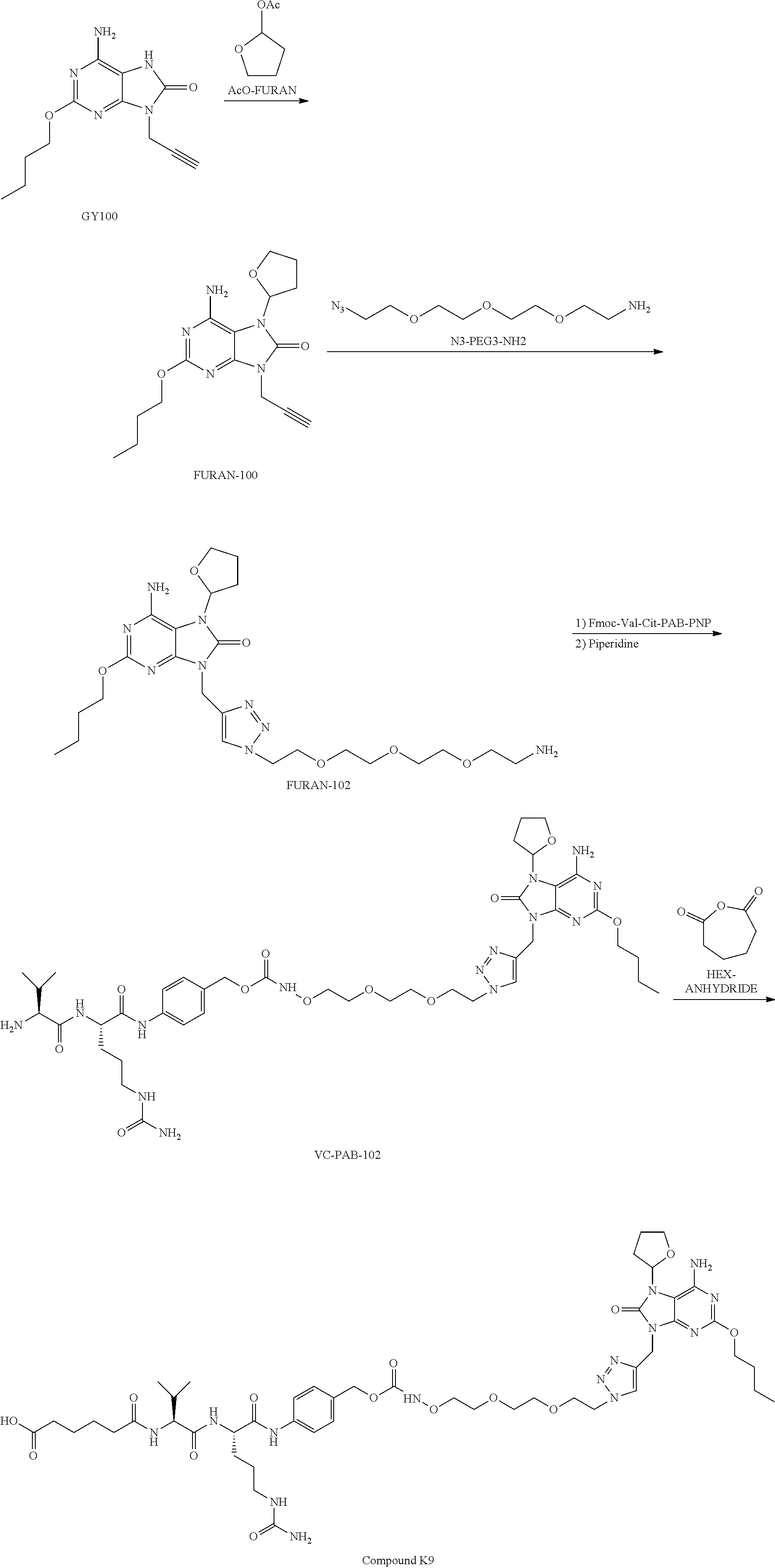

Compound GY100 (525 mg, 2 mmol) was taken and dissolved in 15 mL of DMF, and AcO-FURAN (525 mg, 4 mmol) and DBU (610 mg, 4 mmol) were added. The resultant mixture was heated to 90° C., and was stirred and reacted for 24 hours. The solvent was removed by vacuum distillation, and the residue was extracted with ethyl acetate (30 mL×3). The extracts were combined, the solvent was removed by vacuum distillation, and the residue was recrystallized with ethanol to give FURAN-100 (338 mg, yield 51%), ESI-MS: m/z=332.1[M+H]+.

FURAN-100 (332 mg, 1 mmol) and N3-PEG3-NH2 (220 mg, 1 mmol) were dissolved in 20 mL of H₂O-DMF solvent (volume ratio 3:1), sodium L-ascorbate (220 mg) and copper sulfate pentahydrate (280 mg) were added; the resultant mixture was stirred and reacted at room temperature for 6 hours. The reactant was lyophilized; the lyophilizate was dissolved in a small amount of methanol and filtered; and the filtrate was separated and purified by preparative HPLC to give compound FURAN-102 (286 mg, yield 52%). ESI-MS: m/z=550.3[M+H]+.

Compounds FURAN-102 (276 mg, 0.5 mmol) and Fmoc-Val-Cit-PAB-PNP (383 mg, 0.5 mmol) were dissolved in 10 mL of DMF, and DIPEA (105 μl) was added; the reaction mixture was stirred and reacted at room temperature, and the reaction was monitored by mass spectrum until completion. Piperidine (100 μL) was then added and stirred at room temperature for 3 hours. The reactant was lyophilized; the lyophilizate was separated and purified by HPLC to give the purified product solution; and the product solution was lyophilized to give compound VC-PAB-102 (234 mg, yield 49%), ESI-MS: m/z=955.6[M+H]+.

VC-PAB-102 (192 mg, 0.2 mmol) was taken and dissolved in 5 mL of DMSO, and HEX-ANHYDRIDE (26 mg, 0.2 mmol) was added: the resultant mixture was stirred and reacted at room temperature for 2 hours. DIPEA (40 μL) was added and stirred at room temperature overnight. The reaction mixture was lyophilized; the lyophilizate was separated and purified by HPLC to give the purified product solution; and the product solution was lyophilized to give compound K9 (91 mg, yield 42%), ESI-MS: m/z=542.5[(M/2)+H]+.

### Synthesis of Compound k10

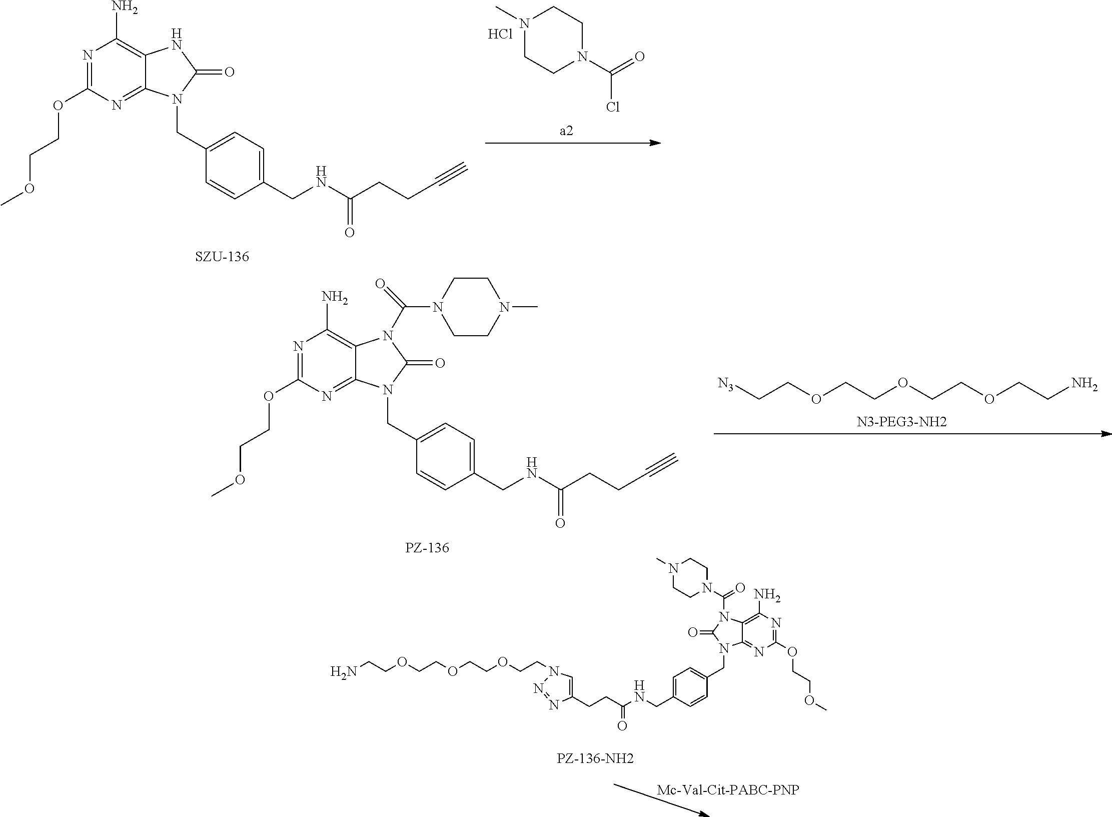

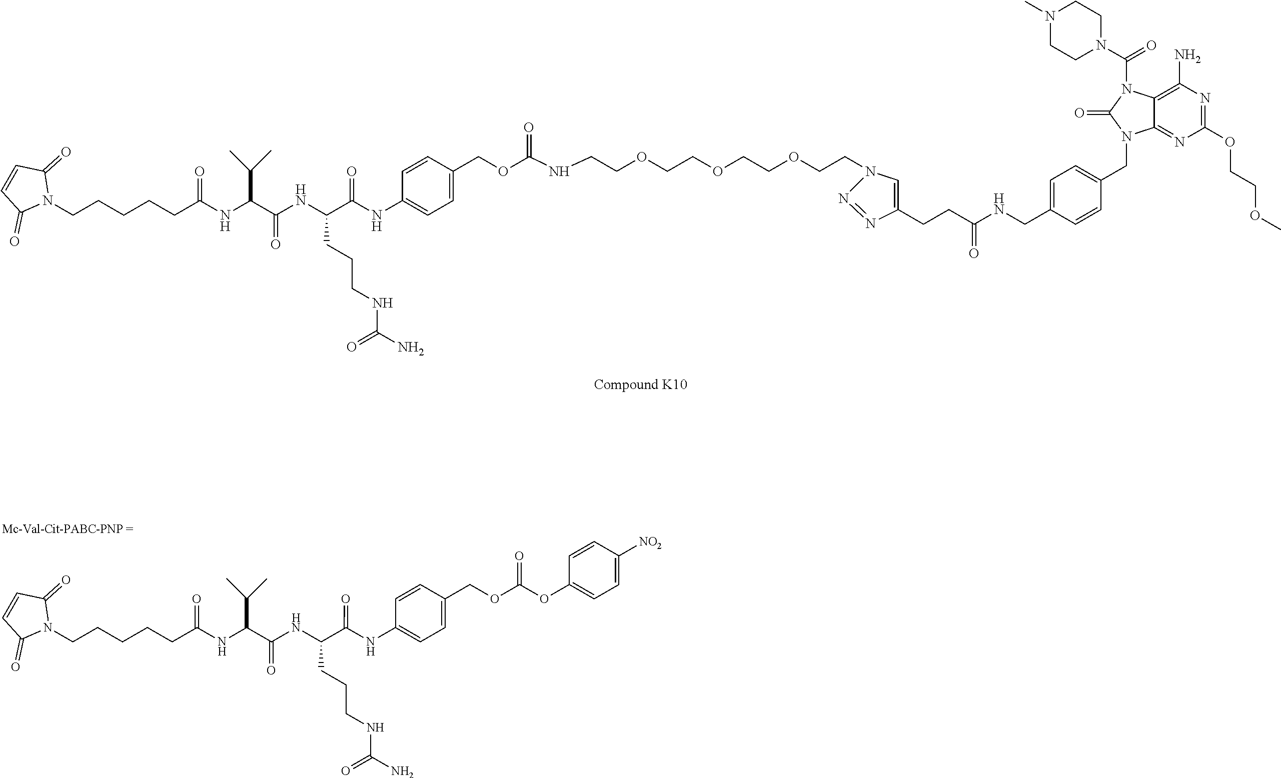 [SMILES: COCCOc1nc(N)c2c(n1)n(Cc1ccc(CNC(=O)CCc3cn(CCOCCOCCOCCNC(=O)OCc4ccc(NC(=O)[C@H](CCCNC(N)=O)NC(=O)[C@@H](NC(=O)CCCCCN5C(=O)C=CC5=O)C(C)C)cc4)nn3)cc1)c(=O)n2C(=O)N1CCN(C)CC1 ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

Compound SZU-136 (128 mg, 0.3 mmol) and DMAP (81 mg) were mixed uniformly with 3 mL of dry dichloromethane to give reaction solution 1. Compound a2 (60 mg, 0.3 mmol) was mixed with 0.2 mL of dichloromethane, and slowly added dropwise to reaction solution 1. After the addition, the mixture was stirred and reacted at room temperature for 3 hours. The reaction solution was lyophilized and the lyophilizate was separated and purified by HPLC to give PZ-136 (145 mg, yield 88%), ESI-MS: r/z=551.2[M+H]+.

PZ-136 (138 mg, 0.25 mmol) and N3-PEG3-NH2 (55 mg, 0.25 mmol) were dissolved in mL of H₂O-DMF solvent (1:3 by volume). Sodium L-ascorbate (55 mg) and copper sulfate pentahydrate (70 mg) were added; according to the treatment method of compound FlURAN-102, PZ-136-NH2 (119 mg, yield 62%) was obtained, ESI-MS: m/z=769.4[M+H]+.

Compound Mc-Val-Cit-PABC-PNP (111 mg, 0.15 mmol) was dissolved in 2 mL of DMF. PZ-136-NH2 (116 mg, 0.15 mmol) was taken and dissolved in 1 mL of DMF, and slowly added dropwise to the Mc-Val-Cit-PABC-PNP solution under cooling in an external bath at 0°; after the addition, the reaction solution was stirred at room temperature overnight. The reaction solution was lyophilized; and the lyophilizate was separated and purified by HPLC to give compound K10 (98 mg, yield 48%), ESI-MS: m/z=684.7[(M/2)+H]+.

### Synthesis of compound K11

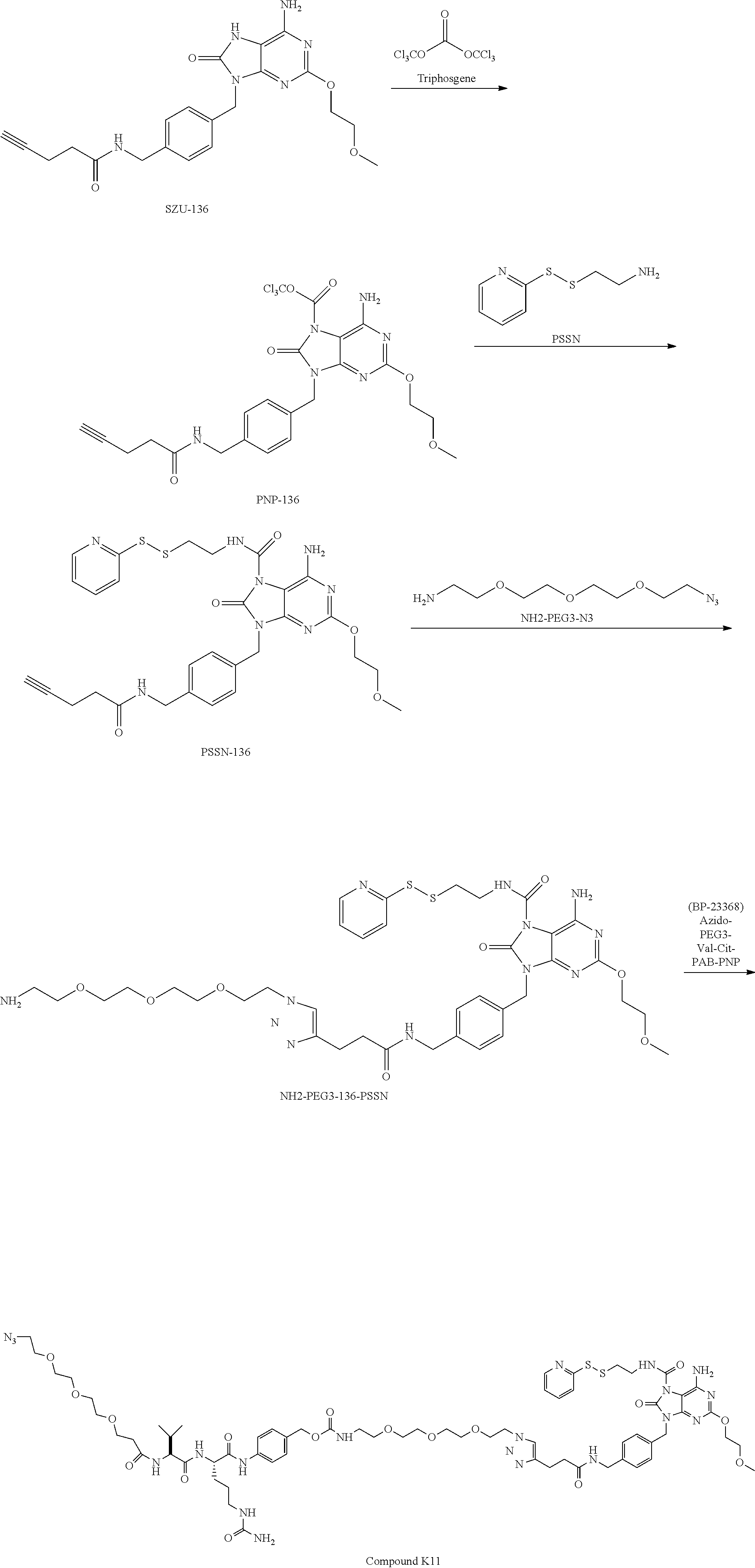

Compound SZU-136 (170 mg, 0.4 mmol) was dissolved in 3 mL of DMF, and DIPEA (175 μL) was added; Triphosgene (119 mg, 0.4 mmol) was taken and dissolved in 1 mL, of dry DMF and slowly added to the solution of SZU-136 in DMF. After the addition, the mixture reacted at room temperature for 3 hours; 0.5 mL of the solution of PSSN (75 mg) in DMF was added, and then the mixture was stirred and reacted for 3 hours. The reactant was lyophilized to remove the solvent, and the residue was separated by silica gel column chromatography (DCM/MeOH=10:1) to give compound PSSN-136 (92 mg, yield 36%), ESI-MS: m/z=637.2[M+H]+.

PSSN-136 (64 mg, 0.1 mmol) and NH2-PEC3-N3 (22 mg, 0.1 mmol) were taken and dissolved in 2 mL of DMF, 0.5 mL of pure water was added, and CuSO₄·5H₂O (5 mg) and sodium L-ascorbate (5 mg) were added. The resultant mixture was stirred and reacted at room temperature for 6 hours. The reaction solution was lyophilized, and the lyophilizate was separated and purified by HPLC to give NH2-PEG3-136-PSSN (47 mg, yield 55%), ESI-MS: m/z=856.3[M+H]+.

BP-23368 (37 mg) was dissolved in 0.5 mL of DMSO. The solution (0.5 mL) of compound NH2-PEG3-136-PSSN (40 mg, 0.047 mmol) and DIPEA (10 μl) in DMSO was slowly added to the BP-23368 solution, and the mixture was shaken and reacted at room temperature for 8 hours. The reactant was lyophilized, and the lyophilizate was separated and purified by HPLC to give compound K11 (48 mg, yield 69%). ESI-MS: m/z=745.8[(M/2)+H]+.

Synthesis of compound K12: according to the synthesis method of compound K9 except replacing GY100 with SZU-136, compound K12 can be obtained, ESI-MS: m/z=624.2[(M/2)+H]+.

### Synthesis of Compound K13

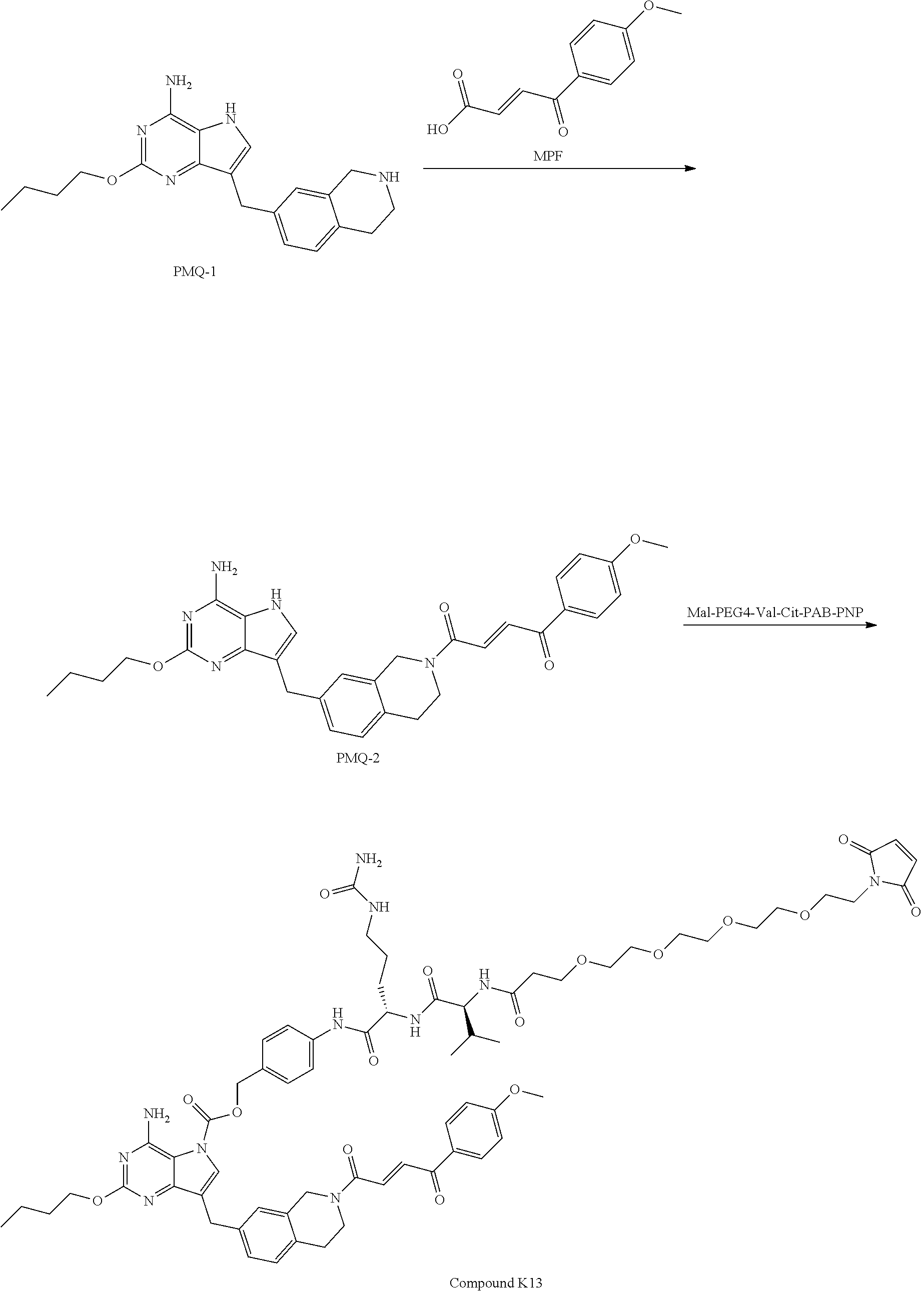

Compound PMQ-1 (106 mg, 0.3 mmol) was dissolved in 1 mL of DMF; MPF (62 mg, 0.3 mmol), DIPEA (52 μL) and the condensing agent PyAOP (157 mg, 0.3 mmol) were dissolved in 2 mL of DMF, mixed uniformly under cooling in an external bath at 10° C. and then slowly added dropwise to the solution of PMQ-1. After the addition, the mixture was stirred and reacted at room temperature for 3 hours. The reaction solution was lyophilized and purified by preparative HPLC to give compound PMQ-2 (100 mg, yield 62%), ESI-MS: m/z=540.2[M+H]+. Compound PMQ-2 (55 mg, 0.1 mmol) and compound Mal-PEG4-Val-Cit-PAB-PNP (88 mg, 0.1 mmol) were mixed in 2 mL of DMF, and the mixture was shaken and reacted at room temperature overnight. The reaction solution was lyophilized in the same way and purified by HPLC to give compound K13 (90 mg, yield 71%), ESI-MS: m/z=636.8 [(M/2)+H]+.

### Synthesis of Compound K14

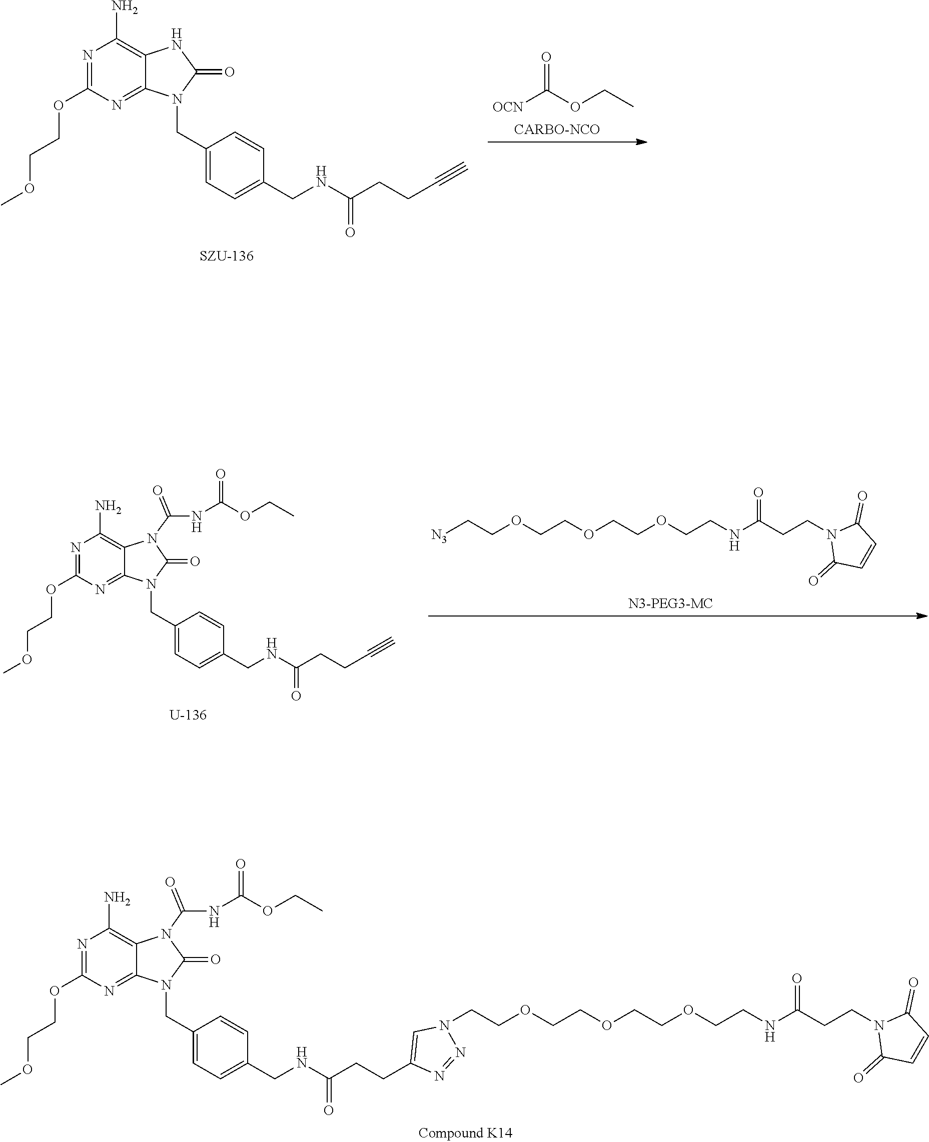

Compound SZU-136 (216 mg, 0.5 mmol) was dissolved in 5 mL of DMF, and CARBO-NCO (58 mg) was added for reaction. The reaction was monitored by mass spectrum until completion. The reaction solution was lyophilized to remove the solvent, and the product was separated by silica gel column chromatography (DCM/MeOH=10:1) to give compound U-136 (237 mg, yield 88%), ESI-MS: m/z=540.2[M+H]+.

Compounds U-136 (108 mg, 0.2 mmol) and N3-PEG3-MC (74 mg, 0.2 mmol) were dissolved in 5 mL of DMF, then 1 mL of pure water, 8 mg of copper sulfate pentahydrate, and 8 mg of sodium L-ascorbate were added. The resultant mixture was stirred at room temperature for 10 hours. The reaction solution was filtered, and the filtrate was lyophilized; and the lyophilization solution was separated and purified by HPLC to give compound K14 (129 Ng, yield 71%), ESI-MS: m/z=909.3[M+H]+.

According to the synthesis method of compound K14 except replacing CARBO-NCO with CARBO-NCS C

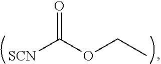

compound K15 can be obtained (ESI-MS: m/z=925.3[M+H]+)

### Synthesis of Compound K16

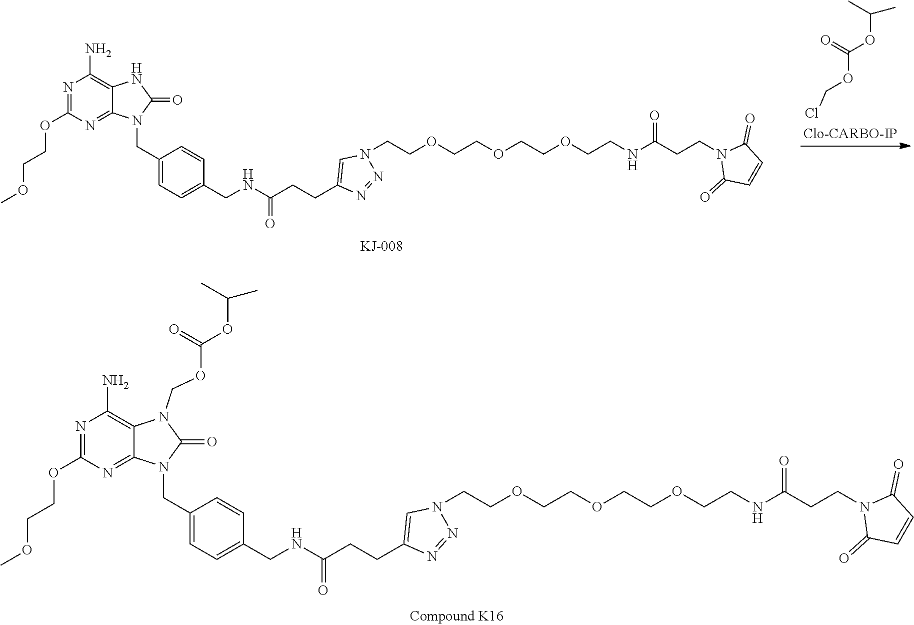

Compound KJ-008 (239 mg, 0.3 mmol) and K₂CO₃ (42 mg) were uniformly mixed in 3 mL of DMF, and Clo-CARBO-IP (46 mg, 0.3 mmol) was added, and the mixture was stirred and reacted at room temperature for 10 hours. The reaction solution was filtered; the filtrate was lyophilized and purified by HPLC to give compound K16 (150 mg, yield 55%), ESI-MS: m/z=910.4[M+H]⁺.

### Synthesis of Compound K17

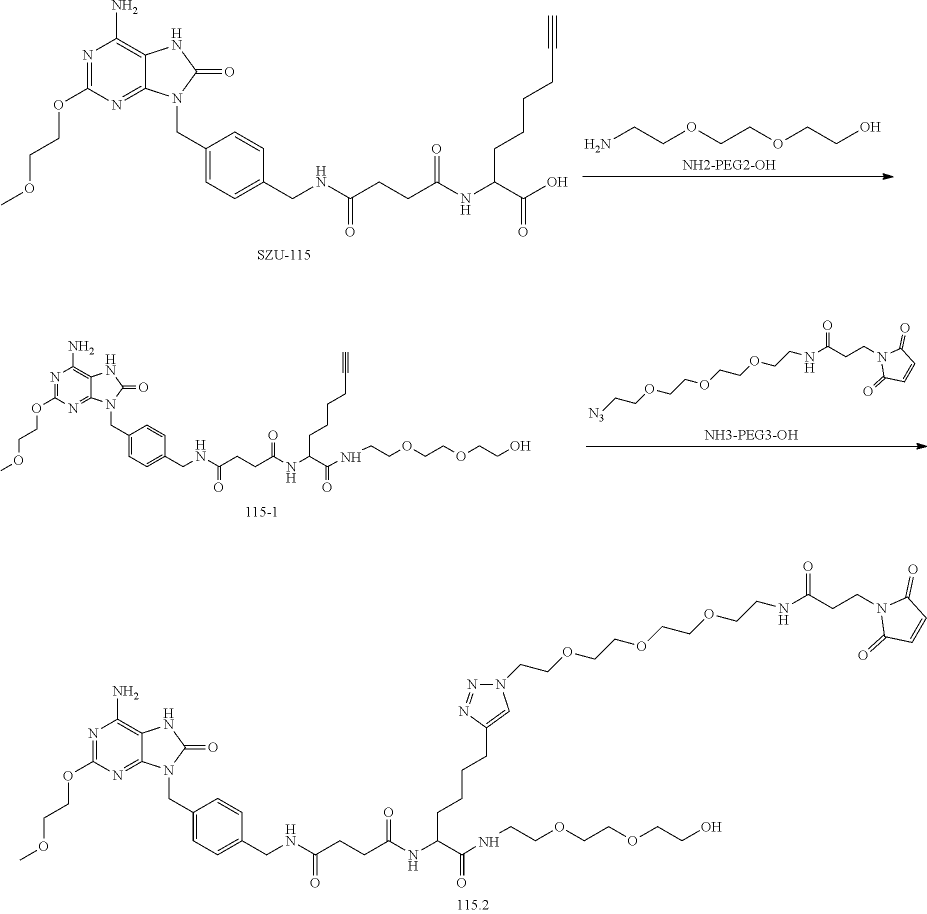 [SMILES: C#CCCCCC(NC(=O)CCC(=O)NCc1ccc(Cn2c(=O)[nH]c3c(N)nc(OCCOC)nc32)cc1)C(=O)O ; NCCOCCOCCO ; C#CCCCCC(NC(=O)CCC(=O)NCc1ccc(Cn2c(=O)[nH]c3c(N)nc(OCCOC)nc32)cc1)C(=O)NCCOCCOCCO ; [N-]=[N+]=NCCOCCOCCOCCNC(=O)CCN1C(=O)C=CC1=O ; COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)NCCOCCOCCO)cc3)c2n1]

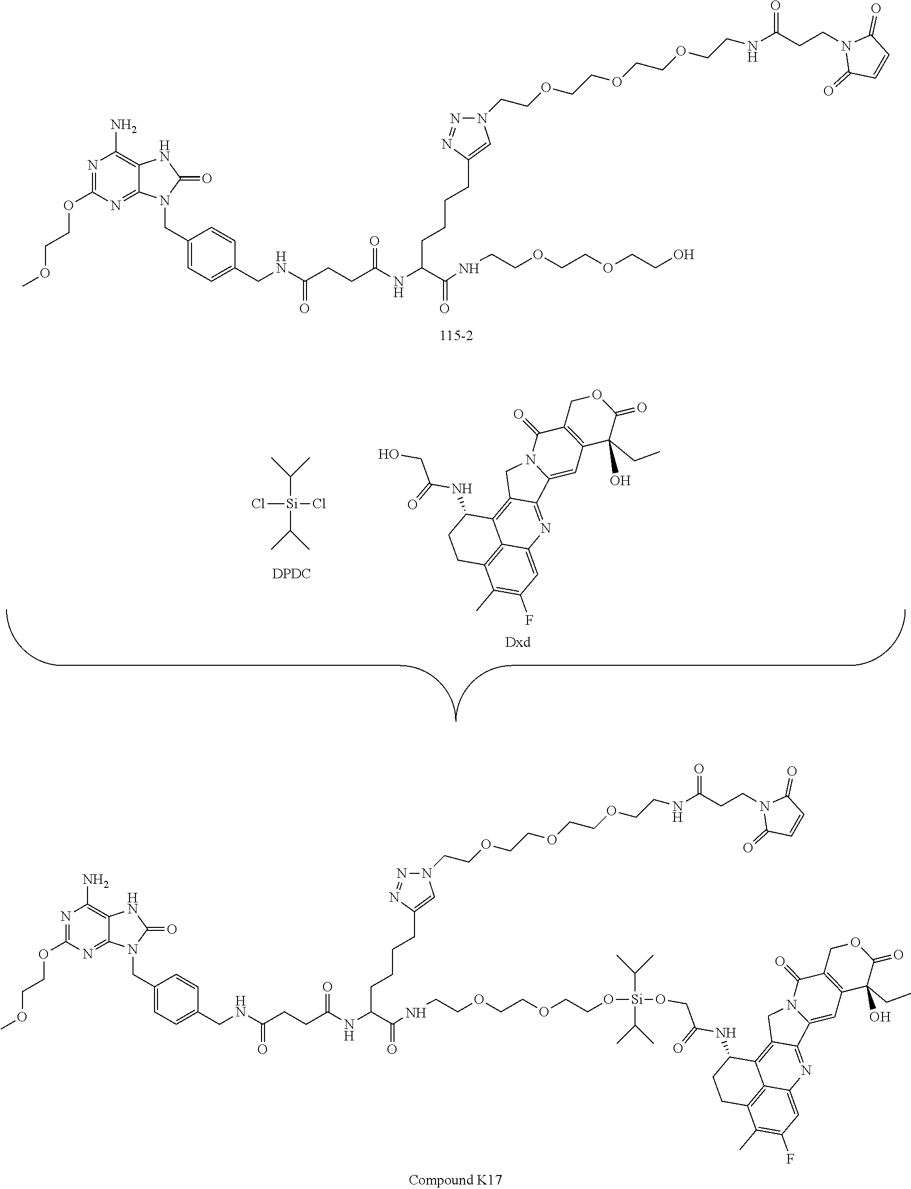

Compound SZU-115 (582 mg, 1 mmol), DPEA (180 μL) and PyAOP (522 mg, 1 mmol) were dissolved in DMF (10 mL) and stirred uniformly, and NH2-PEG2-OH (150 mg, 1 mmol) was added and stirred at room temperature for 3 hours. The reaction solution was concentrated under reduced pressure and subjected to silica gel column chromatography (DCM:MeOH=10:1) to give compound 115-1 (471 mg, yield 66%), ESI-MS: 5/z=713.3[M+H]+. Compound 15-1 (357 mg, 0.5 mmol) and N3-PEG3-MC (185 mg, 0.5 mmol) were dissolved in DMF-H2O=4:1 (5 mL), and copper sulfate pentabydrate (12 mg) and sodium L-ascorbate (121 mg) were added; the resultant mixture was stirred at room temperature for 6 hours. The reactant was concentrated under reduced pressure and separated by silica gel column chromatography (DCM/MeOH=10:1) to give compound 115-2 (254 mg, yield 47%), ESI-MS: m/z=1082.5[M+=H]+.

Compound DPDC (20 μL, 01 mmol) was dissolved in 0.5 mL of dry DMF; compound 115-2 (110 mg, 0.1 mmol), imidazole (15 mg) and DMAP (25 mg) were dissolved in dry DMF (1 mL), and slowly added to the solution of DPDC. The resultant mixture was stirred and reacted for 60 minutes. Compound Dxd (50 ag, 0.1 mmol) was dissolved in 1 mL of DMF, and then slowly added to the reaction mixture. After completion of the reaction was monitored by mass spectrum, the solvent was evaporated under reduced pressure, and the residue was separated by silica gel column chromatography (DCM:MeOH=10:1) to give compound K17 (106 mg, yield 63%), ESI-MS: m/z=844.4[(M/2)+H]+.

### Synthesis of Compound K18

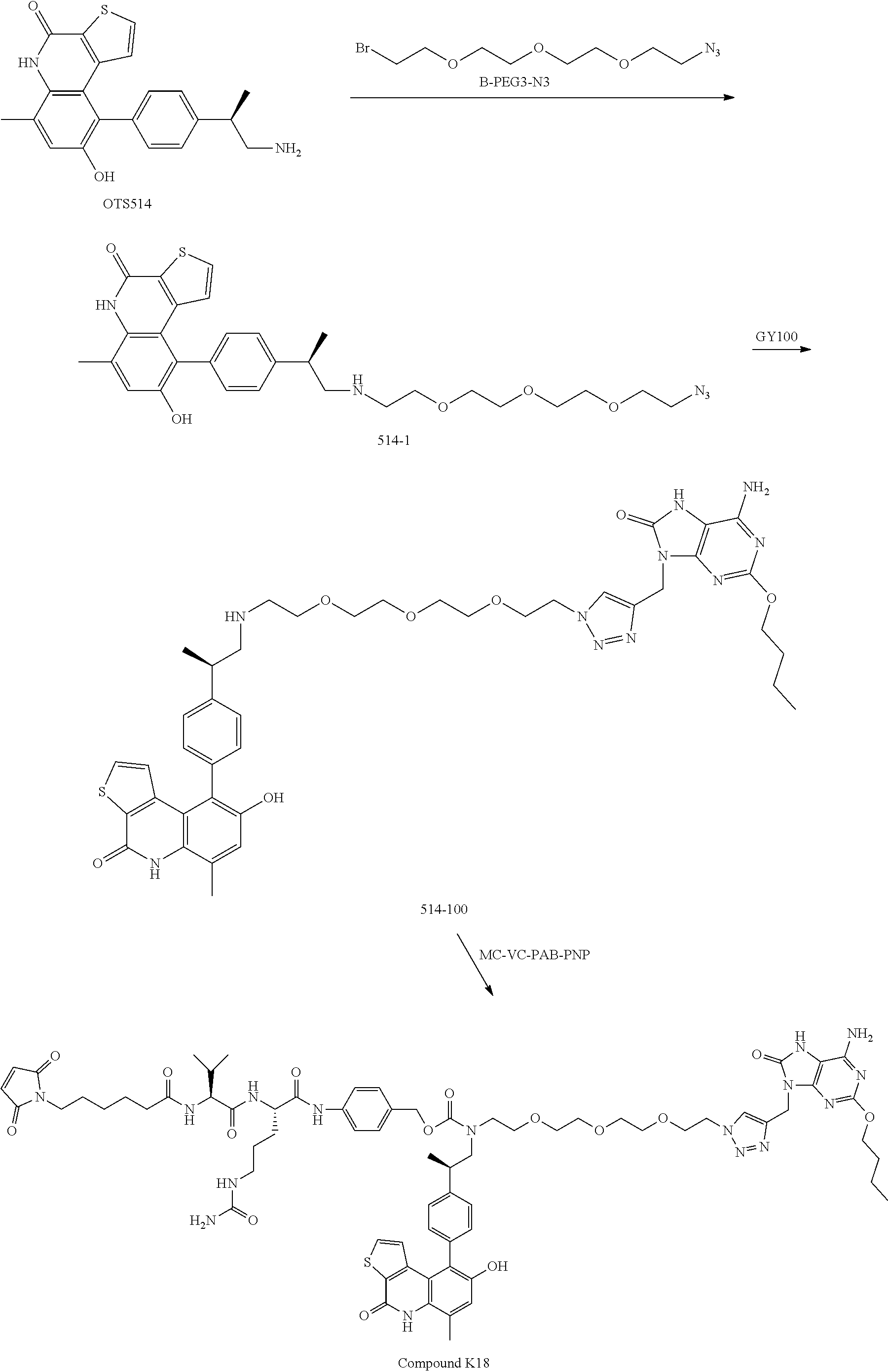

Compounds OTS514 (365 mg, 1 mmol) and B-PEG3-N3 (283 mg, 1 mmol) were mixed and dissolved in 5 mL of DMF, and K₂CO₃ (150 mg) was added; the mixture was stirred and reacted at room temperature for 6 hours. The reaction mixture was lyophilized, and the lyophilizate was subjected to silica gel column chromatography (DCM/MeOH=5:1); the solution containing the product was concentrated and dried to give compound 514-1 (175 mg, yield 31%). Compound 514-1 (147 mg, 0.26 mmol) and GY100 (68 mg, 0.26 mmol) were dissolved in 2 mL of DMF, then 0.5 ml, of water, copper sulfate pentahydrate (7 mg) and sodium L-ascorbate (7 mg) were added, and the resultant mixture was stirred and reacted for 6 hours. The reactant was lyophilized, and the lyophilizate was separated by silica gel column chromatography (DCM:MeOH=5:1) to give compound 514-100 (128 mg, yield 58%), ESI-MS: m/z=827.4[M+H]+.

Compound 514-100 (100 mg, 0.12 mmol) was dissolved in 1.5 mL of DMF, and DMAP (15 mg, 0.123 mmol) was added; after mixing and stirring uniformly, MC-VC-PAB-PNP (89 mg, 0.12 mmol) was added. The resultant mixture was stirred and reacted at room temperature for 3 hours. The solvent was removed by vacuum distillation; the residue was separated and purified by preparative HPLC; and the purified solution was concentrated under reduced pressure, and lyophilized to give compound K18 (104 mg, yield 61%). ESI-MS: m/z=713.8[(M/2)+H]+.

### Synthesis of Compound K19

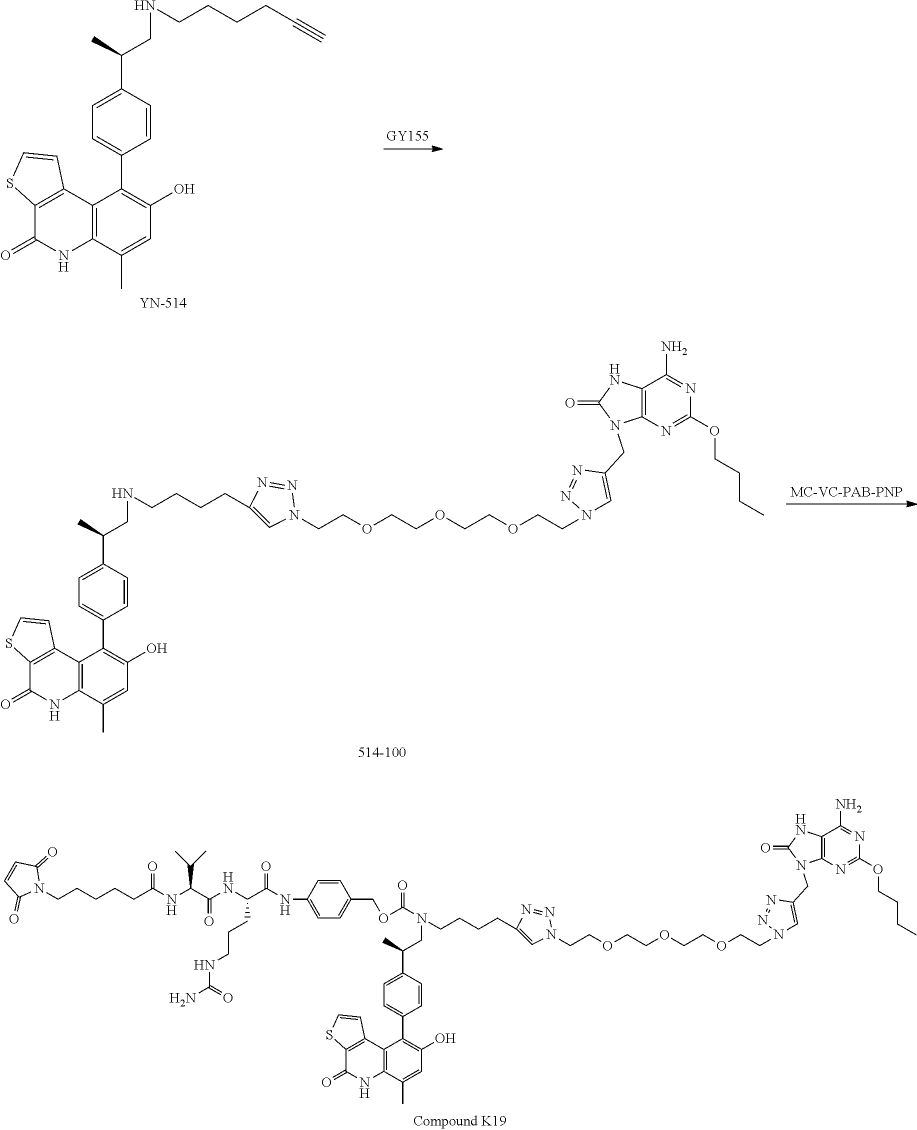 [SMILES: C#CCCCCNC[C@H](C)c1ccc(-c2c(O)cc(C)c3[nH]c(=O)c4sccc4c23)cc1 ; CCCCOc1nc(N)c2[nH]c(=O)n(Cc3cn(CCOCCOCCOCCn4cc(CCCCNC[C@H](C)c5ccc(-c6c(O)cc(C)c7[nH]c(=O)c8sccc8c67)cc5)nn4)nn3)c2n1 ; CCCCOc1nc(N)c2[nH]c(=O)n(Cc3cn(CCOCCOCCOCCn4cc(CCCCN(C[C@H](C)c5ccc(-c6c(O)cc(C)c7[nH]c(=O)c8sccc8c67)cc5)C(=O)OCc5ccc(NC(=O)[C@H](CCCNC(N)=O)NC(=O)[C@@H](NC(=O)CCCCCN6C(=O)C=CC6=O)C(C)C)cc5)nn4)nn3)c2n1]

Compounds GY155 (506 mg, 1 mmol) and YN-514 (445 mg, 1 mmol) were mixed and dissolved in 15 mL of DMF, then 3 mL of pure water, copper sulfate pentahydrate (25 mg) and sodium L-ascorbate (25 mg) were added: the resultant mixture was stirred and reacted for 6 hours under nitrogen protection, separated and purified by silica gel column chromatography (DCM:MeOH=5:1) to give compound GY191 (551 mg, yield 58%), ESI-MS: m/z=950.4 [M+H]+. GY191 (100 mg, 0.1 mmol) was taken and dissolved in 2 mL of DMF, and DIPEA (34 μL) was added; after stirring uniformly, the solution of MC-VC-PAB-PNP (75 mg, 0.1 mmol) in DMF (0.5 mL) was slowly added under cooling in an external bath at 0° C. After the addition, the mixture reacted at room temperature, and monitored by mass spectrum until completion. The reaction mixture was lyophilized, and the lyophilizate was separated and purified by preparative HPLC to give compound K19 (68 mg, yield 44%), ESI-MS: m/z=775.4[(M/2)+H]+.

Synthesis of compound K₂O: according to the synthesis method of compound K19 except replacing compound GY100 with SZU-136, compound K20 can be obtained, ESI-MS: m/z=787.4 [(M/2)+H]+.

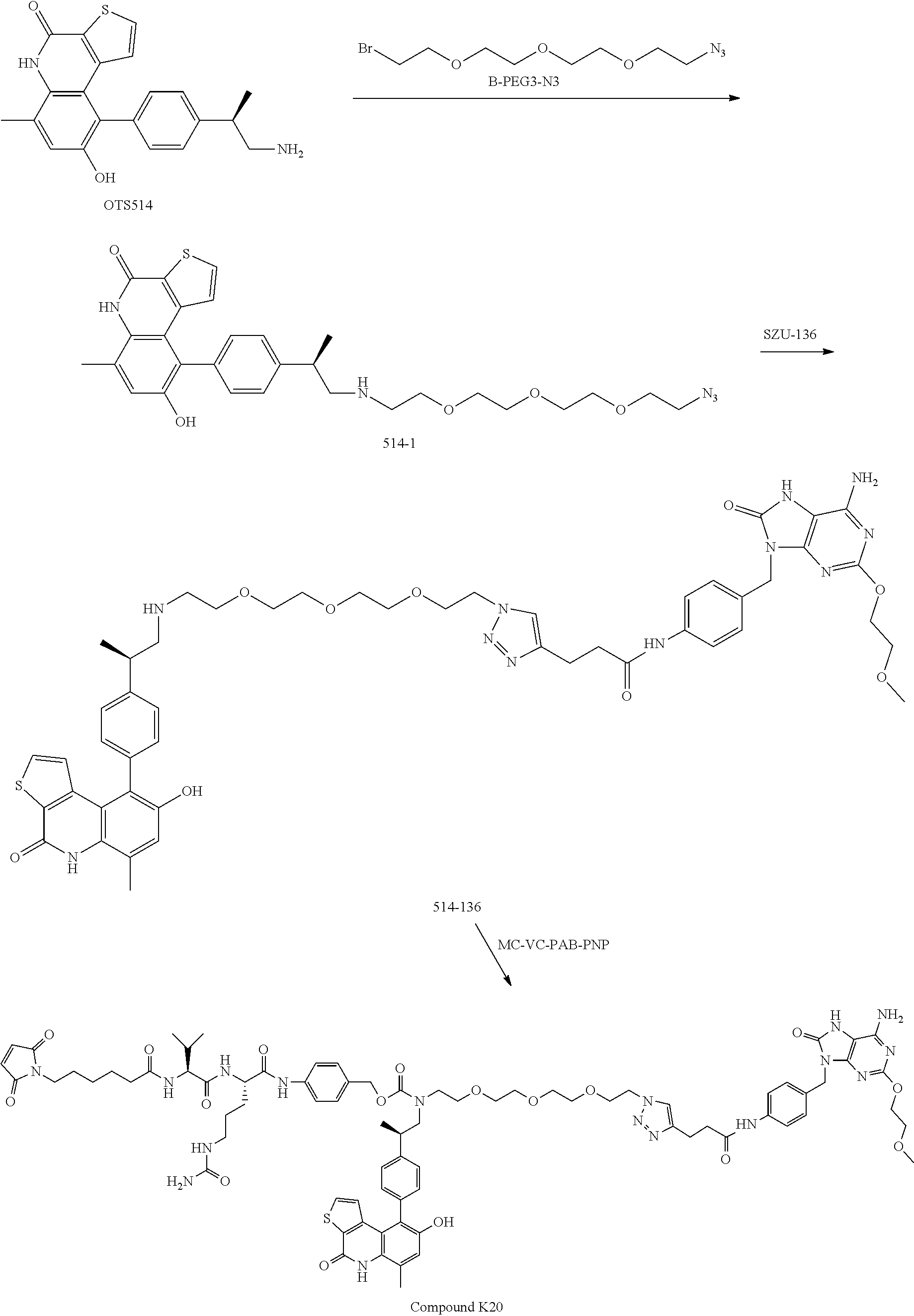

### Synthesis of Compound K21

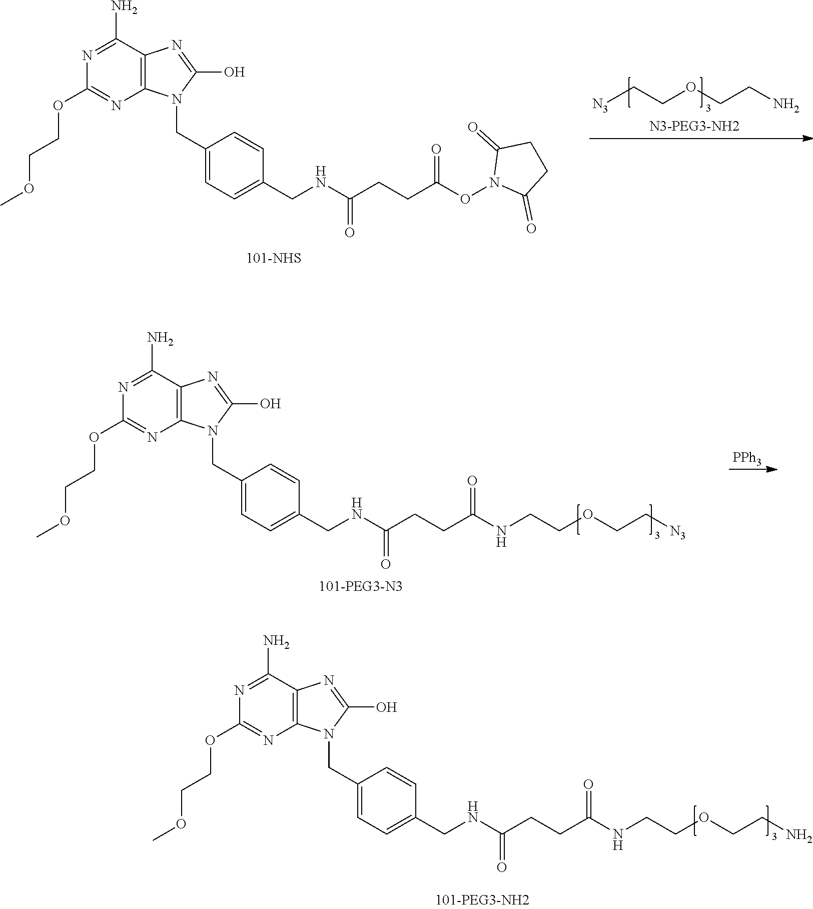

101-NHS (667 mg) was dissolved in 2 mL of anhydrous DMF, and N35-PEG3-NH2 (300 m g) was added and stirred at room temperature overnight. After the reaction was completed, the reaction solution was poured into water, and a white solid precipitated out and dried to give 101-PEC3-N3.

101-PEG3-N3 obtained in the previous step was dissolved in 10 m of anhydrous THF, triphenylphosphine (484 mg) was added under ice bath and stirred at room temperature overnight, and the reaction was monitored by LC-MS. After the reaction was completed, 20 mL of water was added and stirred for 30 minutes; and the mixture was filtered under reduced pressure, washed with water twice, and dried to give 101-PEG3-NH2 (625 mg, combined yield of the two steps 79%), ESI-MS: m/z=619.3[M+H]+;

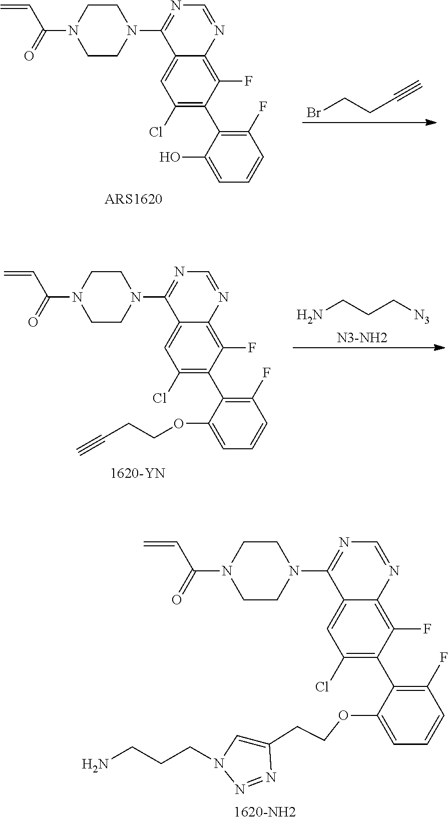

ARS1620 (108 mg, 0.25 mmol) and 4-bromobutyne (24 μL) were dissolved in DMF (2 mL), and K₂CO₃ (35 mg) was added, and the mixture was stirred and reacted at room temperature for 12 hours. The reaction solution was filtered, and 0.4 mL of pure water, N3-NH2 (25 mg, 0.25 mmol), CuSO₄·5H₂O (8 mg) and sodium L-ascorbate (8 mg) were added to the filtrate; the resultant mixture reacted at room temperature for 6 hours. The reaction solution was filtered; the filtrate was lyophilized; and the lyophilizate was separated and purified by preparative HPLC to give 1620-NH2 (77 mg, yield 53%/), ESI-MS: m/z=584.2[M+H]+.

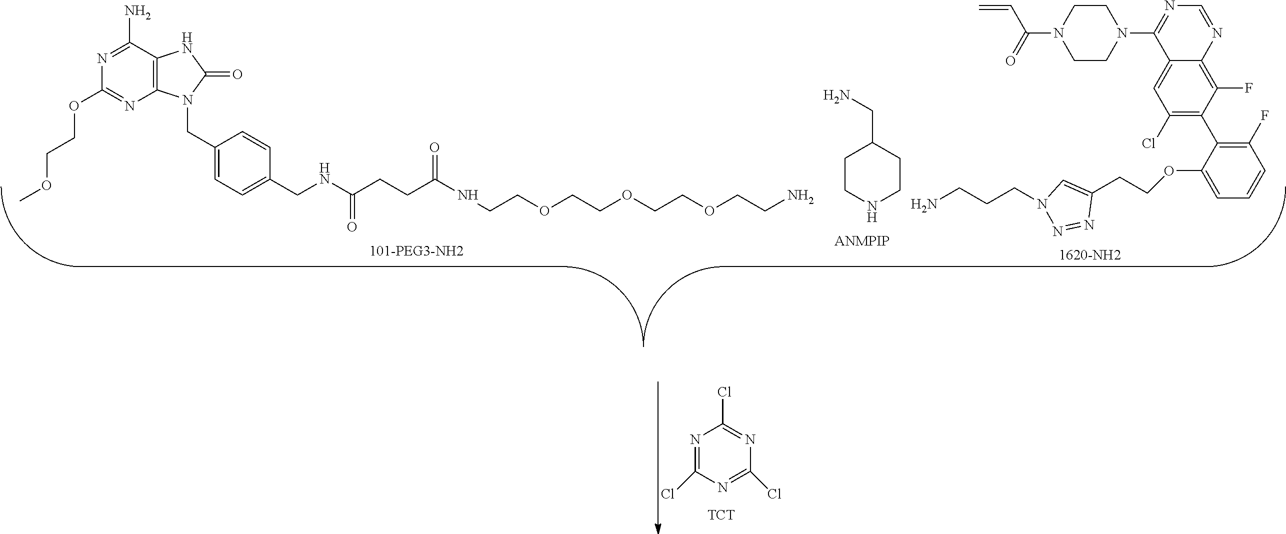

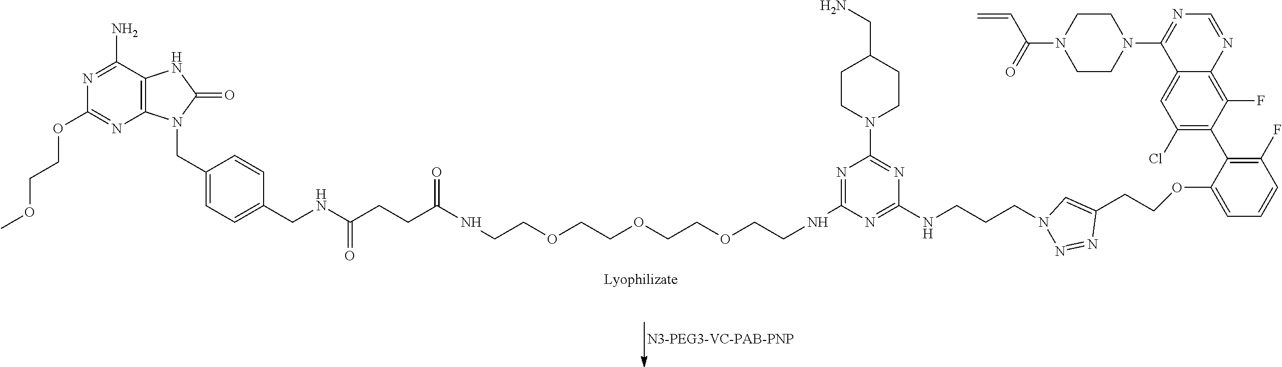 [SMILES: C=CC(=O)N1CCN(c2ncnc3c(F)c(-c4c(F)cccc4OCCc4cn(CCCNc5nc(NCCOCCOCCOCCNC(=O)CCC(=O)NCc6ccc(Cn7c(=O)[nH]c8c(N)nc(OCCOC)nc87)cc6)nc(N6CCC(CN)CC6)n5)nn4)c(Cl)cc23)CC1]

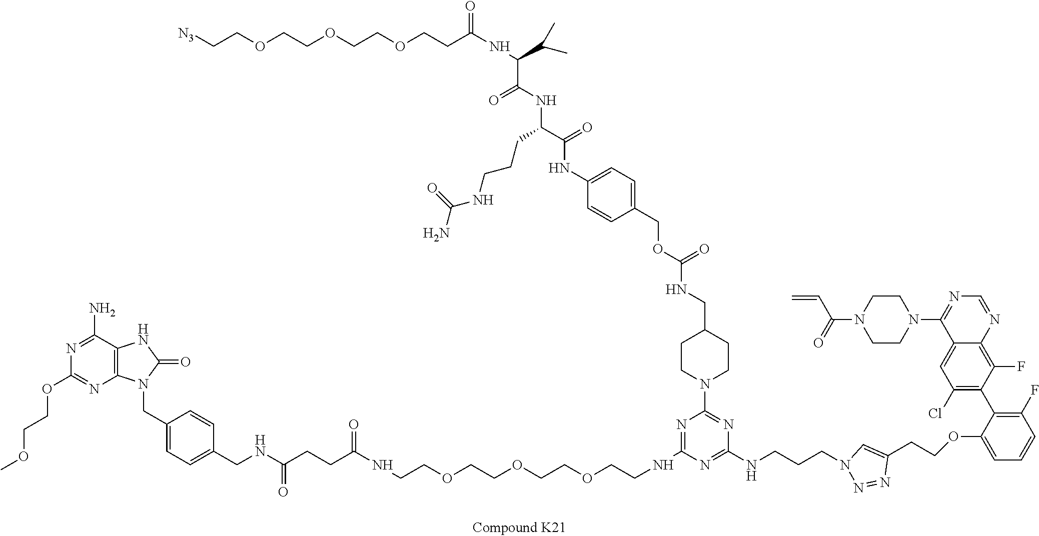 [SMILES: C=CC(=O)N1CCN(c2ncnc3c(F)c(-c4c(F)cccc4OCCc4cn(CCCNc5nc(NCCOCCOCCOCCNC(=O)CCC(=O)NCc6ccc(Cn7c(=O)[nH]c8c(N)nc(OCCOC)nc87)cc6)nc(N6CCC(CNC(=O)OCc7ccc(NC(=O)[C@H](CCCNC(N)=O)NC(=O)[C@@H](NC(=O)CCOCCOCCOCCN=[N+]=[N-])C(C)C)cc7)CC6)n5)nn4)c(Cl)cc23)CC1]

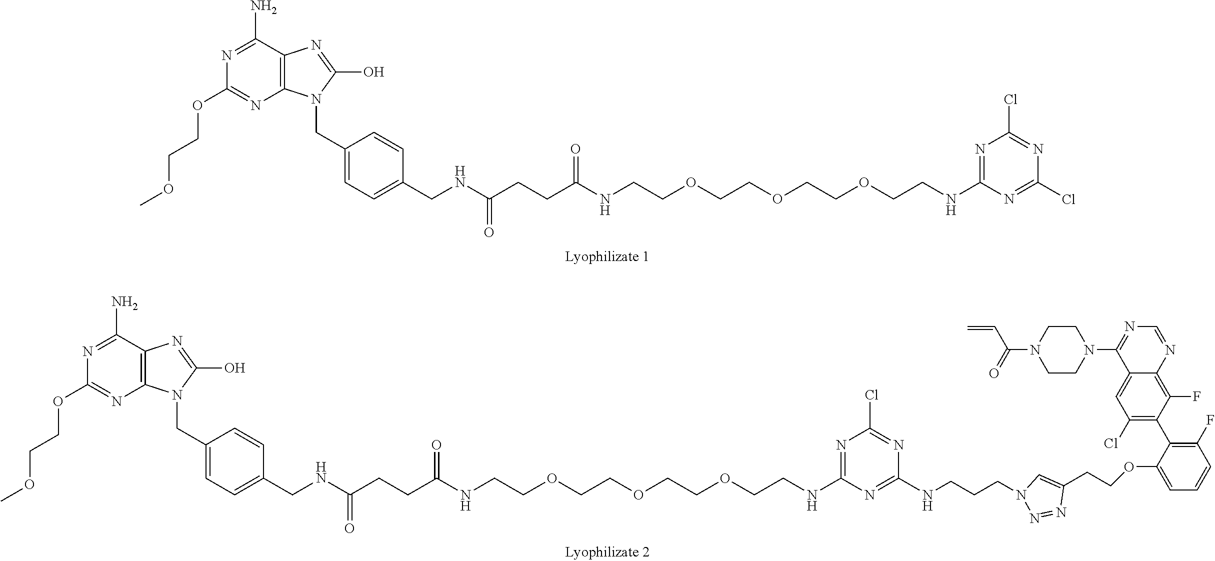 [SMILES: COCCOc1nc(N)c2nc(O)n(Cc3ccc(CNC(=O)CCC(=O)NCCOCCOCCOCCNc4nc(Cl)nc(Cl)n4)cc3)c2n1 ; C=CC(=O)N1CCN(c2ncnc3c(F)c(-c4c(F)cccc4OCCc4cn(CCCNc5nc(Cl)nc(NCCOCCOCCOCCNC(=O)CCC(=O)NCc6ccc(Cn7c(O)nc8c(N)nc(OCCOC)nc87)cc6)n5)nn4)c(Cl)cc23)CC1]

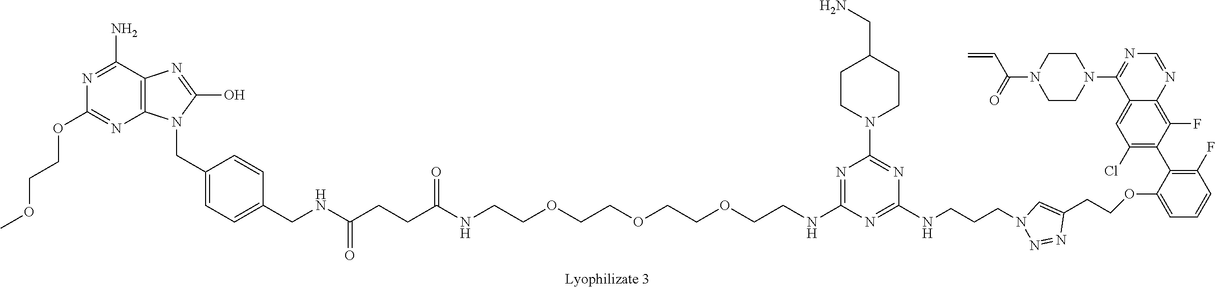 [SMILES: C=CC(=O)N1CCN(c2ncnc3c(F)c(-c4c(F)cccc4OCCc4cn(CCCNc5nc(NCCOCCOCCOCCNC(=O)CCC(=O)NCc6ccc(Cn7c(O)nc8c(N)nc(OCCOC)nc87)cc6)nc(N6CCC(CN)CC6)n5)nn4)c(Cl)cc23)CC1]

TCT (185 mg, 1 mmol) was dissolved in 5 mL of dry DMF, and DIPEA (175 t L) was slowly added under cooling in an external bath at 0° C. Compound 101-PEG3-NH2 (619 mg, 1 mmol) was taken and dissolved in 3 mL of dry DMF, and slowly added to the solution of TCT under cooling in an external bath at 0° C. After the addition, the mixture continued to react for 3 hours. The reactant was lyophilized; the lyophilizate was separated by preparative HPLC and dried; and the product separation solution was lyophilized to give lyophilizate 1 (276 mg, yield 36%), ESI-MS: m/z=767.2[M+H]+. Following the same method as that for the synthesis of lyophilizate 1, lyophilizate 1 and 1620-NH2 reacted to give lyophilizate 2 (ESI-MS: m/z=657.6[(M/2)+H]+); lyophilizate 2 and AMPIP reacted to give lyophilizate 3 (ESI-MS: m/z=696.4[(M/2)+H]+).

Lyophilizate 3 (139 mg, 0.1 mmol) was taken and dissolved in 3 mL of DMF, and DIPEA (175 μL) was added. N3-PEG3-VC-PAB-PNP (77.4 mg, 0.1 mmol) was dissolved in 0.5 mL of DMF and slowly added to the solution of lyophilizate 3 under cooling and stirring at 0° C.; after the addition, the mixture reacted at room temperature for 6 hours. The reaction solution was lyophilized, and the lyophilizate was separated and purified by preparative HPLC; the purified solution containing the product was lyophilized to give compound K21 (135 mg, yield 67%), ESI-MS: m/z=676.2[(M/3)+H]+.

### Synthesis of Compound K22

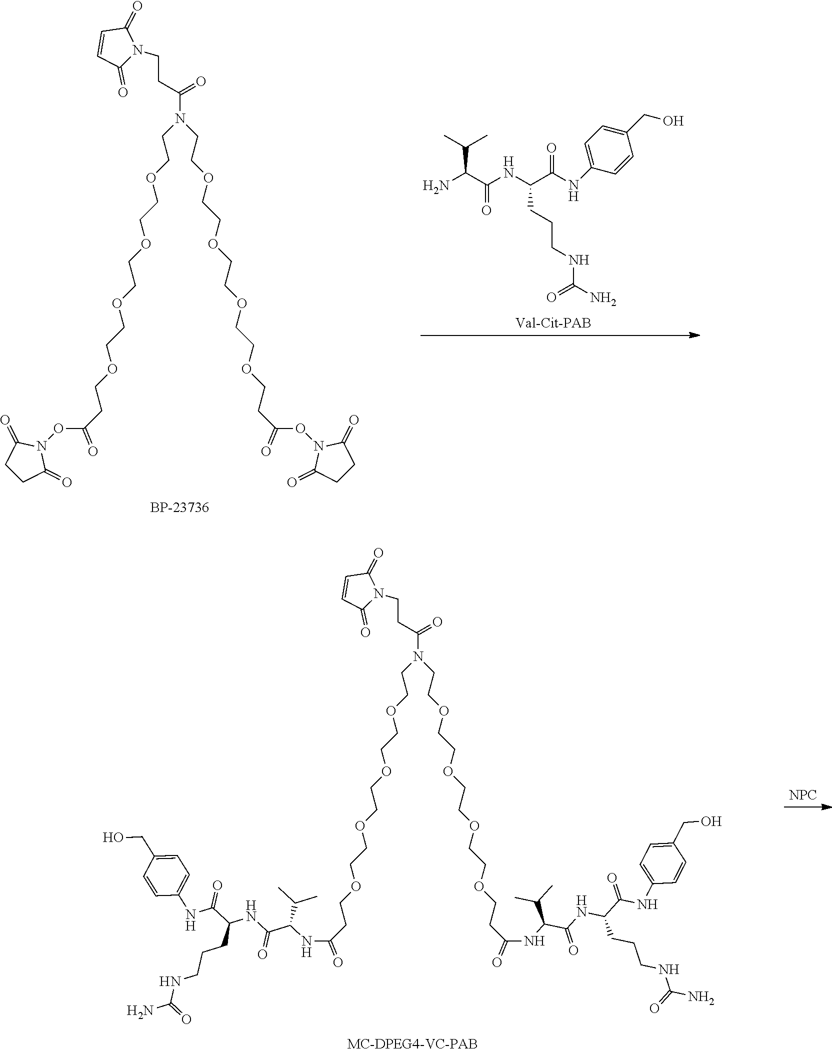 [SMILES: O=C(CCOCCOCCOCCOCCN(CCOCCOCCOCCOCCC(=O)ON1C(=O)CCC1=O)C(=O)CCN1C(=O)C=CC1=O)ON1C(=O)CCC1=O ; CC(C)[C@H](N)C(=O)N[C@@H](CCCNC(N)=O)C(=O)Nc1ccc(CO)cc1 ; CC(C)[C@H](NC(=O)CCOCCOCCOCCOCCN(CCOCCOCCOCCOCCC(=O)N[C@H](C(=O)N[C@@H](CCCNC(N)=O)C(=O)Nc1ccc(CO)cc1)C(C)C)C(=O)CCN1C(=O)C=CC1=O)C(=O)N[C@@H](CCCNC(N)=O)C(=O)Nc1ccc(CO)cc1 ; "NPC"]

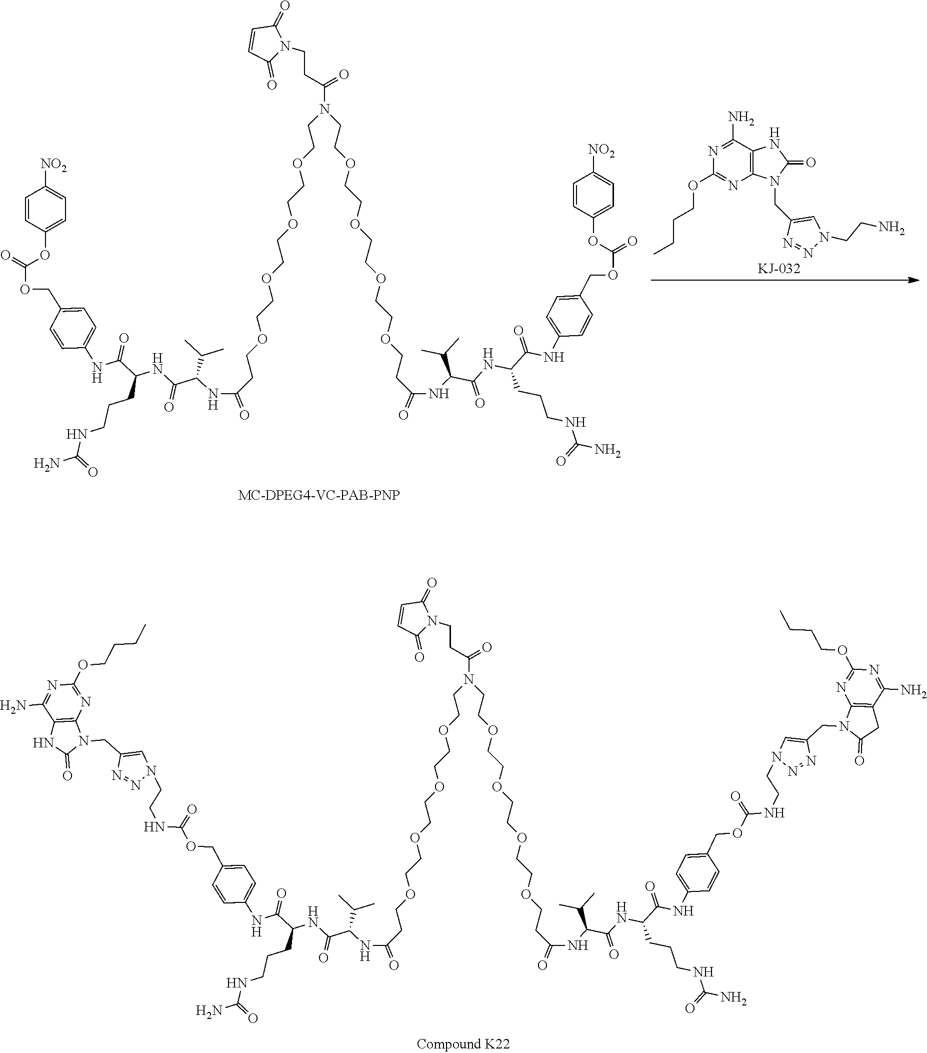

Compounds BP-23736 (129 mg, 0.15 mmol) and V al-Cit-PAB (114 mg, 0.30 mmol) were dissolved in 1 mL of DMF and stirred at room temperature for 8 hours. The reaction solution was lyophilized, separated and purified by semi-preparative HPLC to give compound MC-DPEG4-VC-PAB (108 mg, yield 52%), ESI-MS: m/z 694.0 [(M/2)+H]+. NPC (43 mg, 014 mmol) was dissolved in 0.5 mL of anhydrous THF, and 1 mL of the solution of compound MC-DPEG4-VC-PAB (100 mg, 0.07 mmol) in DMF was taken and slowly added dropwise to the solution of NPC; after the addition, the reaction solution was shaken and reacted at room temperature for 3 hours; 0.5 mL of the solution of compound KJ-032 (50 mg, 0.14 mmol) was added dropwise to the reaction solution, and the reaction solution continued to react at room temperature overnight. The reaction solution was lyophilized, and the lyophilizate was separated and purified by HPLC to give compound K22 (55 mg, yield 36%), ESI-MS: m/z=712.0[(M/3)+H]+.

Compound KJ-032 can be obtained by the click reaction of GY100 and compound 2-azidoethylamine (ESI-MS: m/z=34.2[M+H]+).

### Synthesis of Compound K23

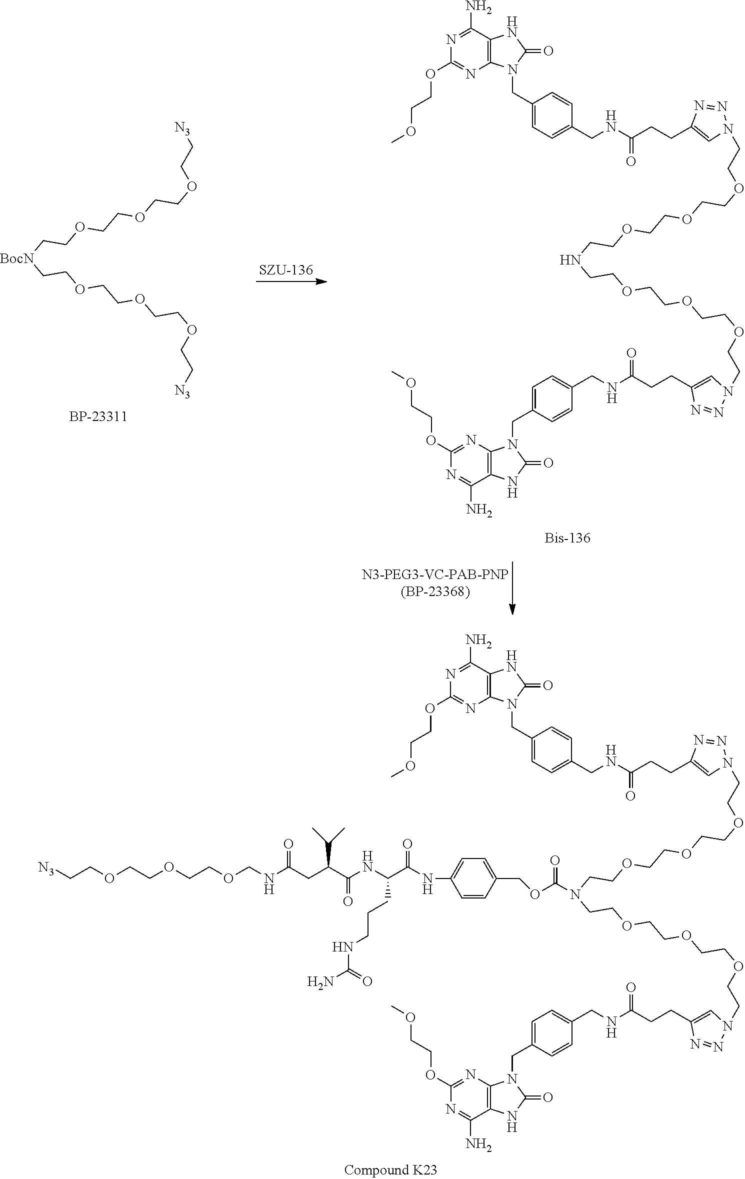 [SMILES: CC(C)(C)OC(=O)N(CCOCCOCCOCCN=[N+]=[N-])CCOCCOCCOCCN=[N+]=[N-] ; COCCOc1nc(N)c2[nH]c(=O)n(Cc3ccc(CNC(=O)CCc4cn(CCOCCOCCOCCNCCOCCOCCOCCn5cc(CCC(=O)NCc6ccc(Cn7c(=O)[nH]c8c(N)nc(OCCOC)nc87)cc6)nn5)nn4)cc3)c2n1 ; COCCOc1nc(N)c2[nH]c(=O)n(Cc3ccc(CNC(=O)CCc4cn(CCOCCOCCOCCN(CCOCCOCCOCCn5cc(CCC(=O)NCc6ccc(Cn7c(=O)[nH]c8c(N)nc(OCCOC)nc87)cc6)nn5)C(=O)OCc5ccc(NC(=O)[C@H](CCCNC(N)=O)NC(=O)[C@@H](CC(=O)NCOCCOCCOCCN=[N+]=[N-])C(C)C)cc5)nn4)cc3)c2n1]

Compound BP-23311 (78 mg, 0.15 mmol) and SZU-136 (128 mg, 0.3 mmol) were mixed and dissolved in a 1:4 H₂O-DMF solvent (2 mL), and CuSO₄·5H₂O (10 mg) and sodium L-ascorbate (10 mg) were added; after mixing uniformly, the mixture was stirred and reacted at room temperature overnight under nitrogen protection. The reaction solution was filtered; the filtrate was lyophilized; the lyophilizate was added with 1:3 TFA-DCM solvent (1:3, 2 mL), and the mixture was stirred and reacted for 1 hour. The solvent was removed by vacuum distillation, and the residue was separated and purified by HPLC to give compound Bis-136 (105 mg, yield 55%), ESI-MS: m/z=635.1 [(M/2)+H]+.

Compound Bis-136 (101 mg, 0.08 mmol) was taken and dissolved in 1.5 ml, of DMF, and N3-PEG3-VC-PAB-PNP (62 mg, 0.08 mmol) and DIPEA (18 μL) were added and mixed, and the mixture reacted for 6 hours. The reaction solution was lyophilized, and the lyophilizate was separated and purified by HPLC to give compound K23 (59 mg, yield 39%), ESI-MS: m/z=952.0[(M/2)+H]+.

### Synthesis of Compound K24

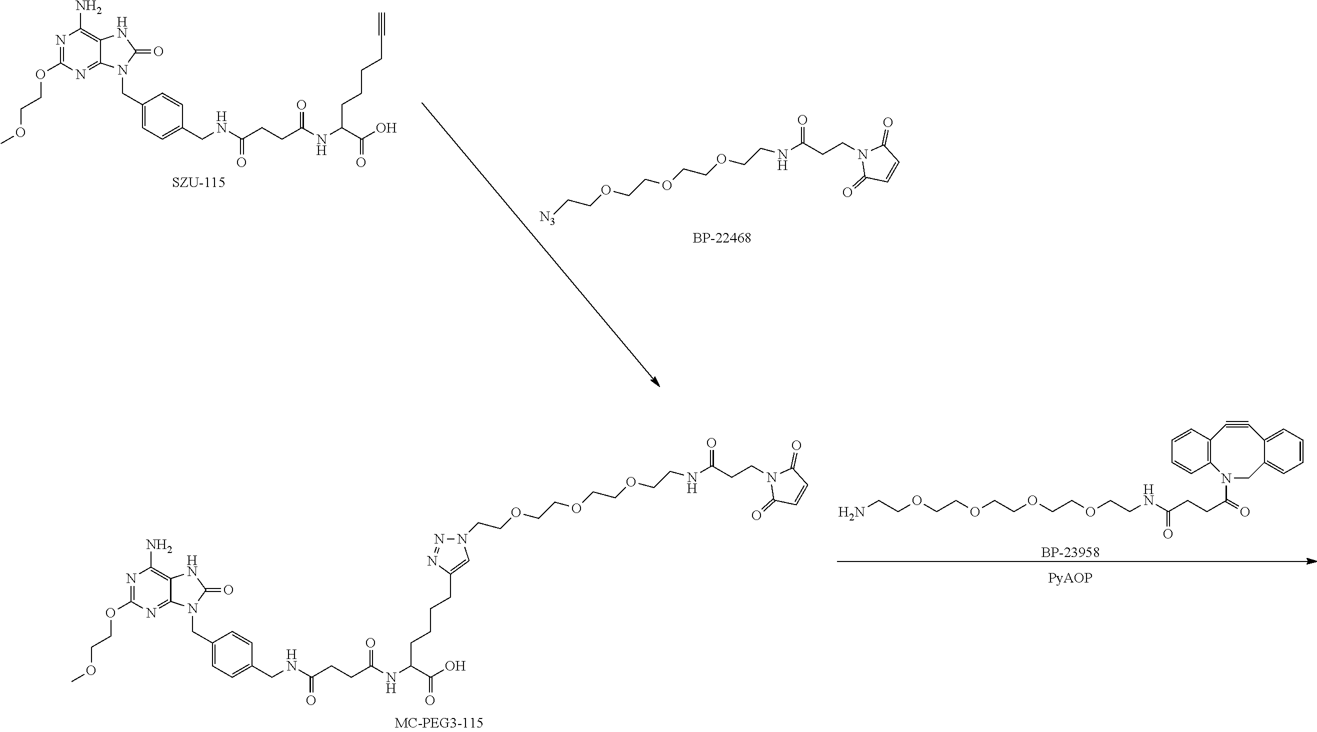 [SMILES: C#CCCCCC(NC(=O)CCC(=O)NCc1ccc(Cn2c(=O)[nH]c3c(N)nc(OCCOC)nc32)cc1)C(=O)O ; [N-]=[N+]=NCCOCCOCCOCCNC(=O)CCN1C(=O)C=CC1=O ; COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)O)cc3)c2n1 ; NCCOCCOCCOCCOCCNC(=O)CCC(=O)N1Cc2ccccc2C#Cc2ccccc21]

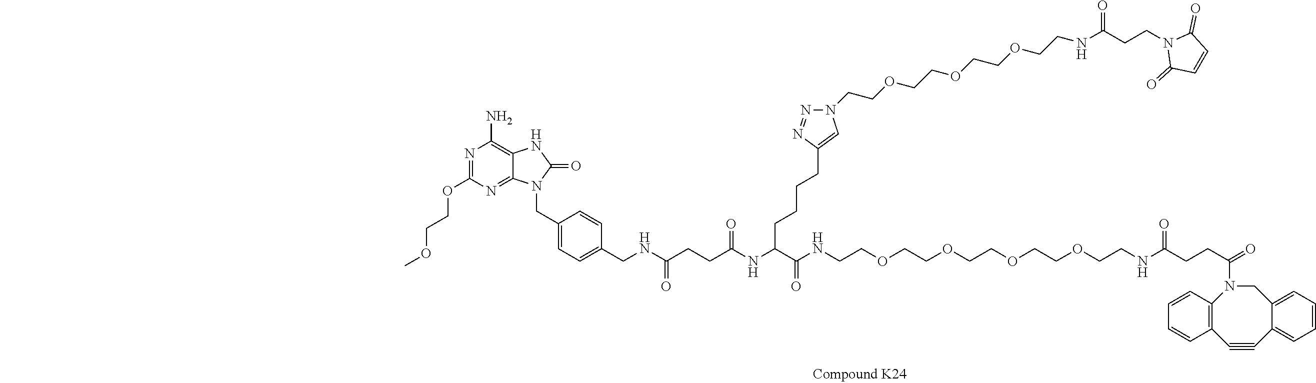

Compounds SZU-115 (100 mg, 0.17 mmol) and BP22468 (64 mg, 0.17 mmol) were dissolved in 2 mL of DMF-H2O solvent (4:1 by volume), and CuSO₄·5H₂O (5 mg) and sodium L-ascorbate (5 mg) were added; the mixture reacted at room temperature overnight under nitrogen protection. The reaction solution was filtered; the filtrate was lyophilized, then separated and purified by HPLC to give compound MC-PEG3-115 (63 mg, yield 39%), ESI-MS: m/z=: 951.4[M+H]+. Compound MC-PFG3-115 (60 mg, 0.063 mmol), DIPEA (12 μL) and PyAOP (35 mg, 0.067 mmol) were dissolved in 1 mL of DMF, after stirring uniformly, BP-23958 (33 mg, 0.063 mmol) was added, and the mixture was stirred and reacted at room temperature for 10 hours. The reaction product was separated and purified by semi-preparative HPLC to give compound K24 (48 mg, yield 52%), ESI-MS: m/z=729.1[(M/2)+H]+.

### Synthesis of Compound K25

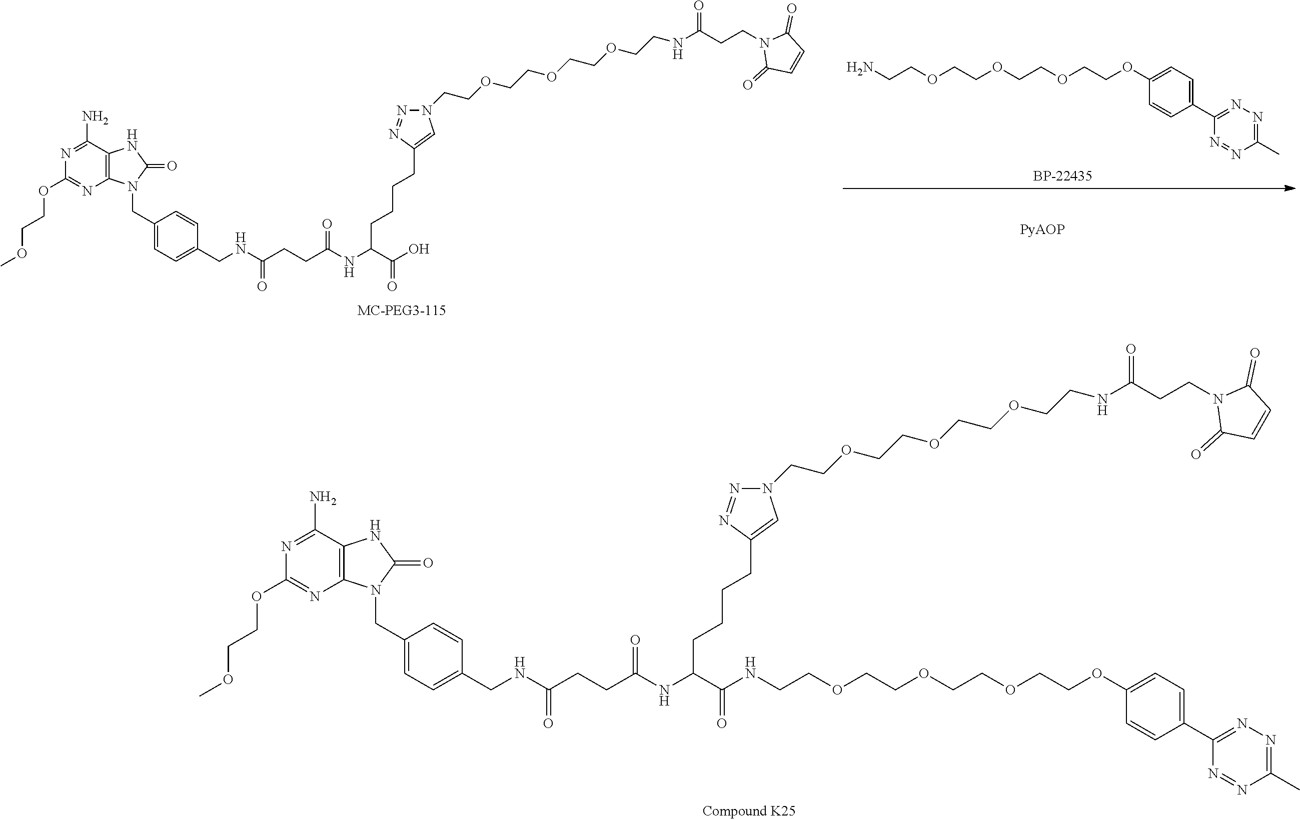

Compound BP-22435 was dissolved in water, neutralized to pH 8 with saturated sodium carbonate solution, and extracted with dichloromethane to give the free base BP-22435. According to the method for preparing K24 with compounds MC-PEG3-115 and BP-23958, compound K25 can be obtained (ESI-MS: m/z=648.8[(M/2)+H]+).

### Synthesis of Compound K26

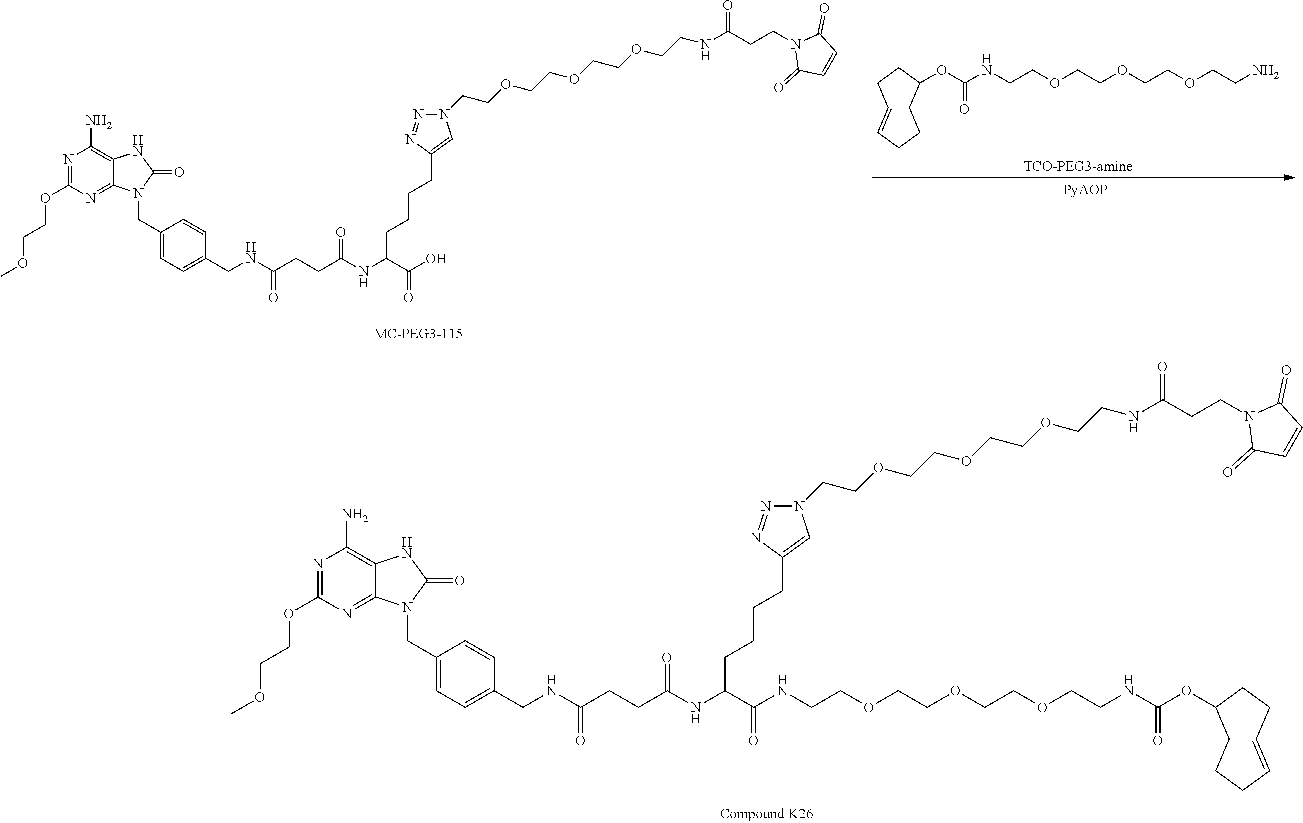 [SMILES: COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)O)cc3)c2n1 ; NCCOCCOCCOCCNC(=O)OC1CC/C=C/CCC1 ; COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)NCCOCCOCCOCCNC(=O)OC4CC/C=C/CCC4)cc3)c2n1]

According to the synthesis method of compound K24 except replacing BP-23958 with compound TCO-PEG3-amine, compound K26 can be obtained (ESI-MS: m/z=639.0[(M/2)+H]+).

### Synthesis of Compound K27

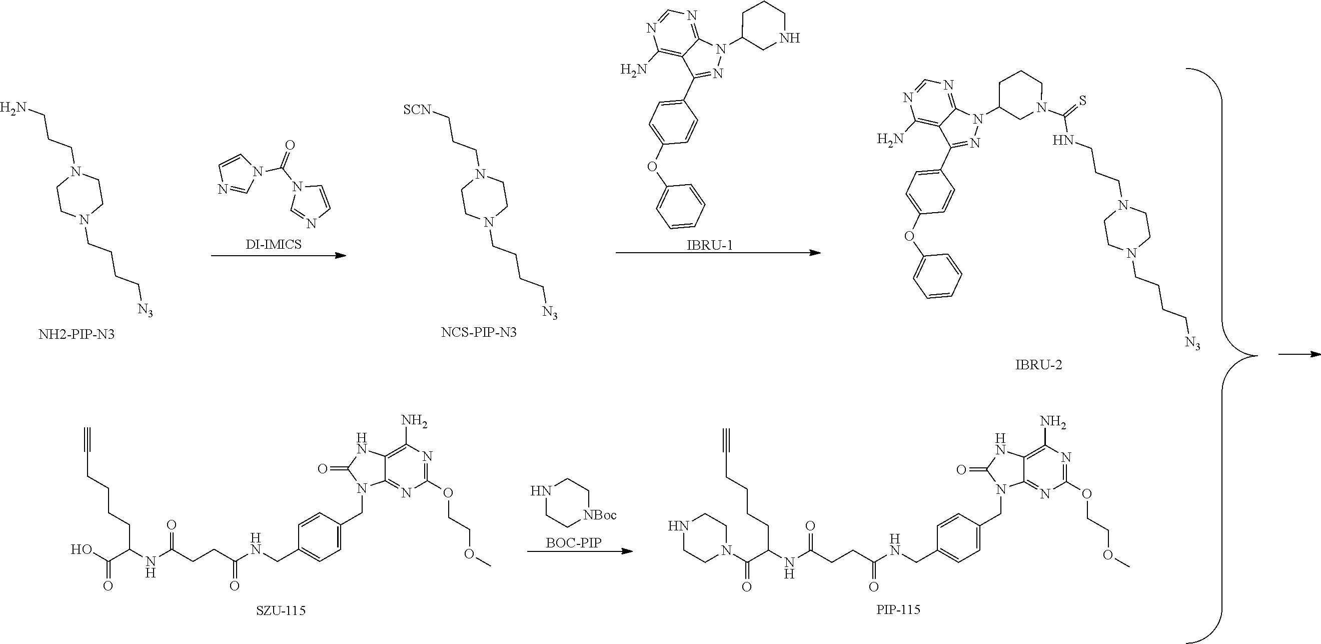

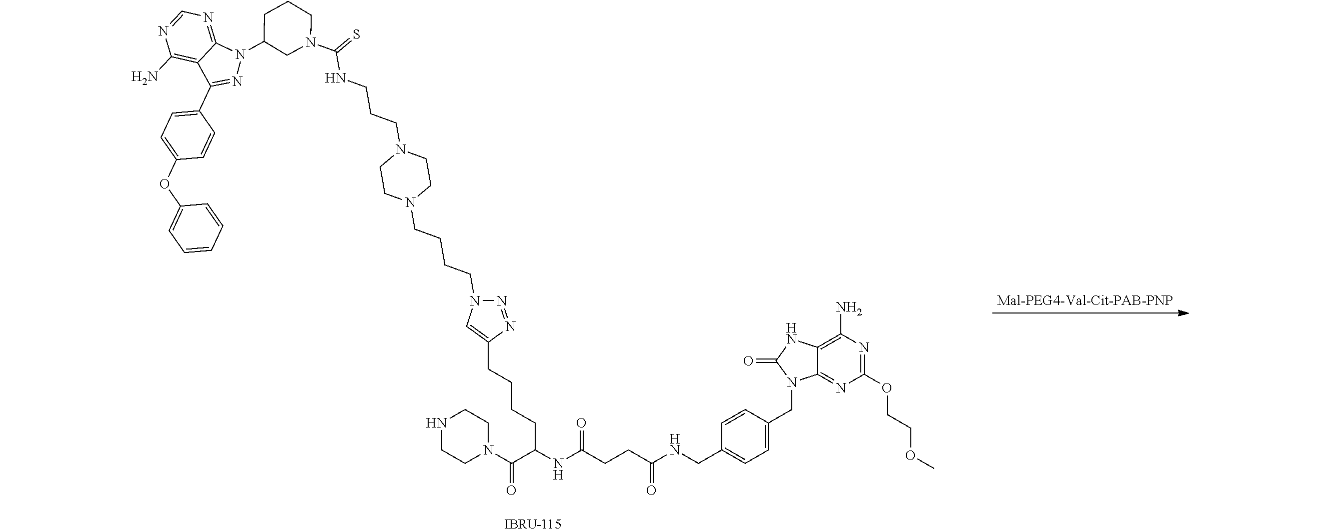 [SMILES: COCCOc1nc(N)c2[nH]c(=O)n(Cc3ccc(CNC(=O)CCC(=O)NC(CCCCc4cn(CCCCN5CCN(CCCNC(=S)N6CCCC(n7nc(-c8ccc(Oc9ccccc9)cc8)c8c(N)ncnc87)C6)CC5)nn4)C(=O)N4CCNCC4)cc3)c2n1]

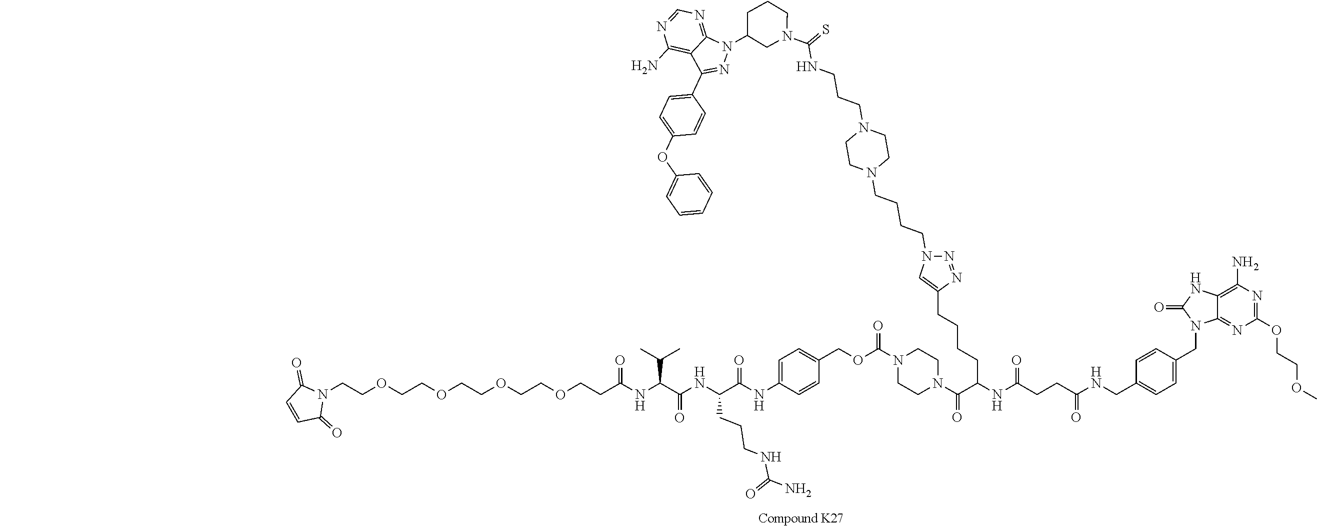 [SMILES: COCCOc1nc(N)c2[nH]c(=O)n(Cc3ccc(CNC(=O)CCC(=O)NC(CCCCc4cn(CCCCN5CCN(CCCNC(=S)N6CCCC(n7nc(-c8ccc(Oc9ccccc9)cc8)c8c(N)ncnc87)C6)CC5)nn4)C(=O)N4CCN(C(=O)OCc5ccc(NC(=O)[C@H](CCCNC(N)=O)NC(=O)[C@@H](NC(=O)CCOCCOCCOCCOCCN6C(=O)C=CC6=O)C(C)C)cc5)CC4)cc3)c2n1]

Compounds NH2-PIP-N3 (120 mg, 0.5 mmol) and DI-IMICS (89 mg, 0.5 mmol) were mixed and dissolved in 2 ml of dry DMSO, and the mixture was stirred at 40° C. for 6 hours. Then IBRU-1 (194 mg, 0.5 mmol) was added, and the mixture was stirred and reacted at room temperature for 6 hours. The reactant was lyophilized and purified by preparative HPLC to give compound IBRU-2 (164 mg, yield 49%), ESI-MS: m/z=669.3[M+H]+.

SZU-115 (291 mg, 0.5 mmol) and PyAOP (261 mg, 0.5 mmol) were taken and dissolved in 3 mL of DMF; DIPEA (88 μL) was added, mixed and stirred for 30 minutes; BOC-PIP (93 mg, 0.5 mmol) was then added; and the mixture reacted at room temperature for 3 hours; the reaction solution was lyophilized, the mixed solvent of TFA/DCM (4 mL, 1:3 by volume) was added and stirred for 1 hour; and the solvent was removed by vacuum distillation, and purified by preparative HPLC to give compound PIP-115 (214 mg, yield 66%), ESI-MS: m/z=650.3 [M+H]+.

IBRU-2 (100 mg, 0.15 mmol) and PIP-115 (98 mg, 0.15 mmol) were dissolved in 3 mL of DMF-H2O (4:1 by volume), CuSO₄·5H₂O (5 mg) and sodium L-ascorbate (5 mg) were added, and the resultant mixture was stirred and reacted at room temperature for 8 hours. The reaction solution was filtered, and the filtrate was lyophilized and purified by preparative HPLC to give compound IBRU-115 (77 mg, yield 39%), ESI-MS: m/z=659.8[(M/2)+H]+.

Compound IBRU-115 (70 mg, 0.053 mmol) was dissolved in 0.5 mL of DMF; Mal-PEG4-Val-Cit-PAB-PNP (46 mg, 0.053 mmol) was dissolved in 0.5 ml, of DMF, and slowly added dropwise to the solution of IBRU-115 at room temperature; after the addition, the mixture was shaken and reacted at room temperature for 6 hours. The reaction solution was lyophilized to remove the solvent, separated and purified by preparative HPLC to give compound K27 (67 mg, yield 62%), ESI-MS: m/z=684.3[(M/3)+H]+.

### Synthesis of Compound K28

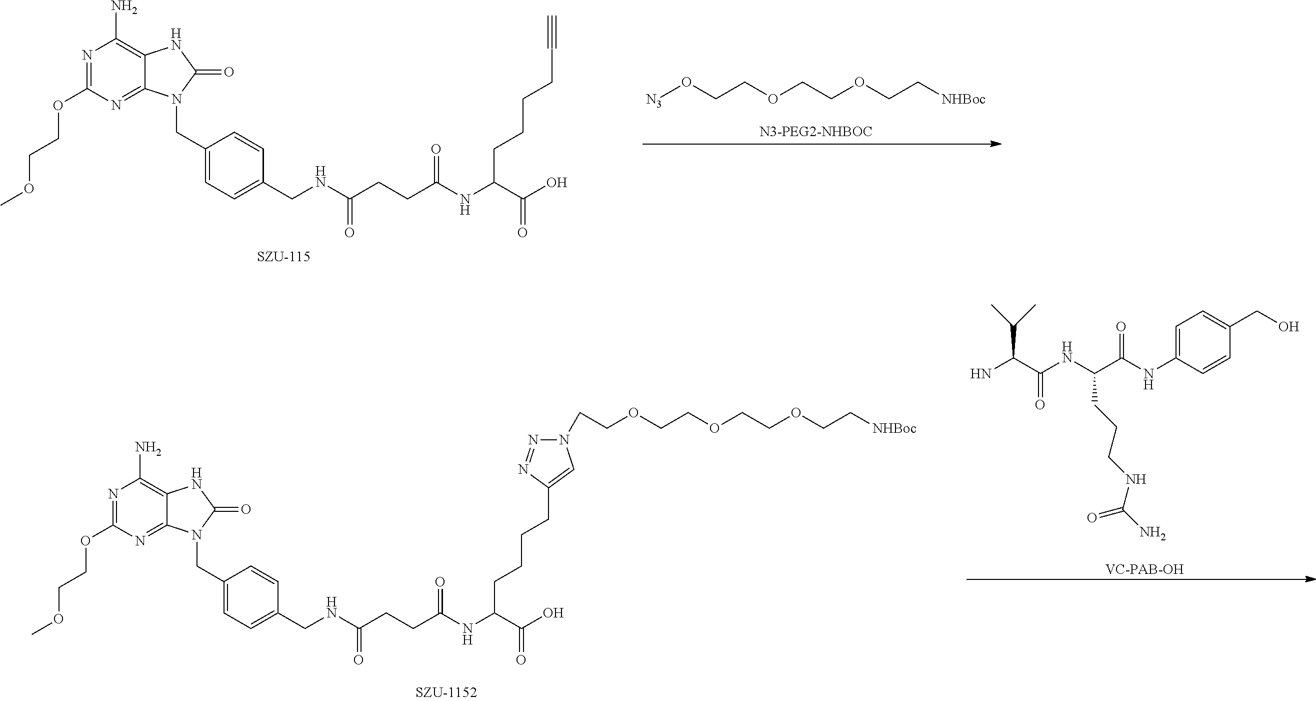

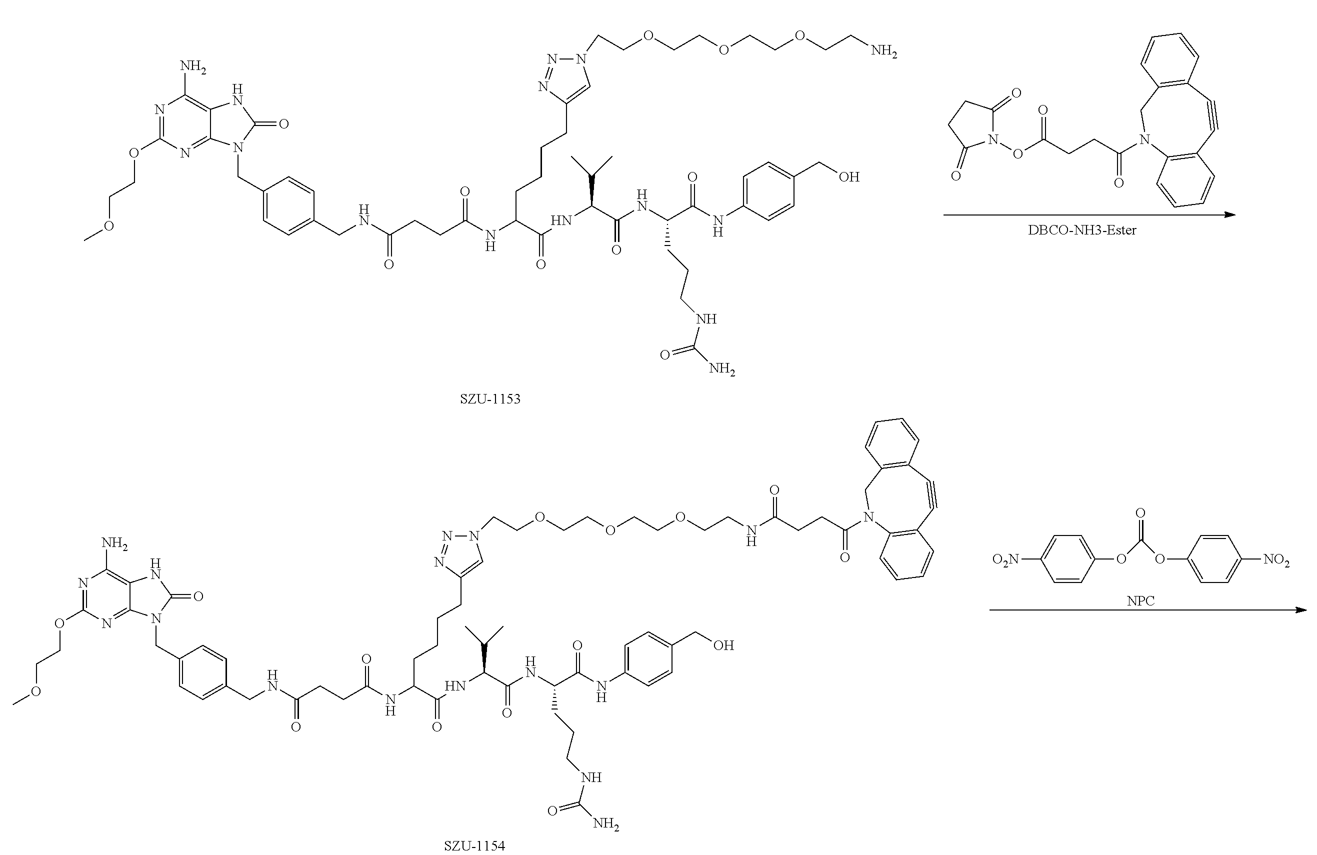

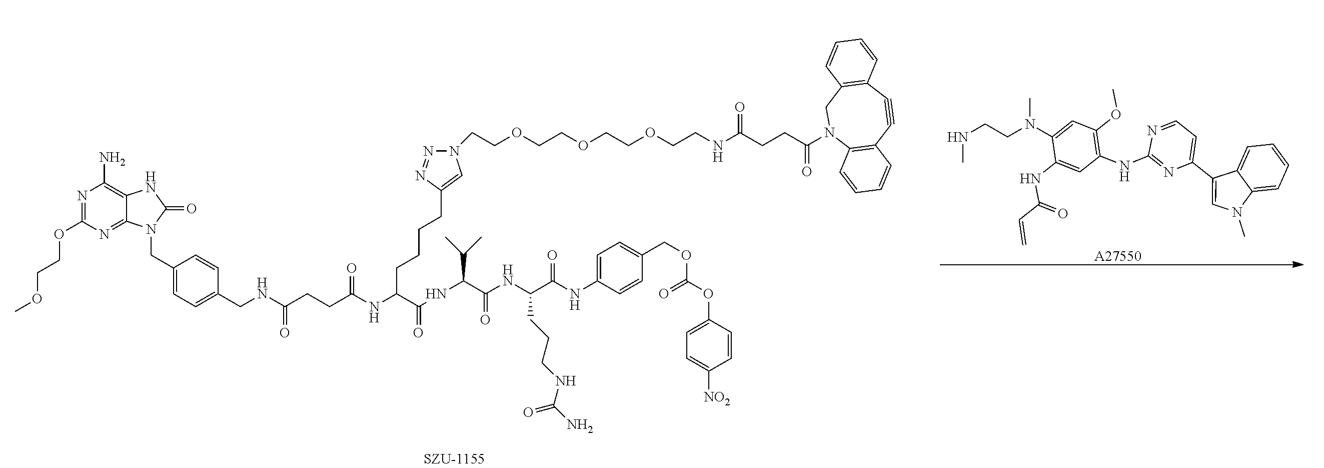 [SMILES: COCCOc1nc(N)c2[nH]c(=O)n(Cc3ccc(CNC(=O)CCC(=O)NC(CCCCc4cn(CCOCCOCCOCCNC(=O)CCC(=O)N5Cc6ccccc6C#Cc6ccccc65)nn4)C(=O)N[C@H](C(=O)N[C@@H](CCCNC(N)=O)C(=O)Nc4ccc(COC(=O)Oc5ccc([N+](=O)[O-])cc5)cc4)C(C)C)cc3)c2n1 ; C=CC(=O)Nc1cc(Nc2nccc(-c3cn(C)c4ccccc34)n2)c(OC)cc1N(C)CCNC ; "A27550"]

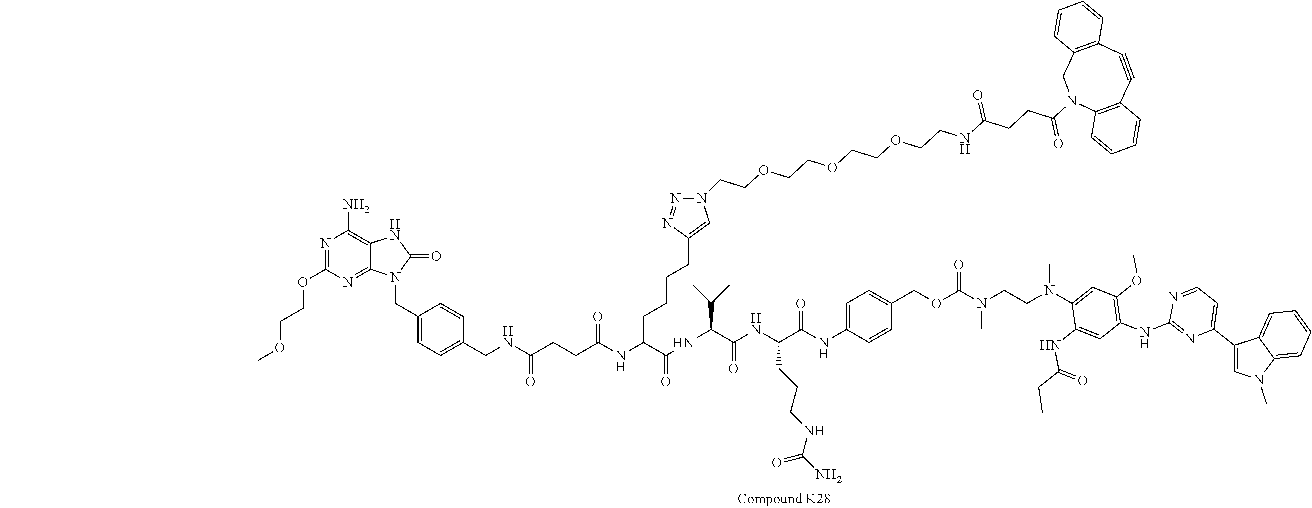

Compound SZU-115 (1 mmol) and compound N3-PEG3-NHBOC (1 mmol) were mixed with CuSo₄·5H₂O (25 mg) and sodium L-ascorbate (25 mg), then dissolved in 10 mL of DMF-H2) (4:1 by volume, 10 mL): the mixture was stirred and subjected to a click reaction at room temperature for 8 hours. The reaction solution was filtered, the solvent was removed by vacuum distillation, and the residue was separated by silica gel column chromatography to give compound SZU-1152 (396 mg, yield 44%, ESI-MS: m/z=900.5[M+H]+). Compound SZU-1152 (300 mg, 0.33 mmol) and DMAP (40 mg) were dissolved in 5 mL of DMF, PyAOP (172 mg, 0.33 mol) was added, and the mixture was stirred and reacted at room temperature for 30 minutes; VC-PAB-OH (125 mg, 0.33 mmol) was then added, and the Mixture was stirred and reacted at room temperature for 6 hours. The reaction solution was lyophilized, the lyophilizate was added with 10 mL of cold water and mixed uniformly, and pH was adjusted to 5 with dilute hydrochloric acid; the resultant solution was extracted with dichloromethane (5×5 mL), and the extraction solution was concentrated under reduced pressure to half its volume; 5 mL of TFA was added, and the mixture was stirred at room temperature for 1 hour. The solvent was removed by vacuum distillation, and the residue was separated and purified by preparative HPLC to give compound SZU-1153 (146 mg, yield 38%), ESI-MS: m/z=1161.6[M+H]+. 1 mL of the solution of compound SZU-1153 (116 mg, 0.1 mmol) and DIPEA (10 μL) in DMF was taken and added with DBCO-NHS-Ester (40 mg, 0.1 mmol), and the mixture was shaken and reacted at room temperature for 3 hours, lyophilized and then purified by preparative HPLC to give compound SZU-1154 (100 mg, 69%). ESI-MS: m/z=724.8[(M/2)+H]+.

0.2 mL of the solution of compound NPC (19 mg, 0.062 mmol) in DMF was taken and slowly added with 1 mL of the solution of SZU-1154 (90 mg, 0.062 mmol), and the mixture was shaken and reacted at room temperature overnight. The reaction solution was lyophilized and purified by preparative HPLC to give compound SZU-1155 (75 mg, yield 75%), ESI-MS: m/z=807.4[(M/2)+H]+. Compound SZU-1155 (60 mg, 0.037 mmol) and DMAP (5 mg) were dissolved in 0.5 mL of DMF, and slowly added with 0.5 mL of the solution of AZ7550 (18 mg, 0.037 mmol) in DMF; after the addition, the mixture was shaken and reacted at room temperature overnight. The reaction solution was lyophilized and purified by preparative HPLC to give compound K28 (39.9 mg, yield 55%), ESI-MS: m/z=980.5[(M/2)+H]+.

### Synthesis of Compound 1K28-1

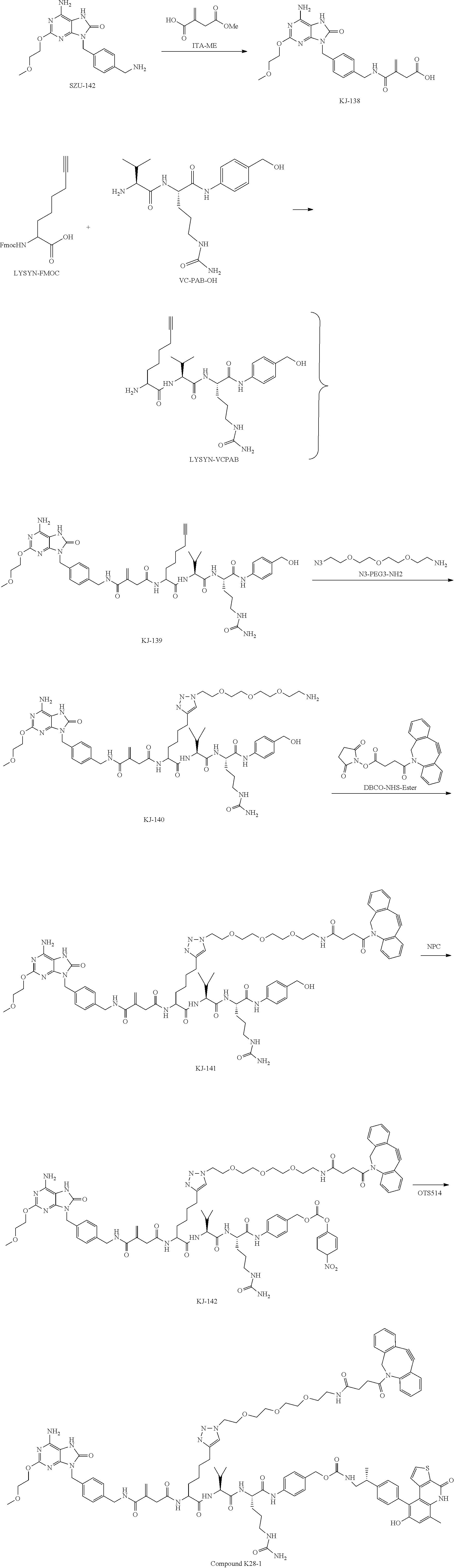

Compounds ITA-ME (1 mmol), PyAOP and DIPEA were dissolved in DMF solvent, and added with SZU-142 (1 mmol) for amidation condensation to give compound KJ-138 (ESI-MS: m/z=457.2[M+H]+). Using the same method, compounds LYSYN-FMOC and VC-PAB-OH underwent condensation reaction to give compound LYSYN-VCPAB (ESI-MS: m/z=517.3[M+H]+). Continuing with the same method, KJ-138 and LYSYN-VCPAB were condensed to give compound KJ-139 (ESI-MS: m/z=955.5[M+H]+). Compounds KJ-139 and N3-PEG3-NH2 underwent a copper-catalyzed Click reaction to give compound KJ-140 (ESI-MS: m/z=1173.6[M+H]+). Equimolar amounts of compounds KJ-140 and DBCO-NHS-Ester were directly reacted in DMF to give compound KJ-141 (ESI-MS: m/z=730.9[(M/2)+H]+). According to the synthesis method of compound SZU-1155, compound KJ-142 can be obtained (ESI-MS: m/z=813.4[(M/2)+H]+). Compound KJ-142 and compound OTS514 were mixed in equimolar amounts in DMF and reacted at room temperature, and after removing DMF by lyophilization, the residue was purified by HPLC to give compound K28-1, ESI-MS: m/z=925.9[(M/2)+H]+.

Synthesis of compound K29: according to the synthesis method of compound K2 except replacing DBCO-PEG4-amine TFA salt with BP-22435 (Methyltetrazine-PEG4-amine HCl salt), compound K29 can be obtained, ESI-MS: m/z=758.9[(M/2)+H]+.

Synthesis of compound K30: according to the synthesis method of compound K2 except replacing DBCO-PEG4-amine TFA salt with TCO-PEG3-amine, compound K30 can be obtained, ESI-MS: m/z=749.4[(M/2)+H]+.

Synthesis of compound K31: according to the synthesis method of compound K28 except replacing DBCO-NHS-Ester with endo-BCN-PEG2-NIHS ester and replacing compound AZ7550 with OTS514, compound K31 can be obtained, ESI-MS: m/z=944.0 [(M/2)+H]+.

### Synthesis of Compound K32

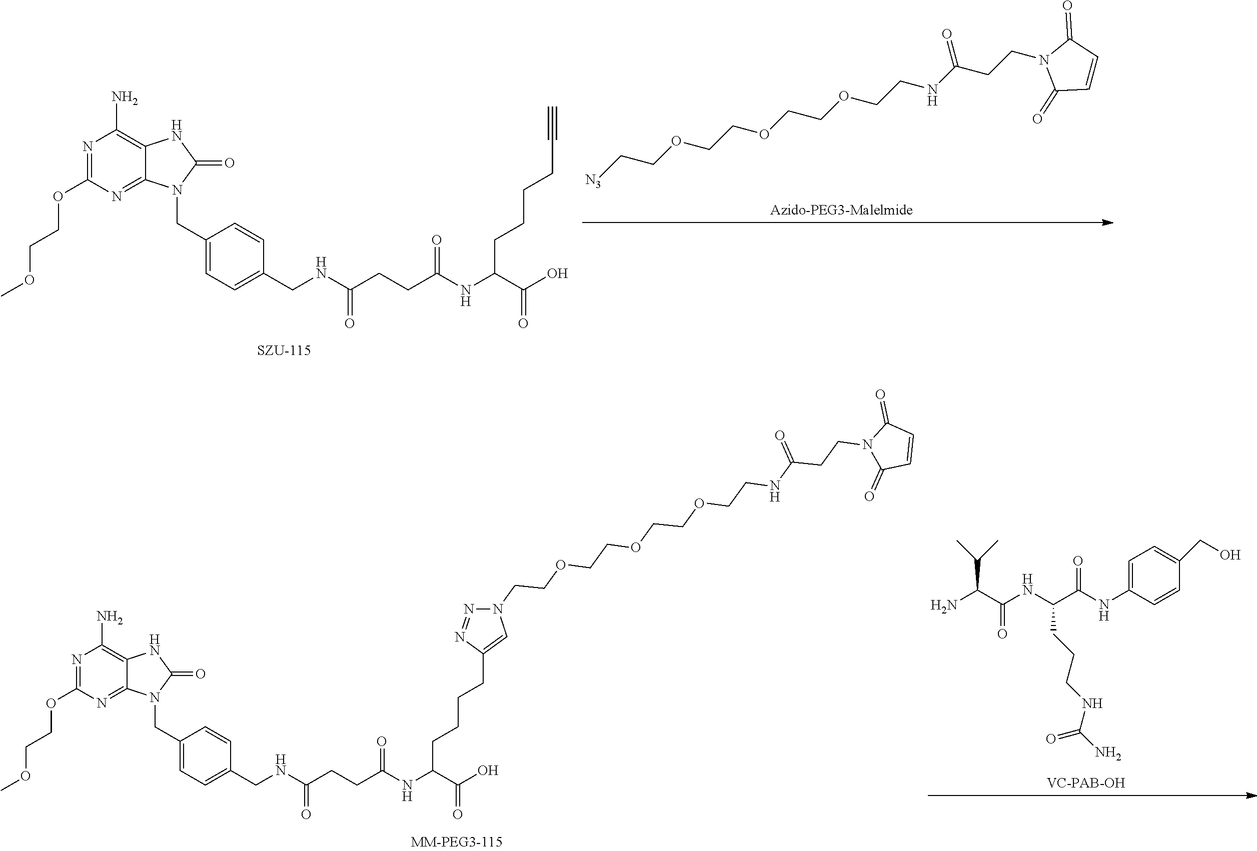 [SMILES: C#CCCCCC(NC(=O)CCC(=O)NCc1ccc(Cn2c(=O)[nH]c3c(N)nc(OCCOC)nc32)cc1)C(=O)O ; [N-]=[N+]=NCCOCCOCCOCCNC(=O)CCN1C(=O)C=CC1=O ; COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)O)cc3)c2n1 ; CC(C)[C@H](N)C(=O)N[C@@H](CCCNC(N)=O)C(=O)Nc1ccc(CO)cc1]

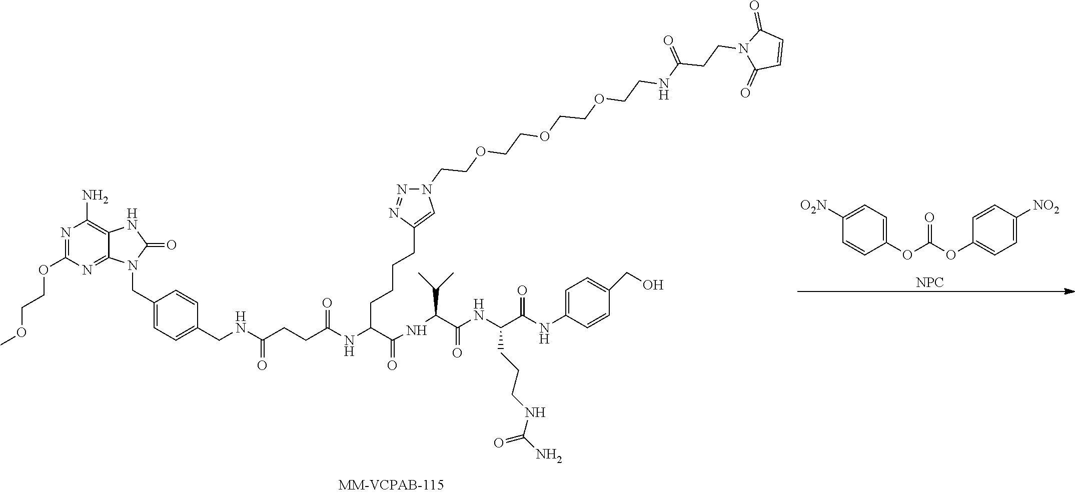

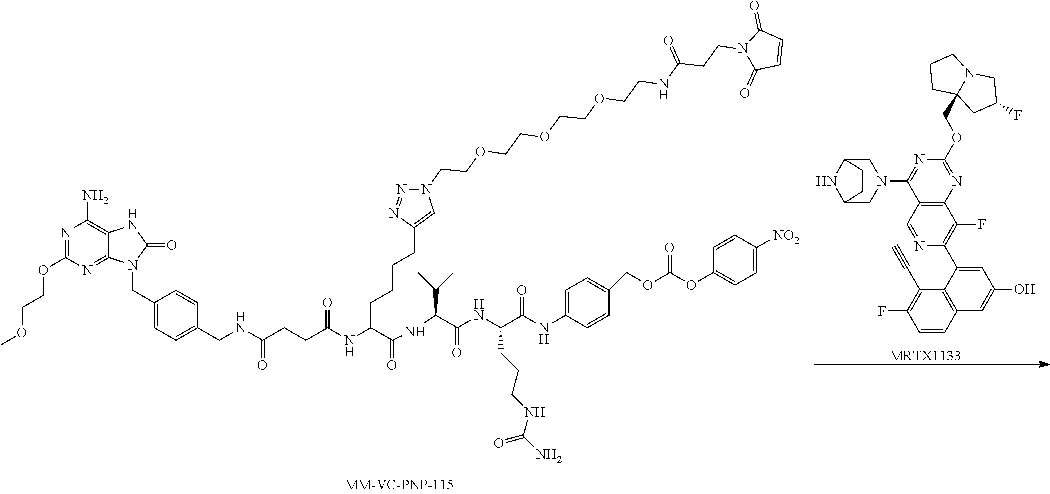

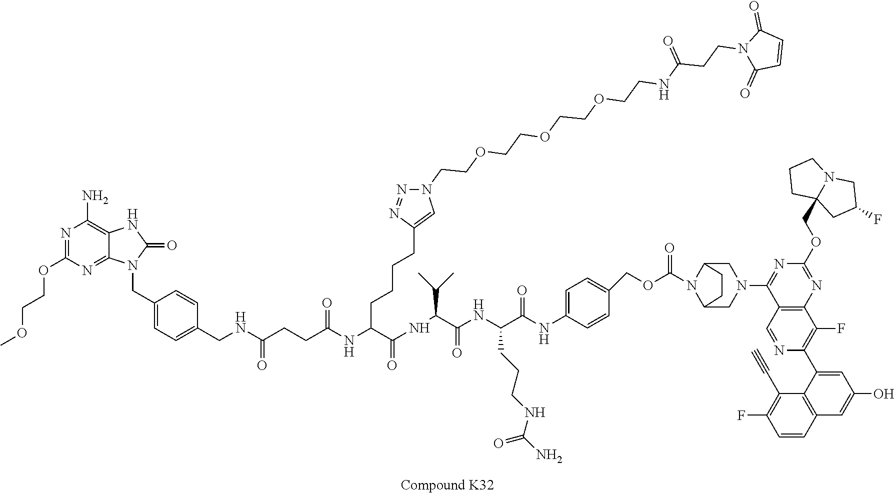 [SMILES: C#Cc1c(F)ccc2cc(O)cc(-c3ncc4c(N5CC6CCC(C5)N6C(=O)OCc5ccc(NC(=O)[C@H](CCCNC(N)=O)NC(=O)[C@@H](NC(=O)C(CCCCc6cn(CCOCCOCCOCCNC(=O)CCN7C(=O)C=CC7=O)nn6)NC(=O)CCC(=O)NCc6ccc(Cn7c(=O)[nH]c8c(N)nc(OCCOC)nc87)cc6)C(C)C)cc5)nc(OC[C@@]56CCCN5C[C@H](F)C6)nc4c3F)c12]

Compound SZU-115 (1 mmol) and compound Azido-PEG3-Maleimide (1 mmol) were mixed in 10 ml of DMF-H2O (4:1 by volume), CuSO₄·5H₂O (25 ng) and sodium L-ascorbate (25 mg) were added, and the resultant mixture was stirred and reacted at room temperature for 6 hours. The mixture was filtered, and the filtrate was lyophilized and purified by preparative HPLC to give compound MM-PEG-3-115 (600 mg, yield 63%), ESI-MS: m/z=951.4[M+H]+. Compound MM-PEG3-115 (190 mg, 0.2 mmol), compound DIPEA (35 μL) and PyAOP (105 mg) were mixed and dissolved in 2 mL of DMF and stirred uniformly for 30 minutes; VC-PAB-OH (76 mg) was added; the mixture was stirred and reacted at room temperature overnight. The reaction solution was lyophilized and purified by preparative HPLC to give compound MM-VCPAB-115 (186 mg, yield 71%), ESI-MS: m/z=6573[(M/2)+H]+.

Compound NPC (31 mg, 0.1 mmol) was dissolved in 0.5 mL of anhydrous DMF; compound MM-VCPAB-115 (132 mg, 0.1 mmol) and DMAP (12 mg) were taken, mixed and dissolved in 1 mL of DMF, and slowly added to the solution of NPC in DMF; after the addition, the mixture was stirred and reacted at room temperature overnight. The reaction solution was lyophilized, separated and purified by silica gel column chromatography (ethyl acetate) to give compound MM-VC-PNP-115 (72 mg, yield 49%). ESI-MS: m/z=739.3[(M/2)+H]+.

Compound MM-VC-PNP-115 (59 mg, 0.04 mmol) and DMAP (5 mg) were dissolved in 1 ml, of dry DMF, MRTX1133 (24 mg, 0.04 mmol) was added, and the mixture was stirred and reacted at room temperature overnight. The reaction solution was lyophilized, the residue was dissolved in a small amount of methanol and purified by semi-preparative HPLC to give compound K32 (45 mg, yield 58%), ESI-MS: m/z=970.0[(M/2)+H]+.

Synthesis of compound K33: compound K33 can be obtained using the same method as that for the synthesis of K32, ESI-MS: m/z=986.4[(M/2)+H]+.

### Synthesis of Compound K34

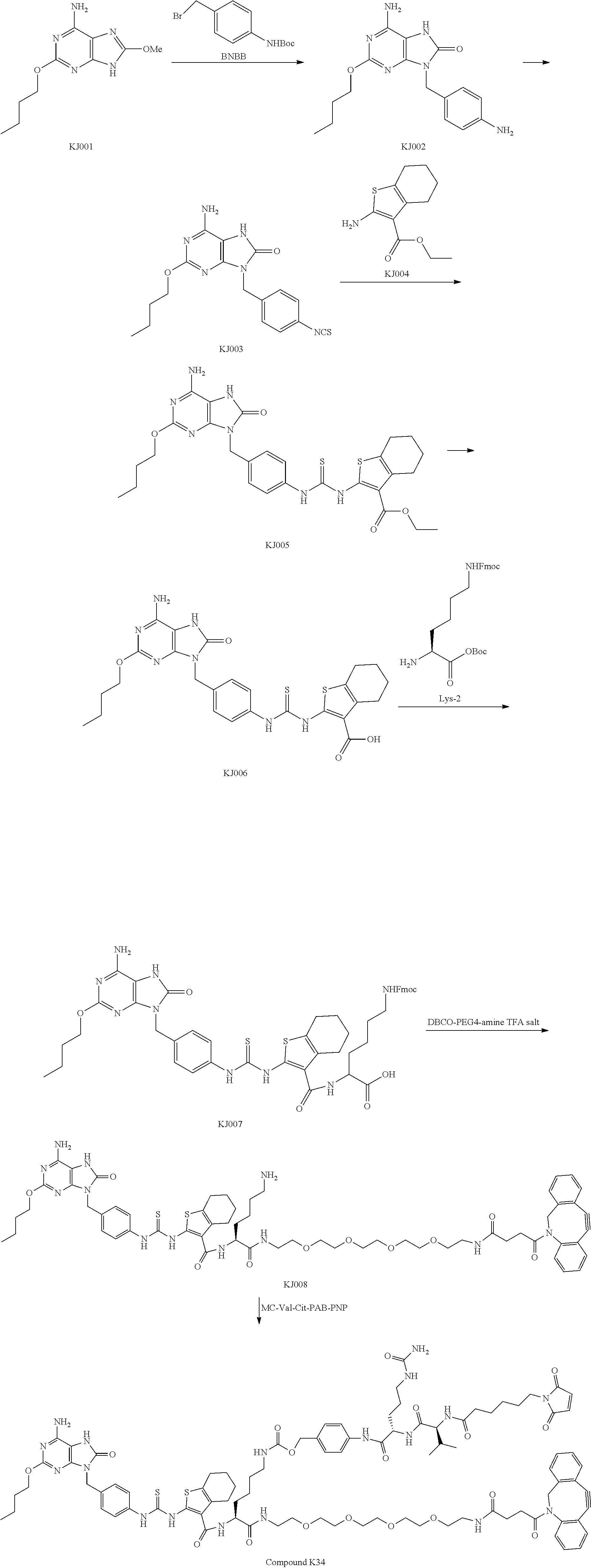

Compound KJ001 (120 mg, 0.5 mmol) and compound BBNB (143 mg, 0.5 mmol) were mixed and dissolved in 3 mL of DMF, K₂CO₃ (100 mg) was added, and the mixture was stirred and reacted at room temperature for 8 hours. The reaction solution was filtered, and the filtrate was subjected to vacuum distillation to remove the solvent and added with 5 mL of concentrated hydrochloric acid; the mixture was stirred at room temperature for 3 hours and neutralized to pH 8 with Na₂CO₃, to precipitate a solid product; the solid product was dried under vacuum to give KJ002 (123 mg, yield 75%), ESI-MS: m/z=329.2[M+H]+. Compound KJ002 (100 mg, 0.3 mmol) was taken and dissolved in 2 mL of chloroform solvent, triethylamine (100 μL) was added, and under cooling in an external bath at 0° C., 0.5 mL of solution of thiophosgene (25 μL) in chloroform was slowly added; after the addition, the mixture was stirred and reacted at room temperature overnight. After cold water was added for sufficient washing (3×2 mL), the chloroform was separated, dried over anhydrous sodium sulfate and filtered, and the filtrate was subjected to vacuum distillation to remove the solvent to give compound KJ003 (71 mg, yield 64%), ESI-MS: m/z=371.1[M+H]+. Compound KJ003 (60 mg, 0.16 mmol) and compound KJ004 (36 mg, 0.16 mmol) were taken and dissolved in 0.5 mL of DMF, triethylamine (60 μL) was added, and the mixture was shaken and reacted at room temperature overnight. The reaction solution was lyophilized, and the lyophilizate was separated and purified by HPLC to give compound KJ005 (63 mg, yield 66%), ESI-MS: m/z=596.1[M+H]+. Compound KJ005 (60 mg) was taken and mixed with 1 mL of concentrated hydrochloric acid, and the mixture was stirred at room temperature overnight; the reaction solution was neutralized to pH 4 with Na₂CO₃ and filtered to give a solid, and solid was washed with pure water three times and then dried under vacuum to give compound KJ006 (51 mg, yield 89%). ESI-MS: m/z=568.1 [M+H]+.

According to the synthesis method of compound 101-Lys-2, compound KJ007 can be obtained (ESI-MS: m/z=918.3[M+H]+).

According to the synthesis method of compound 101-Lys-DBCO, compound KJ008 can be obtained (ESI-MS: m/z=1201.5[M+H]+).

In the final step, according to the synthesis method of compound K2, KJ008 and MC-Val-Cit-PAB-PNP reacted to give compound K34 {ESI-MS: m/z=900.4 [(M/2)+H]+}.

### Synthesis of Compound K35

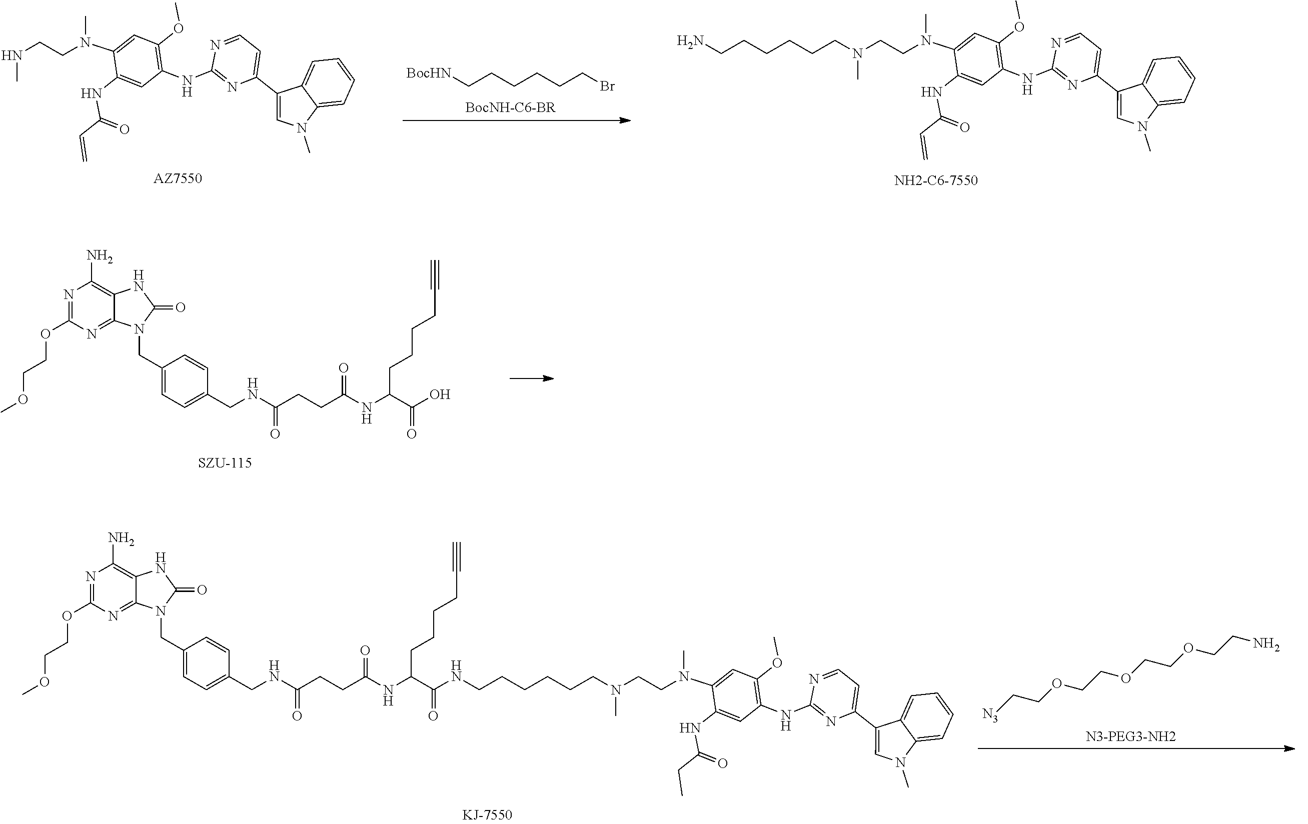 [SMILES: C=CC(=O)Nc1cc(Nc2nccc(-c3cn(C)c4ccccc34)n2)c(OC)cc1N(C)CCNC ; CC(C)(C)OC(=O)NCCCCCCBr ; C=CC(=O)Nc1cc(Nc2nccc(-c3cn(C)c4ccccc34)n2)c(OC)cc1N(C)CCN(C)CCCCCCN ; C#CCCCCC(NC(=O)CCC(=O)NCc1ccc(Cn2c(=O)[nH]c3c(N)nc(OCCOC)nc32)cc1)C(=O)O ; C#CCCCCC(NC(=O)CCC(=O)NCc1ccc(Cn2c(=O)[nH]c3c(N)nc(OCCOC)nc32)cc1)C(=O)NCCCCCCN(C)CCN(C)c1cc(OC)c(Nc2nccc(-c3cn(C)c4ccccc34)n2)cc1NC(=O)CC ; [N-]=[N+]=NCCOCCOCCOCCN]

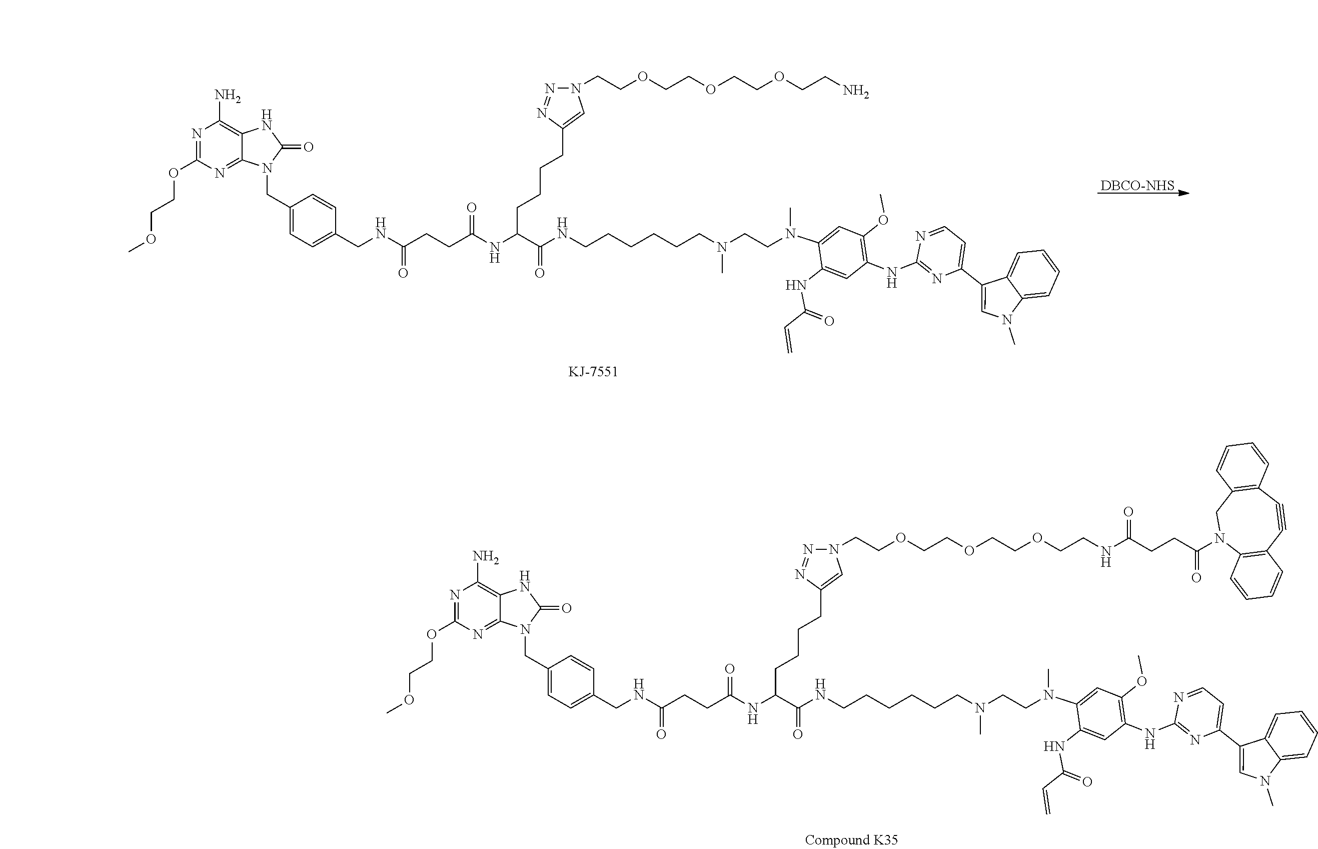 [SMILES: C=CC(=O)Nc1cc(Nc2nccc(-c3cn(C)c4ccccc34)n2)c(OC)cc1N(C)CCN(C)CCCCCCNC(=O)C(CCCCc1cn(CCOCCOCCOCCN)nn1)NC(=O)CCC(=O)NCc1ccc(Cn2c(=O)[nH]c3c(N)nc(OCCOC)nc32)cc1 ; "DBCO-NHS" ; C=CC(=O)Nc1cc(Nc2nccc(-c3cn(C)c4ccccc34)n2)c(OC)cc1N(C)CCN(C)CCCCCCNC(=O)C(CCCCc1cn(CCOCCOCCOCCNC(=O)CCC(=O)N2Cc3ccccc3C#Cc3ccccc32)nn1)NC(=O)CCC(=O)NCc1ccc(Cn2c(=O)[nH]c3c(N)nc(OCCOC)nc32)cc1]

Compound AZ7550 was dissolved in DMF, and one equivalent of anhydrous K₂CO₃ was added; an equimolar amount of BocNH-C6-Br was dissolved in an appropriate amount of DMF, and slowly added to the solution of AZ7550 in DMF at room temperature (over approximately 1 hour); after the addition, the mixture reacted at room temperature for 6 hours. The reaction solution was filtered and lyophilized, and the lyophilizate was added to 1:3 TFA/DCM mixed solvent; and the mixture was stirred at room temperature for 1 bour, then the solvent, was removed by vacuum distillation, and the residue was dissolved in an appropriate amount of pure water; the reaction solution was adjusted to pH 9 with saturated sodium carbonate solution, and extracted with an equal volume of dichloromethane 3 times; the combined extraction solution were dried over anhydrous K₂CO₃ and filtered; the filtrate was subjected to vacuum distillation to give a solid, and the solid was dried under vacuum to give compound NH2-C6-7550 (ESI-MS: m/z=585.4[M+H]+).

Compound SZU-115 (120 mg, 0.2 mmol) and DIPEA (35 μL) were Mixed in 2 mL of DMF, PyAOP (105 mg, 0.2 mmol) was added and mixed, and the mixture was stirred for 0.5 hours; NH2-C6-7550 (117 mg, 0.2 mmol) was added; and the mixture reacted at room temperature for 6 hours. The reaction solution was lyophilized and purified by preparative HPLC to give compound KJ-7550 (144.7 mg, yield 63%), ESI-MS: m/z=1148.6[M+H]+.

Compound KJ-7550 (100 mg, 0.087 mmol) and compound N3-PEG3-NH2 (20 mg, 0.087 mmol) were mixed and dissolved in DMF-H2O (4:1 by volume, 2 mL), CuSO₄·5H₂O (5 mg) and sodium L-ascorbate (5 mg) were added, and the resultant mixture was stirred and reacted at room temperature overnight. The reaction solution was filtered and lyophilized, the lyophilizate was separated and purified by HPLC to give the purified solution containing the product, and the purified solution was concentrated under reduced pressure and lyophilized to give compound KJ-7551 (50 mg, yield 42%), ESI-MS: m/z=683.9[(M/2)+H]+.

Compound KJ-7551 (45 mg, 0.033 mmol) was taken and dissolved in 0.5 mL of DMF, DIPEA (10 μL) and DBCO-NHS (14 mg, 0.035 mmol) were added, and the mixture was shaken and reacted at room temperature for 3 hours. The reaction solution was purified by semi-preparative HPLC to give a product solution, and the product solution was lyophilized to give compound K35 (38.8 mg, yield 71%), ESI-MS: m/z=827.4[(M/2)+H]+.

The synthesis of compound K36 was performed according to the following synthesis route: according to the synthesis method and reaction conditions of compound K35 except simply replacing SZU-115 with 102-N3, compound KJ-7552 can be obtained (ESI-MS: m/z=608.3[(M/2)+H]+); by replacing N3-PEG3-MC with Propargyl-PEG4-anine, compound KJ-7553 can be obtained (ESI-MS: m/z=723.9[(M/2)+H]+); then K1-7553 and DBCO-NHS reacted to give compound K36 (ESI-MS: n/z: 867.5[(M/2)+H]+).

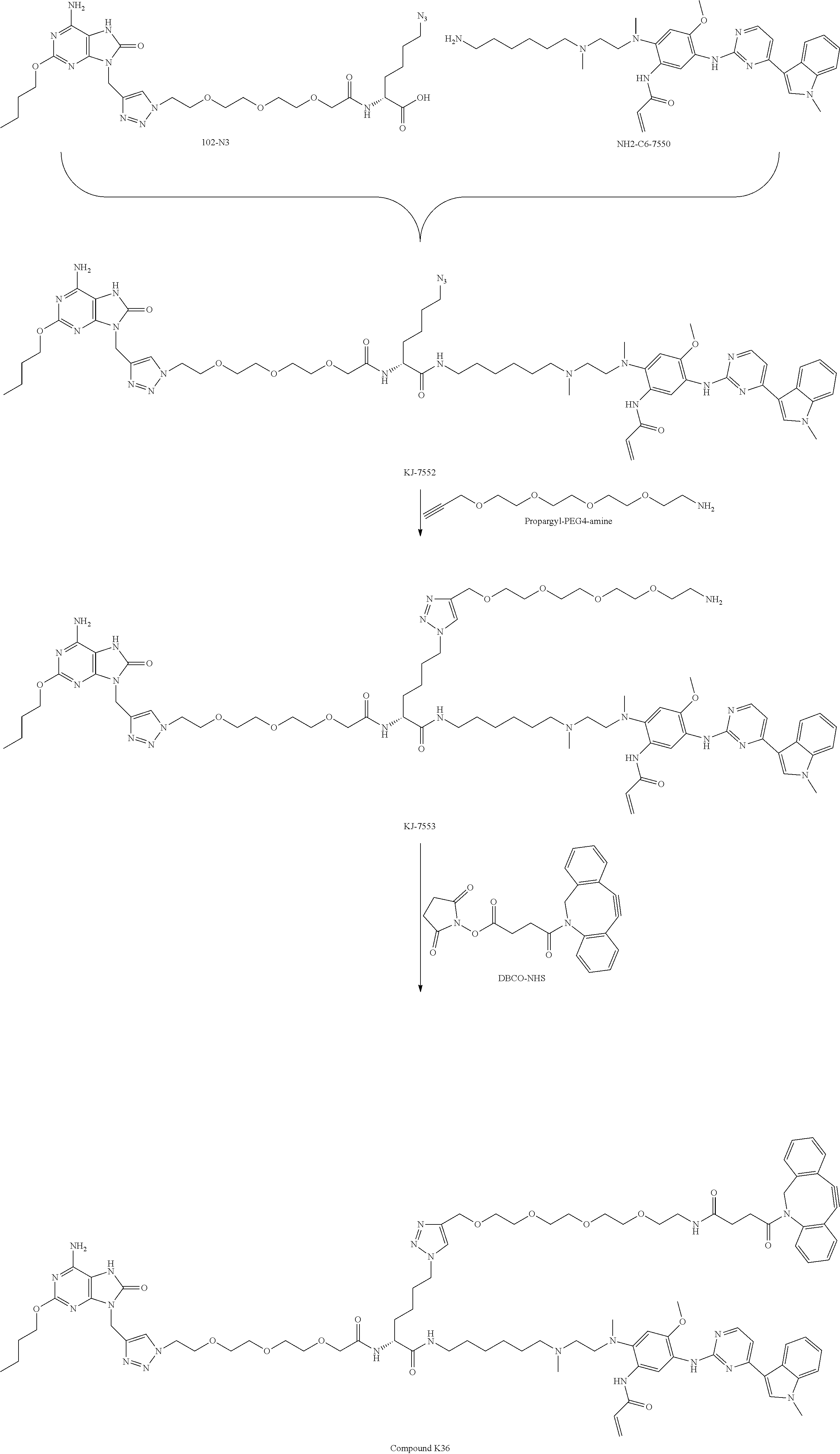

The synthesis of compound K37 was performed according to the following synthesis route: according to the synthesis method and reaction conditions of compound K2 except simply replacing DBCO-PEG4-amine TFA salt with NH2-C6-7550, compound KJ-7554 can be obtained (ESI-MS: m/z=570.3 [(M/2)+H]+); by replacing MC-Val-Cit-PA B-PNP with DBCO-PEG4-Val-Cit-PAB-PNP, compound K37 can be obtained (ESI-MS: m/z=: 693.7[(M/3)+H]+).

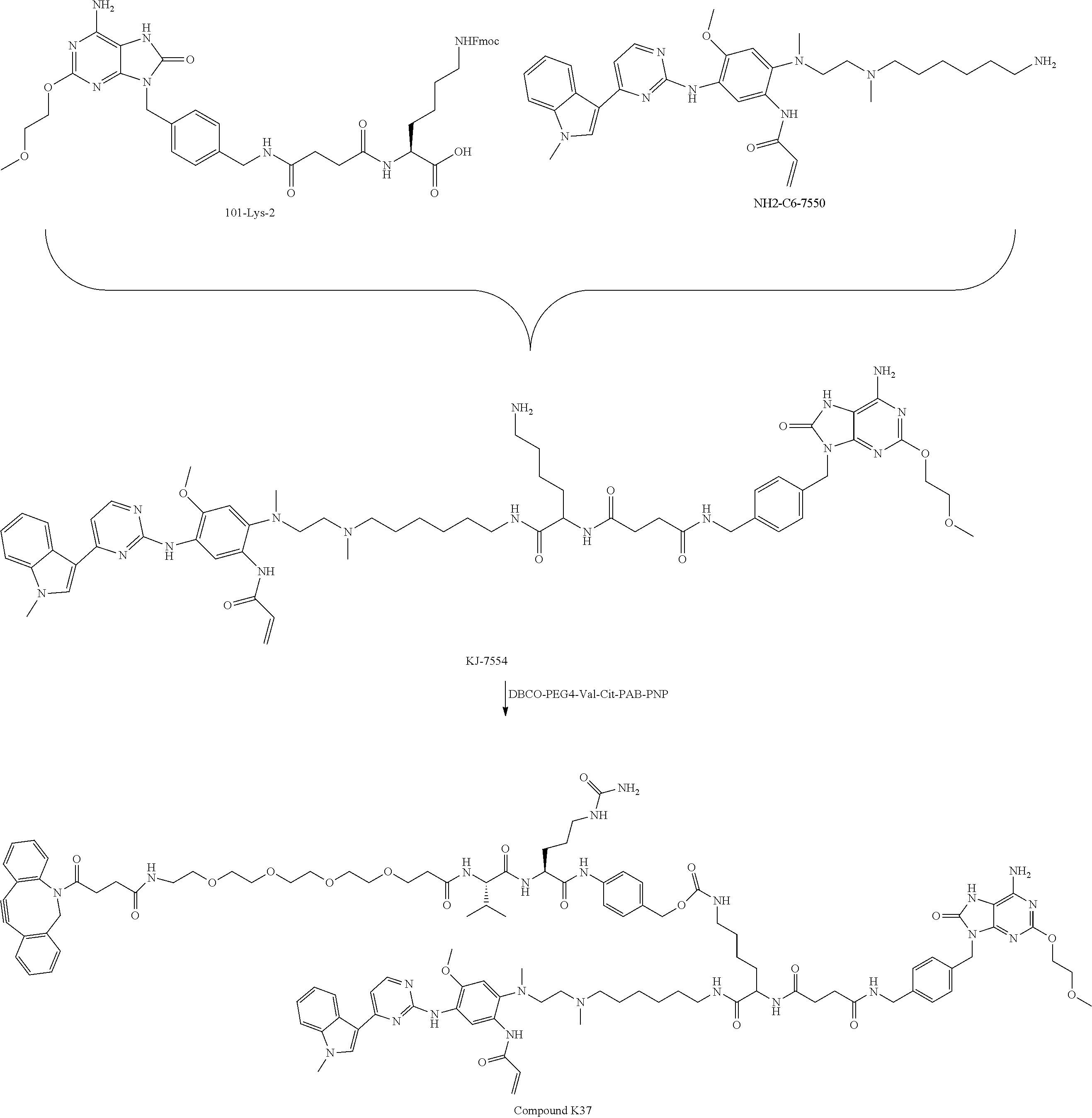

### Synthesis of Compound K38

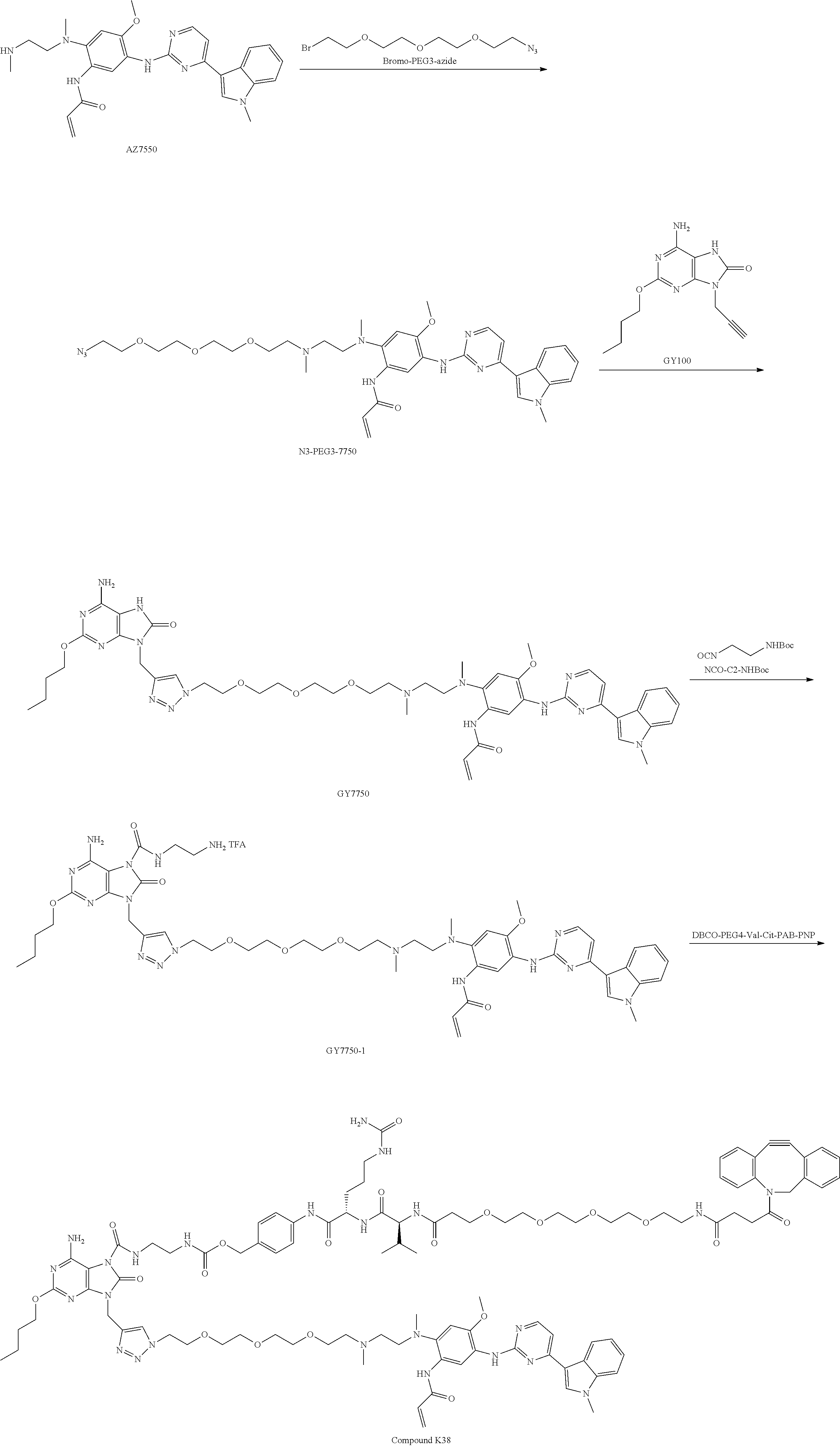

Compound AZ7550 (98 mg, 0.2 mmol) was dissolved in 0.5 mL of DMF; compound bromo-PEG3-azide (57 mg, 0.2 mmol) was dissolved in another 1 mL of DMF, and slowly added into the solution of AZ7550 with stirring. After the addition, the mixture was stirred and reacted at room temperature overnight. The reaction solution was lyophilized and purified by HPLC to give a purified solution, and the purified solution was lyophilized to give compound N3-PEG3-7550 (90.6 mg, 669% yield), ESI-MS: m/z=687.4[M+H]⁺.

Compound N3-PEG3-7550 (83 mg, 0.12 mmol) and compound GY100 (32 mg, 0.12 mmol) were dissolved together in 2 mL of DMF-H2O (4:1 volume), CuSO₄·5H₂O (5 mg) and sodiumL-ascorbate (5 mg) were added, and the resultant mixture was stirred and reacted at room temperature overnight. The reaction solution was filtered, the filtrate was lyophilized and purified by HPLC to give a product solution, and the product solution was lyophilized to give compound GY7550 (66.5 mg, 53%) yield), ESI-MS: m/z=948.5 [M+H]⁺.

Compound GY7550 (60 mg, 0.057 mmol) and DBU (23 μL) were dissolved in 1 mL of dry DMF, NCO-C2-NHBOC (12 μL, 0.07 mmol) was added, and the mixture was reacted at room temperature overnight. The reaction solution was lyophilized, TFA/DCM (1:3 volume, 1 mL) was added, and the mixture was stirred and reacted at room temperature for 1 hour. The solvent was removed by vacuum distillation, the residue was purified by HPLC to give a purified product, and the purified product was lyophilized to give compound GY7550-1 (33.5 mg, 52% yield), ESI-MS: m/z=1034.5[M+H]⁺.

Compound GY7550-1 (30 mg, 0.0265 mmol) and DBCO-PEG4-Val-Cit-PAB-PNP (30 mg, 0.0278 mmol) were dissolved in 1 mL of DMF, the mixture was stirred to mix uniformly, and then DMAP (6 mg) was added; and the resultant reaction solution was shaken and reacted at room temperature overnight. The reaction solution was lyophilized to remove the solvent, the lyophilizate was separated and purified by semi-preparative HPLC, and the purified solution containing the product was lyophilized to give compound K38 (37 mg, 71% yield), ESI-MS: m/z=987.5[(M/2)+H]⁺.

### Synthesis of Compound K39

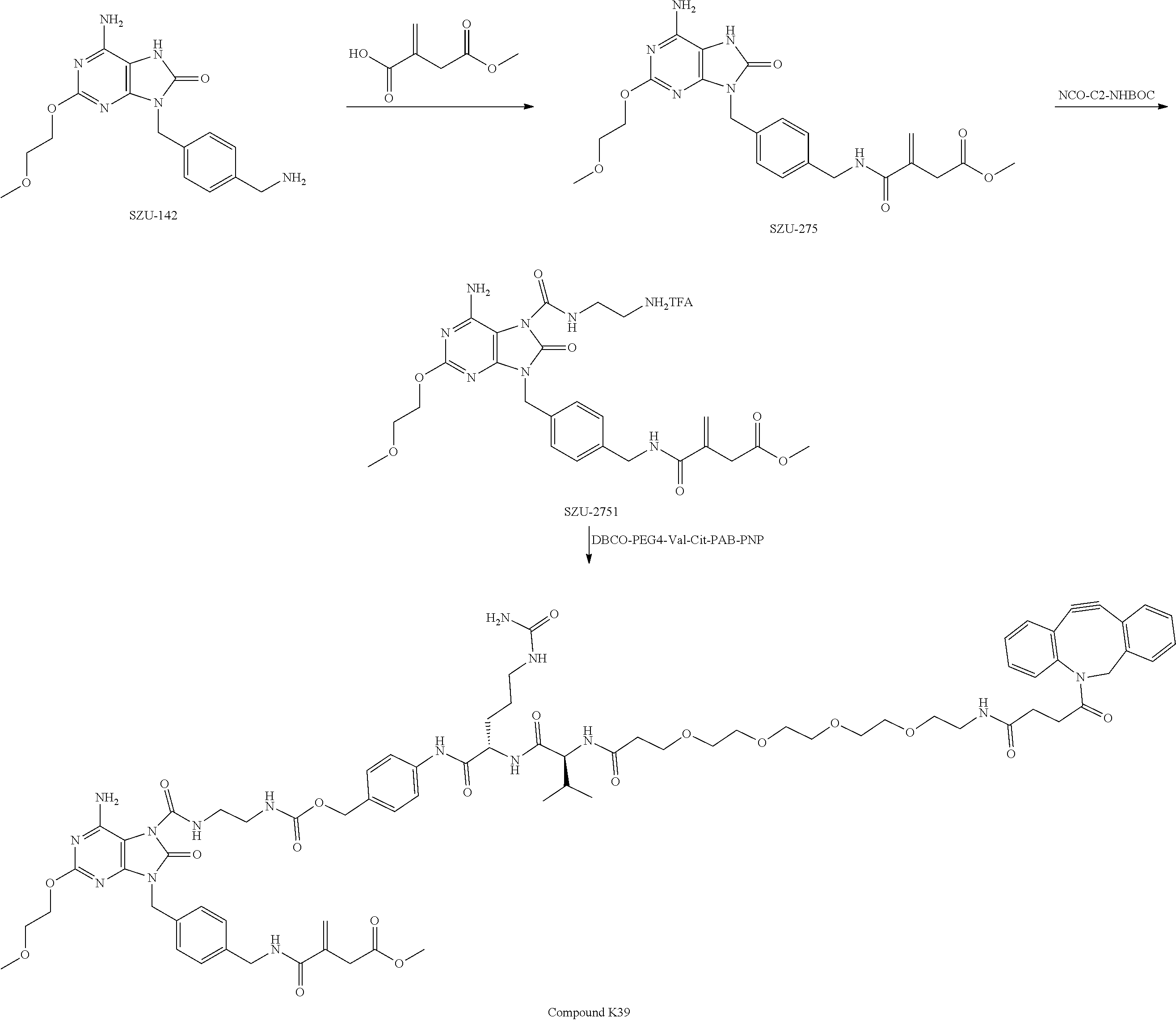

SZU-142 and monomethyl itaconate in equimolar ratio was subject to a condensation reaction under the action of PyAOP and DIPEA, to give compound SZU-275 (ESI-MS: m/z=471.2[M+H]+). According to the synthesis methods of GY7550-1 and compound K38, compound K39 can be obtained, ESI-MS: m/z=748.8[(M/2)+H]⁺.

### Synthesis of Compound K40

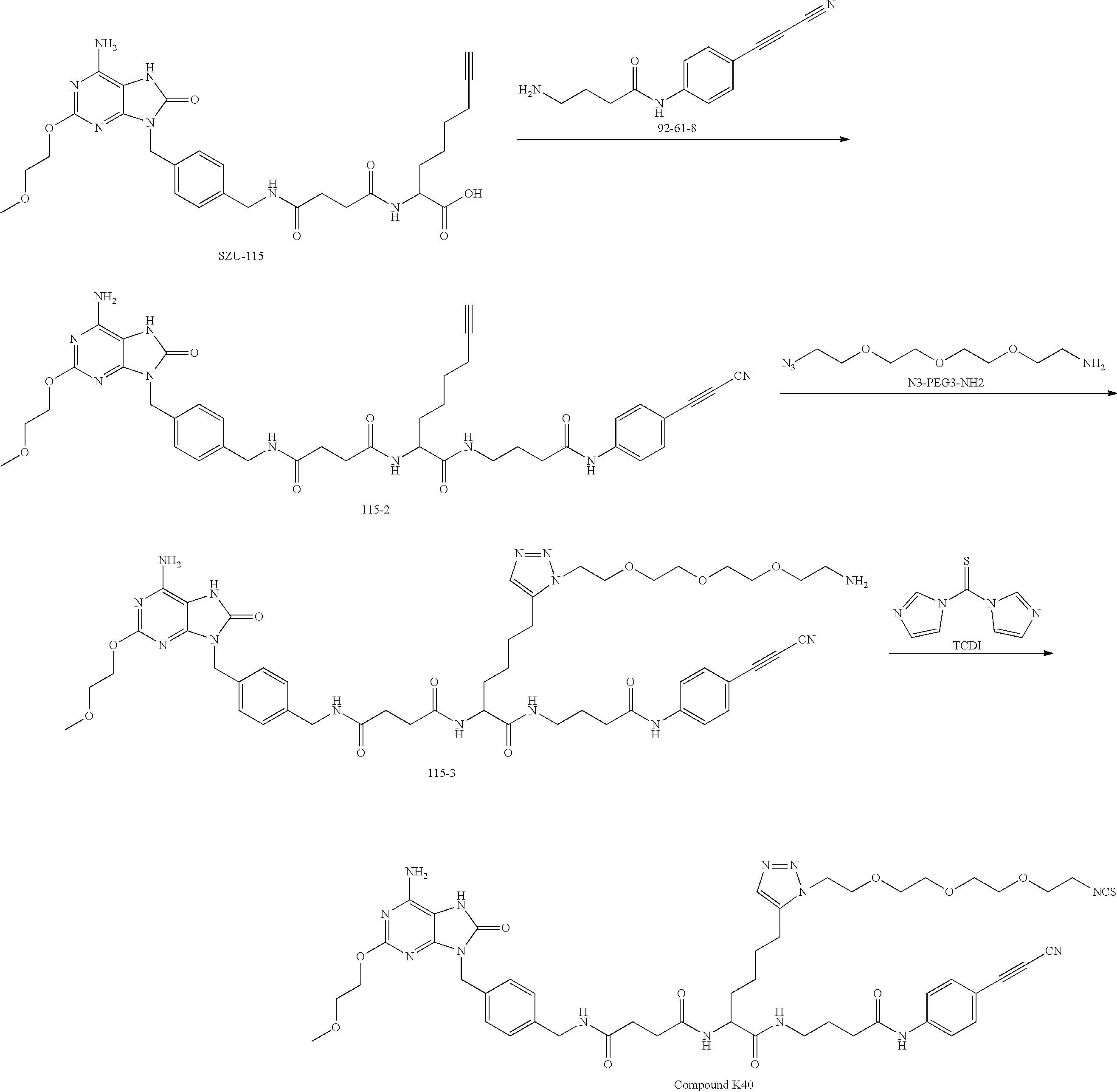

Compound SZU-115 (111 mg, 0.19 mmol) and compound PyAOP (99 mg, 0.19 mmol) were dissolved in 3 mL of DMF DIPEA (36 μL) was added and mixed uniformly, and the mixture was stirred for 30 minutes. 0.2 mL, of the solution of compound 92-61-9 (44 mg, 0.19 mmol) in DMF was added, and the mixture was stirred and reacted at room temperature overnight. The reaction solution was lyophilized and purified by HPLC to give compound 115-2 (95 mg, 63% yield), ESI-MS: m/z=791.3[M+H]+.

Compound 115-2 (90 mg) and N3-PEG3-NH2 (25 mg) were taken and dissolved in 2 ml of H2O-DMF (1:3), CuSO₄·5H₂O (10 mg) and sodium L-ascorbate (10 mg) were added and mixed, and the mixture was stirred and reacted for 8 hours. The reaction solution was filtered, and the filtrate was lyophilized and purified by HPLC to give compound 115-3 (79 mg, 690 yield), ESI-MS: m/z=1009.5[M+H]⁺.

Compound 115-3 (75 mg, 0.075 mmol) was dissolved in 1 mL of N-methylpyrrolidone, TCDI (13.5 mg) was added, and the mixture reacted at room temperature ovemnight. The reaction solution was lyophilized and purified by HPLC to give compound K40 (59 mg, 75% yield), ESI-MS: m/z=1051.5[M+H]⁺.

### Synthesis of Compound K41

According to the synthesis method of compound K16 except replacing KJ-008 with compound KJ-140, compound K41 can be obtained, ESI-MS: m/z=775.4[M+H]1. Compound KJ-140 was synthesized by the click reaction of compound GY121 and N3-PEG3-MC (ESI-MS: m/z=659.3[M+H]⁺):

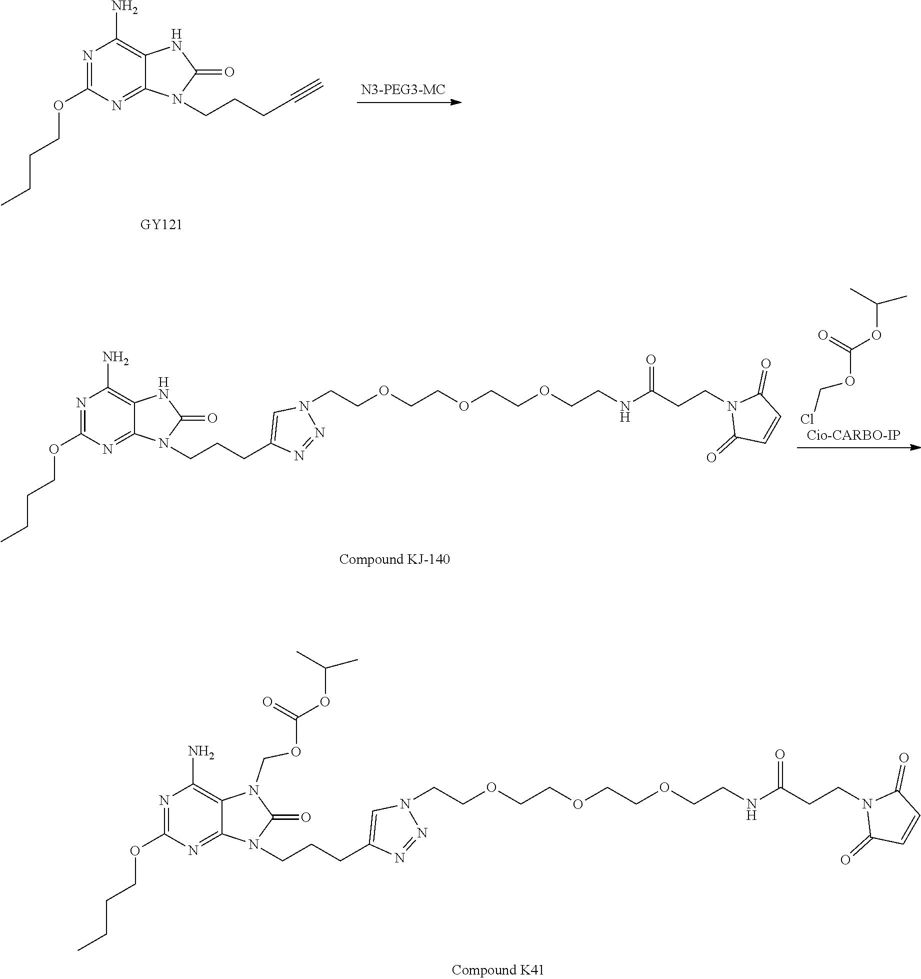

### Synthesis of Compound K42

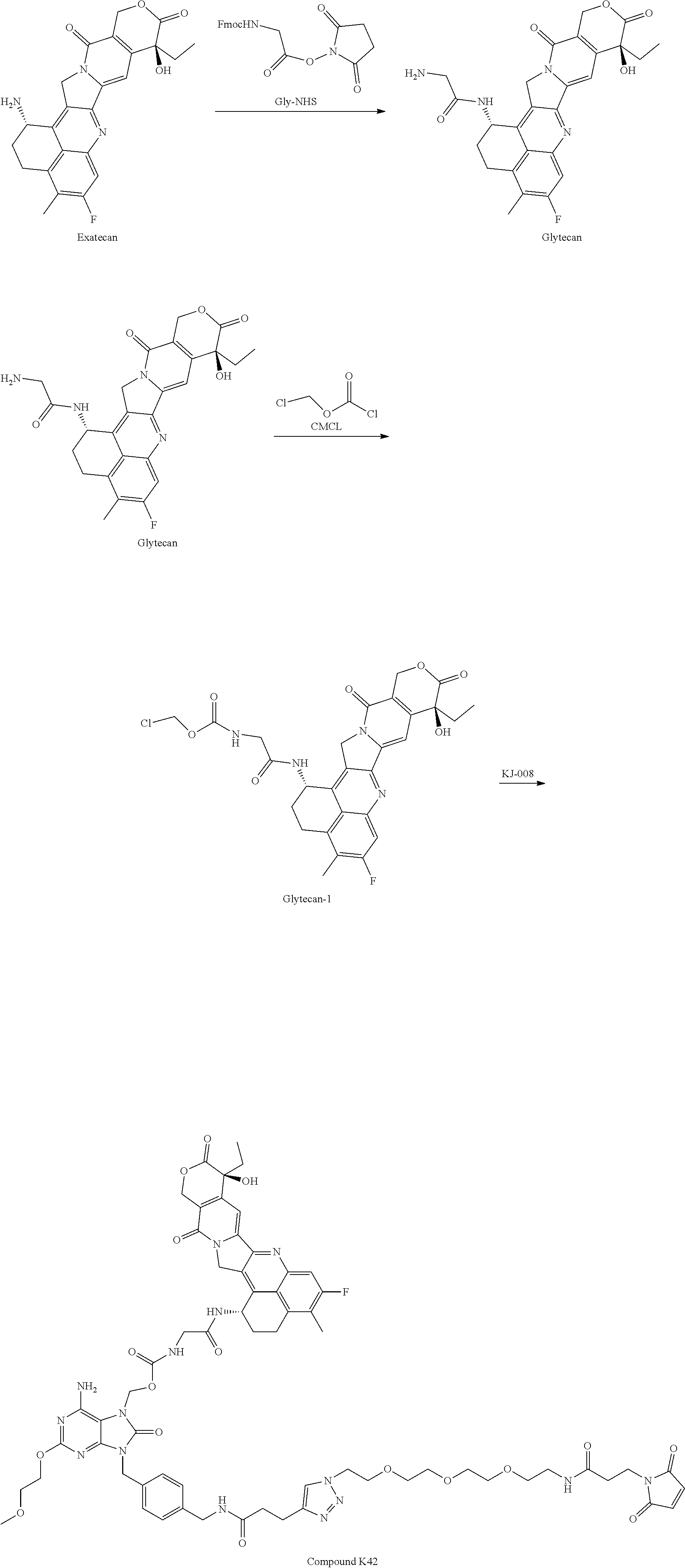

Equimolar amounts of compound Exatecan and compound Gly-NHS were dissolved together in an appropriate amount of DMF, one equivalent of DIPEA were added, and the mixture was stirred and reacted until reaction completion. Then one equivalent of piperidine was added, and the mixture was stirred at room temperature for 3 hours. The solvent was removed by lyophilization, and the residue was separated by silica gel column chromatography (DCM/MeOH: 5:1) to give compound Glytecan (ESI-MS: m/z=493.2[M+H]^{F}). Equimolar amounts of Glytecan and DMAP were dissolved in DMSO, one equivalent of CMCL in DMSO was added dropwise into the glytecan solution slowly, and then the mixture was stirred and reacted at room temperature for 1 hour. The reaction solution was lyophilized, and the residue was separated by silica gel column chromatography (DCM/MeOH: 5:1) to give compound Glytecan-1 (ESI-MS: m/z=585.2[M+H]+).

Compound Glytecan-1 (50 mg, 0.085 mmol) and compound KJ-008 (68 mg, 0.085 mmol) were mixed and dissolved in 1.5 mL of DMF, anhydrous K₂CO₃ (12 mg) was added, and the mixture was stirred and reacted at room temperature for 6 hours. The reaction solution was filtered, the filtrate was lyophilized, and the lyophilizate was purified by semi-preparative HPLC to give compound K42 (43 mg, 38% yield), ESI-MS: m/z=671.8[(M/2)+H]+.

### Synthesis of Compound KJ-008

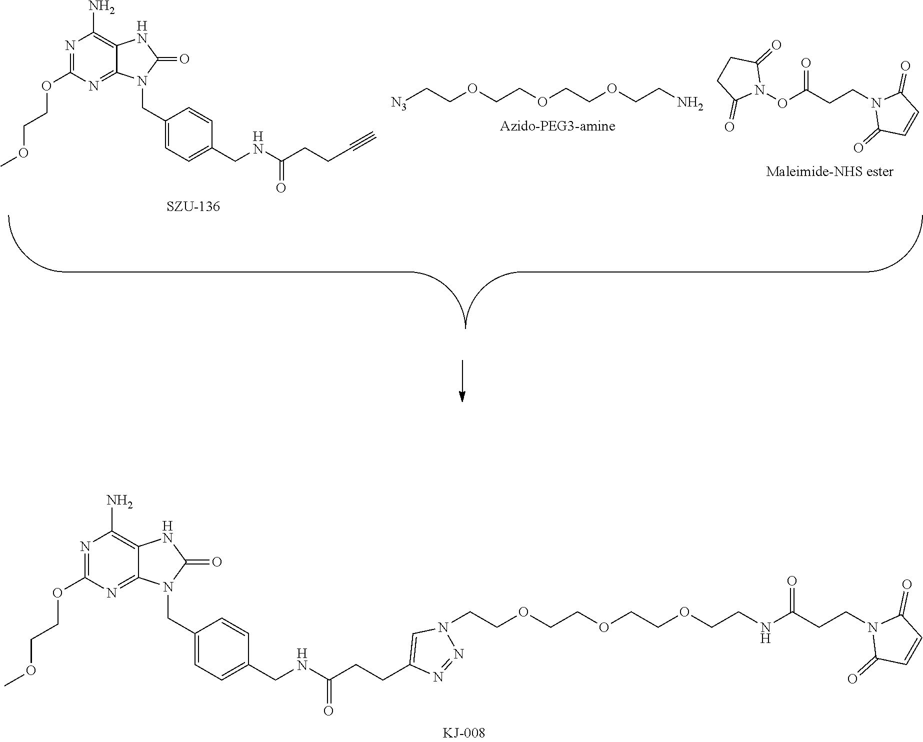 [SMILES: C#CCCC(=O)NCc1ccc(Cn2c(=O)[nH]c3c(N)nc(OCCOC)nc32)cc1 ; [N-]=[N+]=NCCOCCOCCOCCN ; O=C(CCN1C(=O)C=CC1=O)ON1C(=O)CCC1=O ; COCCOc1nc(N)c2[nH]c(=O)n(Cc3ccc(CNC(=O)CCc4cn(CCOCCOCCOCCNC(=O)CCN5C(=O)C=CC5=O)nn4)cc3)c2n1]

Compound Azido-PEG3-amine (110 mg. 0.5 mmol) and compound Maleimide-NHS ester (133 mg, 0.5 mmol) were mixed in 2 mL of DMF, and the mixture was stirred at room temperature for 30 minutes; the reaction solution was added with 0.5 mL of pure water, CuSO₄·5H₂O (13 mg), and sodium L-ascorbate (13 mg), and the resultant mixture was stirred and reacted for 6 hours. The reaction solution was filtered to remove insoluble substances, and the filtrate was lyophilized and purified by HPLC to give compound KJ-008 (288 mg, 73% yield), ESI-MS: m/z=794.4[M+H]⁺.

### Synthesis of Compound K43

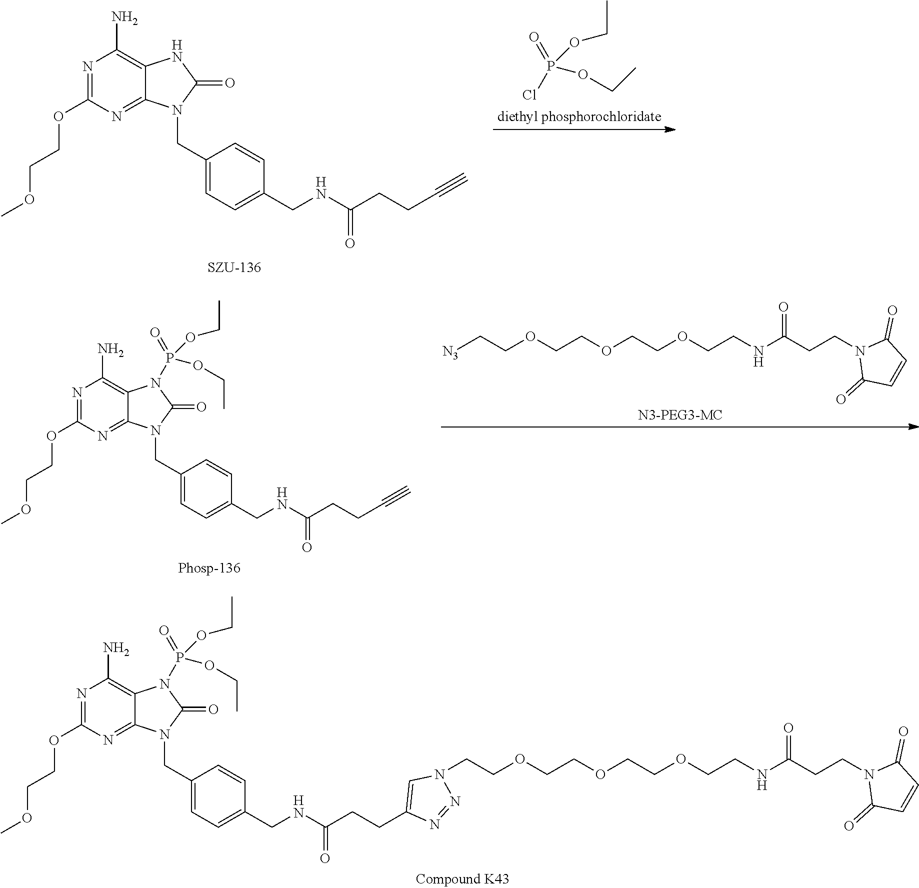

Compound SZU-136 (213 mg, 0.5 mmol) and compound diethyl phosphorochloridate (87 mg, 0.5 mmol) were dissolved in 3 mL of chloroform, DMAP (62 mg) was added, and the mixture was stirred and reacted overnight. The reaction solution was washed with cold water three times (3×5 mL), and the chloroform layer was separated, dried with anhydrous sodium sulfate, and filtered; and the filtrate was concentrated under reduced pressure to remove chloroform and left to stand to give solid compound Phosp-136 (258 mg, 92% yield), ESI-MS: m/z=561.2 [M+H]⁺.

Compounds Phosp-136 (101 mg, 0.18 mmol) and N3-PEG3-MC (67 mg, 0.18 mmol) were mixed and dissolved in 3 mL of DMF-H2O (4:1 volume), CuSO₄·5H₂O (12 mg) and sodium L-ascorbate (12 mg) were added, and the resultant mixture was stirred and reacted at room temperature for 6 hours. The reaction solution was lyophilized, and the lyophilizate was dissolved in a small amount of methanol; the mixture was filtered to remove insoluble substances, and the filtrate was separated and purified by HPLC to give compound K43 (94 mg, 56% yield), ESI-MS: m/z=930.4 [M+H]⁺.

### Synthesis of Compound K44

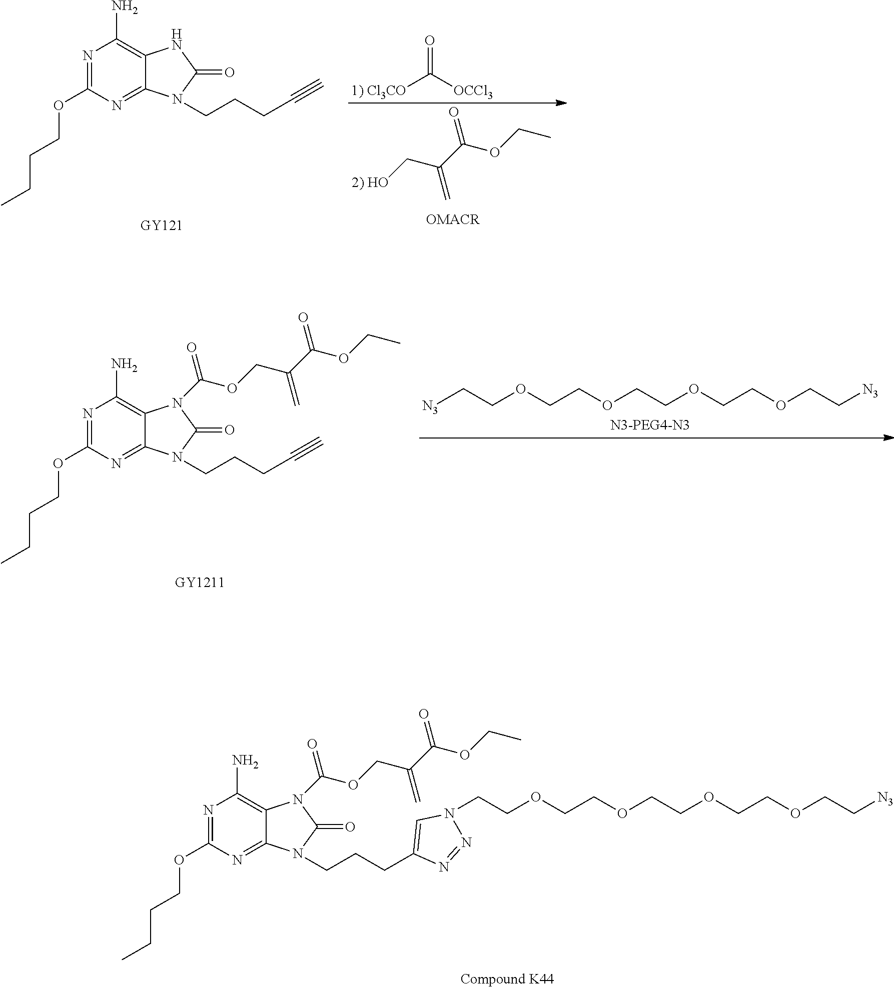

The solution of compound GY121 (145 mg, 0.5 mmol) in 2 mL of chloroform was added slowly to 1 mL of the solution of triphosgene (150 mg, 0.5 mmol) in chloroform, the resultant reaction solution was stirred at room temperature for 2 hours, and then OMACR (65 mg, 0.5 mmol) was added; the reaction mixture reacted at room temperature overnight. The reaction solution was concentrated under reduced pressure and separated by silica gel column chromatography (10:1 dichloromethane-methanol) to give compound GY1211 (143 mg, 64% yield), ESI-MS: m/z=446.2[M+H]⁺.

Compound GY1211 (98 mg, 0.22 mmol) and compound N3-PEG4-N3 (64 mg) were mixed and dissolved in 5 mL of DMF-H2O (4:1 by volume), CuSO₄·5H₂O (8 mg) and sodium L-ascorbate (8 mg) were added, and the resultant mixture was stirred and reacted at room temperature for 6 hours. The reaction solution was filtered, and the filtrate was lyophilized and purified by HPLC to give compound K44 (50 mg, 31% yield), ESI-MS: m/z=734.4 [M+H]⁺.

Synthesis of compound K45: according to the synthesis route and method of compound K44 except replacing compound GY121 with SZU-136, compound K45 was obtained, ESI-MS: m/z=869.4[M+H]⁺.

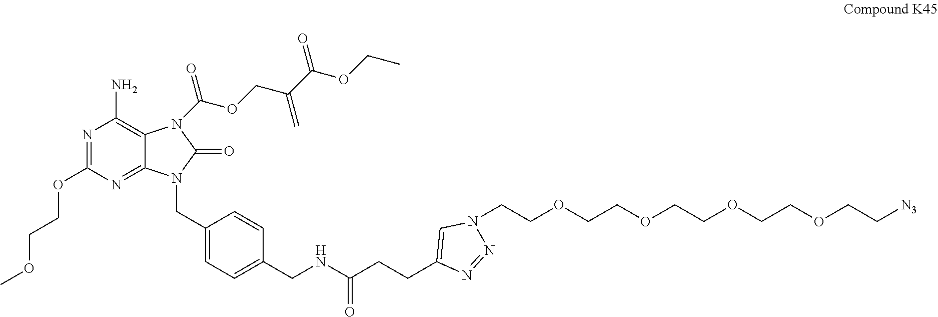 [SMILES: C=C(COC(=O)n1c(=O)n(Cc2ccc(CNC(=O)CCc3cn(CCOCCOCCOCCOCCN=[N+]=[N-])nn3)cc2)c2nc(OCCOC)nc(N)c21)C(=O)OCC]

### Synthesis of Compound K46

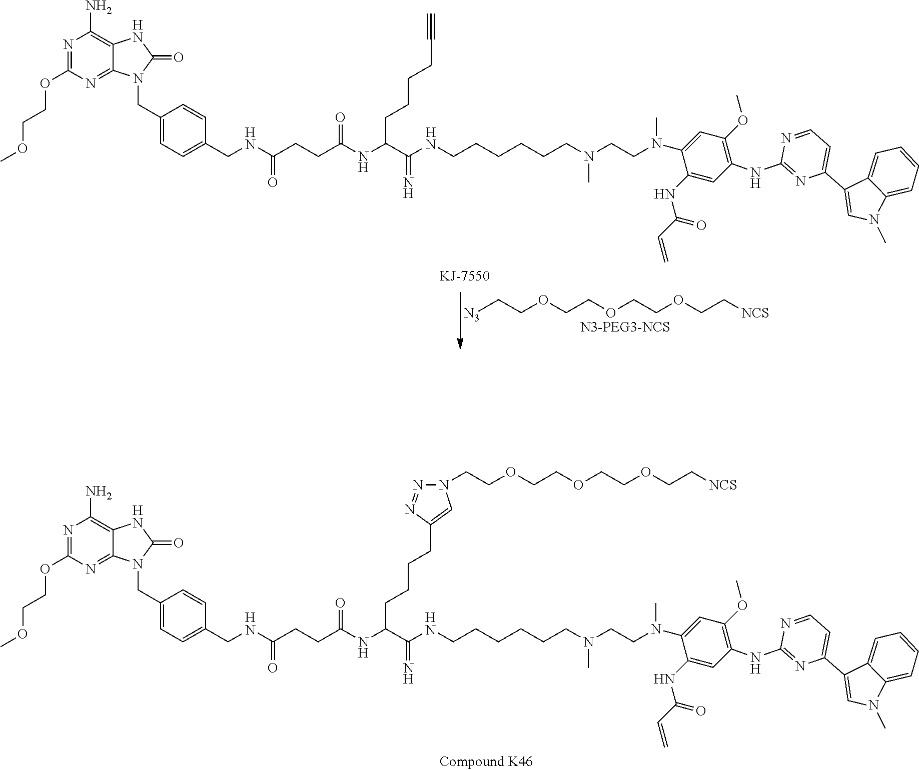

Compound KJ-7550 (100 mg, 0.087 mmol) and compound N3-PEG3-NCS (23 mg, 0.087 mmol) were dissolved in 2 mL of DMF-H2O (4:1 by volume) CuSO₄·5H₂O (5 mg) and sodium L-ascorbate (5 mg) were added, and the resultant mixture was stirred and reacted at room temperature for 12 hours. The reaction solution was filtered, the filtrate was lyophilized, and the lyophilizate was purified by HPLC to give compound K46 (85.8 mg, 70% yield), ESI-MS: m/z=705.4 [(M/2)+H]⁺.

### Synthesis of Compound K47

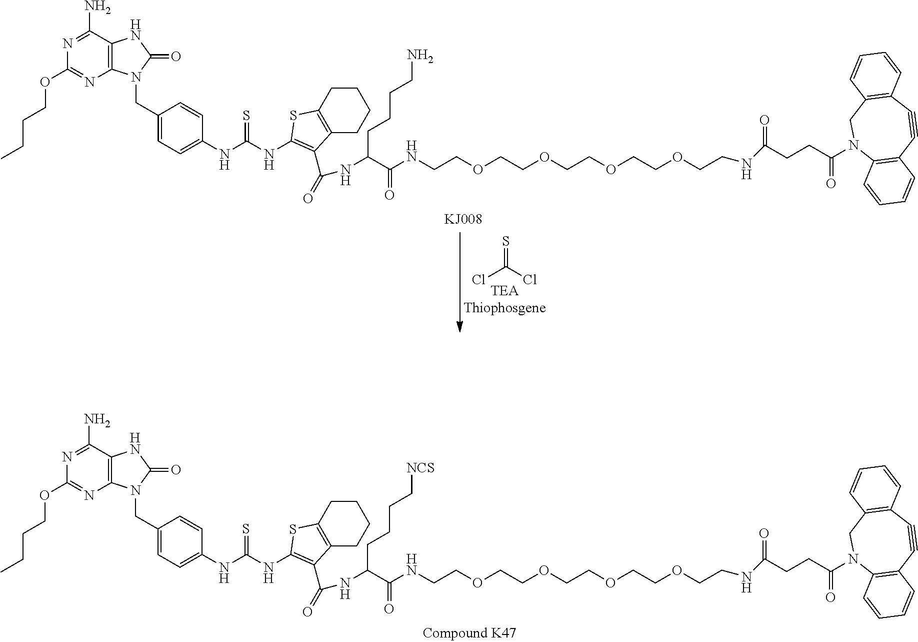

Equimolar amounts of compound KJ008 and thiophosgene were mixed in an appropriate amount of DMF, two equivalents of TEA were added, and the mixture was stirred and reacted at room temperature overnight. The solvent was removed by lyophilization, the residue was purified by preparative liquid chromatography, and the purified solution containing the product was collected and lyophilized to give compound K47, ESI-MS: m/z=622.2[(M/2)+H]⁺.

### Synthesis of Compound K48

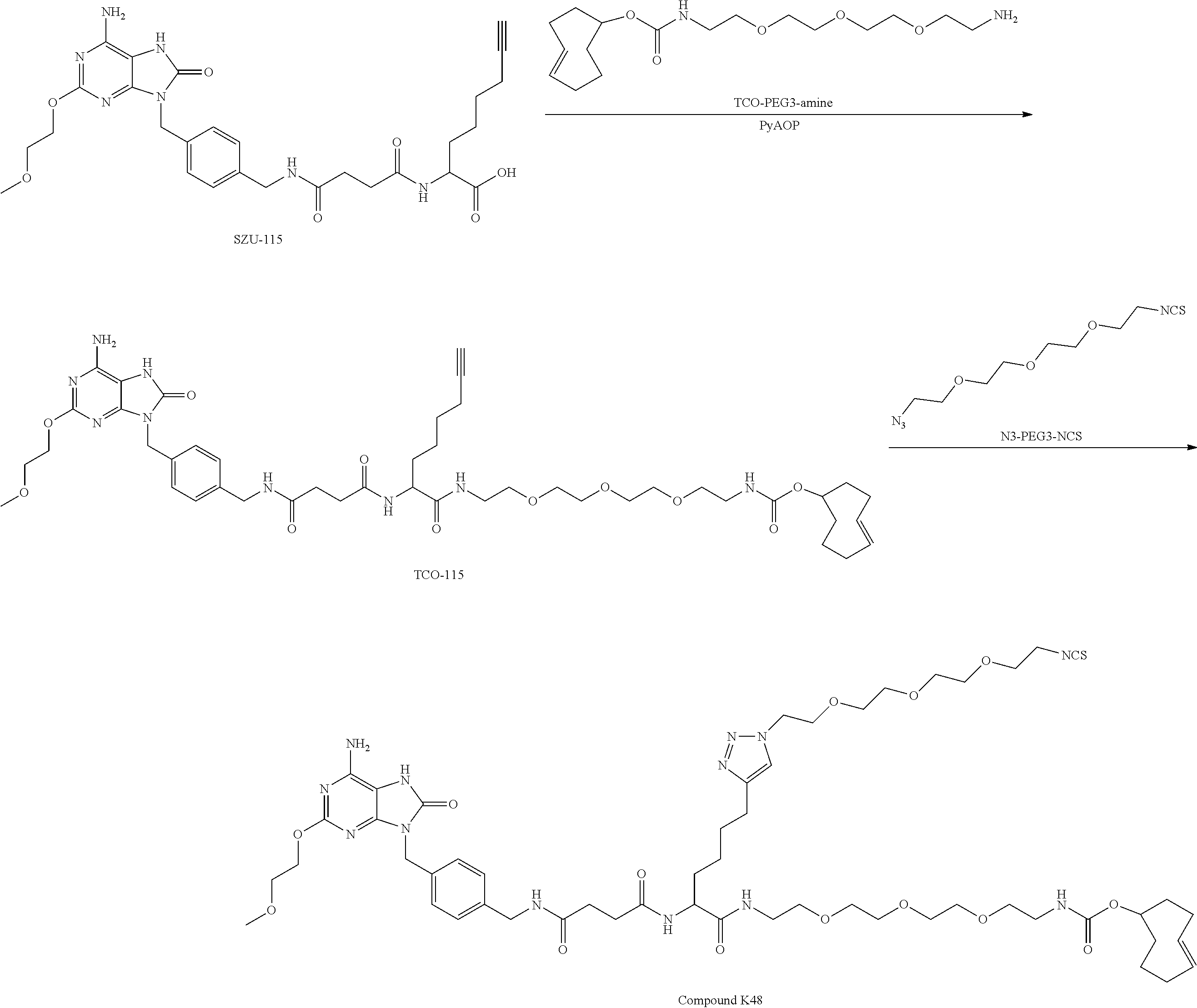

According to the synthesis method of KJ-7550, compound TCO-115 can be obtained, ESI-MS: m/z=908.5 [M+H]⁺; according to the synthesis method of KJ-7551, compound K48 was obtained, ESI-MS: m/z=1168.6[M+H]⁺.

### Synthesis of Compound K49

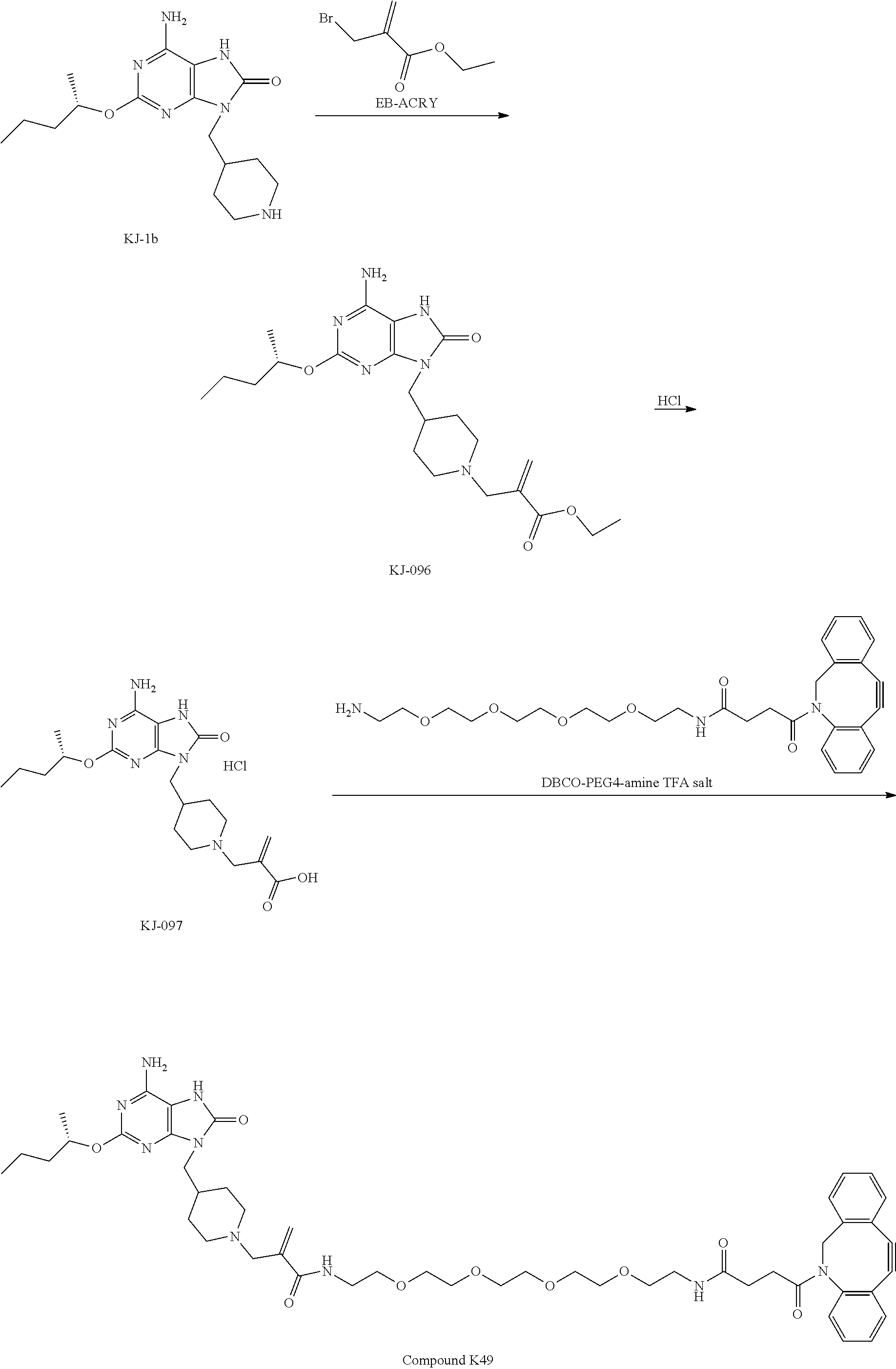

Compound KJ-1b (100 mg, 0.3 nmol) and compound EB-ACRY (58 mg, 0.3 mmol) were dissolved in 1 mL of DMF, anhydrous K₂CO₃ (100 mg) was added, and the mixture was stirred and reacted at room temperature for 6 hours. The reaction solution was filtered and lyophilized, and the lyophilizate was separated and purified by HPLC to give compound KJ-096 (70 mg, 52% yield), ESI-MS: m/z=447.2[M+H]⁺. 1 mL of concentrated hydrochloric acid was added to compound KJ-096 (65 mg), and the mixture was stirred at room temperature overnight and concentrated under reduced pressure. Pure water was added, and the mixture was stirred and then filtered to give the hydrochloride of compound KJ-097 (60 mg), ESI-MS: m/z=419.2[M+H]⁺. The hydrochloride of compound KJ-097 (58 mg, 0.127 mmol) and PyAOP (70 mg, 0.134 mmol) were mixed in 1.5 mL of DMF, DIPEA (88 μL) was added, and the mixture was stirred uniformly for 30 min. Then DBCO-PEG4-amine TFA salt (67 mg, 0.128 mmol) was added, and the resultant mixture was stirred and reacted at room temperature overnight. The reaction solution was lyophilized, the lyophilizate was purified by HPLC, and the purified solution containing the product was lyophilized to give compound K49 (81 mg, 69% yield), ESI-MS: m/z=924.5[M+H]⁺.

Synthesis of compound K50: according to the synthesis route and method of compound K49 except replacing compound KJ-1b with compound KJ-1a, compounds K50 {ESI-MS: m/z=910.5[M+H]⁺}, KJ-098 {ESI-MS: m/z=433.3[M+H]⁺} and KJ-099 {ESI-MS: m/z=405.2[M+H]⁺} can be obtained.

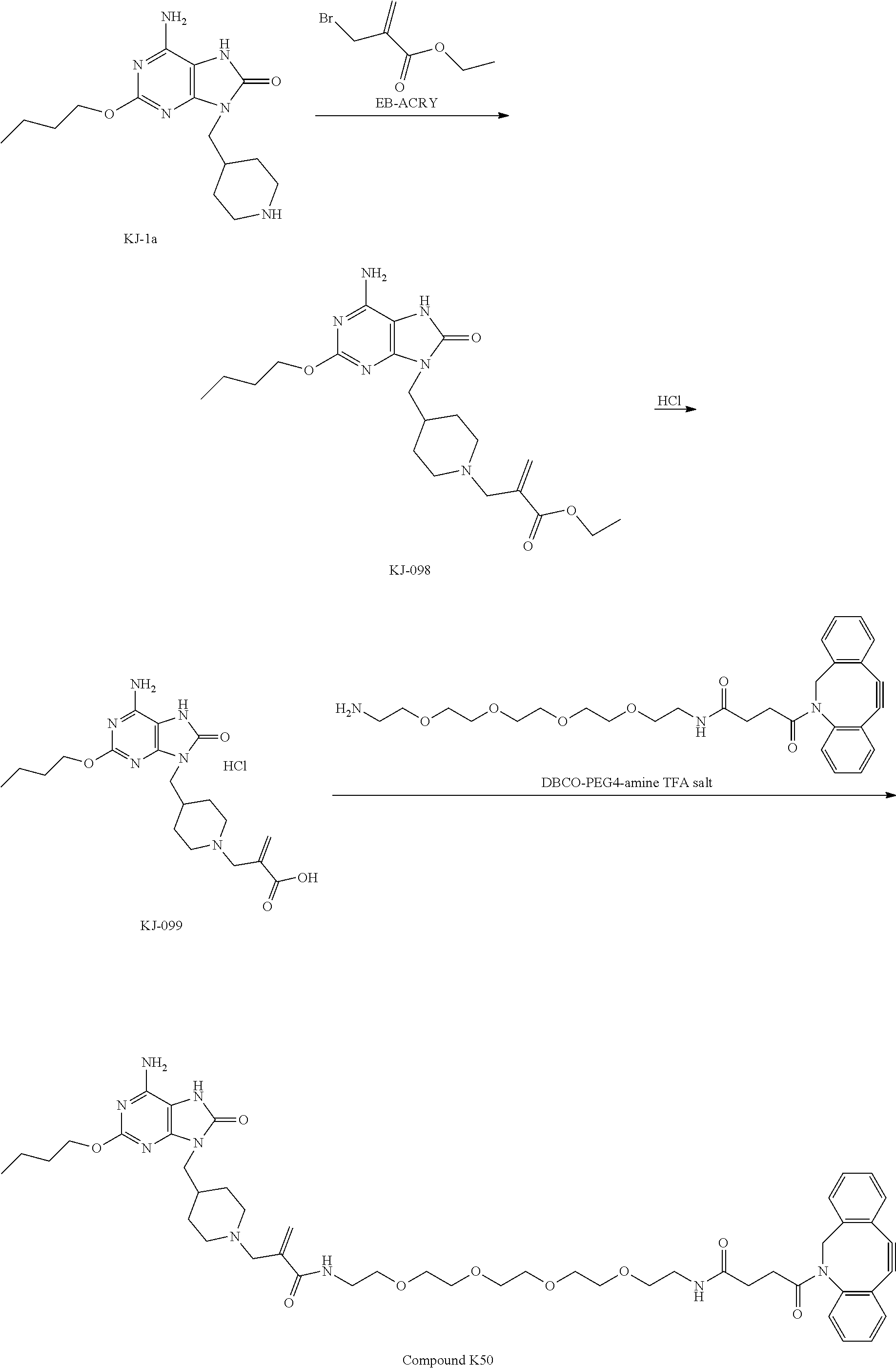

### Synthesis of Compound K51 and Compound K52

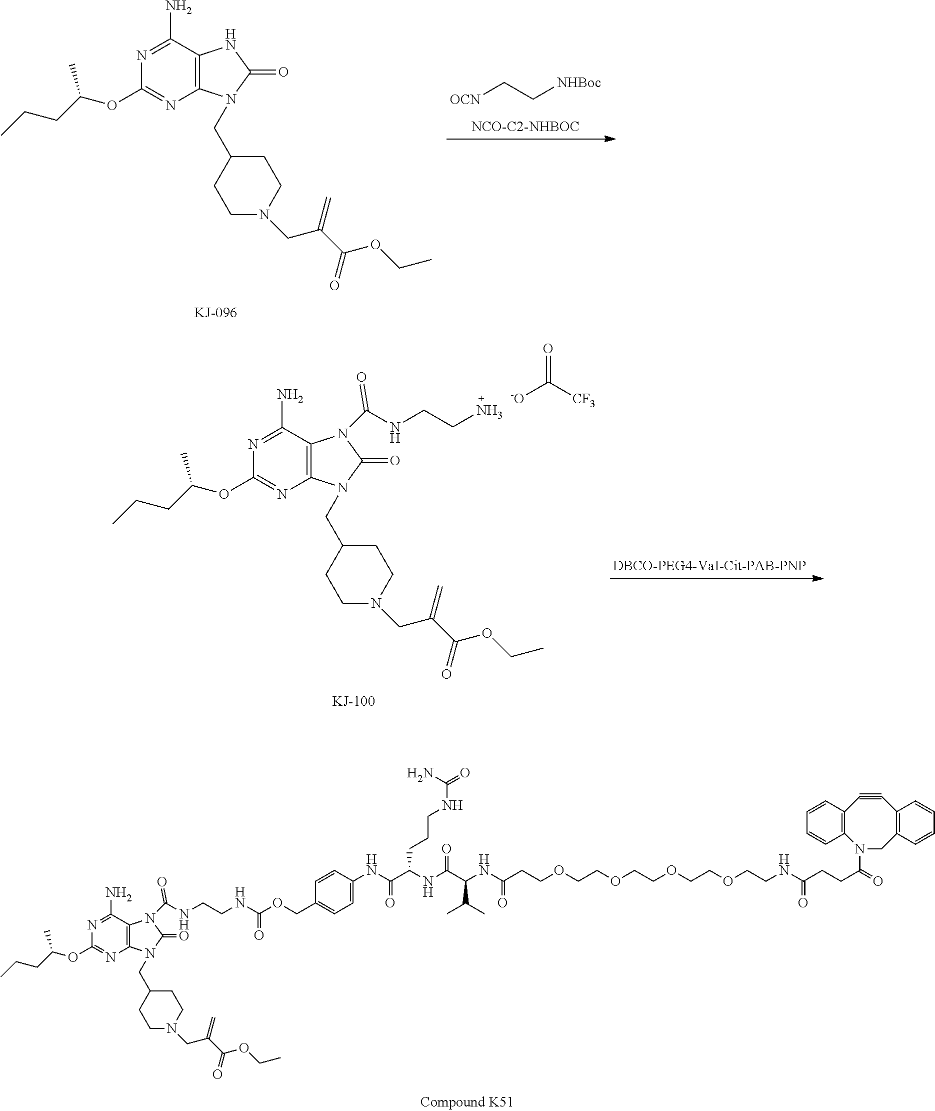

According to the synthesis method of GY7550-1 during the synthesis of compound K38, compound KJ-096 was reacted with compound NCO-C2-NHBOC to give compound KJ-100 {ESI-MS: m/z=533.3[M+H]⁺}; compound KJ-100 was reacted with DBCO-PEG4-Val-Cit-PAB-PNP to give compound K51 {ESI-MS: m/z=736.8[(M/2)+H]⁺}.

Similarly, compound KJ-096 was replaced by compound KJ-098 to give compound KJ-101 {ESI-MS: m/z=519.3[M+H]⁺}; and compound K52 {ESI-MS: m/z=729.9[(M/2)+H]⁺}.

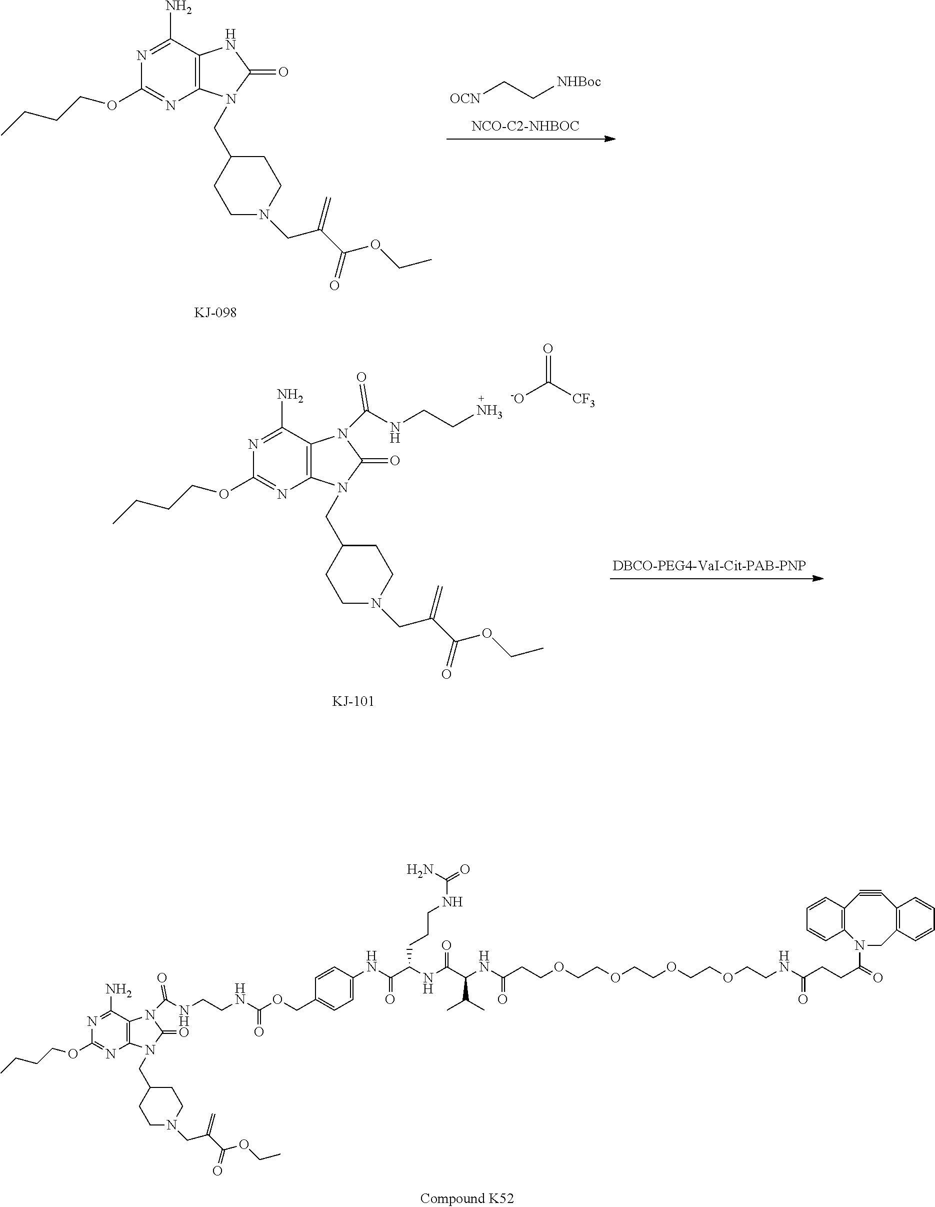

### Synthesis of Compound K53

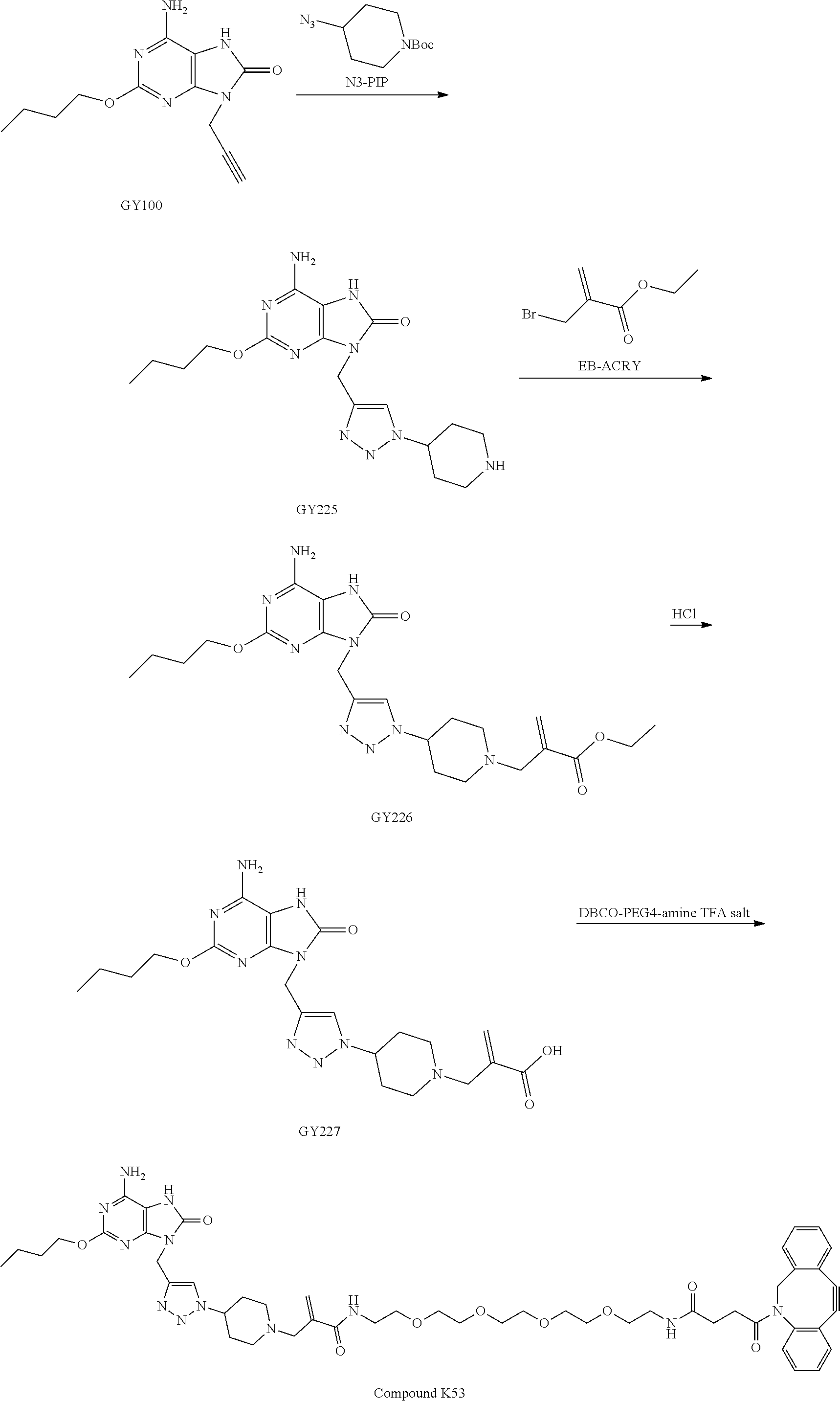

Compound GY100 (100 mg, 0.42 mmol) and compound N3-PIP (95 mg, 0.42 mmol) were dissolved in 2 mL of DMF-H₂O (4:1 volume), and CuSO₄·5H₂O (10 mg) and sodium L-ascorbate (10 mg) were added; the resultant mixture was shaken and reacted at room temperature for 6 hours. The solvent was removed by vacuum distillation, 4 mL of TFA-DCM (1:3 volume) was added, and the mixture was stirred at room temperature for 2 hours. The solvent was removed by vacuum distillation, and the residue was purified by HPLC to give compound GY225 (112 mg, 69% yield), ESI-MS: m/z=388.2 [M+H]⁺.

According to the synthesis methods of compound KJ-096 and compound KJ-097, compounds GY226 {ESI-MS: m/z=500.3[M+H]} and GY227 {ESI-MS: m/z=472.2[M+H]⁺} can be obtained. According to the synthesis method of compound K49, compound K53 can be obtained{ESI-MS: m/z=977.5[M+H]⁺}.

### Synthesis of Compound K54

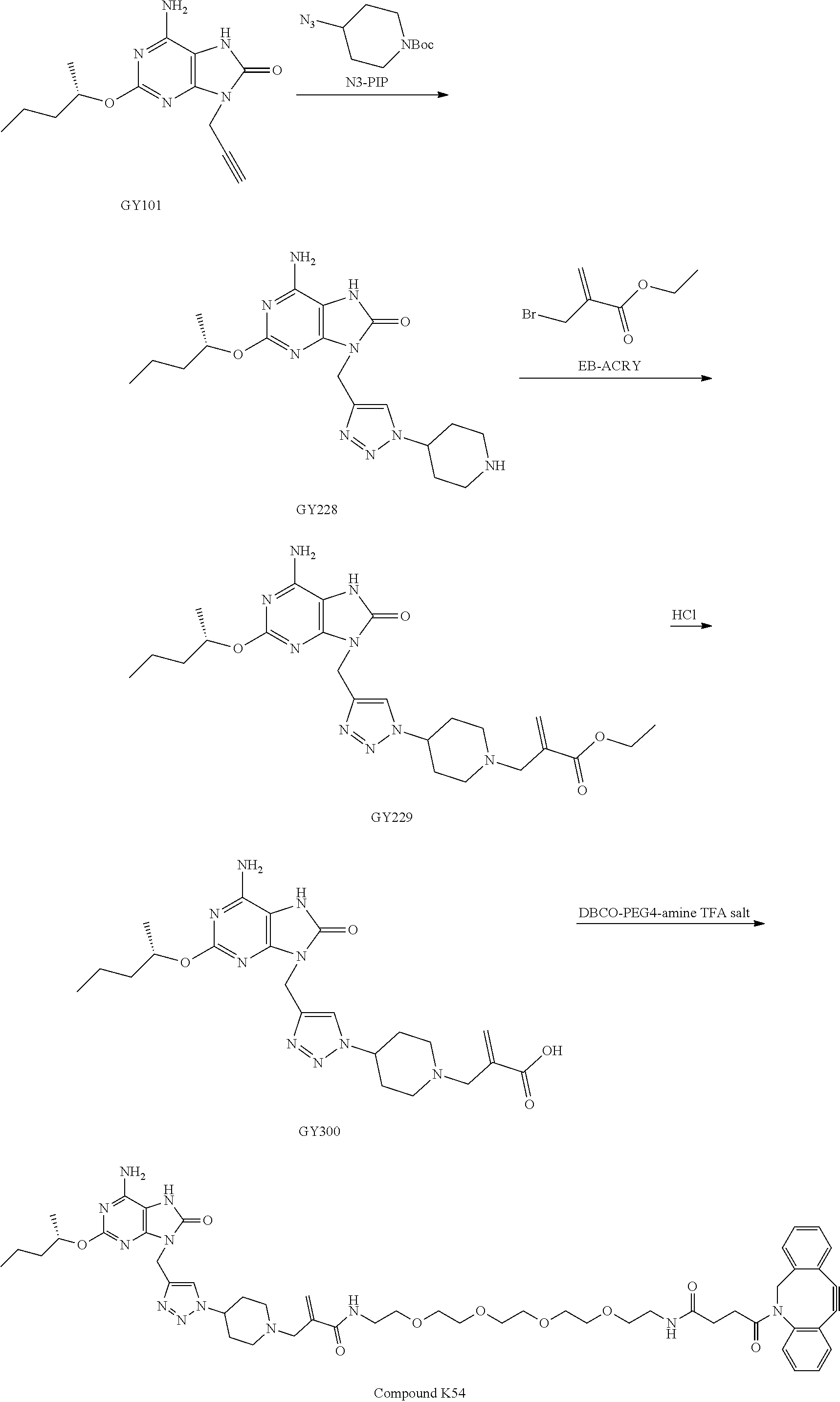

Similarly, according to the synthesis methods of compounds GY225, KJ-096, and compound KJ-097, compounds GY228 {ESI-MS: m/z=402.2[M+H]⁺}, GY229 {ESI-MS: m/z=514.3[M+H]⁺}, and GY300 {ESI-MS: m/z=486.2[M+H]⁺} can be obtained. According to the synthesis method of compound K49, compound K54 can be obtained {ESI-MS: m/z=991.5[M+H]⁺}.

### Synthesis of Compound K55

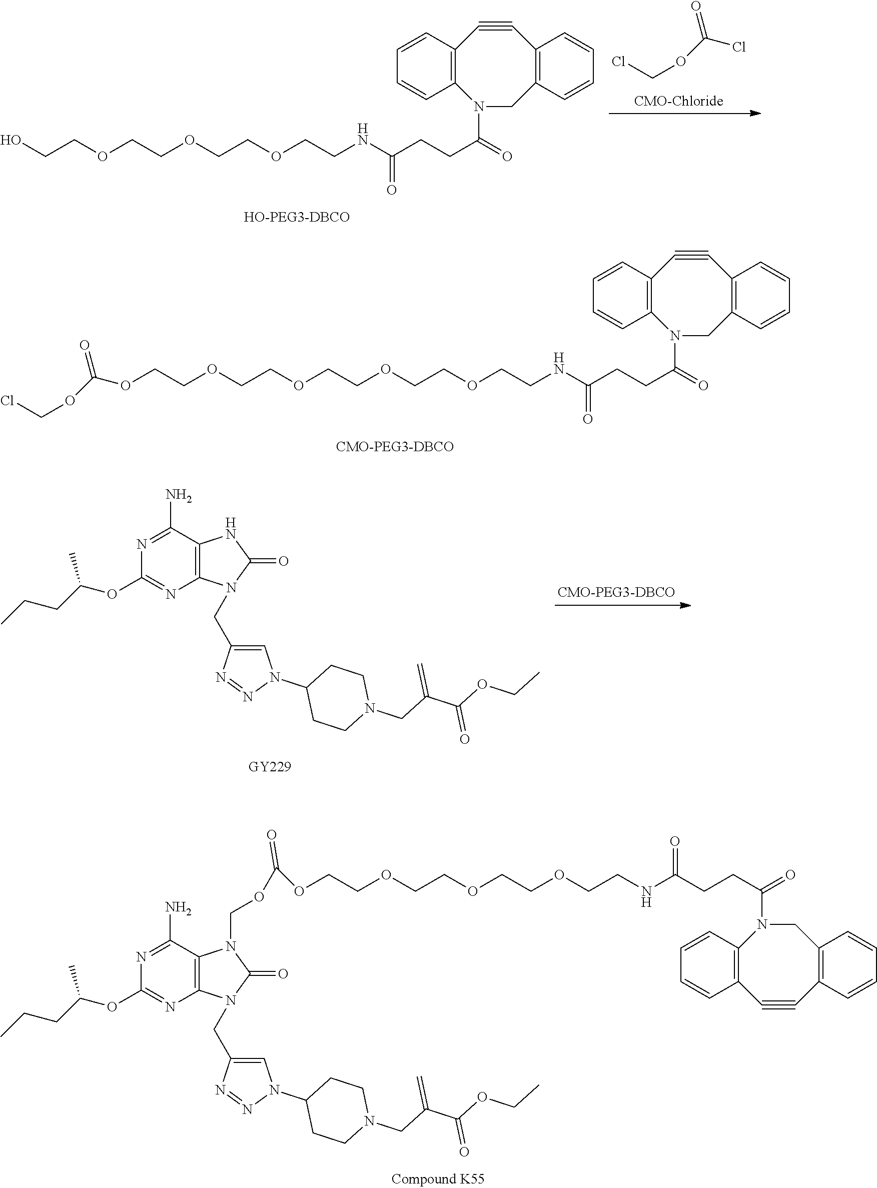

Compound HO-PEG3-DBCO (481 mg, 1 mmol) was dissolved in 5 mL of anhydrous THF, CMO-chloride (86 μL) was added slowly, and the mixture reacted at room temperature under nitrogen protection for 3 hours. The solvent was removed by vacuum distillation to give compound CMO-PEG3-DBCO (616 mg, 100% yield), ESI-MS: m/z=617.2[M+i-H]¹. Compound GY229 (103 mg, 0.2 mmol) and compound CMO-PEG3-DBCO (123 mg, 0.2 mmol) were mixed and dissolved in 3 mL of dichloromethane, and the mixture was stirred and reacted at room temperature overnight. The solvent was removed by vacuum distillation, and the residue was separated by silica gel column chromatography (chloroform) to give compound K55 (155 mg, 71% yield), ESI-MS: m/z=1050.5[M+H]⁺.

Synthesis of compound K56: according to the synthesis methods of compound GY7550-1 and compound K38, compound K56 (ESI-MS: m/z=763.4[(M/2)+H]⁺) can be obtained from compound GY226.

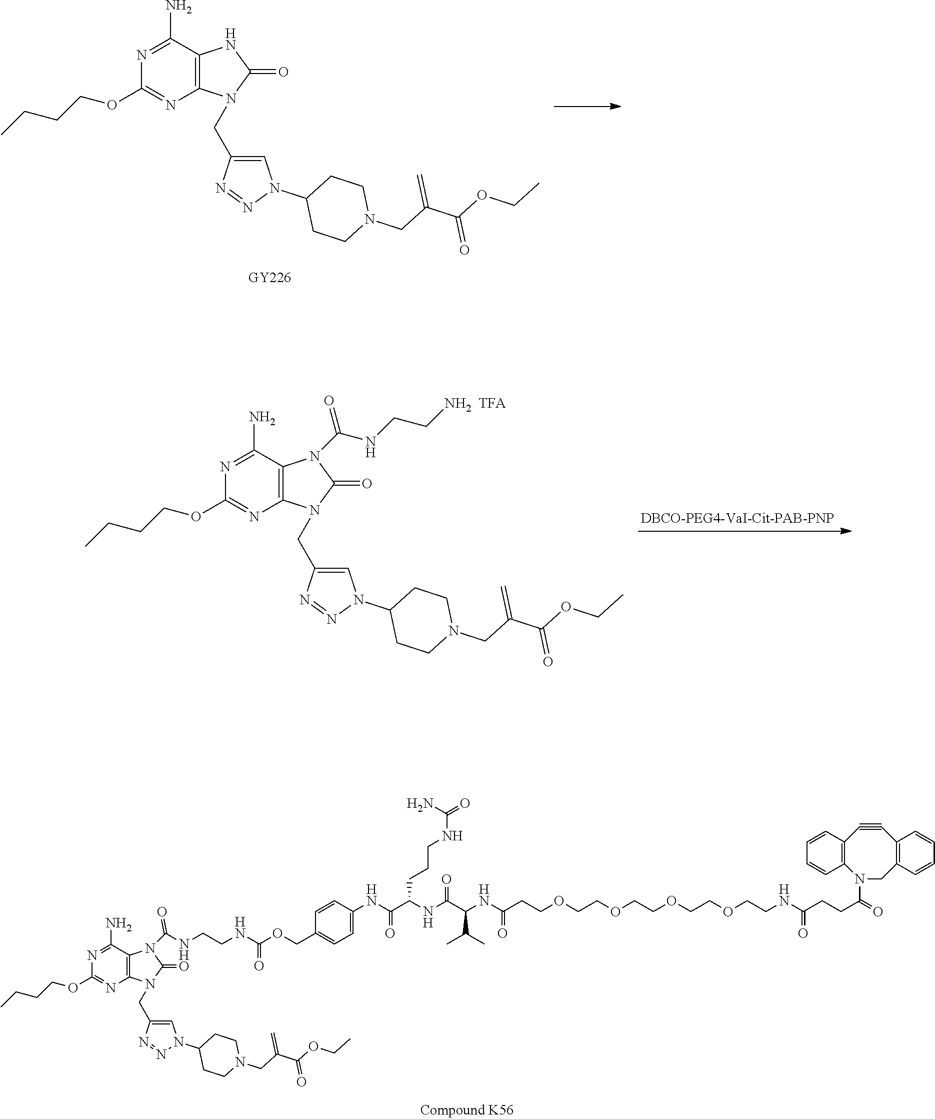

### Synthesis of Compound K57

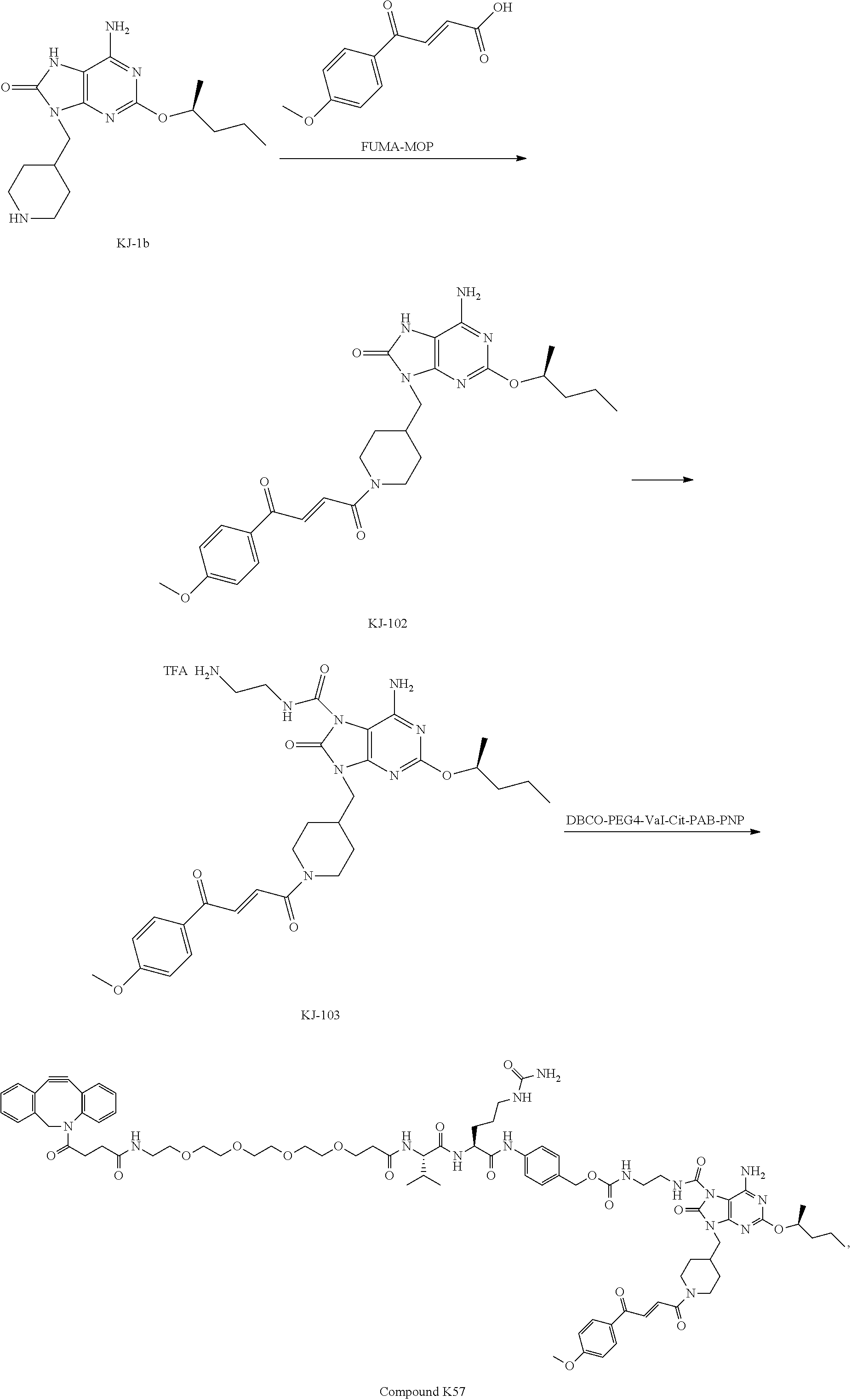

Compound FUMA-MOP (83 mg, 0.4 mmol) was dissolved in 2 mL of DMF, PyAOP (210 mg, 0.4 mmol) and DIPEA (70 μL) were added and stirred uniformly, and then KJ-1 b (134 mg, 0.4 mmol) was added; the resultant mixture was stirred and reacted at room temperature for 3 hours. The reaction solution was distilled under reduced pressure to remove the solvent, and purified by silica gel column chromatography (MeOH-DCM=1:5) to give compound KJ-102 (142 mg, 68% yield), ESI-MS: m/z=523.3[M+H]⁺. According to the synthesis methods of compound GY7550-1 and compound K38, compound K57 can be obtained {ESI-MS: m/z=774.9 [(M/2)+H]⁺}.

Synthesis of compound K58: according to the synthesis method of compound K57 except replacing compound KJ-1 b with KJ-1a, compound KJ-104 (ESI-MS: m/z 509.2[M+H]⁺) and compound K58 (ESI-MS: m/z=767.9[(M/2)+H]⁺) can be obtained.

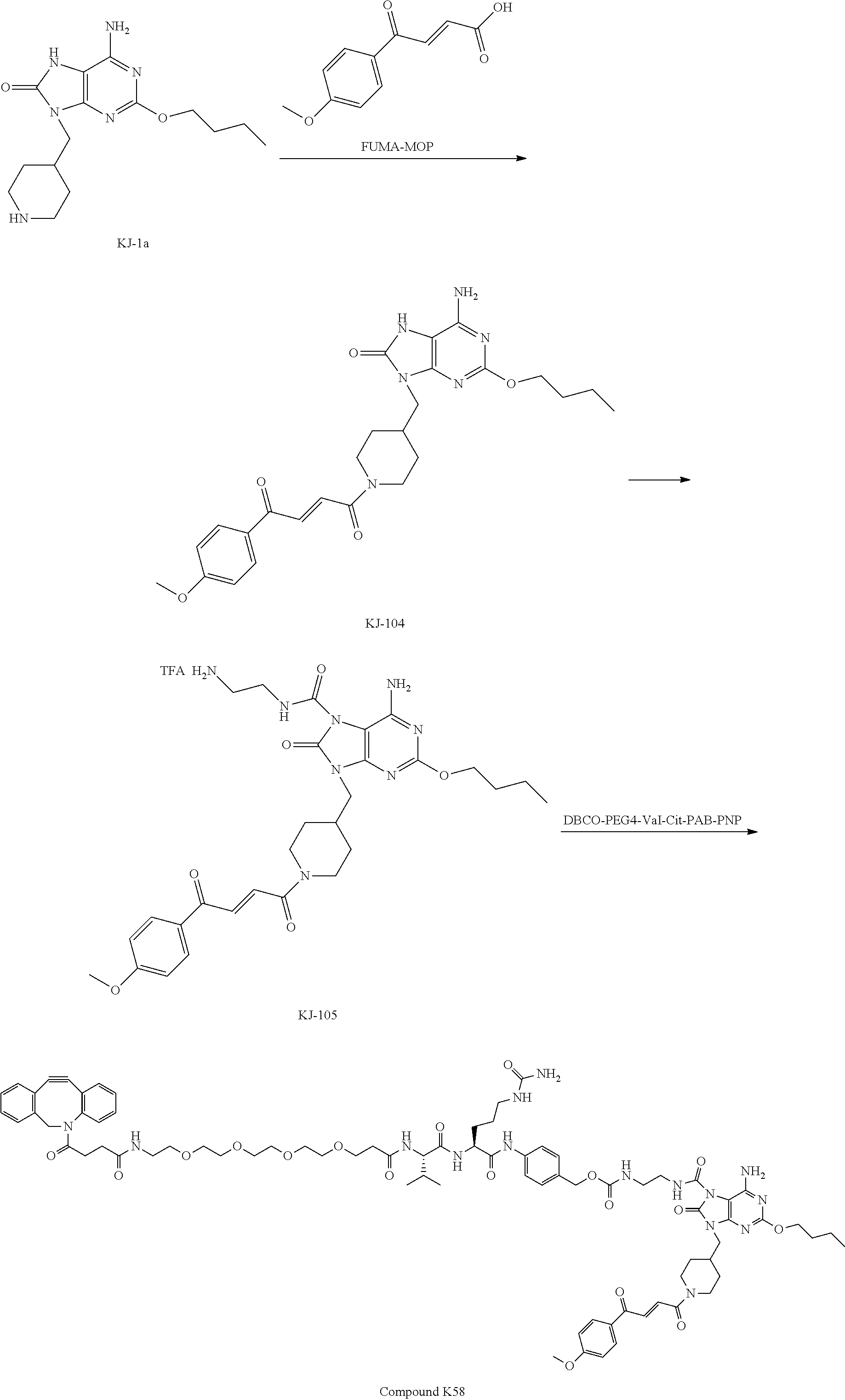

### Synthesis of Compound K59

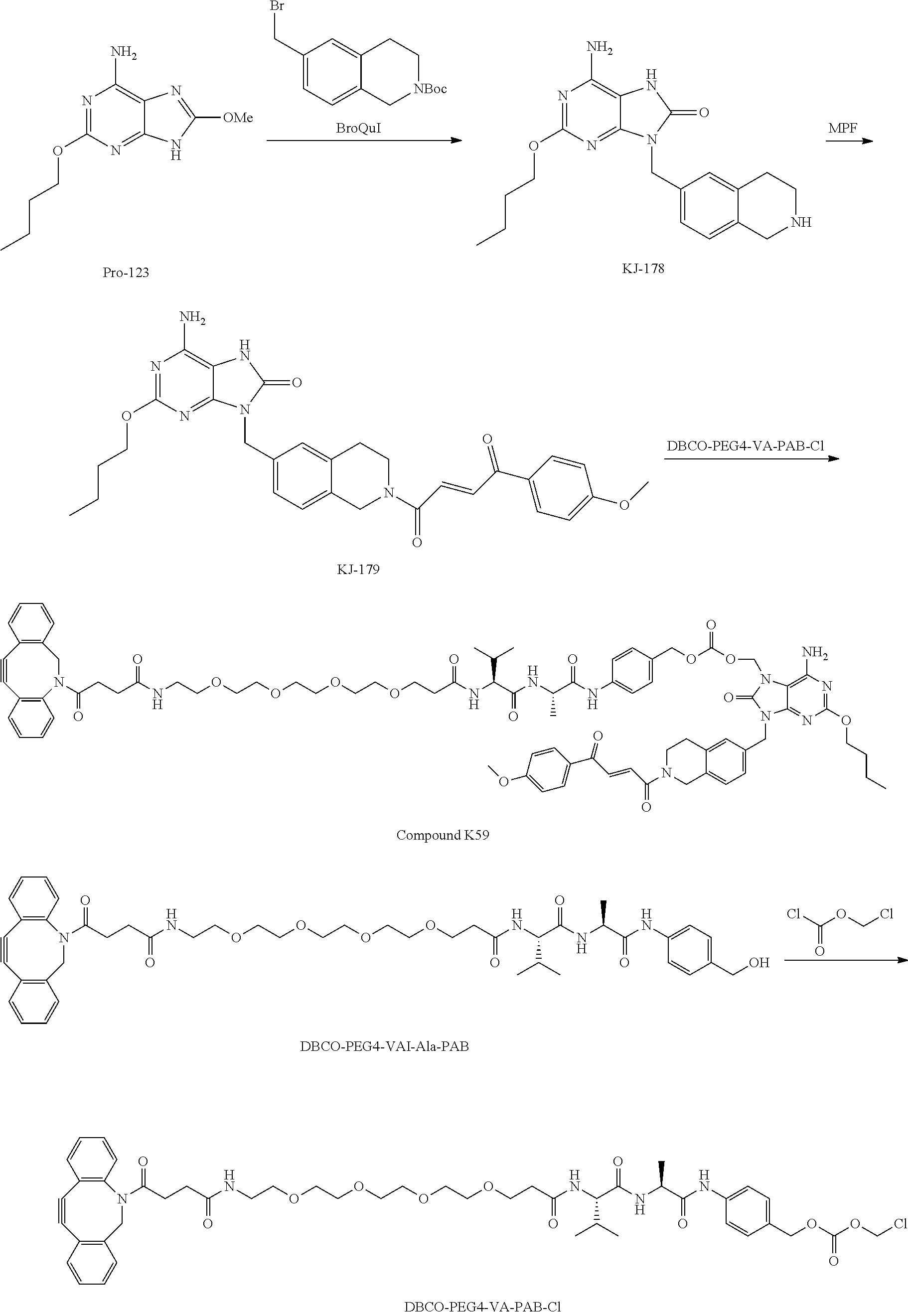

Compound Pro-123 (119 mg, 0.5 mmol) and BroQui (164 mg, 0.5 mmol) were mixed and dissolved in 3 mL of DMF, K₂CO₃ (140 mg) was added, and the mixture was stirred at room temperature for 10 hours. The reaction solution was filtered, the filtrate was lyophilized, and the lyophilizate was added with 10 mL of concentrated hydrochloric acid; the mixture was stirred at room temperature overnight, and neutralized to pH 8 with sodium carbonate. The precipitated solid was filtered and dissolved in methanol, and further separated and purified by HPLC to give compound KJ-178 (107 mg, 58% yield), ESI-MS: m/z=369.2[M+H]⁺. According to the synthesis method of PMQ-2, compound KJ-179 can be obtained (ESI-MS: m/z=557.2 [M+H]⁺). Compound KJ-179 (84 mg, 0.15 mmol) and K₂CO₃ (28 mg) were taken and mixed in 2 mL of DMF, DBCO-PEG4-VA-PAB-Cl (138 mg, 0.15 mmol) was added, and the mixture was stirred and reacted at room temperature overnight. The reaction solution was filtered, and the filtrate was lyophilized and purified by HPLC to give compound K59 (101.5 mg, 47% yield), ESI-MS: m/z=720.8[(M/2)+H]⁺.

Synthesis of DBCO-PEG4-VA-PAB-C1: quimolar amounts of DBCO-PEG4-Val-Ala-PAB and chloromethyl chloroformate were reacted in dry DCM at room temperature for 10 hours. The solvent was removed completely by vacuum distillation to give compound DBCO-PEG4-VA-PAB-C1 (ESI-MS: m/z 9204[M+H]⁺).

Synthesis of compound K60: according to the synthesis method of compound K59, SZU-103 was used as the starting material to give compound K60 {ESI-MS: m/z=756.8[(M/2)+H]⁺}.

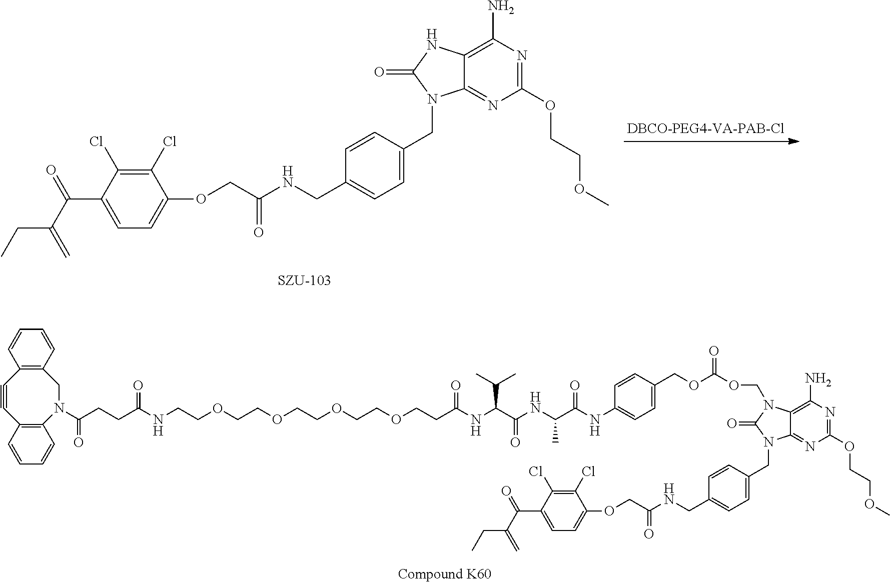 [SMILES: C=C(CC)C(=O)c1ccc(OCC(=O)NCc2ccc(Cn3c(=O)[nH]c4c(N)nc(OCCOC)nc43)cc2)c(Cl)c1Cl ; C=C(CC)C(=O)c1ccc(OCC(=O)NCc2ccc(Cn3c(=O)n(COC(=O)OCc4ccc(NC(=O)[C@H](C)NC(=O)[C@@H](NC(=O)CCOCCOCCOCCOCCNC(=O)CCC(=O)N5Cc6ccccc6C#Cc6ccccc65)C(C)C)cc4)c4c(N)nc(OCCOC)nc43)cc2)c(Cl)c1Cl]

### Synthesis of Compound K61

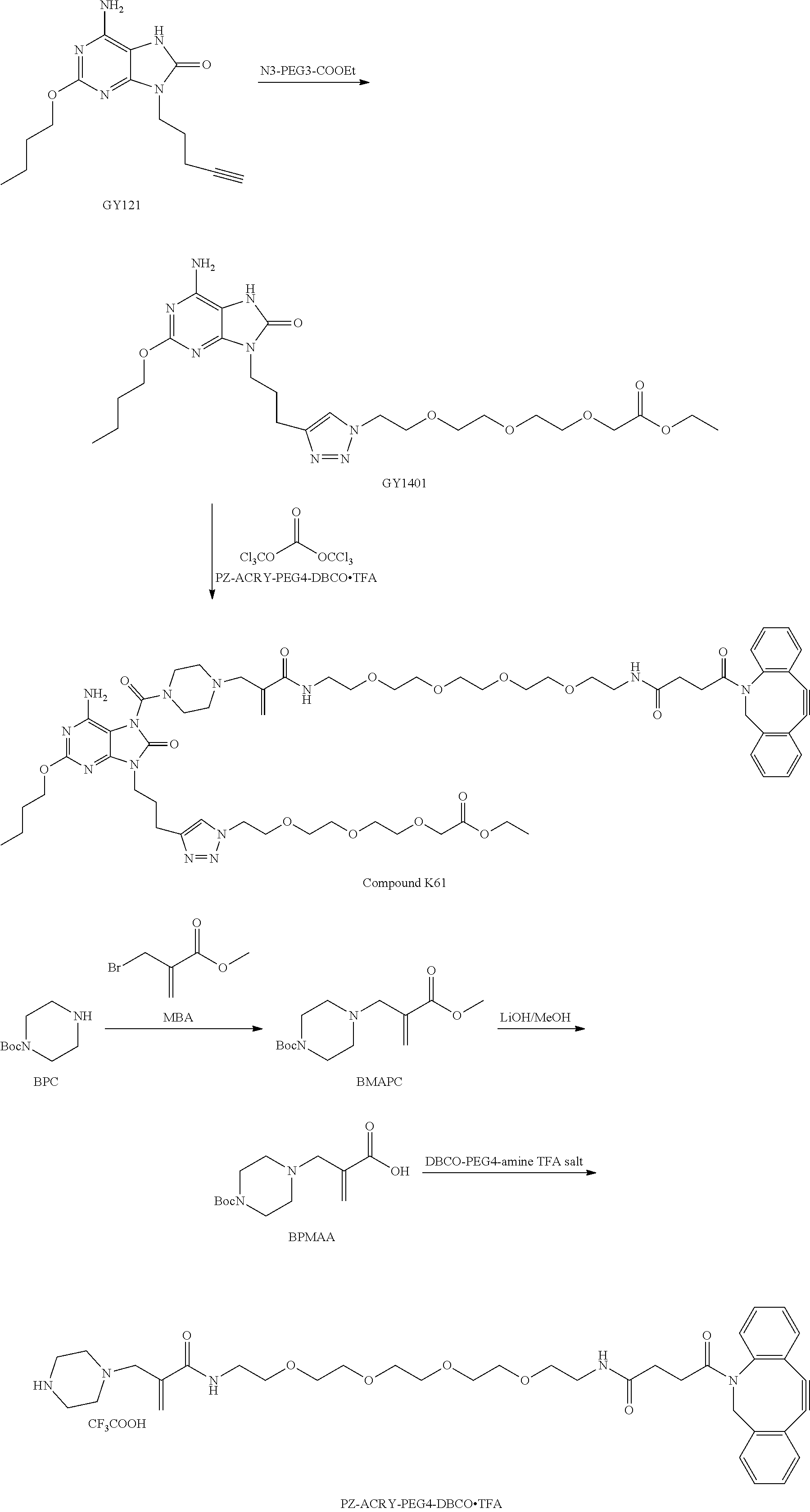

Compound GY121 (87 mg, 0.3 mmol) and N3-PEG3-COOEt (79 mg, 0.3 mmol) were dissolved in 2 mL of H2O-DMF (1:4), CuSO₄·5H₂O (10 mg) and sodium L-ascorbate (10 mg) were added, and the resultant mixture was subjected to a click reaction at room temperature for 6 hours; the reaction solution was filtered, and the filtrate was lyophilized, and separated and purified by HPLC to give compound GY1401 (111 mg, 67% yield), ESI-MS: m/z=551.3[M+H]⁺. Compound GY1401 (100 mg, 0.18 mmol) and triphosgene (32 μL, 0.19 mmol) were taken and dissolved in 3 mL of DMF, DMAP (22 mg) was added, and the mixture was mixed uniformly and reacted at room temperature for 3 hours; then PZ-ACRY-PEG4-DBCO. TFA (142 mg, 0.18 mmol) and DMAP (33 mg) were added sequentially, and the mixture was stirred and reacted at room temperature overnight. The reaction solution was lyophilized, and the lyophilizate was added with 10 mL of cold water; the mixture was extracted with ethyl acetate (3×15 mL), the organic phases were separated and combined; the combined organic phase was washed with 30 mL of saturated sodium bicarbonate solution once, and washed with saturated sodium chloride once. The organic phase was separated and dried with anhydrous sodium sulfate. The desiccant was filtered off, the filtrate was distilled under reduced pressure to remove the solvent, and the residue was purified by HPLC to give compound K61 (79 mg, 35% yield), ESI-MS: m/z=626.8[(M/2)+H]⁺.

### Synthesis of Compound PZ-ACRY-PEG4-DBCO.TFA

Equimolar amounts of compound BPC and compound MBA were subjected to a substitution reaction in DMF at room temperature. After reaction completion as monitored by mass spectrometry, the solvent was removed by vacuum distillation, and saturated sodium bicarbonate solution was added and stirred thoroughly; ethyl acetate was added to extract the product, and the organic phase was washed with water, dried with anhydrous sodium sulfate, and filtered; and the filtrate was distilled to dryness to give compound BMAPC; BMAPC was dissolved in methanol, three equivalents of LiOH·H₂O were added, and the mixture was stirred and reacted at room temperature until reaction completion; the solvent was removed by vacuum distillation, the residue was added with pure water and adjusted to pH 6 with acetic acid to precipitate a solid compound; the solid compound was filtered and dried to give compound BPMAA (ESI-MS: m/z=271.2[M+H]4).

Compound BPMAA (81 mg, 0.3 mmol) and compound DBCO-PEG4-amine TFA salt (191 mg, 0.3 mmol) were dissolved in 3 mL of DMF, DIPEA (157 μL), NHS (38 mg, 0.33 mmol), and EDC·HCl (64 mg, 0.33 mmol) were added, and the resultant mixture was stirred and reacted at room temperature overnight. The reaction solution was lyophilized, the lyophilizate was added with 10 mL of solvent TFA-DCM (1:3 volume), and the mixture was stirred at room temperature for 2 hours; the solvent was removed by vacuum distillation, and the residue was separated and purified by preparative HPLC to give the TFA salt of compound PZ-ACRY-PEG4-DBCO (104 mg, 44% yield), ESI-MS: m/z=676.3[M+H]⁺.

### Synthesis Route of Compound K62

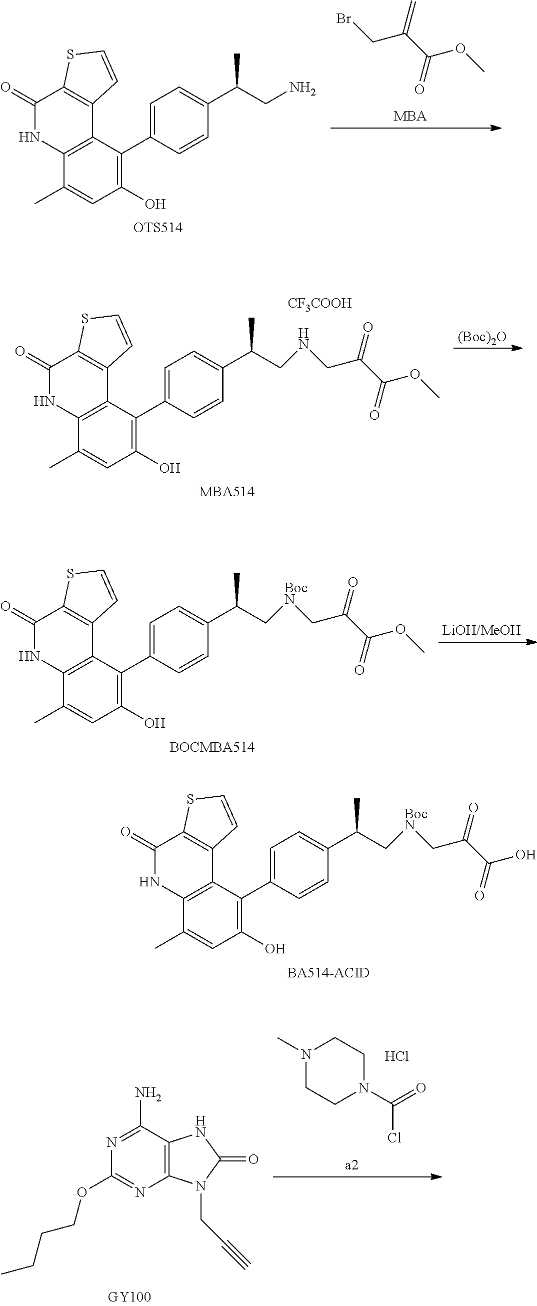

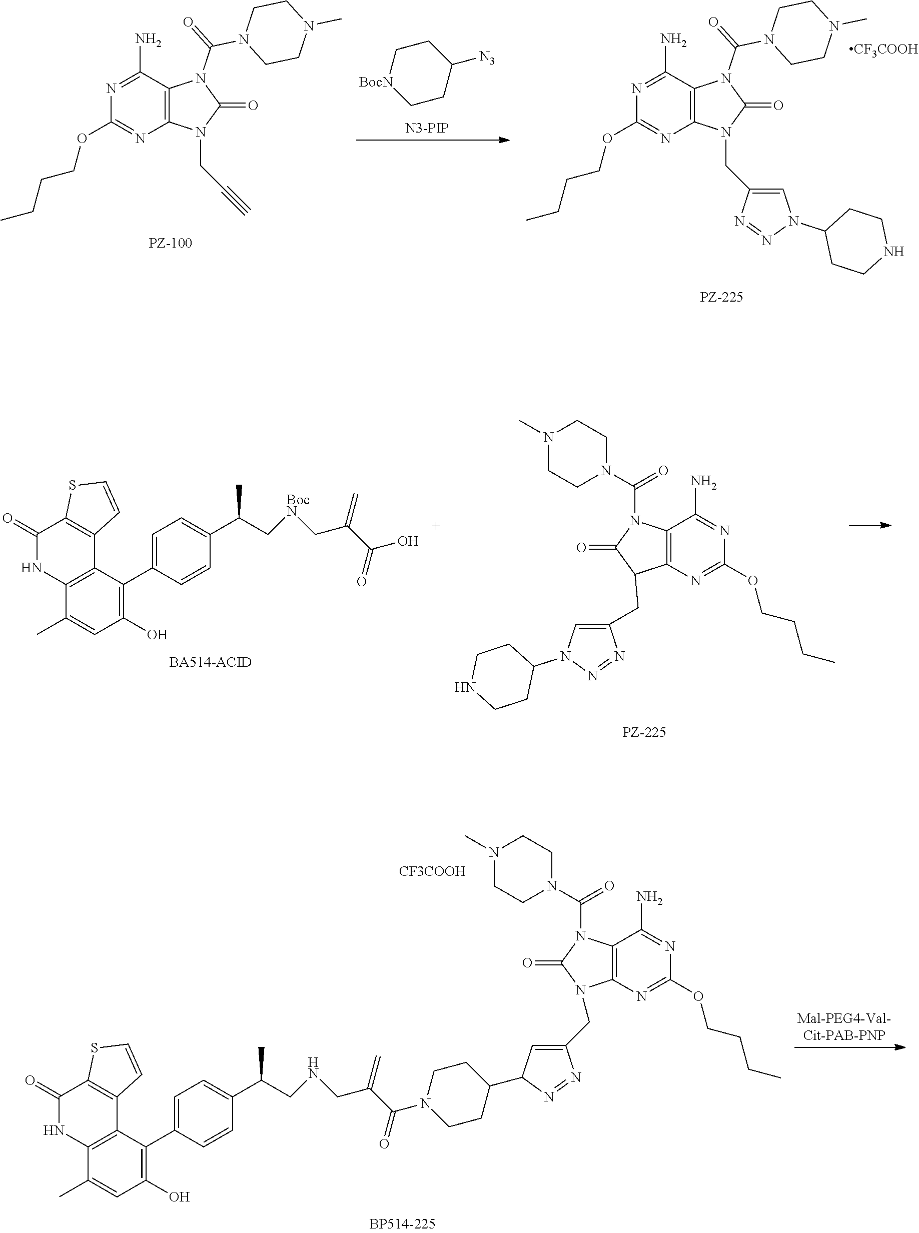

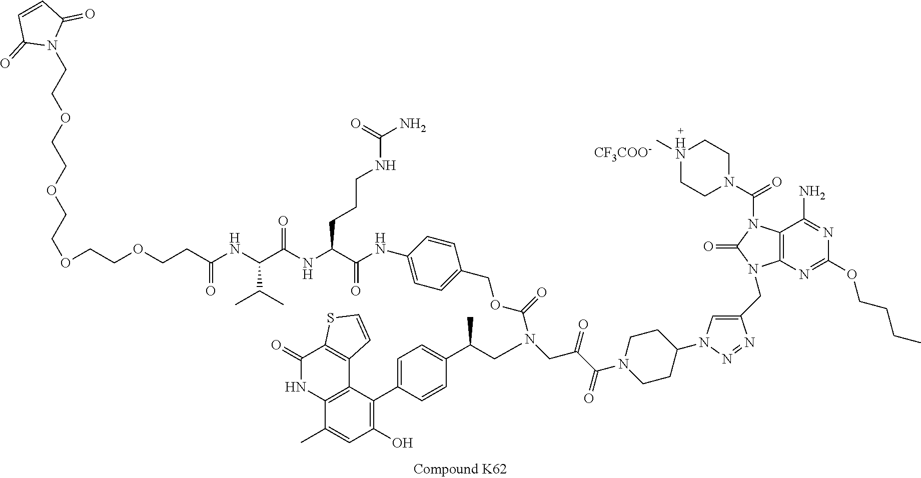

Synthesis of compound BA514-ACID: compound OTS514 (110 mg. 0.3 mmol) and compound MBA (54 mg, 0.3 mmol) were mixed and dissolved in 2 mL of DMF, Cs₂CO₃ (98 mg, 0.3 mmol) was added, and the mixture was stirred and reacted at room temperature until reaction completion as monitored by mass spectrometry. The reaction solution was lyophilized, and the lyophilizate was separated and purified by HPLC to give compound MBA514.TFA (58 mg, ESI-MS: m/z=463.2[M+H]⁺); MBA514.TFA (50 mg, 0.086 mmol) was taken and dissolved in 1 mL of DMF, the mixture was cooled to 5° C., and DMAP (22 mg) was added; (Boc)₂O (20 μL) was dissolved in another 0.5 ml of DMF, the mixture was slowly added into the solution of MBA514, and the resultant mixture was allowed to warm up naturally to room temperature, and was stirred and reacted for 12 hours; the reaction solution was lyophilized, and the lyophilizate was separated and purified by HPLC to give a separated solution containing the product; the separated solution was lyophilized to give compound BOCMBA514 (28 mg); compound BOCMBA514 was dissolved in 1 mL of methanol, 0.1 nml, of the aqueous solution of LiOH·H₂O (10 mg) was added, and the mixture was shaken and reacted at room temperature until reaction completion; the solvent was removed by vacuum distillation, 1 mL of water was added, and the mixture was adjusted to pH 3 with dilute hydrochloric acid under cooling. The precipitated solid was filtered by centrifugation and dried under vacuum to give compound BA514-ACID (25 mg, ESI-MS: m/z=549.2[M+H]⁺).

Synthesis of compound PZ-100 and compound P-Z225: according to the synthesis method of compound PZ-136 except replacing compound SZU-136 with GY100, compound PZ-100 can be obtained (ESI-MS: m/z=388.2[M+H]⁺). According to the synthesis method of GY225, the TFA salt of PZ-225 can be obtained (ESI-MS: m/z=514.3[M+H]⁺).

Synthesis of compound BP514-225: compound BA514-ACID (100 mg, 0.18 mmol) and condensing agent PyAOP (100 mg, 0.19 mmol) were dissolved in 2.5 mL of DMF, DIPEA (50 μL) was added, and the mixture was mixed uniformly; another solution of compound PZ-225 TFA salt (125 mg, 0.18 mmol) and DIPEA (50 μL) in DMF (2.5 mL) was slowly added into the solution of BA514-ACID above, and the resultant reaction solution was shaken and reacted at room temperature overnight. The reaction solution was lyophilized, the lyophilizate was added with 10 mL of TFA-DCM (1:3 by volume), and the mixture was stirred naturally at room temperature for 1 hour; the solvent was removed by vacuum distillation, and the residue was purified by HPLC to give the TFA salt of compound BP514-225 (93 mg, 49% yield), ESI-MS: m/z=945.4[M+H]⁺.

Synthesis of compound K62: compound BP514-225.TFA (53 mg, 0.05 mmol) and DMAP (10 mg) were taken and dissolved in 1 ml, of DMF, and the solution of Mal-PEG4-Val-Cit-PAB-PNP (44 mg, 0.05 mmol) in DMF (1 mL) was added dropwise into the solution of BP514-225.TFA slowly at room temperature; after the addition, the mixture was stirred and reacted at room temperature overnight. The reaction solution was lyophilized, and the lyophilizate was purified by HPLC to give the TFA salt of compound K62 (28 mg, 31% yield), ESI-MS: m/z=838.5[(M/2)+H]⁺.

### Synthesis of Compound K63

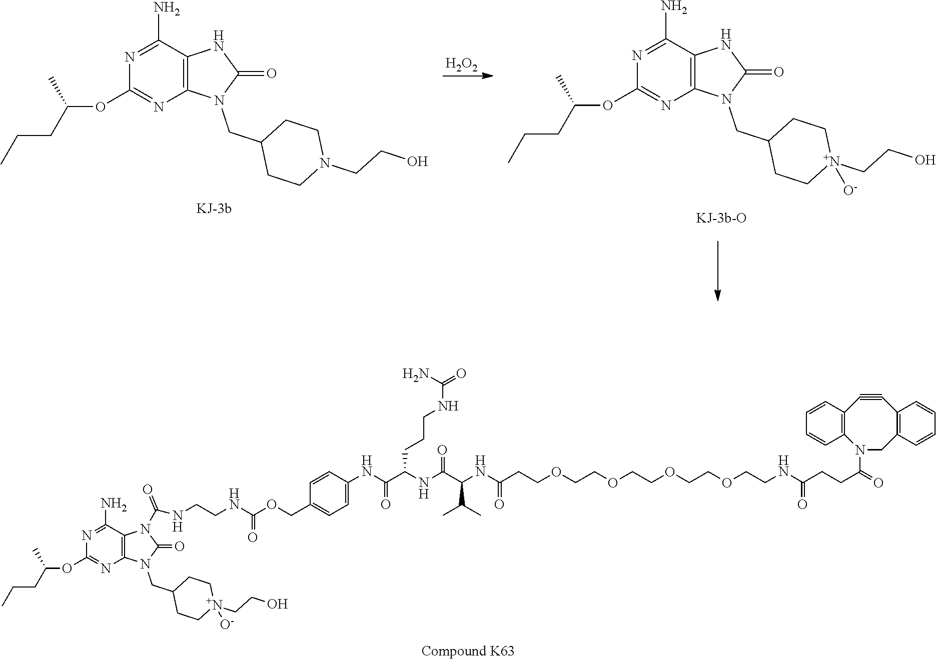 [SMILES: CCC[C@H](C)Oc1nc(N)c2[nH]c(=O)n(CC3CCN(CCO)CC3)c2n1 ; "H2O2" ; CCC[C@H](C)Oc1nc(N)c2[nH]c(=O)n(CC3CC[N+]([O-])(CCO)CC3)c2n1 ; CCC[C@H](C)Oc1nc(N)c2c(n1)n(CC1CC[N+]([O-])(CCO)CC1)c(=O)n2C(=O)NCCNC(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1]

Compound KJ-3b (95 mg, 0.25 mmol) was dissolved in 2 mL of anhydrous ethanol, 0.2 mL of 30% H₂O₂ solution was added, and the mixture was heated to 70° C. and reacted for 5 hours; then 0.2 mL of 30% H₂O₂ solution was added, and the mixture reacted overnight. A small amount of 10% Pd/C was added, and the mixture was heated at 70° C. for 1 hour. After filtration, the filtrate was concentrated under reduced pressure, and separated and purified by HPLC to give compound KJ-3b-O (57 mg, 58% yield), ESI-MS: m/z=395.2[M+H]⁺.

According to the synthesis methods of compound GY7550-1 and compound K38, compound K63 can be obtained {ESI-MS: m/z=710.81 (M/2)+H]⁺}.

### Synthesis of Compound K64

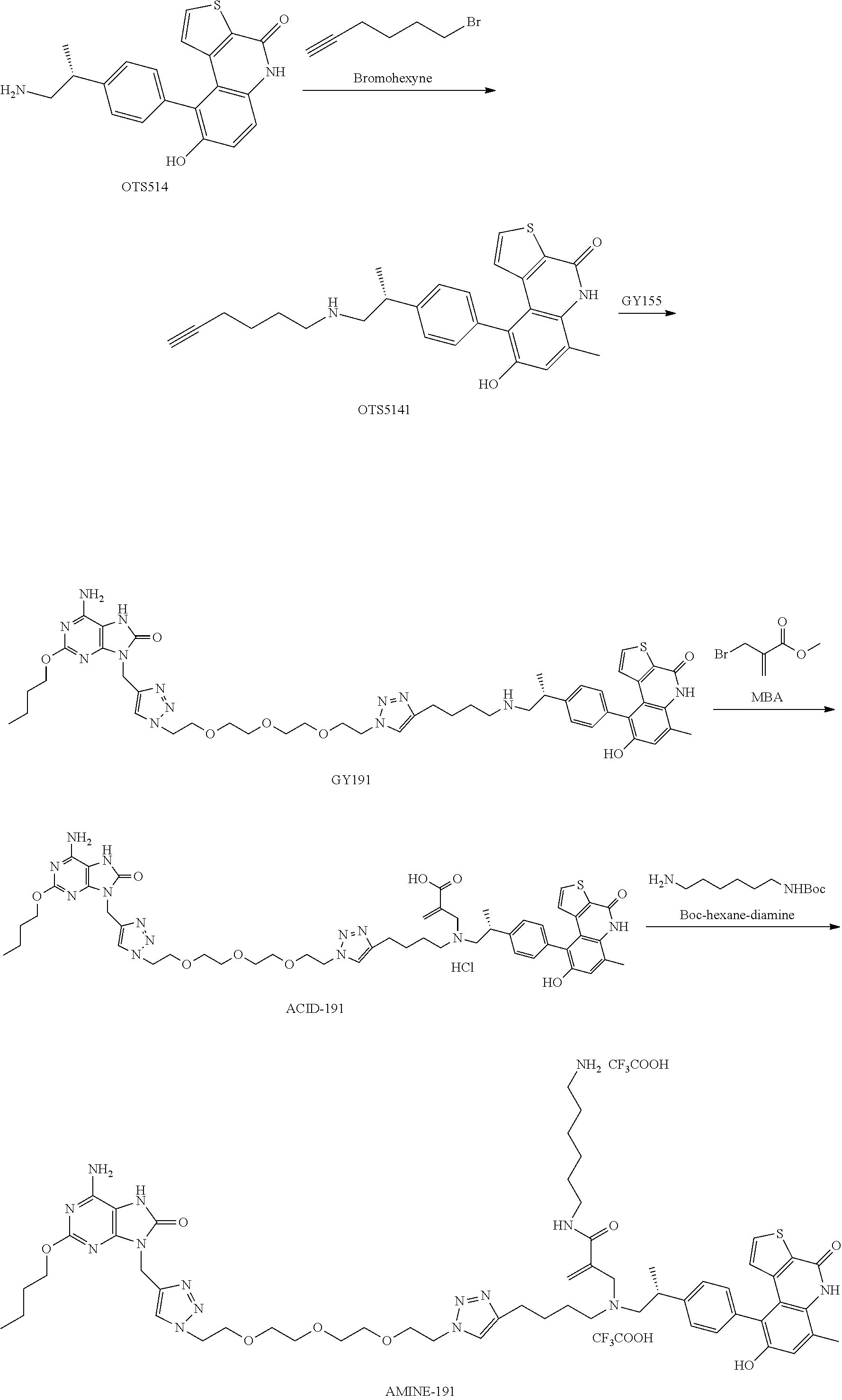

OTS514 (182 mg, 0.5 mmol) was dissolved in 3 mL of DMF, K₂CO₃ (70 mg) was added, and the mixture was stirred uniformly. Compound bromohexyne (81 mg, 0.5 mmol) was dissolved in another 1 mL of DMF, and added dropwise into the solution of OTS514 slowly with stirring at room temperature, and the mixture reacted naturally overnight. The reaction solution was filtered, concentrated under reduced pressure, and purified by preparative HPLC to give compound OTS5141 (147 mg, 66% yield), ESI-MS: m/z=445.2[M+H]⁺. Compound OTS5141 (120 mg, 0.27 mmol) was taken and dissolved in 2 mL of H2O-DMF (3 mL, 1:3 volume ratio), GY155 (137 mg, 0.27 mmol), CuSO₄·5H₂O (8 mg), and sodium L-ascorbate (8 mg) were added, and the resultant mixture was stirred and reacted at room temperature for 6 hours. The reaction solution was filtered, the filtrate was lyophilized, and the lyophilizate was puriied by preparative IHPLC to give compound GY191 (198 mg, 77% yield), ESI-MS: m/z=950.4[M+H]⁺. GY191 (190 mg, 0.2 mmol) was dissolved in 1 mL of DMF, 50 mg of anhydrous K₂CO₃ was added, and the mixture was stirred uniformly. MBA (36 mg, 0.2 mmol) was dissolved in another 2 mL of DMF, and slowly added into the solution of GY191 at room temperature within 3 hours; after the addition, the mixture was stirred and reacted at room temperature overnight. The reaction solution was filtered, the filtrate was lyophilized, and the lyophilizate was dissolved in 3 mL of methanol; then LiOH·H₂O (50 mg) was added, and the mixture was stirred and reacted at room temperature for 10 hours: methanol was removed by vacuum distillation, 5 mL of pure water was added, and the mixture was adjusted to pH 5 with dilute hydrochloric acid; the mixture was stirred uniformly and then lyophilized, and the lyophilizate was purified by HPLC to give the hydrochloride of compound ACID-191 (103 mg), ESI-MS: m/z=1034.5[M+H]⁺. ACID-191 hydrochloride (95 mg, 0.09 mmol) and PyAOP (52 mg, 0.1 mmol) were dissolved in 2 mL of DMF, and DIPEA (70 μL) was added and stirred uniformly, then Boc-hexane-diamine (20 μL) was added, and the mixture was stirred and reacted at room temperature overnight. After the reaction solution was lyophilized, 3 mL of solvent TFA-DCM (1:3) was added, and the mixture was stirred for 1 hour to remove Boc; the solvent was removed by vacuum distillation, and the residue was purified by HPLC to give the TFA salt of compound AMINE-191.2 (67 mg, 55% yield), ESI-MS: m/z=1132.6[M+H]⁺.

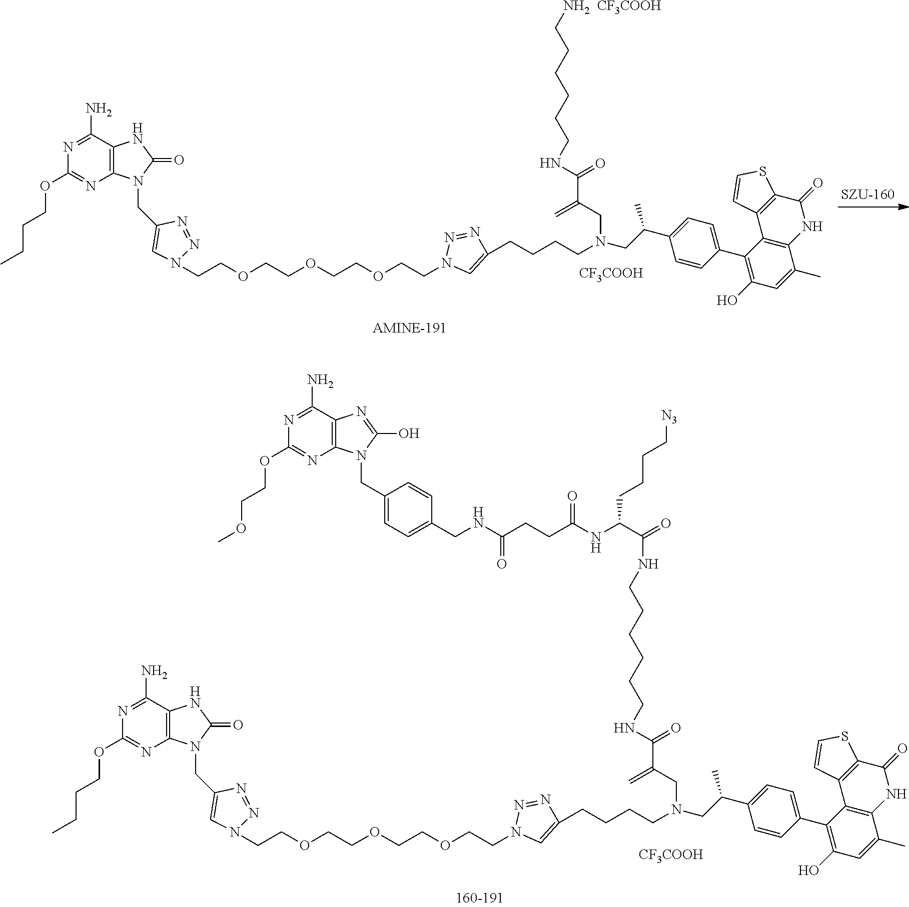

According to the synthesis reaction of ACID-191 hydrochloride and Boc-hexane-diamine, AMINE-191.2 TFA salt and SZU-160 in equimolar amounts reacted to give compound 160-191 {ESI-MS: m/z=857[(M/2)+H]⁺}.

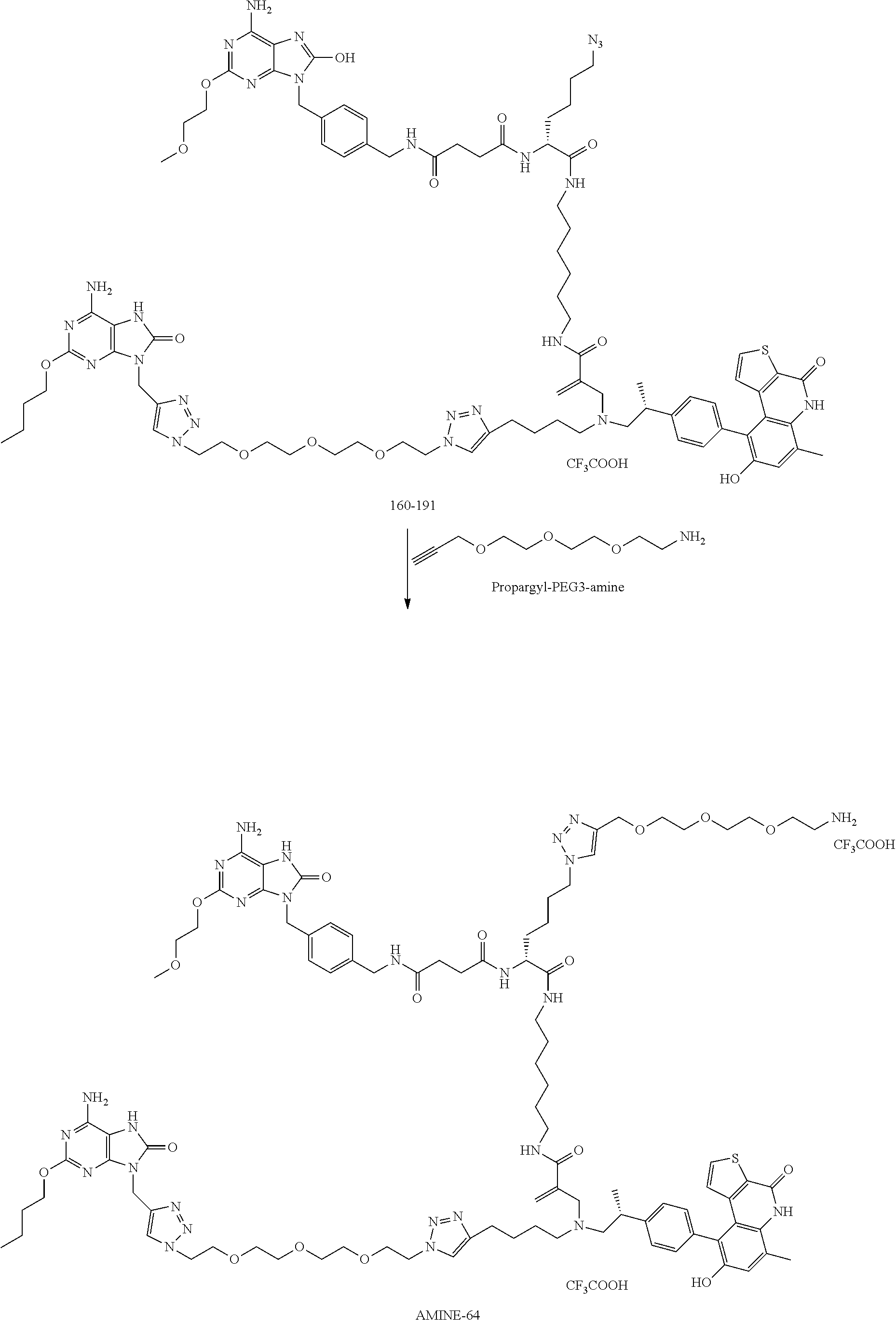

Compound 160-191 and compound propargyl-PEG3-amine were subjected to a standard click reaction catalyzed by copper sulfate and sodium L-ascorbate in the DMF-H2O (3:1) system, and the product was purified by HPLC to give compound AMINE-64 {ESI-MS: m/z=950[(M/2)+H]⁺}.

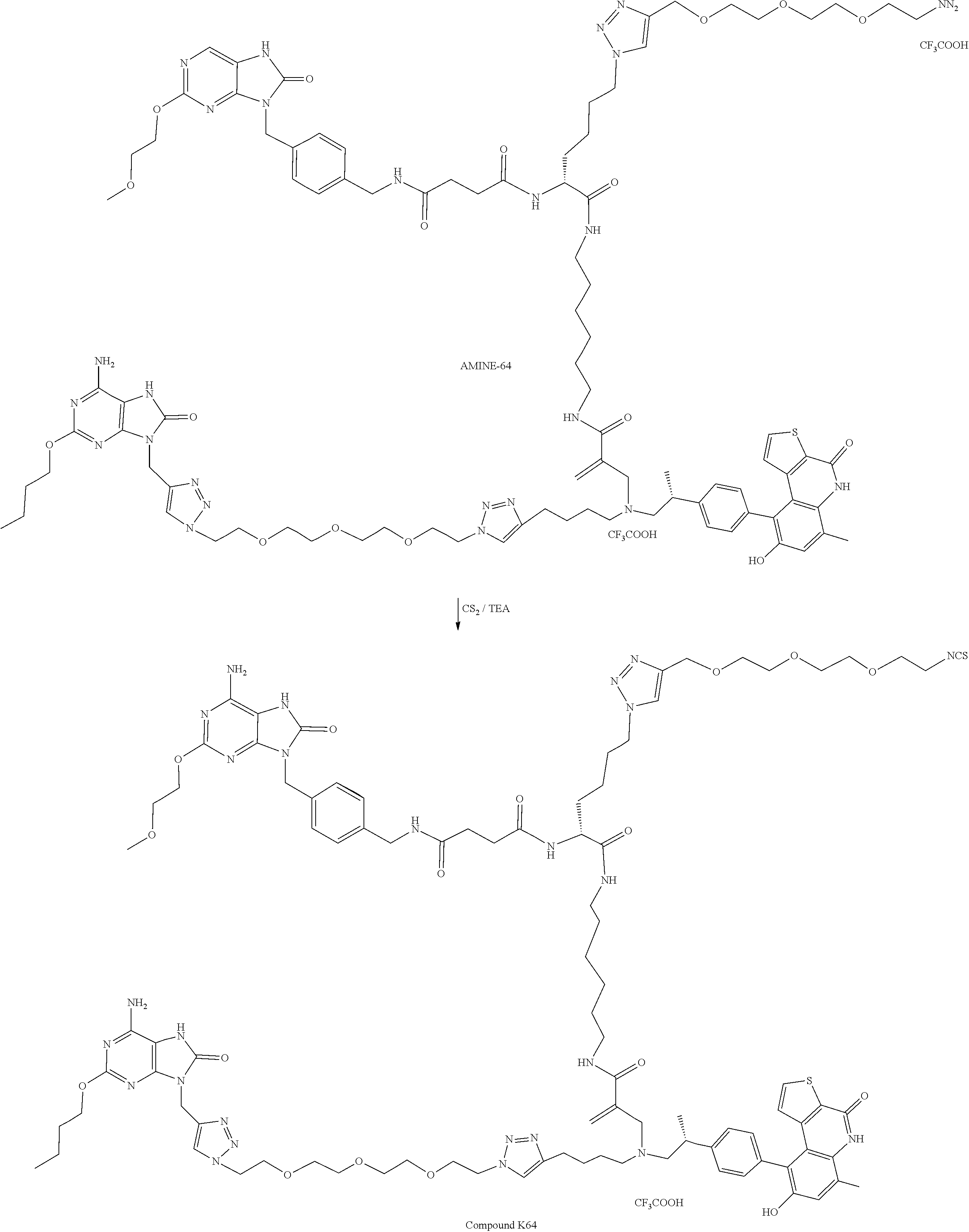

The TFA salt of compound AMINE-64 (106 mg 0.05 mmol) was dissolved in 1 mL of DMF, CS₂ (20 mg) and TEA (35 μL) were added, and the mixture was stirred and reacted at room temperature overnight; TsCl (tosyl chloride, 10 mg) was added, and the mixture continued to react at room temperature until reaction completion (monitored by MS). The reaction solution was filtered, the filtrate was lyophilized, and the resultant product was purified by HPLC to give the TFA salt of compound K64 (43 mg, 42% yield), ESI-MS: m/z=972.2[(M/2)+H]⁺.

Synthesis of compound K65: according to the synthesis route and method of compound K24 except replacing compound BP-22468 with compound SZU-191 {ESI-MS: m/z=1042.5[M+H]⁺}, compound K65 can be obtained {ESI-MS: m/z=774.8[(M/2)+H]⁺}.

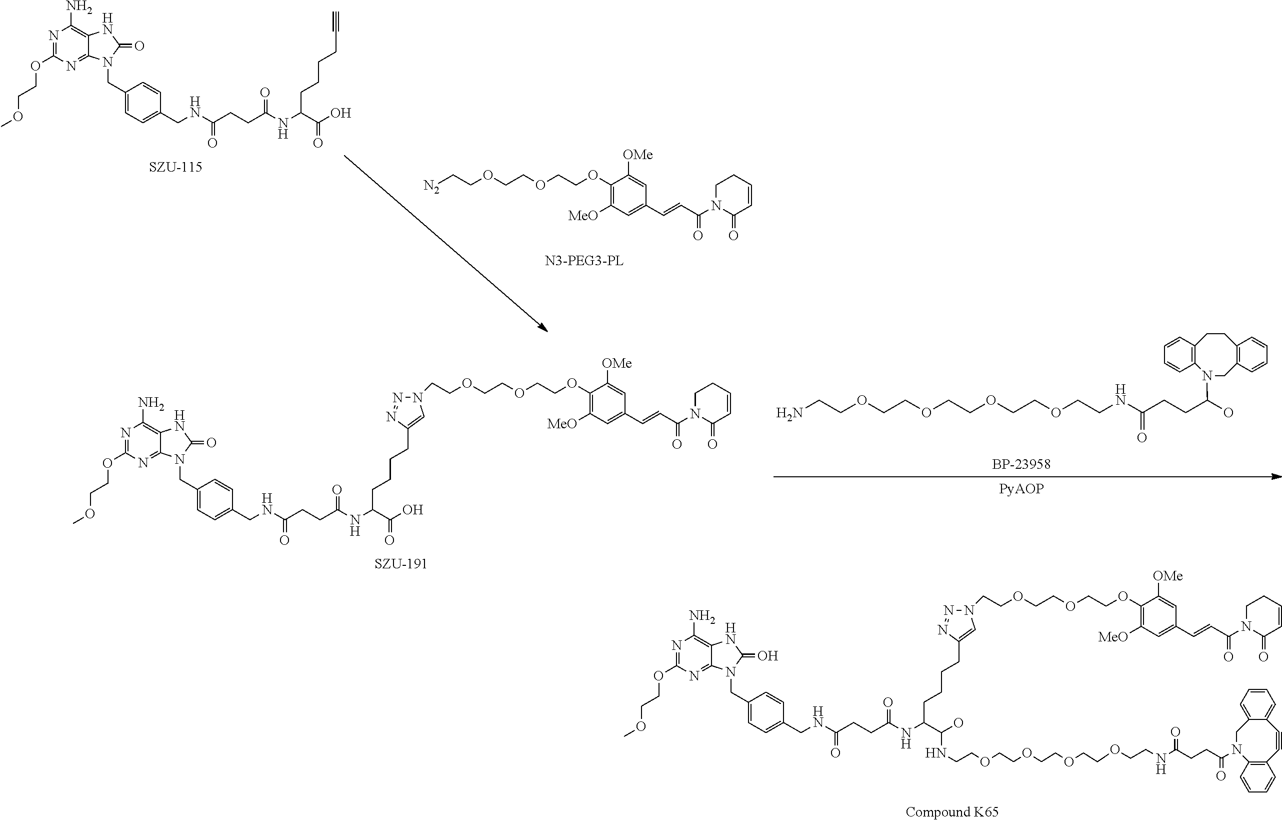

### Synthesis of Compound K-66

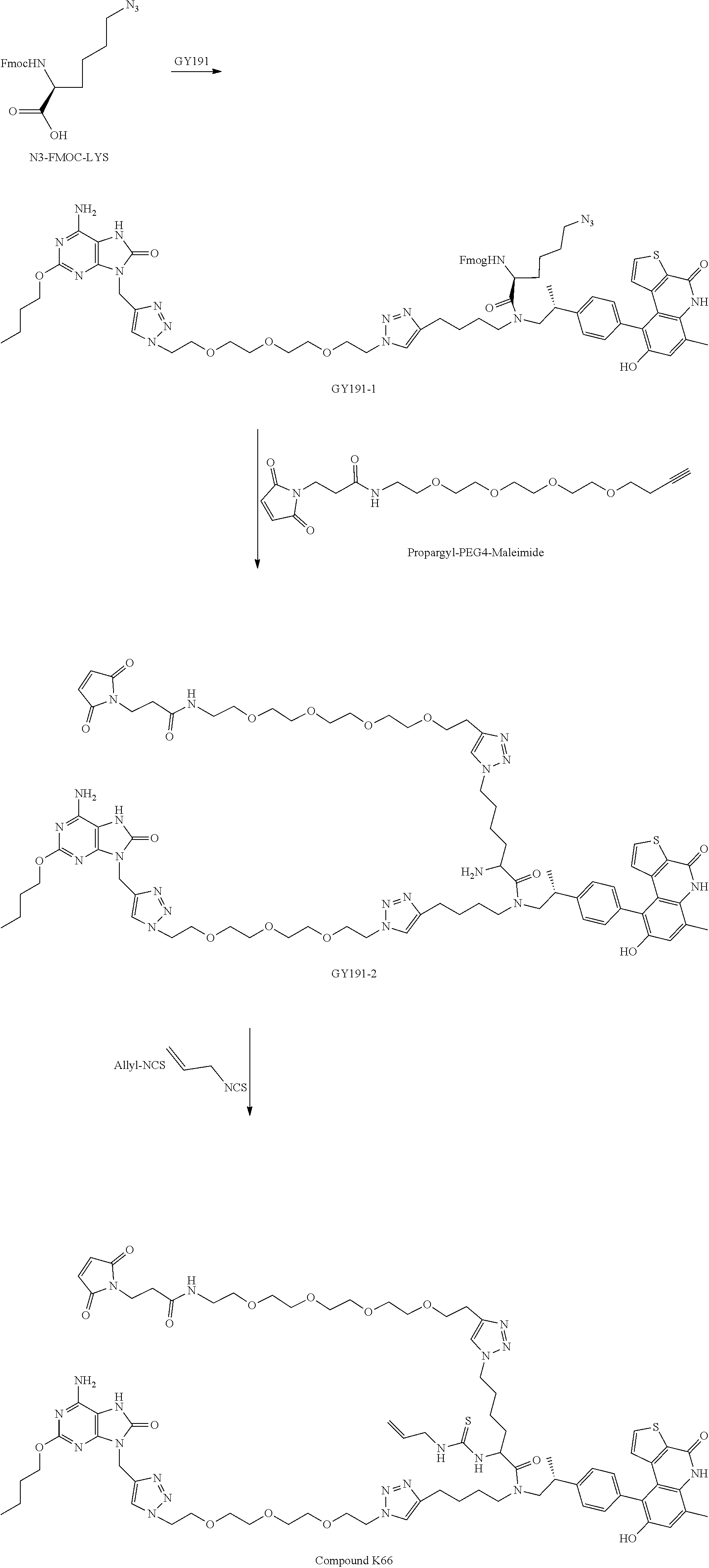

Compounds N3-FMOC-LYS (119 mg, 0.3 mmol), NHS (38 mg, 0.33 mmol), and EDCI (63 mg) were sequentially dissolved in dry DMF (3 mL), and the mixture was stirred at room temperature for 1 hour; then GY191 (285 mg, 0.3 mmol) was added; and the mixture continued to react at room temperature overnight. The reaction solution was lyophilized, and the lyophilizate was purified by silica gel column chromatography (DCM/MeOH=5:1) to give compound 191-1 (247 mg, 62% yield), ESI-MS: m/z=664.3[(M/2)+H]⁺.

Compound 191-1 (200 mg, 0.15 mmol) and compound propargyl-PEG4-maleimide (60 mg, 0.15 mmol) were taken and dissolved in 5 mL of DMF/H₂O (3:1 by volume), CuSO₄·5H₂O (10 mg) and sodium L-ascorbate (10 mg) were added, and the resultant mixture was stirred and reacted at room temperature for 3 hours. The reaction solution was filtered, the filtrate was lyophilized, and the lyophilizate was added with 3 mL of DMF and piperidine (0.5 mL); and the mixture was stirred and reacted at room temperature overnight. The reaction solution was lyophilized, and the lyophilizate was purified by HPLC to give compound 191-2 (124 mg, 55% yield), ESI-MS: m/z=751.4[(M/2)+H]⁺.

Compound 191-2 (100 mg, 0.067 mmol) was dissolved in 2 mL of DMF, and TEA (0.1 mL) and allyl-NCS (10 μL) were added; the resultant mixture was shaken and reacted at room temperature overnight. The reaction solution was lyophilized, and the lyophilizate was separated and purified by HPLC to give compound K66 (51.5 mg, 48% yield), ESI-MS: m/z=801.0[(M/2)+H]⁺.

### Synthesis of Compound K67

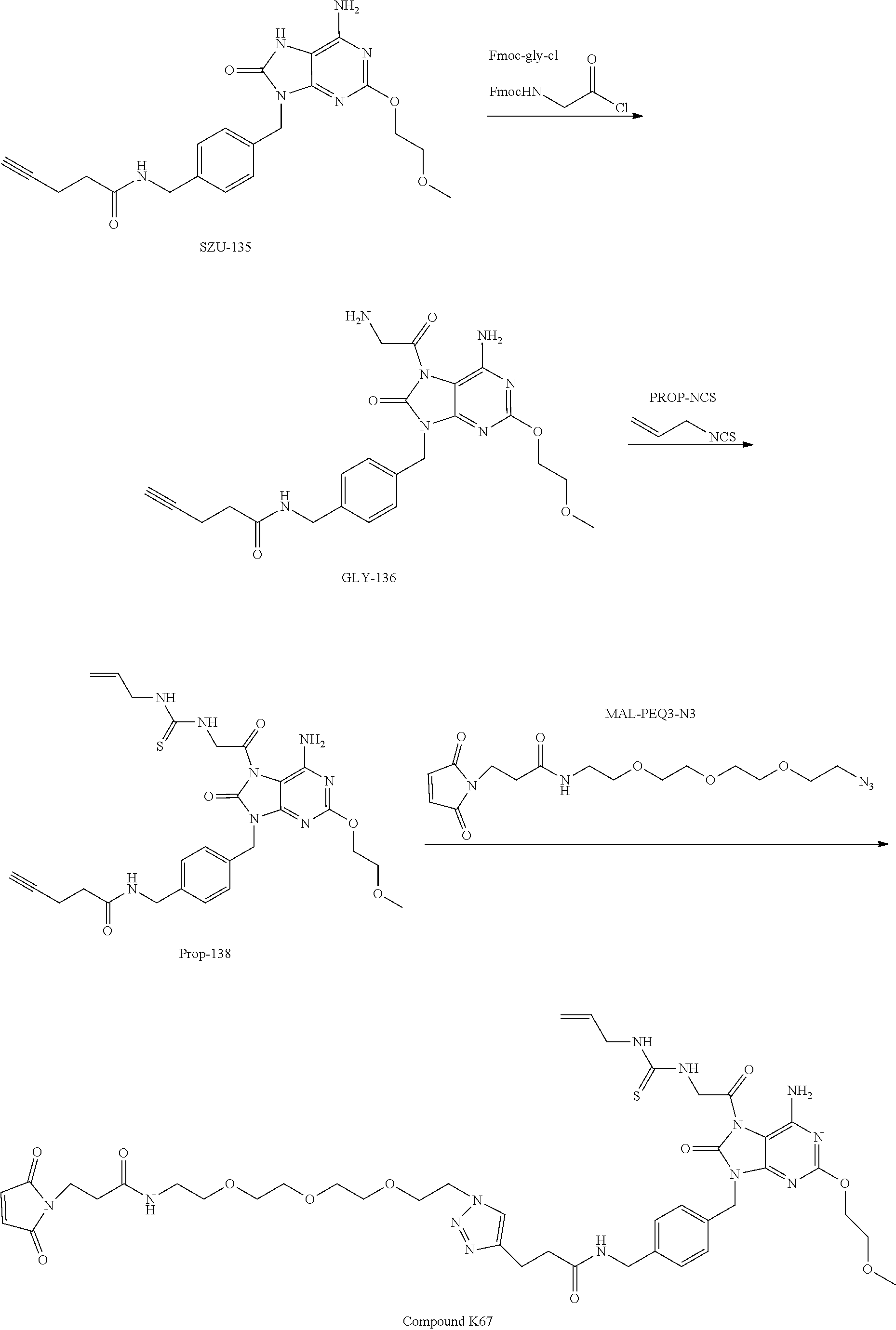

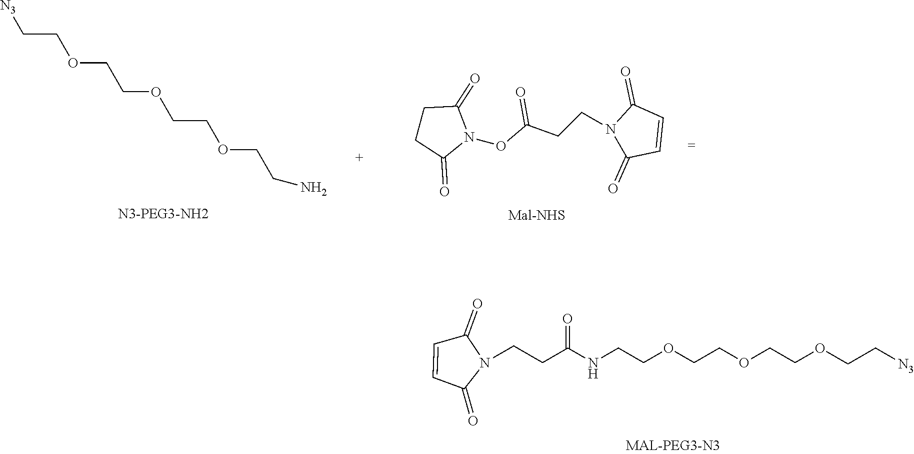 [SMILES: [N-]=[N+]=NCCOCCOCCOCCN ; O=C(CCN1C(=O)C=CC1=O)ON1C(=O)CCC1=O ; [N-]=[N+]=NCCOCCOCCOCCNC(=O)CCN1C(=O)C=CC1=O]

Compound SZU-136 (425 mg, 1 mmol) was dissolved in 10 mL of THF-DCM (1:1, v/v), DMAP (60 mg) was added and dissolved with stirring, then Fmoc-Gly-C1 (316 mg, 1 mmol) was added, and the mixture reacted at room temperature overnight. The reaction solution was added with 11 mL of piperidine, and was stirred and reacted at room temperature for 3 hours. The reaction solution was distilled under reduced pressure to remove the solvent, and the residue was separated by silica gel column chromatography (MeOH/DCM=1:5, v/v) to give compound GLY-136 (265 mg, 55% yield), ESI-MS: m/z=4822[M+H]. Compound GLY-136 (240 mg, 0.5 mmol) was taken and dissolved in DMSO (3 mL), 0.2 mL of triethylamrine (TEA) and PROP-NCS (50 mg) were added, and the resultant mixture was stirred and reacted at room temperature for 6 hours. The reaction solution was lyophilized, and the lyophilizate was separated by silica gel column chromatography (MeOH/DCM=1:5, v/v) to give compound PROP-136 (215 mg, 74% yield), ESI-MS: m/z=581.2 [M+H]⁺.

Compound N3-PEG3-NH42 (66 mg, 0.3 mmol) and compound Mal-NHS (80 mg, 0.3 mmol) were mixed and dissolved in 3 mL of DMF-H2O (2:1, v/v), and the mixture was stirred at room temperature for 1 hour; then PROP-136 (175 mg, 0.3 mmol), CuSO₄·5H₂O (10 mg), and sodium L-ascorbate (10 mg) were added; the resultant mixture was stirred and reacted at room temperature overnight. The reaction solution was filtered, the filtrate was lyophilized, and the lyophilizate was purified by preparative HPLC; the purified solution was concentrated under reduced pressure; and the concentrated solution was lyophilized to give compound K67 (111 mg, 39% yield), ESI-MS: m/z=950.4[M+H]⁺.

### Example 2

In the following Examples, PD-L1 antibody was purchased from MedChemExpress (Atezolizumab, CAS number: 1380723-44-3; for its sequence, please refer to: https://go.drugbank.com/drugs/DB11595 https://www.genome.jp/dbget-bin/www_bget?dr:D10773); pure products were treated by dialysis.

The preparation procedure of antibody samples for detection is as follows: 20 μg of the sample to be tested was placed in a centrifuge tube, 2 μL of GlycoBuffer 2 (10×) was added, and then ultrapure water was added so that the final volume of the system reached 20 μL. The above sample was diluted 10 times (the final concentration was 0.1 mg/mL), and the sample to be tested was tested according to the detection parameters below.

Detection parameters and mass spectrometry conditions for the antibodies are as follows:

### I. Mass Spectrometry Conditions:

### 1. Instruments and Devices

| | | | Device |
|---|---|---|---|
| Instrument name | Instrument model | Manufacturer | status |
| liquid | ExionLC | Shimadzu | normal |
| chromatography | | | |
| mass spectrometry | X500B QTOF | AB SCIEX | normal |
| chromatographic | 2.1 mm*50 mm | Waters | normal |
| column | BEH300 | | |
| | C4 1.7 mm, | | |
| | Waters Corp, | | |
| | Milford, MA | | |

### 2. Reagents and Reagent Solutions

| | Catalog | | |
|---|---|---|---|
| Reagent name | number | Grade | Manufacturer |
| acetonitrile | NA | LC-MS | Merck |
| formic acid | F298780 | LC-MS | aladdin |
| pure water | NA | NA | watsons |

### 3. Liquid Chromatography Conditions

### 3.1 Mobile Phase Gradient

| | | |
|---|---|---|
| | A phase (0.1% | B phase (0.1% formic |
| Time | formic acid in water) | acid in acetonitrile) |

| | | | |
|---|---|---|---|
| 1 | min | 80% | 5% |
| 8 | min | 20% | 80% |
| 12 | min | 20% | 80% |
| 12.1 | min | 80% | 5% |

### 3.2 Detection Parameters

| Parameter | Parameter value | |
|---|---|---|
| detection wavelength | NA | |
| flow rate | 0.3 | mL/min |
| injection volume | 10 | μL |
| column temperature | 60° | C. |
| acquisition time | 12 | min |

| | |
|---|---|
| elution mode | gradient elution |

### 4. Mass Spectrometry Conditions

| Parameter | Parameter value | |
|---|---|---|
| ion source | ESI | |
| scan mode | TOF MS | |
| scan range (Da) | 1000-4000 | |
| nebulizer gas | 50 | psi |
| auxiliary heating gas | 50 | psi |
| curtain gas | 30 | psi |
| temperature | 500° | C. |
| declustering potential | 275 | V |

### II. Mass Spectrometric Analysis of Molecular Weight of a Naked Antibody and Conjugated Antibodies (Taking PD-L1 Antibody Atezolizumab as an Example)

20 μg of the antibody sample to be tested was placed in a centrifuge tube, 2 μL of GlycoBuffer 2 (10×) was added, and then ultrapure water was added so that the final volume of the system reached 20 μL. The above sample was diluted 10 times (the final concentration was 0.1 mg/mL), and the sample to be tested was tested according to the instrument detection parameters above. The molecular weight of Atezolizumab was measured to be 144590.5 by mass spectrometry deconvolution. The method for mass spectrometric analysis of molecular weight of conjugated antibodies was the same as that of a naked antibody.

### III. Illustration by Maleimide Group Conjugation

The conjugation method of compound K3 and antibody PD-L1:

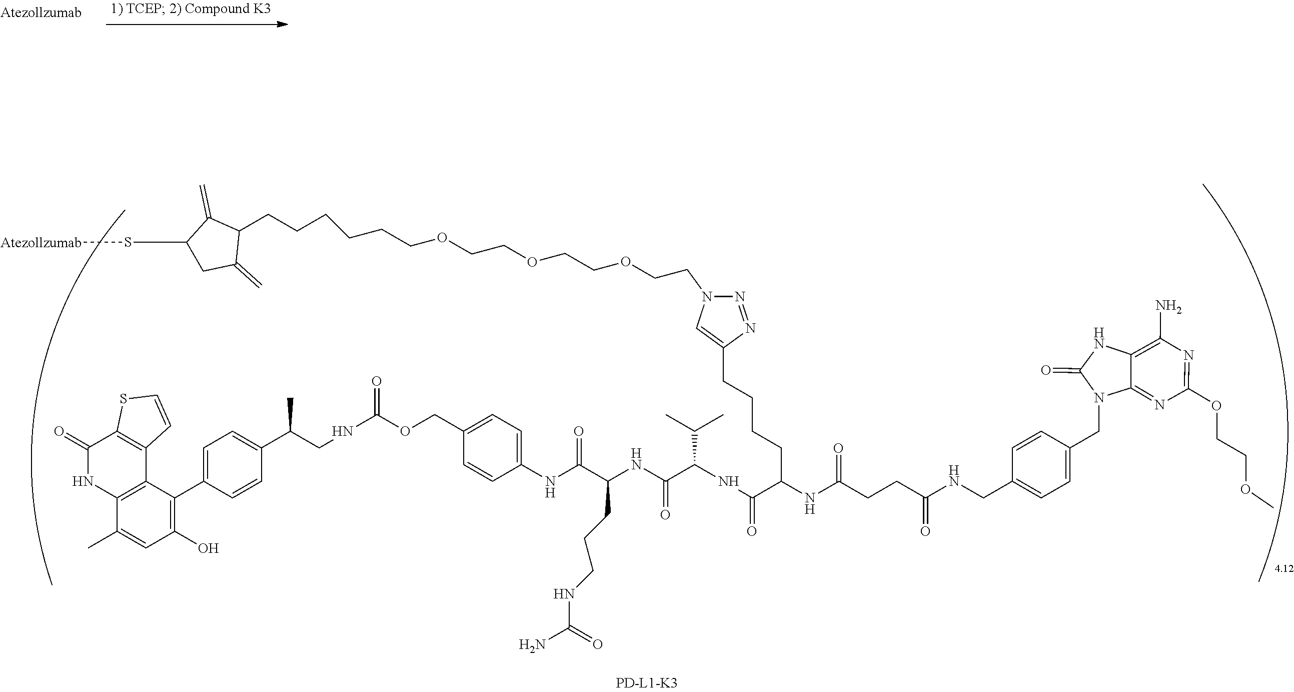

The PD-L1 antibody (Atezolizumab, 90 mg) was dissolved in 20 mL of DPBS (20 mg/mL), and the mixture was adjusted to pH 17.0 with an EDTA solution (concentration: 5 mM). The mixture was added with 12 equivalents of TCEP solution (5 mMN of TCEP aqueous solution) and reduced at room temperature for 3 hours. The resultant solution was filtered and purified by a 30 KD Vivaspin ultrafiltration centrifuge tube (Vivaspin centrifugal concentrator), and the solvent was directly exchanged to 20 mL of 4.2 mM histidine solution.

The pH value of the histidine solution of the above reduced antibody (5 mL) was adjusted to 7.5-8 with Tris buffer; and the solution of compound K3 (12 mg) in N,N-dimethylacetamide (150 μL) was added. This reaction mixture was shaken at 20° C. for 6 hours. The resultant solution was purified by ultrafiltration with a 30 KD Vivaspin ultrafiltration centrifuge tube (Vivaspin centrifugal concentrator), the solvent was exchanged to give Atezolizumab-conjugated K3 (Atezolizumab-K3, PD-L1-K3) in 4.2 mM histidine solution. Mass spectrometric analysis of molecular weight and drug-to-antibody ratio (DAR) was performed. As measured by SEC-QTOF-MS, the average molecular weight was 151606, and the average DAR value was 4.12.

Similarly, other K series of compounds were conjugated to Atezolizumab via a maleimide group to form general formula I:

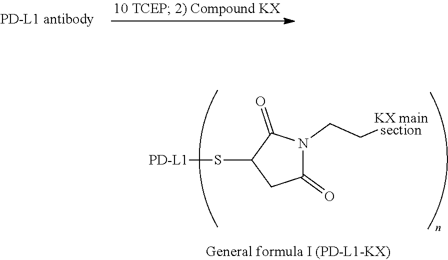

wherein the PD-L1 antibody is Atezolizumab, wherein X represents the serial number of compound K, and n represents the DAR value. DAR values were determined to be 3.5-4.5, as shown in Table 1.

TABLE 1

| | General formula I | | | | | | | | | | | |
|---|---|---|---|---|---|---|---|---|---|---|---|---|
| | PD-L1- | PD-L1- | PD-L1- | PD-L1- | PD-L1- | PD-L1- | PD-L1- | PD-L1- | PD-L1- | PD-L1- | PD-L1- | PD-L1- |
| | K1 | K2 | K3 | K6 | K7 | K8 | K10 | K13 | K14 | K15 | K16 | K17 |
| DAR | 3.56 | 3.90 | 4.12 | 3.43 | 4.32 | 3.46 | 4.51 | 3.58 | 3.74 | 4.35 | 3.48 | 4.31 |
| value | | | | | | | | | | | | |

| | General formula I | | | | | | | | | |
|---|---|---|---|---|---|---|---|---|---|---|
| | PD-L1- | PD-L1- | PD-L1- | PD-L1- | PD-L1- | PD-L1- | PD-L1- | PD-L1- | PD-L1- | PD-L1- |
| | K18 | K19 | K20 | K22 | K24 | K25 | K26 | K27 | K29 | K30 |
| DAR | 4.47 | 4.45 | 4.47 | 4.48 | 3.65 | 3.69 | 3.59 | 3.47 | 3.75 | 4.48 |
| value | | | | | | | | | | |
| | General formula I | | | | | | | | | |
| | PD-L1- | PD-L1- | PD-L1- | PD-L1- | PD-L1- | PD-L1- | PD-L1- | PD-L1- | PD-L1- | PD-L1- |
| | K32 | K33 | K34 | K36 | K41 | K42 | K43 | K62 | K66 | K67 |
| DAR | 3.63 | 4.41 | 3.73 | 3.87 | 4.41 | 3.49 | 3.97 | 3.8 | 3.68 | 4.12 |
| value | | | | | | | | | | |

### IV. Exemplary Azide-Conjugated Antibodies (Illustrated by Compound K23).

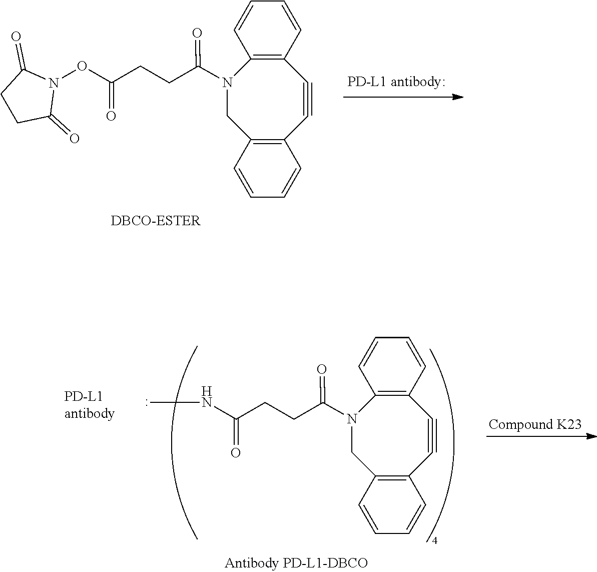

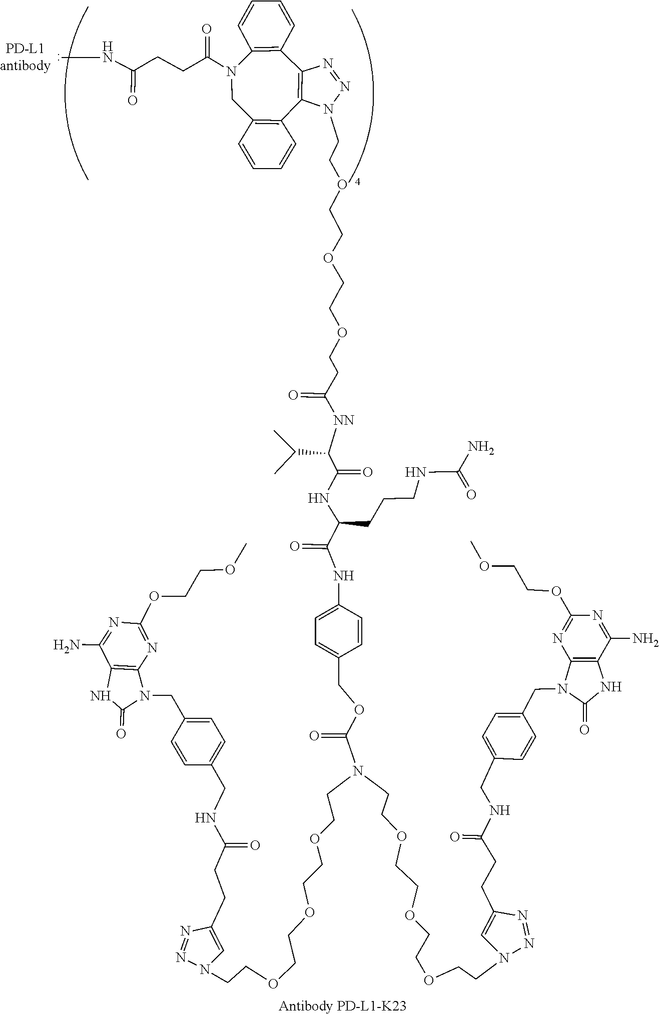

The activated ester of DBCO-acid DBCO-ESTER (20 eq) and the PD-L1 antibody Atezolizumab (1 eq) were mixed in 10% DMSO in pure water, and the mixture was shaken and reacted at 10° C. for 12 hours. The mixture was detected by mass spectrometry until the antibody reacted completely. Small molecules were removed from the reaction mixture with a 10 KD molecule filter membrane, the compound-antibody conjugate PD-L1-DBCO was eluted with DPBS solution, and the drug-to-antibody ratio was determined to be 4. The solution of the conjugated antibody PD-L1-DBCO in DPBS was added with 10% volume of DMSO, then added with 1.5 equivalents of compound K23 was added, and the mixture was shaken and reacted at 25° C. for 12 hours; small molecules were removed with a 20 KD molecule filter membrane. DPBS was used to dissolve the antibody to give the solution of the antibody-conjugated compound PD-L1-K23 in DPBS. As measured by SEC-QTOF-MS, the average molecular weight was 152203, and the average drug-to-antibody ratio was 4.

The same route and method as those for preparing PD-L1-K23 were used to give the conjugates of compounds K11, K21, K44, and K45 respectively conjugated to the PD-L1 antibody (DAR=4), i.e. PD-L1-K11 (average molecular weight: 150549.3), PD-1L1-K21 (average molecular weight: 152693.1), PD-L1-K44 (average molecular weight: 147541.6), and PD-L1-K45 (average molecular weight: 148066.1):

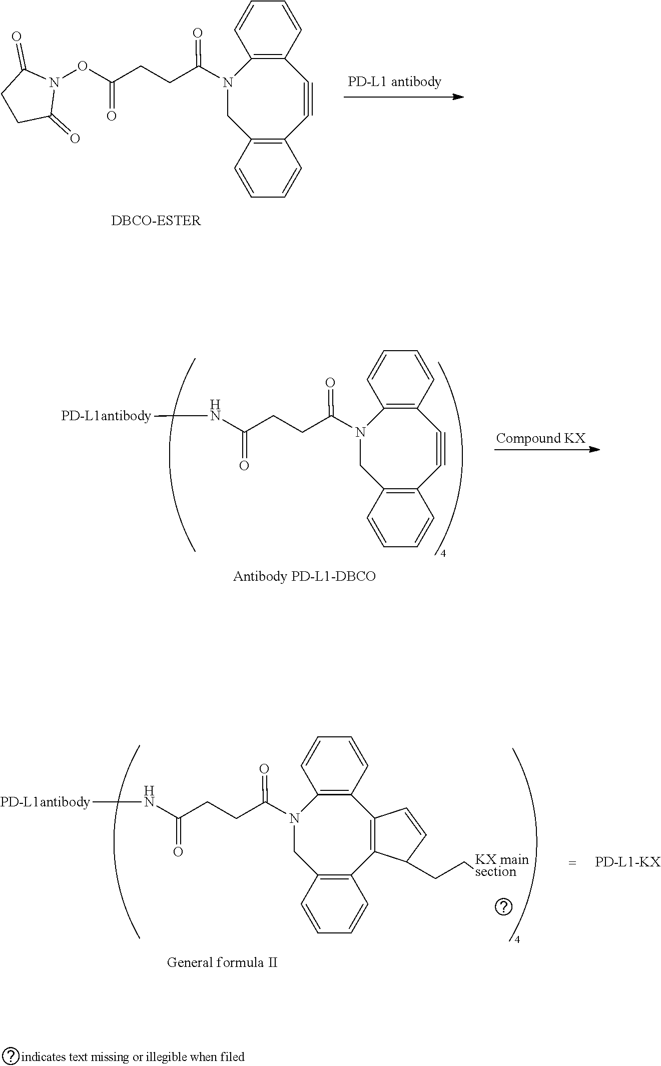

General formula II represents PD-L1-K1, PD-L1-K21, PD-LA-K23, PD-L11-K44, and PD-L1-K45, wherein the PD-L1 antibody is Atezolizumab.

### V. Exemplary Compound-Conjugated Antibodies Using DBCO as Conjugation Group (Illustrated by Compound K57)

According to the preparation method of PD-L1-K23 except replacing compound DBCO-ESTER with Azido-PEG3-NHS ester and replacing compound K23 with compound K57, the antibody conjugation product of conjugating the PD-L1 antibody to compound K57, PD-L1-K57 (as measured by SEC-QTOF-MS, the average molecular weight was 153825.3, and the average drug-to-antibody ratio DAR=4).

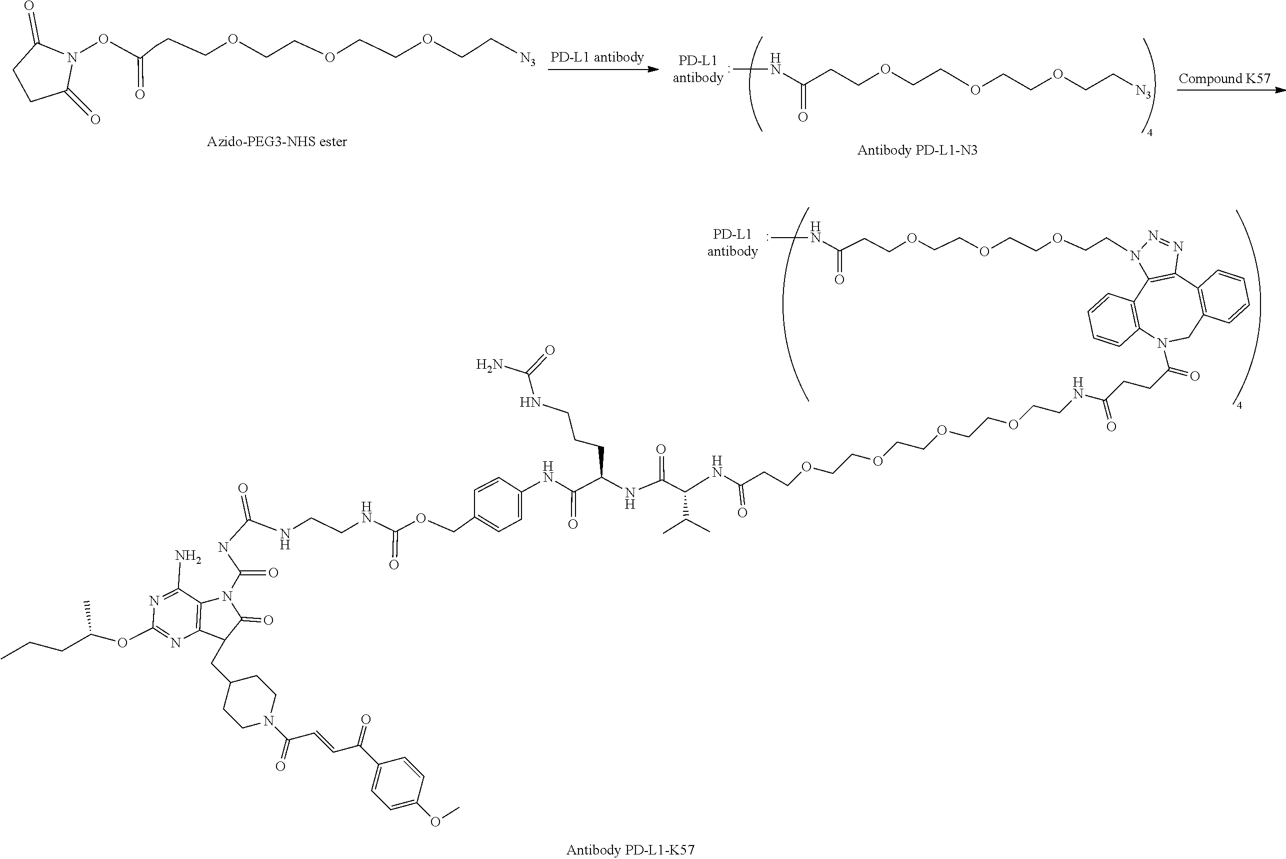

Similarly, by using the same route and method as those for preparing PD-L1-K57, the conjugates of conjugating compounds K28, K28-1, K31, K35, K36, K37, K38, K39, K49, K50, K51, K52, K53, K54, K55, K56, K57, K58, K59, K60 K61, K63, and K65 respectively conjugated to the PD-L1 antibody (DAR:=4) can be obtained, i.e. PD-L1-K28, PD-L1-K28-1, PD-L1-K31, PD-L1-K35, PD-L1-K36, PD-L1-K37, PD-L1-K38, PD-L1-K39, PD-L1-K49, PD-L1-K50, PD-L11-K51, PD-L1-K52, PD-L1-K53, PD-L1-K54, PD-L1-K55, PD-L1-K56, PD-L1-K57, PD-L1-K58, PD-L1-K59, PD-L1-K60, PD-L1-K61, PD-L1-K63, and PD-L1-K65 (see Table 2):

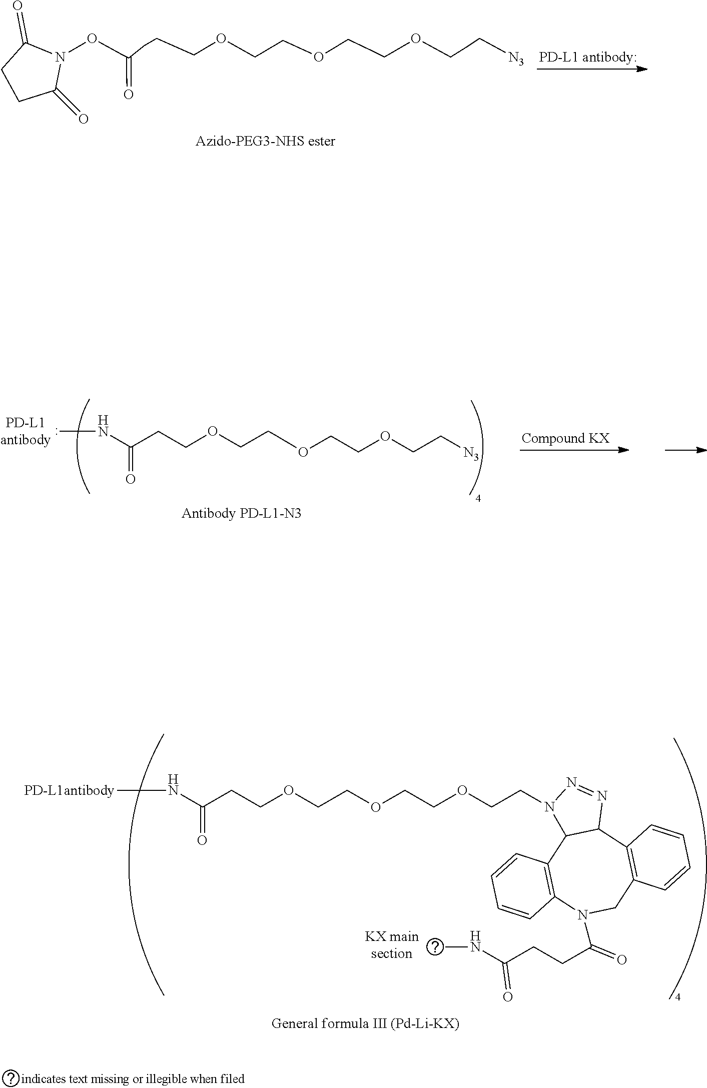

wherein the PD-L1 antibody is Atezolizumab.

TABLE 2

| | General formula III | | | | | | | | |
|---|---|---|---|---|---|---|---|---|---|
| | PD-L1- | PD-L1- | PD-L1- | PD-L1- | PD-L1- | PD-L1- | PD-L1- | PD-L1- | PD-L1- |
| | K28 | K28-1 | K31 | K35 | K36 | K37 | K38 | K39 | K49 |
| Molecular | 153348.5 | 152912.0 | 153056.3 | 152123.4 | 152443.7 | 153825.3 | 153404.6 | 151494.1 | 149204.0 |
| weight | | | | | | | | | |

| | General formula III | | | | | | |
|---|---|---|---|---|---|---|---|
| | PD-L1- | PD-L1- | PD-L1- | PD-L1- | PD-L1- | PD-L1- | PD-L1- |
| | K50 | K51 | K52 | K53 | K54 | K55 | K56 |
| Molecular | 149147.9 | 151398.4 | 151342.3 | 149416.1 | 149472.2 | 151098.0 | 151610.5 |
| weight | | | | | | | |
| | General formula III | | | | | | |
| | PD-L1- | PD-L1- | PD-L1- | PD-L1- | PD-L1- | PD-L1- | PD-L1- |
| | K57 | K58 | K59 | K60 | K61 | K63 | K65 |
| Molecular | 151702.6 | 151646.5 | 151277.9 | 151561.5 | 150517.3 | 151190.1 | 151698.5 |
| weight | | | | | | | |

### VI. Exemplary Compound-Conjugated Antibodies Using Carboxylic Acid as the Conjugation Group (Illustrated by Compound K4)

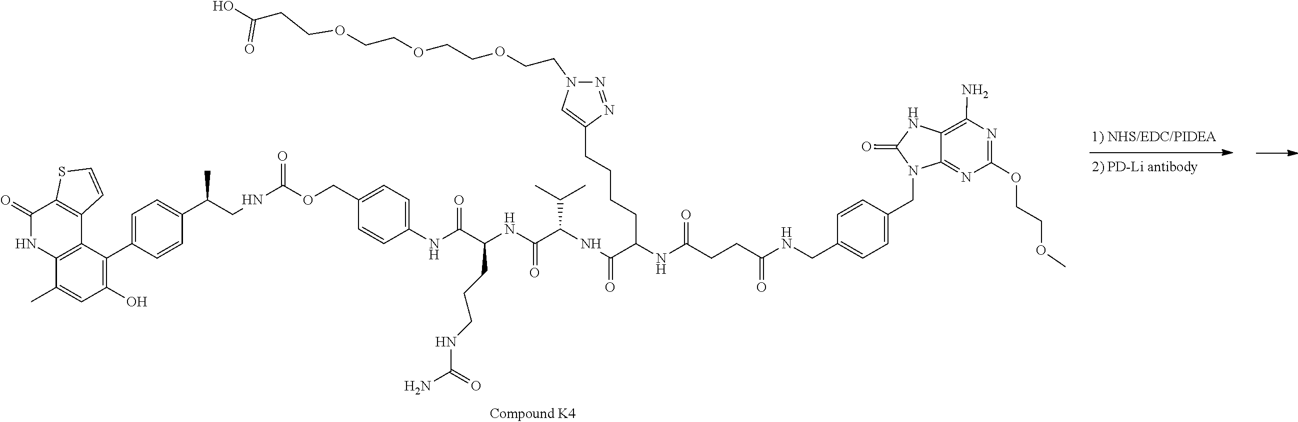 [SMILES: COCCOc1nc(N)c2[nH]c(=O)n(Cc3ccc(CNC(=O)CCC(=O)NC(CCCCc4cn(CCOCCOCCOCCC(=O)O)nn4)C(=O)N[C@H](C(=O)N[C@@H](CCCNC(N)=O)C(=O)Nc4ccc(COC(=O)NC[C@H](C)c5ccc(-c6c(O)cc(C)c7[nH]c(=O)c8sccc8c67)cc5)cc4)C(C)C)cc3)c2n1]

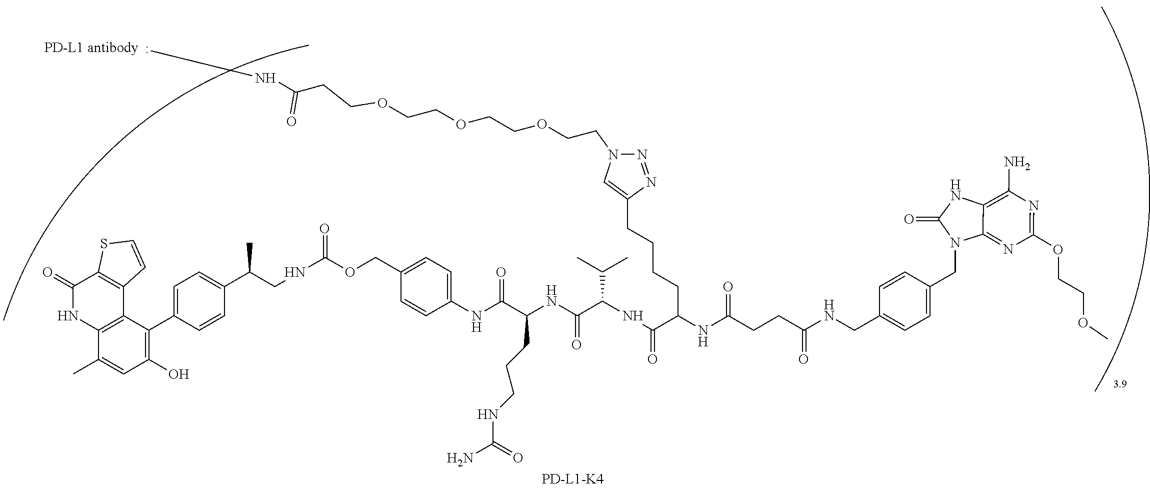

The PD-L1 antibody (Atezolizumab, 100 ng) was dissolved in 20 mL of PBS (the solution of monoclonal antibody PD-L1). To the solution of compound NHS (N-hydroxysuccinimide, 3 mg) in 2 mL of anhydrous DMSO, 6 mg of EDC·HCl and 44 mg of K4 were added, and the resultant mixture solution was stirred at room temperature for 1 hour; then the solution was added into the solution of monoclonal antibody PD-L1 for coupling reaction. The reaction mixture solution was added with 5 μL of DIPEA at 10° C., and the mixture was stirred at 25° C. overnight. Then the mixture was subject to centrifugal ultrafiltration using a 30 kDa MWCO centrifugal ultrafiltration tube, the solvent of the antibody was exchanged to pure water, and the mixture was lyophilized to give the product of conjugating the antibody to K4, PD-L1-K4: 71 mg. As measured by SEC-QTOF-MS, the average molecular weight was 150689, and the drug-to-antibody ratio DAR=3.89.

Similarly, by using the same route and method as those for preparing PD-L1-K4, the conjugates of compounds K5. K9, and K12 respectively conjugated to the PD-L1 antibody can be obtained, i.e. PD-L1-K5 (average molecular weight: 152202, drug-to-antibody ratio DAR=3.97), PD-L1-K9 (average molecular weight: 148620.8, drug-to-antibody ratio DAR=3.78), and PD-L1-K12 (average molecular weight: 149520.4, drug-to-antibody ratio DAR=4.01):

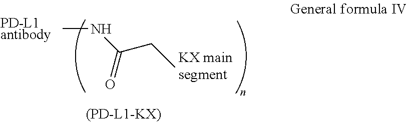

wherein the PD-L1 antibody is Atezolizumab.
   VII. Exemplary Compound-Conjugated Antibodies Using Isothiocyanate as the conjugation Group (Illustrated by Compound K46)

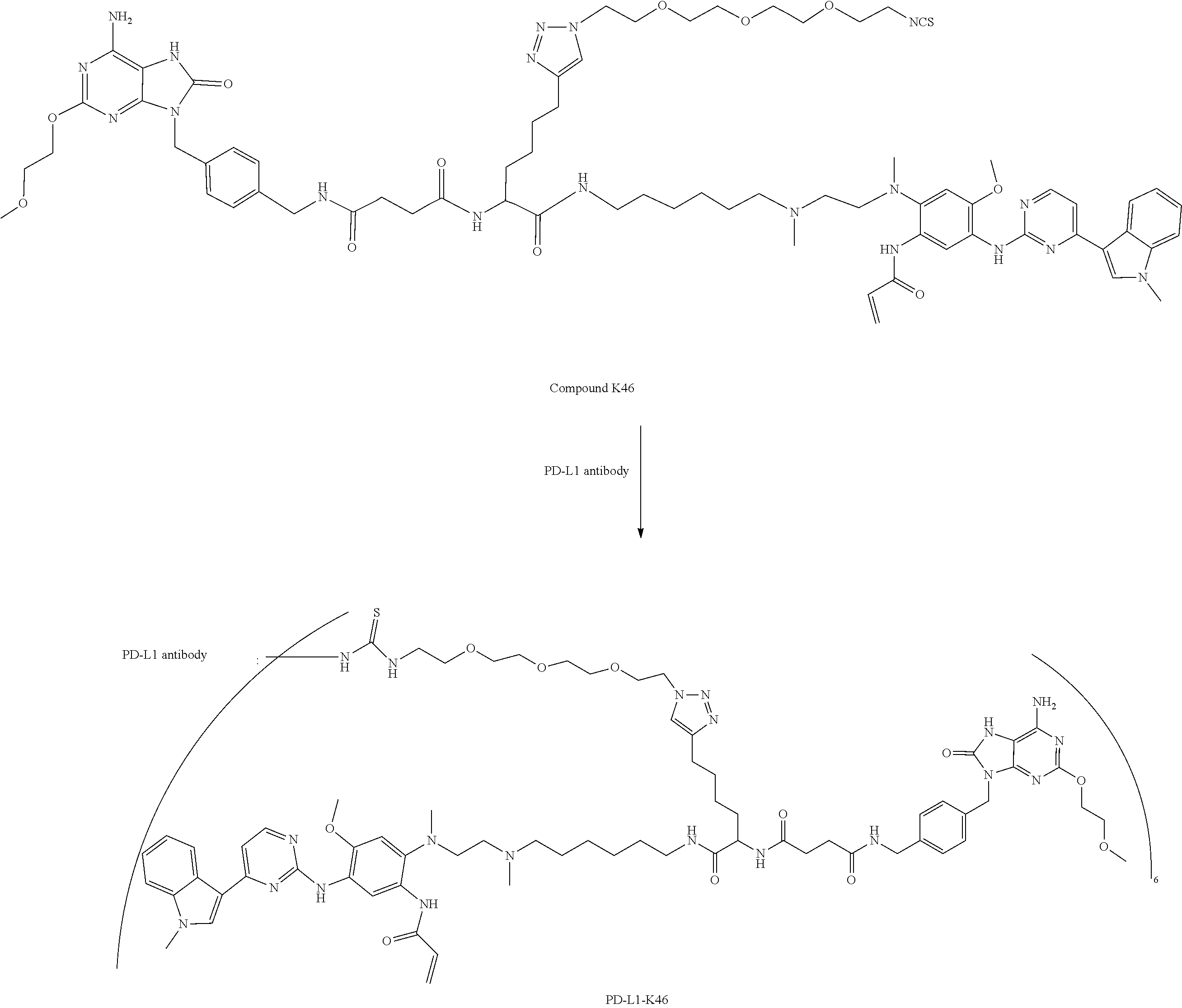

The PD-L1 antibody (Atezolizumab, 100 mg) and 5 μL of TEA were dissolved in 20 mL of PBS (the solution of monoclonal antibody PD-L1); 0.5 mL of the solution of compound K46 (20 mg, 20-fold molar amount) in DMSO was taken and added into the solution of monoclonal antibody PD-L1, and the mixture was stirred and reacted at room temperature 25° C. for 6 hours. The mixture was subject to centrifugal ultrafiltration with a 30 kDa MWCO centrifugal ultrafiltration tube, the solvent of the antibody was exchanged to pure water, and the mixture was lyophilized to give the product of conjugating the antibody to K46, PD-L1-K46: 102 mg. As measured by SEC-QTOF-MS, the average molecular weight was 153042, and the drug-to-antibody ratio DAR=5.99.

Similarly, by using the same route and method as those for preparing PD-L-1-K46, the conjugates of compounds K40, K47, K48, and K64 respectively conjugated to the PD-L1 antibody can be obtained, i.e. PD-L1-K40 (average molecular weight: 150855.6, drug-to-antibody ratio DAR=5.96), PD-L1-K47 (average molecular weight: 152026.8, drug-to-antibody ratio DAR=5.98), PD-L1-K48 (average molecular weight: 151612.5, drug-to-antibody ratio DAR=6.01), and PD-L1-K64 (average molecular weight: 156108.3, drug-to-antibody ratio DAR=5.93):

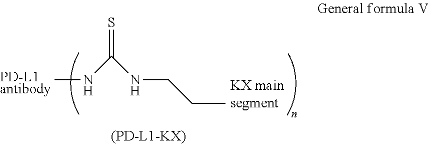

wherein the PD-L1 antibody is Atezolizumab.

### Example 3. ELISA Assay of an Immunocyte Activator (IFN-γ) for the Compounds

The immune activity of each compound is represented by the function of inducing human peripheral blood mononuclear cells (PBMCs) to produce the immune cytokine IFN-γ by, and the EC₅₀ values thereof are shown in Table 3.

### Preparation of Human Peripheral Blood Mononuclear Cells (PBMCs):

1) Peripheral blood samples from healthy human volunteer were collected into heparin anticoagulant tubes to reach 5 mL of fresh blood, and PBS was added at a ratio of 1:1 to dilute the blood (2 mL+2 mL in conventional blood collection tubes).
2) 5 mL of lymphocyte separation medium was put in a 15 mL sterile centrifuge tube for use.
3) The diluted blood was aspirated and slowly added from 1 cm above the separation medium along the tube wall, so that the diluted blood overlaid the separation medium and formed a distinct interface therewith.
4) The centrifuge tube was centrifuged at 2000 rpm at room temperature for 20 min. Now five layers formed in the centrifuge tube: the top layer was plasma layer, and the layer between the plasma layer and the lymphocyte separation medium was the lymphocyte layer as a white film.
5) The mononuclear cell layer as a white film in the middle was aspirated carefully and mononuclear cells were aspirated as many as possible. The mononuclear cells were washed by more than 5 volumes of PBS twice, and each time, the mononuclear cells were centrifuged at 1500 rpm for 10 min.
6) The supernatant was removed, then 1 mL of 1640 medium was added and mixed uniformly. A small amount of the mixture was taken for cell counting.

### General Method:

The prepared PBMCs were plated in a 96-well plate (Costar, 3894) (2×10⁵ cells per well). Each compound in Table 3 was taken and added into the wells with plated cells at ten concentrations with uniform gradient distribution (0.0001-10 μM, diluted with RPMI-1460 medium containing 0.1 DMSO). Then the cells were incubated in a 5% CO₂ incubator at 37° C. for 24 hours; by using a human-IFN-γ kit (cisbio), IFN-γ in the cell culture supernatant was measured according to the procedure in the kit instruction, and EC₅₀ value of each compound was calculated (see Table 3).

TABLE 3

| Compound | K1/SZU-115 | K2/SZU-101 | K3/SZU-115 | K4/SZU-115 |
|---|---|---|---|---|
| EC₅₀ (μM) | 0.1/0.3 | 0.05/0.5 | 0.2/0.3 | 0.15/0.3 |
| IFN-γ ratio | | | | |
| | | K6/GY101/ | | |
| Compound | K5/GY115 | 102-N3 | K7/GY101 | K8/GY101 |
| EC₅₀ (μM) | 0.13/0.3 | 0.10/0.12/ | 0.045/0.12 | 0.11/0.12 |
| IFN-γ ratio | | 0.18 | | |

| Compound | K9^{a}/GY100 | K10^{a}/SZU-136 | K11^{b}/SZU-136 | K12^{a}/SZU-136 |
|---|---|---|---|---|
| EC₅₀ (μM) | 009/0.11 | 0.21/0.37 | 0.19/0.37 | 0.20/0.37 |
| IFN-γ ratio | | | | |
| Compound | K13^{c}/PMQ-1 | K14^{a}/SZU-136 | K15^{a}/SZU-136 | K16^{d}/SZU-136 |
| EC₅₀ (μM) | 0.11/0.32 | 0.01/0.37 | 0.012/0.37 | 0.011/0.37 |
| IFN-γ ratio | | | | |

| Compound | K17/SZU-115 | K18/GY100 | K19/GY155 | K20/SZU-136 |
|---|---|---|---|---|
| EC₅₀ (μM) | 0.21/0.3 | 0.09/0.11 | 0.08/0.33 | 0.07/0.37 |
| IFN-γ ratio | | | | |
| Compound | K21/SZU-101 | K22^{c}/KJ-032 | K23/SZU-136 | K24/SZU-115 |
| EC₅₀ (μM) | 0.006/0.5 | 0.08/0.11 | 0.03/0.37 | 0.003/0.3 |
| IFN-γ ratio | | | | |

| Compound | K25/SZU-115 | K26/SZU-115 | K27/SZU-115 | K28/SZU-115 |
|---|---|---|---|---|
| EC₅₀ (μM) | 0.005/0.3 | 0.002/0.3 | 0.25/0.3 | 0.21/0.3 |
| IFN-γ ratio | | | | |

| | K28-1/SZU-142/ | | | |
|---|---|---|---|---|
| Compound | KJ-138 | K29/SZU-101 | K30/SZU-101 | K31/SZU-115 |
| EC₅₀ (μM) | 0.03/0.41/ | 0.031/0.5 | 0.059/0.5 | 0.029/0.3 |
| IFN-γ ratio | 0.05 | | | |

| | | | K34/KJ002/ | |
|---|---|---|---|---|
| Compound | K32/SZU-115 | K33/SZU-115 | KJ005/KJ006 | K35/SZU-115 |
| EC₅₀ (μM) | 0.16/0.3 | 0.18/0.3 | 0.131/0.22/ | 0.26/0.3 |
| IFN-γ ratio | | | 0.37/0.29 | |
| | K36/GY101/ | | K38^{c}/GY100/ | K39^{c}/SZU-142/ |
| Compound | KJ7552 | K37/SZU-101 | GY7550 | SZU-275 |
| EC₅₀ (μM) | 0.11/0.12/ | 0.22/0.5 | 0.09/0.11/ | 0.001/0.9/ |
| IFN-γ ratio | 0.43 | | 0.37 | 0.001 |

| | K40/SZU-115/ | | K42^{d}/SZU-136/ | |
|---|---|---|---|---|
| Compound | 115-2 | K41^{d}/GY121 | KJ-008 | K43^{c}/SZU-136 |
| EC₅₀ (μM) | 0.001/0.3/ | 0.002/0.05 | 0.007/0.37/ | 0.007/0.37 |
| IFN-γ ratio | 0.01 | | 0.007 | |

| Compound | K44^{b}/GY121 | K45^{b}/SZU-136 | K46/SZU-115 | K47/KJ002 |
|---|---|---|---|---|
| EC₅₀ (μM) | 0.002/0.05 | 0.01/0.37 | 0.09/0.3 | 0.15/0.22 |
| IFN-γ ratio | | | | |

| | | K49/KJ-1b/ | K50/KJ-1a/ | |
|---|---|---|---|---|
| Compound | K48/SZU-115 | KJ-096/KJ-097 | KJ-098/KJ-099 | K51^{a}/KJ-096 |
| EC₅₀ (μM) | 0.089/0.3 | 0.011/0.018/ | 0.023/0.4/ | 0.002/0.003 |
| IFN-γ ratio | | 0.003/0.001 | 0.31/0.42 | |

| | | K53/GY100/GY225/ | K54/GY1001/GY228/ | |
|---|---|---|---|---|
| Compound | K52^{a}/KJ-098 | GY226/GY227 | GY229/GY300 | K55^{a}/GY229 |
| EC₅₀ (μM) | 0.12/0.31 | 0.087/0.11/0.31/ | 0.018/0.11/0.054/ | 0.021/0.028 |
| IFN-γ ratio | | 0.10/0.15 | 0.028/0.031 | |

| | | K57^{a}/KJ-1b/ | K58^{a}/KJ-1a/ | K59^{a}/Pro-123/ |
|---|---|---|---|---|
| Compound | K56^{a}/GY226 | KJ-102 | KJ-104 | KJ-178/KJ-179 |
| EC₅₀ (μM) | 0.10/0.10 | 0.003/0.018/ | 0.009/0.4/ | 0.002/100.3/ |
| IFN-γ ratio | | 0.003 | 0.009 | 0.092/0.002 |

| | | K61^{b}/GY121/ | | K63^{a}/KJ-3b/ |
|---|---|---|---|---|
| Compound | K60^{a}/SZU-103 | GY1401 | K62^{a}/GY100 | KJ-3b-O |
| EC₅₀ (μM) | 0.09/0.1 | 0.002/0.05/ | 0.01/0.11 | 0.08/0.21/ |
| IFN-γ ratio | | 0.002 | | 0.09 |

| | | K65/SZU-115/ | | |
|---|---|---|---|---|
| Compound | K64/SZU-160 | SZU-191 | K66/GY155 | K67^{d}/SZU-136 |
| EC₅₀ (μM) | 0.01/0.05 | 0.05/3.0/0.1 | 0.21/0.33 | 0.26/0.37 |
| IFN-γ ratio | | | | |

Note:
pretreatment before assay of immunocyte activator in the compound sample:
Note ^{a}:
Enzymatic metabolism method
A human liver microsomal enzyme kit (model MVT-1.0, Beijing IPHASE Pharmaceutical Technology Co., Ltd.) was used:
1) each component of the kit was thawed in an ice bath and placed on ice for use;
2) the components to be tested were mixed uniformly according to the set concentrations and ratios, and preincubated at 37° C. for 5 min;
3) the above mixture solutions were distributed into centrifuge tubes (195 μL per tube), and kept warm in a 37° C. water bath; 5 μL of liver microsomes was added into each sample, the mixture was pipetted 3 times for mixing uniformly, and the metabolic reaction was initiated in 37° C. water bath condition; and
4) at time point of two hour, stop solution was added into the incubation system to stop the reaction; the mixture was filtered through a 20K molecule membrane to produce a filtrate, and then each component was assayed according to the general method to determine EC₅₀ value.
Note ^{b}:
Glutathione reduction method
PBS buffer (pH 6) was uniformly mixed with a sample according to the set concentration and ratio, and then an equimolar amount of glutathione was added. After stirring at 37° C. under nitrogen protection for 24 hours, each component was assayed according to the general method to determine EC₅₀ value.
Note ^{c}:
Enzymatic cleavage method
Each compound was uniformly mixed with enzyme Cathepsin-B (10 μg, Cat. #: 10483-H08H, Sino Biological) in PBS according to the set concentration and ratio, results are shown in Table 4.
K-series-compound conjugated PD-L1 antibodies of the present invention can provide significantly improved antitumor effect targeting PD-L1 (the tumor volume inhibition rate against mouse colon cancer MC38-hPD-L1 cells (Atezolizumab as control): 67%), as shown in Table 3 below (wherein PD-L1-KX represents the product of conjugating each K series compound to Atezolizumab in general formulae I-V, and X represents the serial number of the K series compound): and the mixture was co-incubated at room temperature for 3 hours; the mixture was filtered through a 20K molecule membrane to produce a filtrate, and then each component was assayed according to the general method to determine EC₅₀ value.
Note ^{d}:
Hydrolysis method
Acetic acid/sodium acetate buffer (pH 5) was uniformly mixed with a sample according to the set concentration and ratio. After stirring at 37° C. for 72 hours, each component was assayed according to the general method to determine EC₅₀ value.
Note ^{e}:
Phosphodiesterase method
Each compound was uniformly mixed with phosphodiesterase I (10 μg, Cat. #: P4506, Sigma-Aldrich) in PBS according to the set concentration and ratio, and the mixture was co-incubated at room temperature for 3 hours; the mixture was filtered through a 20K molecule membrane to produce a filtrate, and then each component was assayed according to the general method to determine EC₅₀ value.

### Example 4. Verification of Antitumor Effect of the Representative K-Series-Compound Conjugated Antibodies in the Present Invention (PD-L1-KX: X Represents the Serial Number of the K Series Compound)

### Establishment of Tumor Model of Mouse Colon Cancer and Tumor Inhibition Assay

MC38-hPD-L1 cells (a humanized MC38 colon cancer cell line expressing human PD-L1 protein, Shanghai Model Organisms Center, Inc.) in the logarithmic phase were collected by digestion and centrifugation, and washed with PBS twice before counting, and the cell concentration was adjusted to 2.3×10⁵ cells/mL. The mice were 6-week-old SPF-grade humanized C57BL/6 mice. The right back of the mice was shaved completely with a shaver, and 100 μL of the cell suspension was aspirated with a syringe and injected subcutaneously into the back of the mice after removing bubbles. When the tumor diameter reached 4-5 mm, administration was started.

Assessment of therapeutic effect on tumor-bearing mice and antitumor effect of the compounds: tumor-bearing mice (BALB/c) were randomly divided into control group, Atezolizumab antibody group (PD-L1 antibody group), and PD-L1-KX conjugate group (Atezolizumab-conjugated compound KX) (5 mice per group). Injections were formulated according to administration dosages (the solvent composition was a mixture of: 5% DMSO; +40% PEG300; +5% Tween 80; +50% PBS), which were 3 mg/kg for PD-L1 antibody group and 4 mg/kg for PD-L1-KX conjugate group, and a volume of 100 μL was injected intraperitoneally each time. Administration was respectively performed on Day 8 and Day 12 after tumor inoculation, totaling 2 injections. The tumor was measured with a vernier caliper and the survival of the mice was recorded every three days at the beginning of administration and after administration. The tumor volume was calculated by the following method: 0.5×a×b², wherein a is the long-axis diameter and b is the short-axis diameter. When the tumor diameter reached 2 cm in a mouse, the mouse should be executed by cervical dislocation according to animal ethics, and the tumor tissue was excised to measure tumor volume for calculating tumor inhibition rate {(tumor volume in control group −50 tumor volume in conjugate group/tumor volume in control group)×100%}. The results are shown in Table 4.

K-series-compound conjugated PD-L1 antibodies of the present invention can provide significantly improved antitumor effect targeting PD-L1 (the tumor volume inhibition rate against mouse colon cancer MC38-hPD-L1 cells (Atezolizumab as control): 67%), as shown in Table 3 below (wherein PD-L1-KX represents the product of conjugating each K series compound to Atezolizumab in general formulae I-V, and X represents the serial number of the K series compound):

TABLE 4

| PD-L1-KX | PD-L1-K1 | PD-L1-K2 | PD-L1-K3 | PD-L1-K4 | PD-L1-K5 |
|---|---|---|---|---|---|
| Tumor | 100 | 79 | 95 | 92 | 93 |
| inhibition rate % | | | | | |

| PD-L1-KX | PD-L1-K6 | PD-L1-K7 | PD-L1-K8 | PD-L1-K9 | PD-L1-K10 |
|---|---|---|---|---|---|
| Tumor | 94 | 83 | 91 | 78 | 82 |
| inhibition rate % | | | | | |

| PD-L1-KX | PD-L1-K11 | PD-L1-K12 | PD-L1-K13 | PD-LI-K14 | PD-L1-K15 |
|---|---|---|---|---|---|
| Tumor | 86 | 88 | 73 | 82 | 83 |
| inhibition rate % | | | | | |
| PD-L1-KX | PD-L1-K16 | PD-L1-K17 | PD-L1-K18 | PD-L1-K19 | PD-L1-K20 |
| Tumor | 93 | 81 | 79 | 83 | 89 |
| inhibition rate % | | | | | |
| PD-L1-KX | PD-L1-K21 | PD-L1-K22 | PD-L1-K23 | PD-L1-K24 | PD-L1-K25 |
| Tumor | 81 | 91 | 77 | 73 | 77 |
| inhibition rate % | | | | | |

| PD-L1-KX | PD-L1-K26 | PD-L1-K27 | PD-L1-K28 | PD-L1-K28-1 | PD-L1-K29 |
|---|---|---|---|---|---|
| Tumor | 79 | 96 | 95 | 91 | 75 |
| inhibition rate % | | | | | |

| PD-L1-KX | PD-L1-K30 | PD-L1-K31 | PD-L1-K32 | PD-L1-K33 | PD-L1-K34 |
|---|---|---|---|---|---|
| Tumor | 81 | 91 | 73 | 76 | 82 |
| inhibition rate % | | | | | |
| PD-L1-KX | PD-L1-K35 | PD-L1-K36 | PD-L1-K37 | PD-L1-K38 | PD-L1-K39 |
| Tumor | 91 | 93 | 89 | 84 | 76 |
| inhibition rate % | | | | | |
| PD-L1-KX | PD-L1-K40 | PD-L1-K41 | PD-L1-K42 | PD-L1-K43 | PD-L1-K44 |
| Tumor | 87 | 91 | 80 | 72 | 71 |
| inhibition rate % | | | | | |
| PD-L1-KX | PD-L1-K45 | PD-L1-K46 | PD-L1-K47 | PD-L1-K48 | PD-L1-K49 |
| Tumor | 78 | 92 | 79 | 83 | 79 |
| inhibition rate % | | | | | |
| PD-L1-KX | PD-L1-K50 | PD-L1-K51 | PD-L1-K52 | PD-L1-K53 | PD-L1-K54 |
| Tumor | 98 | 79 | 77 | 84 | 73 |
| inhibition rate % | | | | | |
| PD-L1-KX | PD-L1-K55 | PD-LI-K56 | PD-L1-K57 | PD-L1-K58 | PD-L1-K59 |
| Tumor | 74 | 89 | 87 | 75 | 95 |
| inhibition rate % | | | | | |
| PD-L1-KX | PD-L1-K60 | PD-L1-K61 | PD-L1-K62 | PD-L1-K63 | PD-L1-K64 |
| Tumor | 93 | 89 | 87 | 75 | 93 |
| inhibition rate % | | | | | |

| PD-L1-KX | PD-L1-K65 | PD-L1-K66 | PD-L1-K67 |
|---|---|---|---|
| Tumor | 88 | 91 | 97 |
| inhibition rate % | | | |

Described above are only preferred specific embodiments of the present invention, but the protection scope of the present invention is not limited thereto. Those skilled in the art familiar with the technologies in this field could easily envisage changes or alternatives within the technical scope disclosed by the present invention, which shall be encompassed within the protection scope of the present invention. Therefore, the protection scope of the present invention shall be determined by the protection scope of the claims.

1. An antibody-conjugated immune-activating small molecule compound, characterized in that the general formula formed by the antibody and the immune-activating small molecule compound is as shown in the following formula: 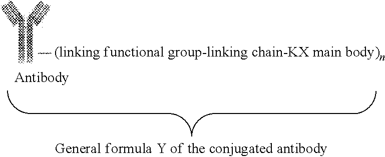
wherein the linking functional group is maleimide group, carboxyl, isothiocyano, azido or alkynyl, the linking chain is alkoxy or alkyl chain, the KX-main body is the main part of the immune-activating small molecule KX, the antibody is PD-L1 antibody, and the antibody-conjugated immune-activating small molecule compound is one of the following compounds:

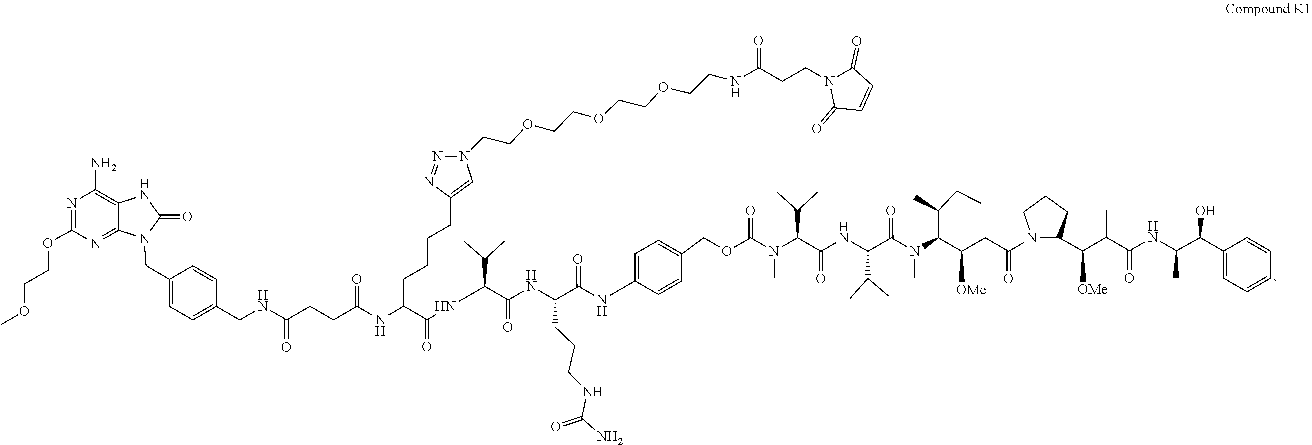

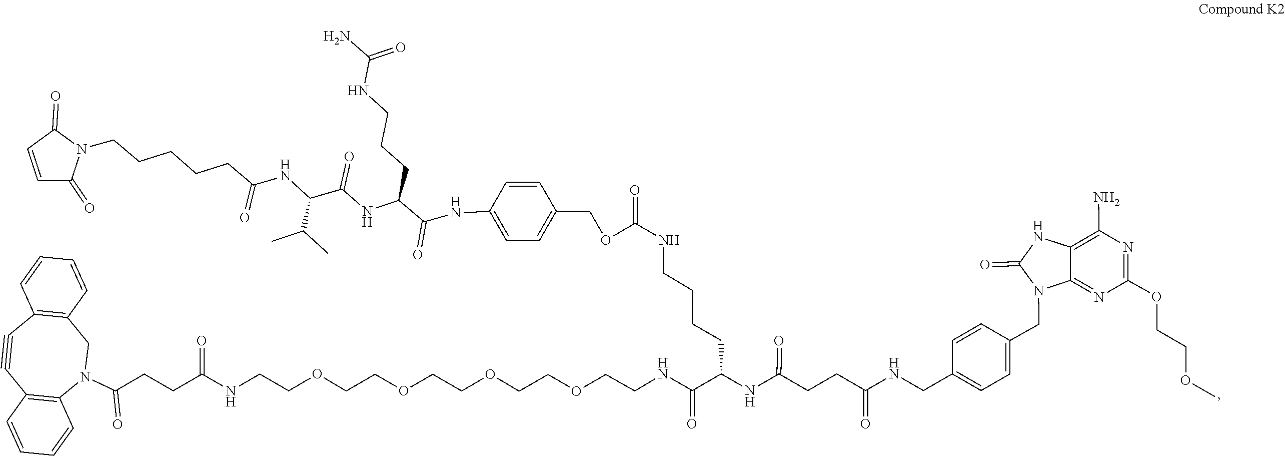 [SMILES: COCCOc1nc(N)c2[nH]c(=O)n(Cc3ccc(CNC(=O)CCC(=O)N[C@@H](CCCCNC(=O)OCc4ccc(NC(=O)[C@H](CCCNC(N)=O)NC(=O)[C@@H](NC(=O)CCCCCN5C(=O)C=CC5=O)C(C)C)cc4)C(=O)NCCOCCOCCOCCOCCNC(=O)CCC(=O)N4Cc5ccccc5C#Cc5ccccc54)cc3)c2n1]

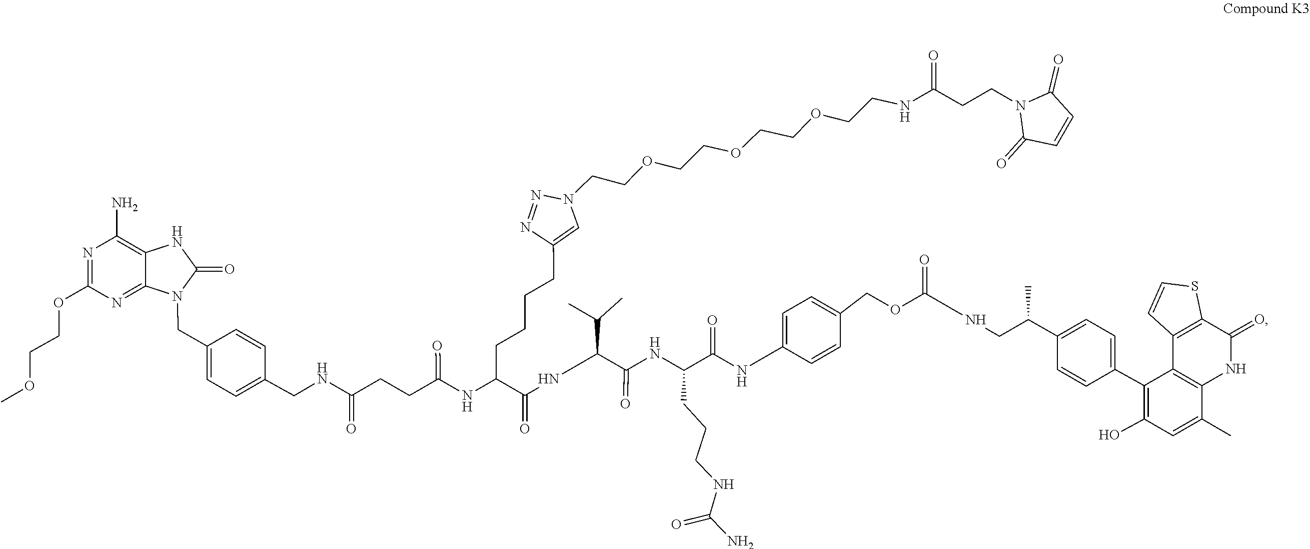 [SMILES: COCCOc1nc(N)c2[nH]c(=O)n(Cc3ccc(CNC(=O)CCC(=O)NC(CCCCc4cn(CCOCCOCCOCCNC(=O)CCN5C(=O)C=CC5=O)nn4)C(=O)N[C@H](C(=O)N[C@@H](CCCNC(N)=O)C(=O)Nc4ccc(COC(=O)NC[C@H](C)c5ccc(-c6c(O)cc(C)c7[nH]c(=O)c8sccc8c67)cc5)cc4)C(C)C)cc3)c2n1]

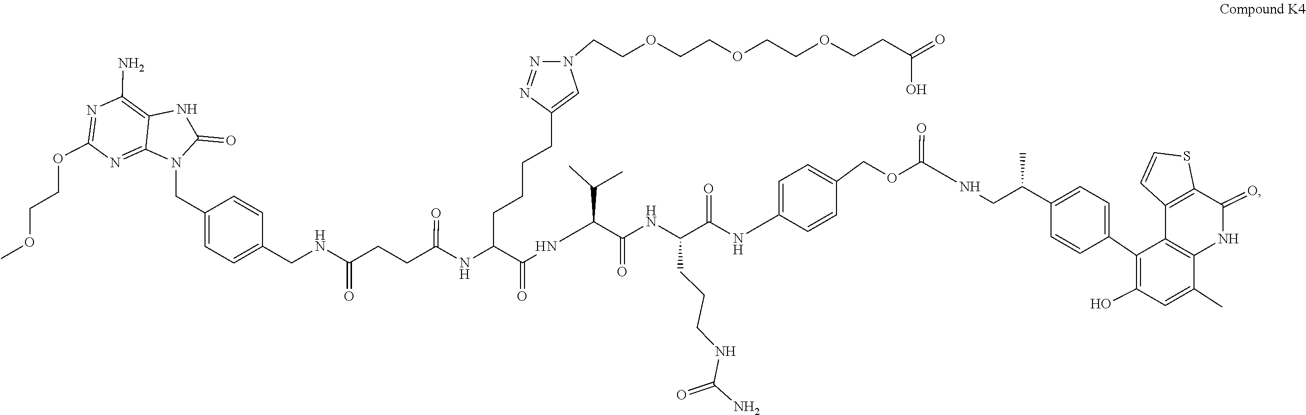 [SMILES: COCCOc1nc(N)c2[nH]c(=O)n(Cc3ccc(CNC(=O)CCC(=O)NC(CCCCc4cn(CCOCCOCCOCCC(=O)O)nn4)C(=O)N[C@H](C(=O)N[C@@H](CCCNC(N)=O)C(=O)Nc4ccc(COC(=O)NC[C@H](C)c5ccc(-c6c(O)cc(C)c7[nH]c(=O)c8sccc8c67)cc5)cc4)C(C)C)cc3)c2n1]

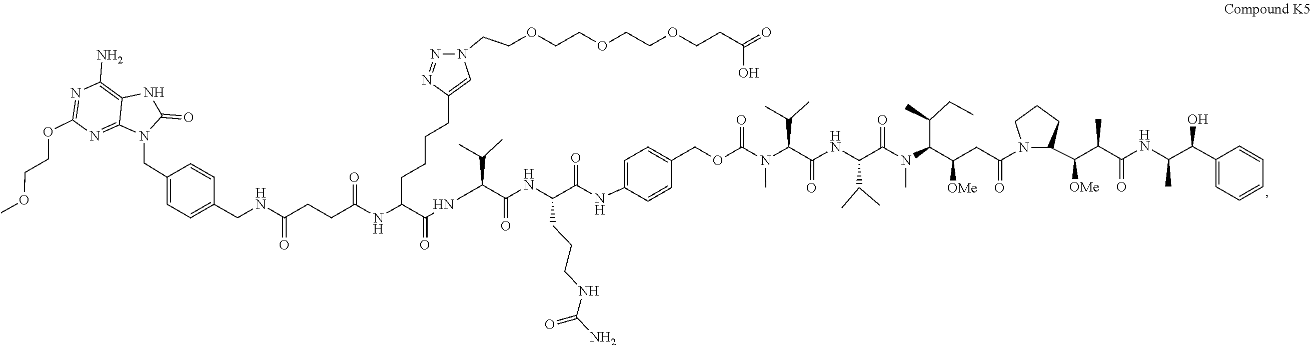

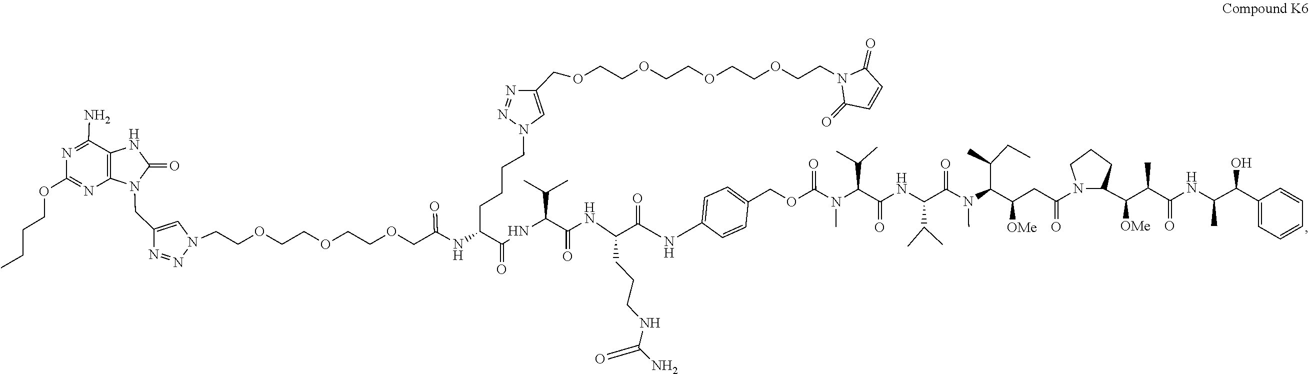

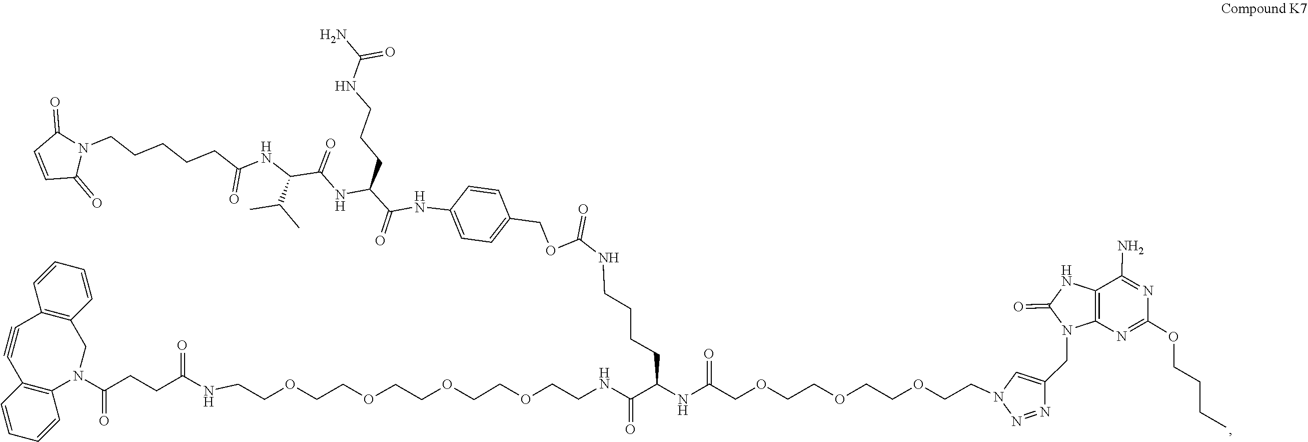 [SMILES: CCCCOc1nc(N)c2[nH]c(=O)n(Cc3cn(CCOCCOCCOCC(=O)N[C@H](CCCCNC(=O)OCc4ccc(NC(=O)[C@H](CCCNC(N)=O)NC(=O)[C@@H](NC(=O)CCCCCN5C(=O)C=CC5=O)C(C)C)cc4)C(=O)NCCOCCOCCOCCOCCNC(=O)CCC(=O)N4Cc5ccccc5C#Cc5ccccc54)nn3)c2n1]

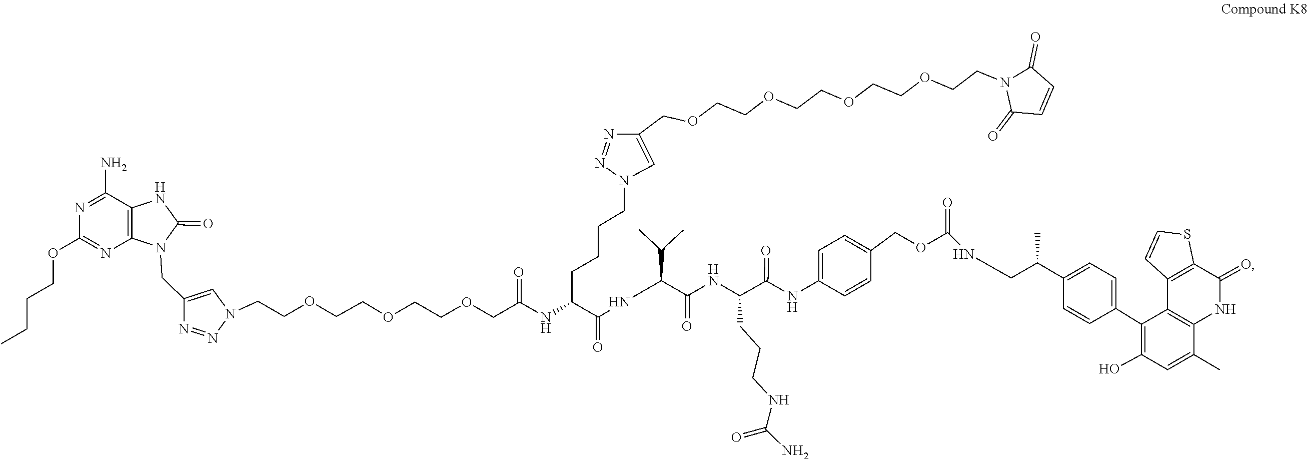 [SMILES: CCCCOc1nc(N)c2[nH]c(=O)n(Cc3cn(CCOCCOCCOCC(=O)N[C@H](CCCCn4cc(COCCOCCOCCOCCN5C(=O)C=CC5=O)nn4)C(=O)N[C@H](C(=O)N[C@@H](CCCNC(N)=O)C(=O)Nc4ccc(COC(=O)NC[C@H](C)c5ccc(-c6c(O)cc(C)c7[nH]c(=O)c8sccc8c67)cc5)cc4)C(C)C)nn3)c2n1]

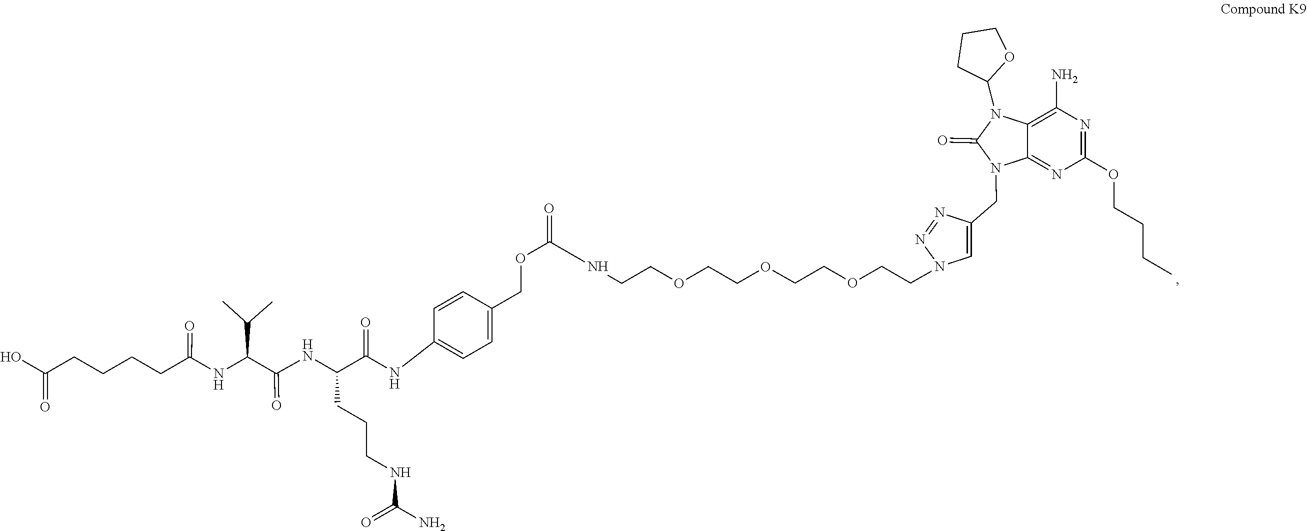

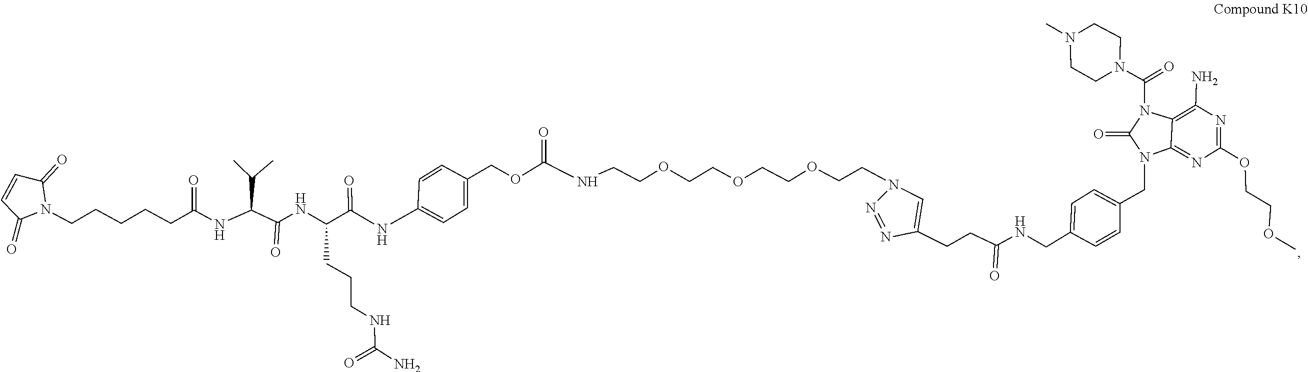 [SMILES: COCCOc1nc(N)c2c(n1)n(Cc1ccc(CNC(=O)CCc3cn(CCOCCOCCOCCNC(=O)OCc4ccc(NC(=O)[C@H](CCCNC(N)=O)NC(=O)[C@@H](NC(=O)CCCCCN5C(=O)C=CC5=O)C(C)C)cc4)nn3)cc1)c(=O)n2C(=O)N1CCN(C)CC1]

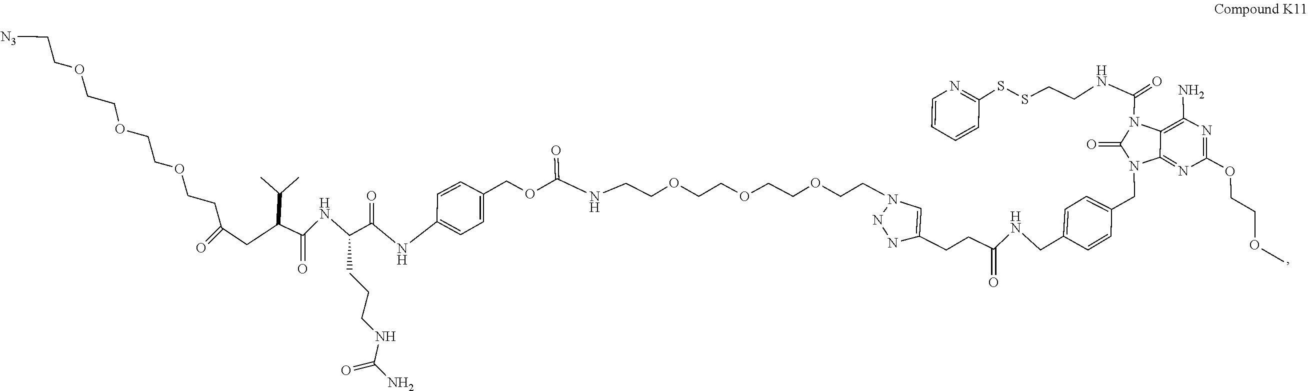

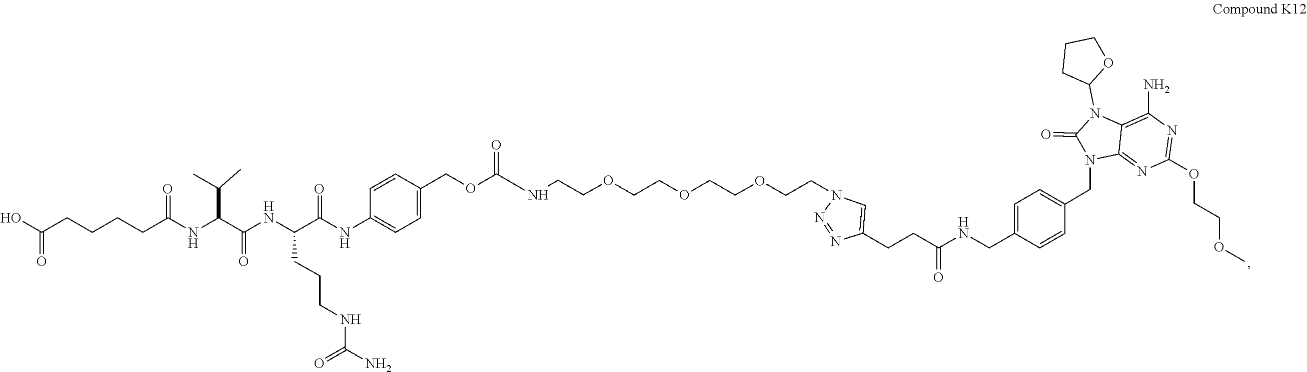

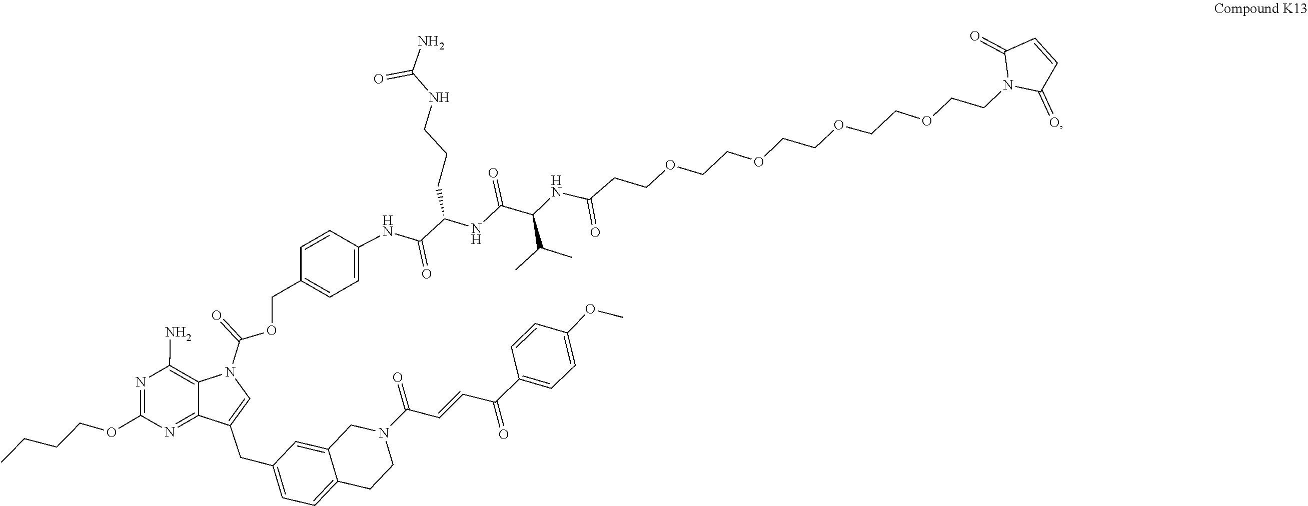 [SMILES: CCCCOc1nc(N)c2c(n1)c(Cc1ccc3c(c1)CN(C(=O)/C=C/C(=O)c1ccc(OC)cc1)CC3)cn2C(=O)OCc1ccc(NC(=O)[C@H](CCCNC(N)=O)NC(=O)[C@@H](NC(=O)CCOCCOCCOCCOCCN2C(=O)C=CC2=O)C(C)C)cc1]

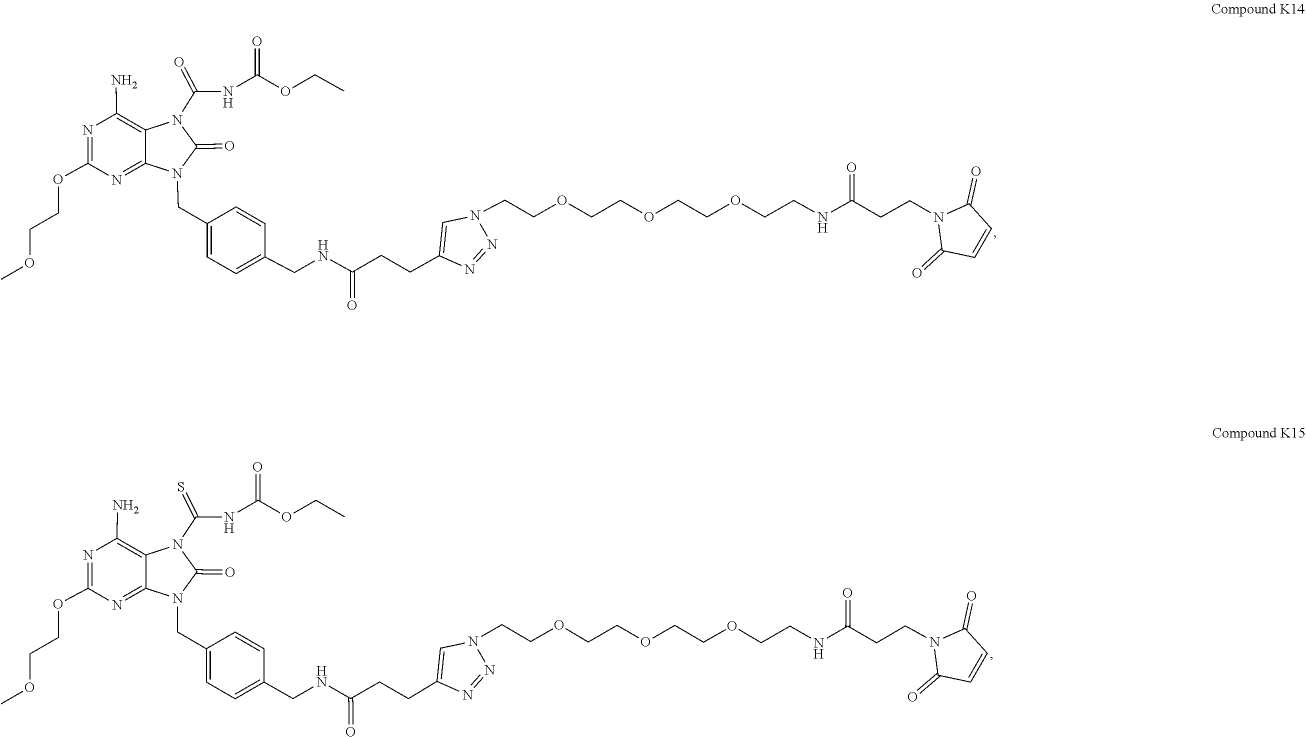 [SMILES: CCOC(=O)NC(=O)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21 ; CCOC(=O)NC(=S)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21]

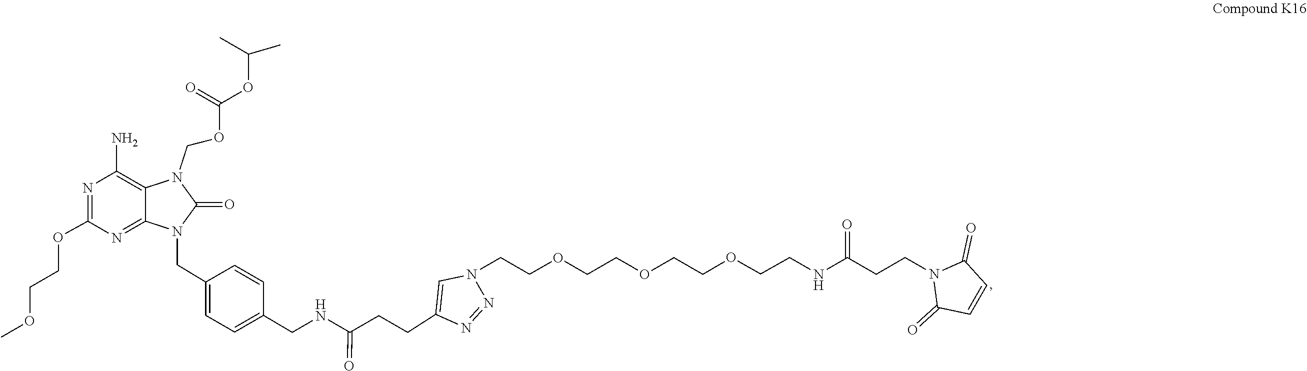 [SMILES: COCCOc1nc(N)c2c(n1)n(Cc1ccc(CNC(=O)CCc3cn(CCOCCOCCOCCNC(=O)CCN4C(=O)C=CC4=O)nn3)cc1)c(=O)n2COC(=O)OC(C)C]

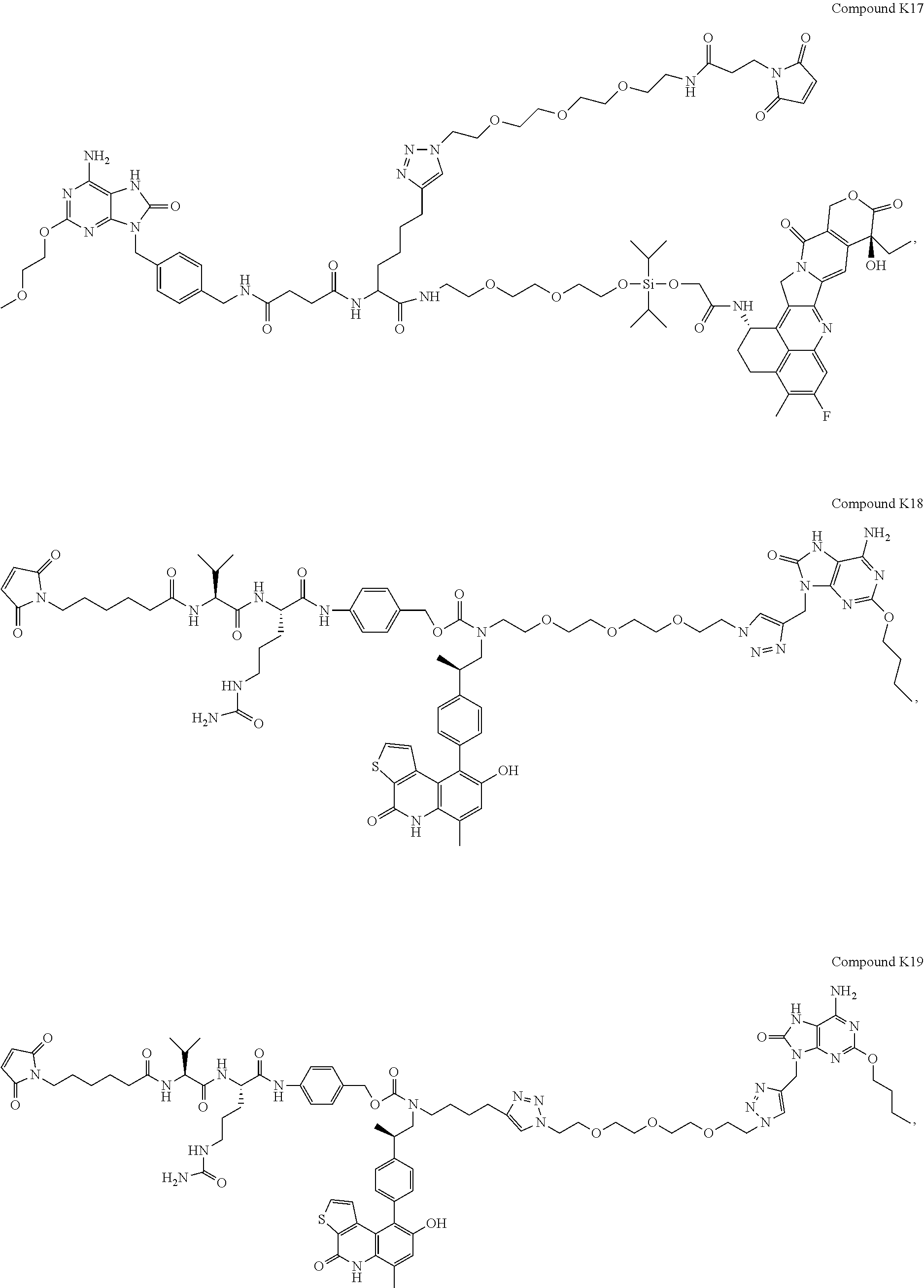

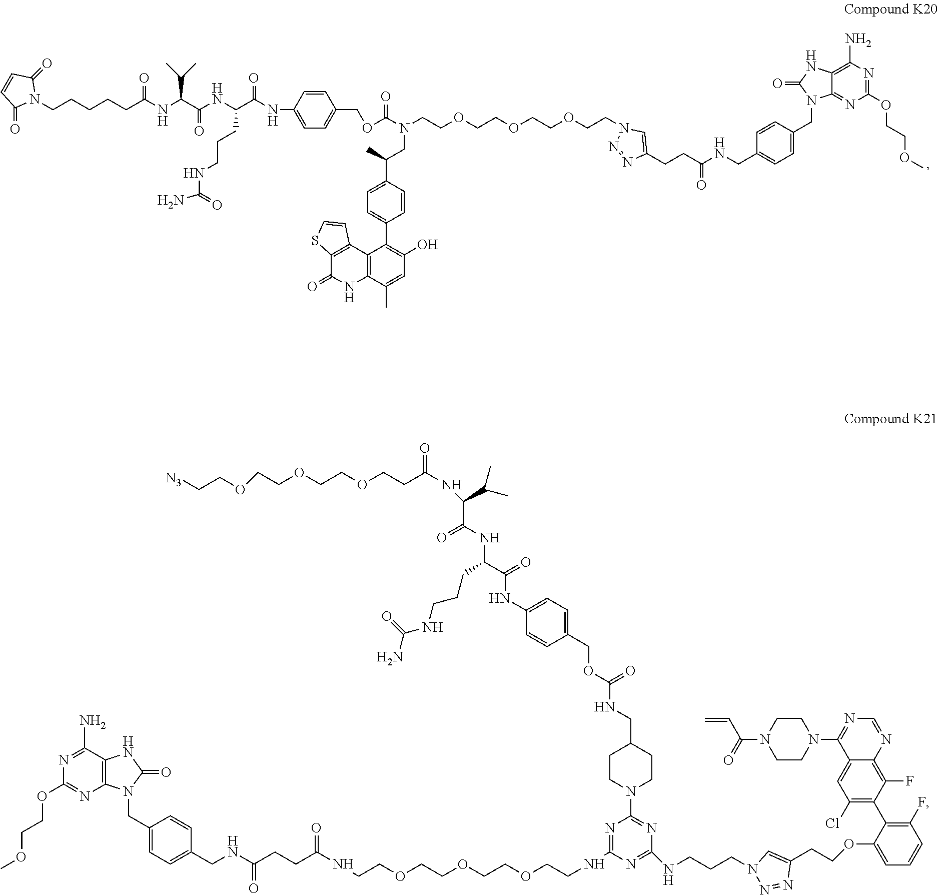

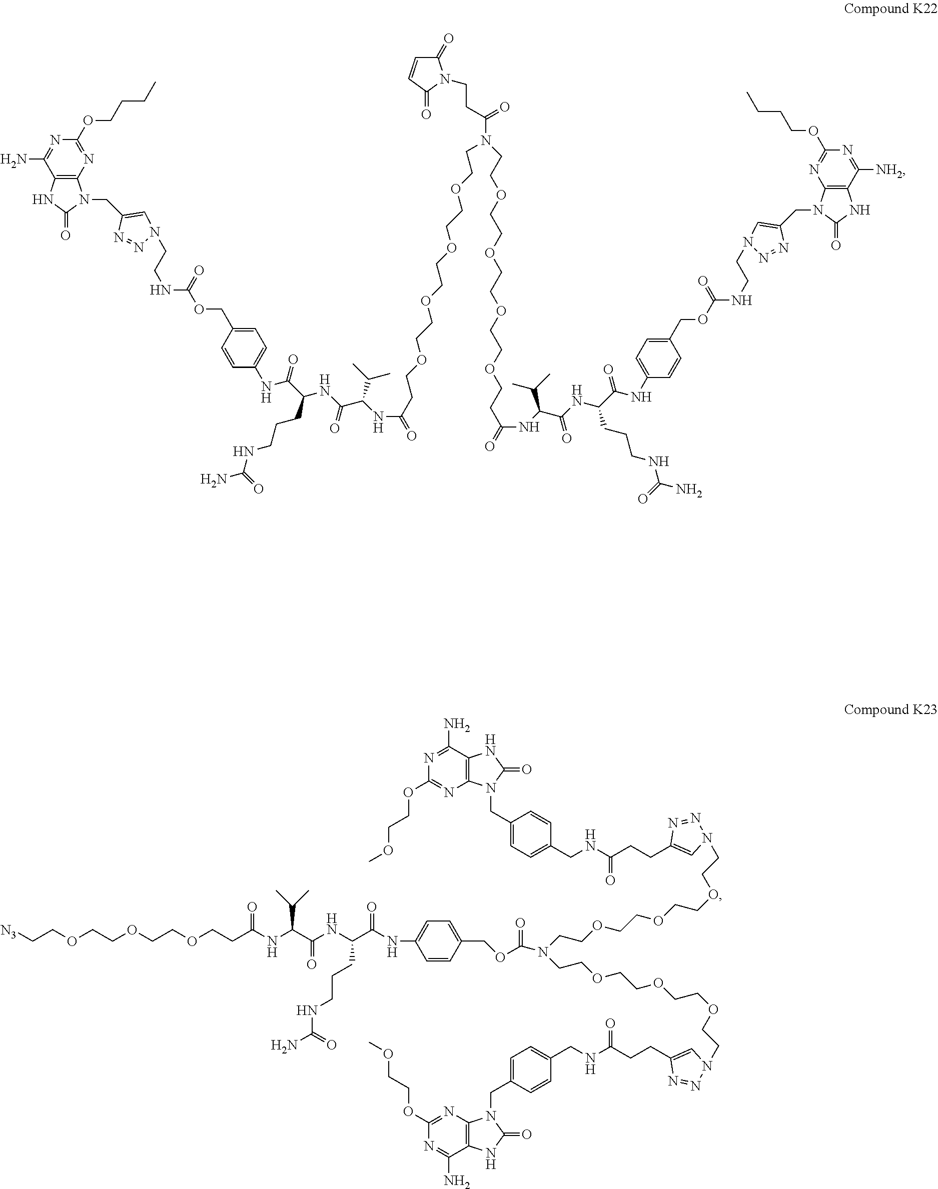

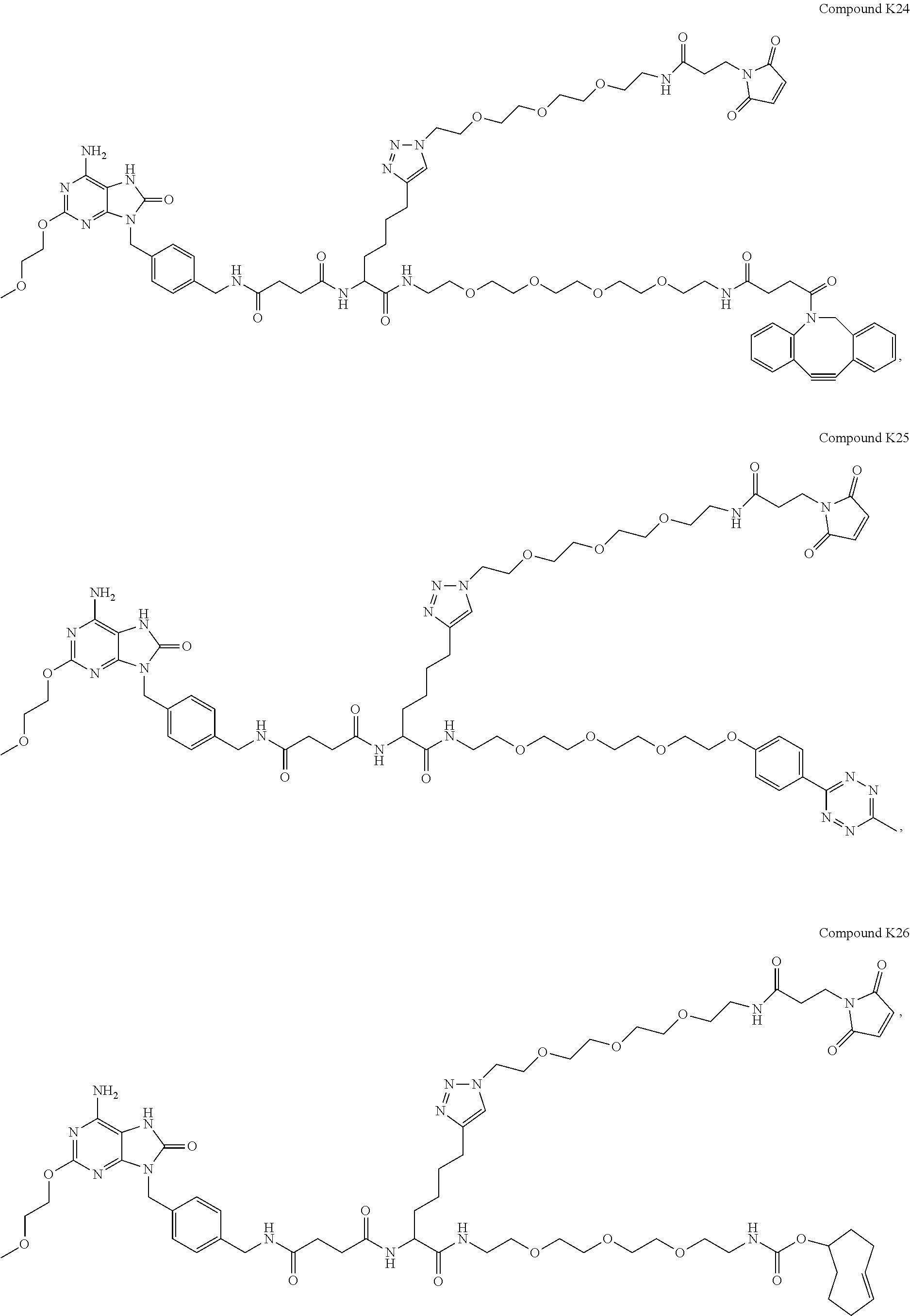

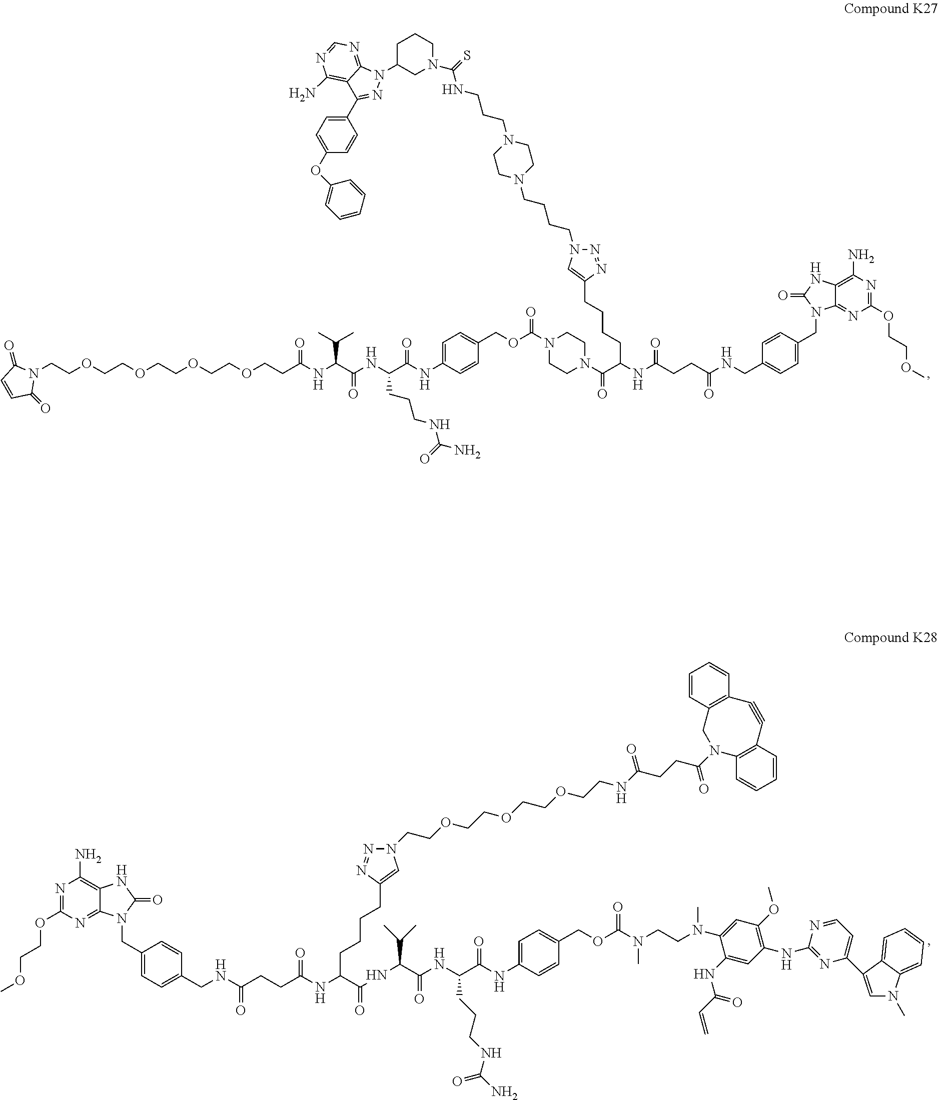

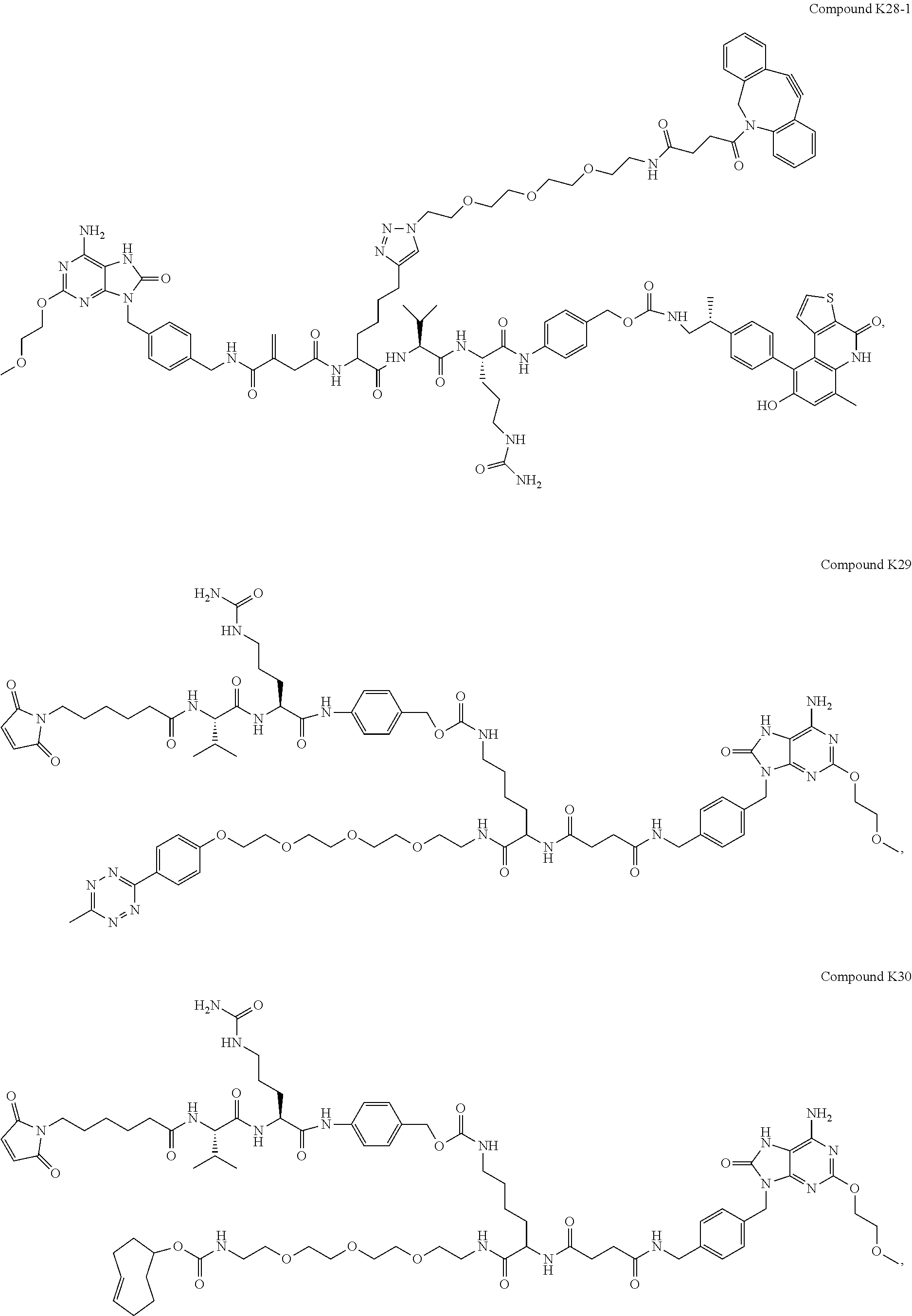

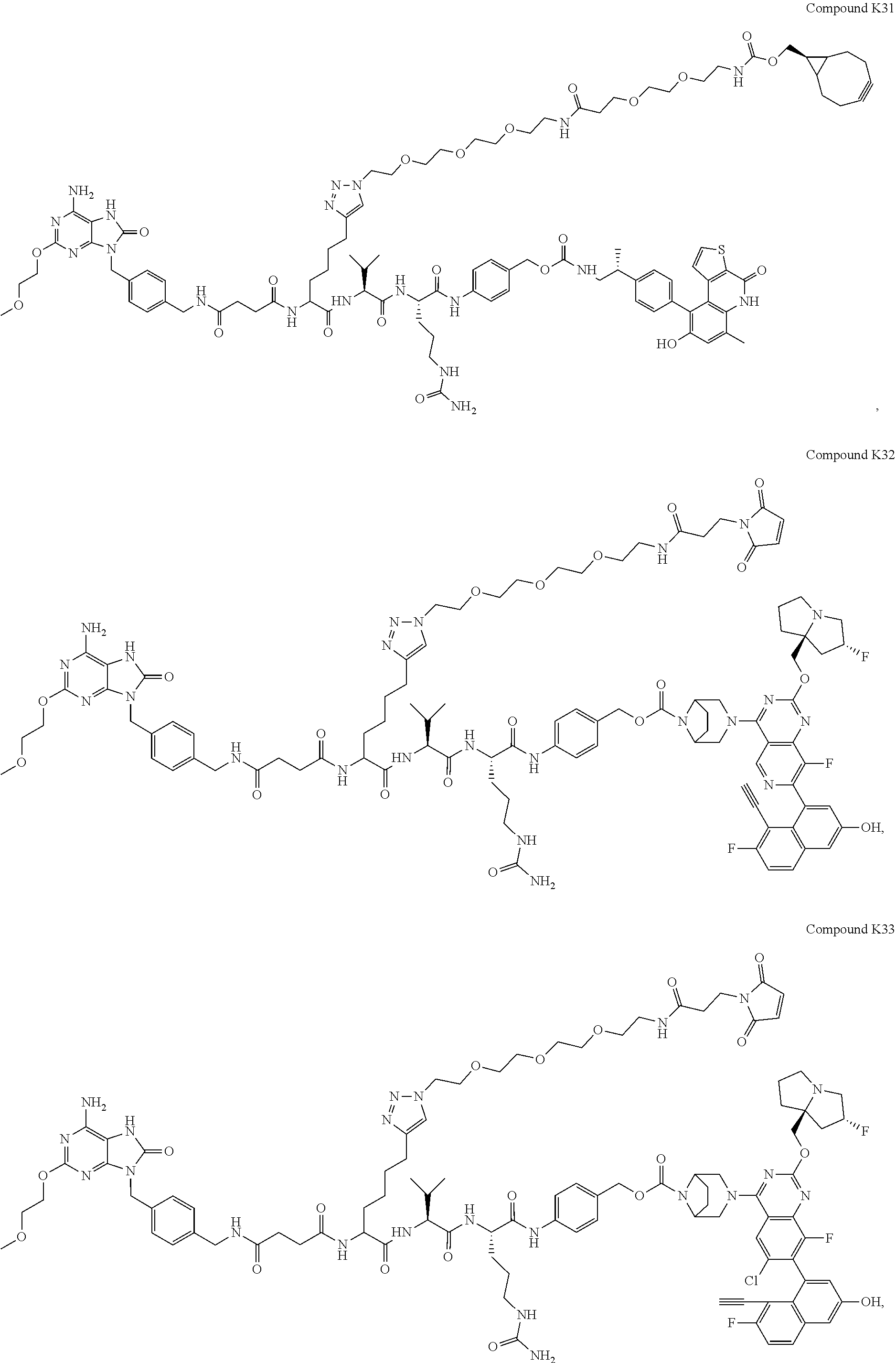

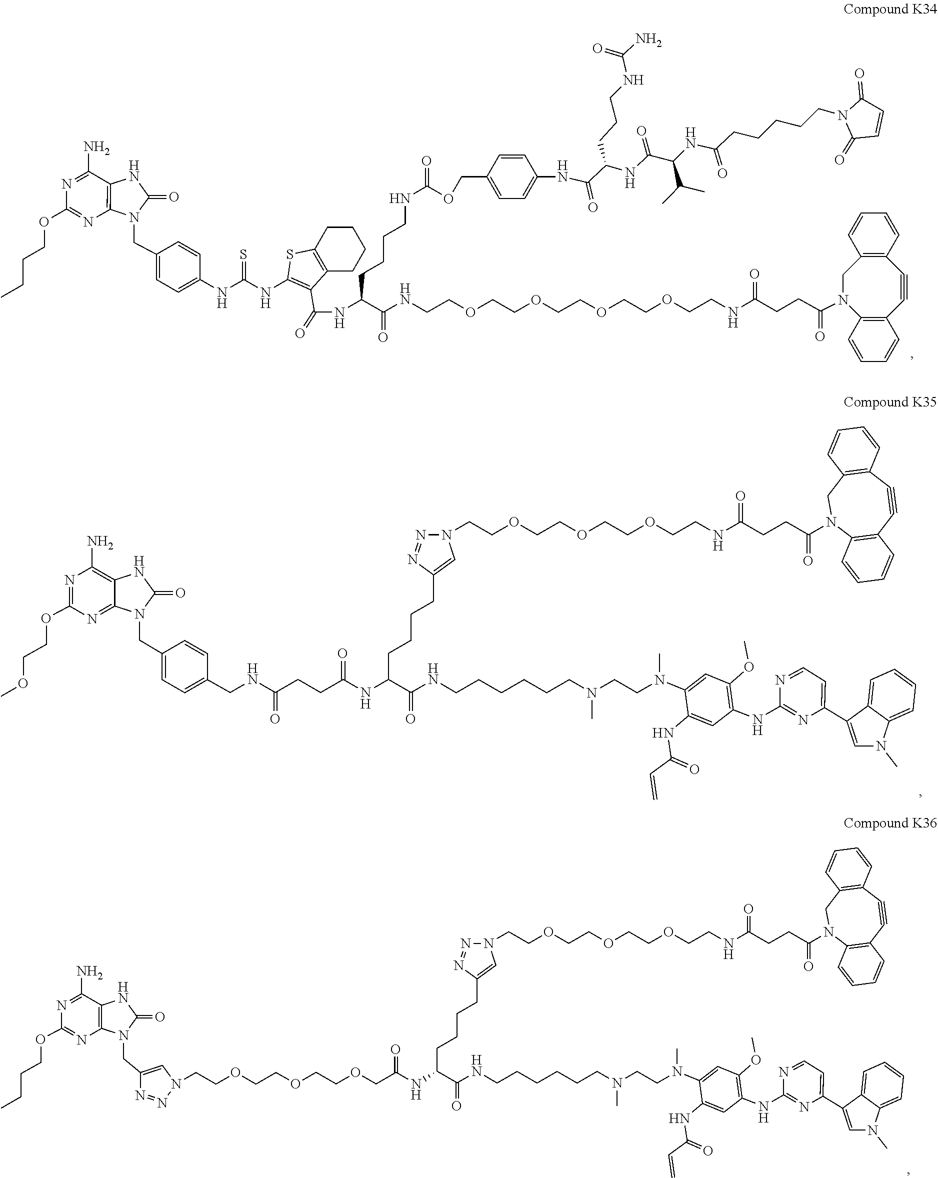

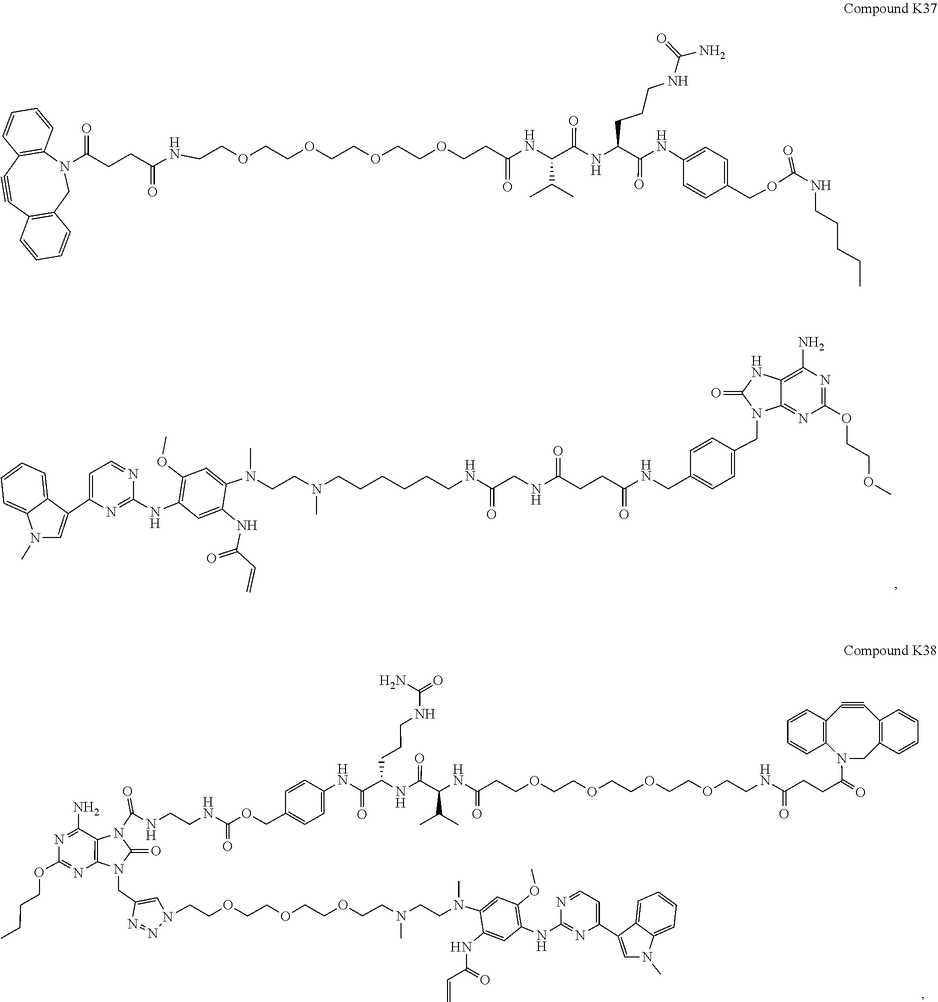

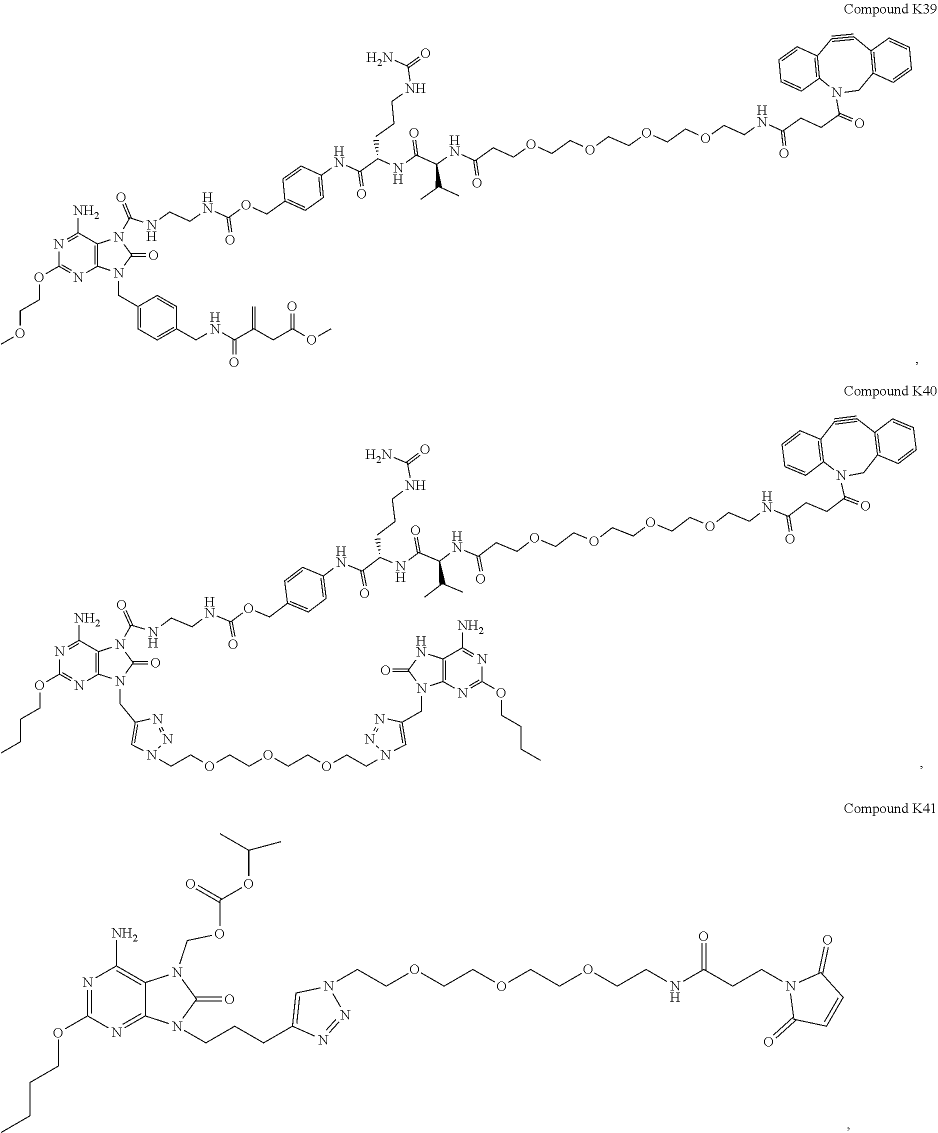 [SMILES: C=C(CC(=O)OC)C(=O)NCc1ccc(Cn2c(=O)n(C(=O)NCCNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)c3c(N)nc(OCCOC)nc32)cc1 ; CCCCOc1nc(N)c2[nH]c(=O)n(Cc3cn(CCOCCOCCOCCn4cc(Cn5c(=O)n(C(=O)NCCNC(=O)OCc6ccc(NC(=O)[C@H](CCCNC(N)=O)NC(=O)[C@@H](NC(=O)CCOCCOCCOCCOCCNC(=O)CCC(=O)N7Cc8ccccc8C#Cc8ccccc87)C(C)C)cc6)c6c(N)nc(OCCCC)nc65)nn4)nn3)c2n1 ; CCCCOc1nc(N)c2c(n1)n(CCCc1cn(CCOCCOCCOCCNC(=O)CCN3C(=O)C=CC3=O)nn1)c(=O)n2COC(=O)OC(C)C]

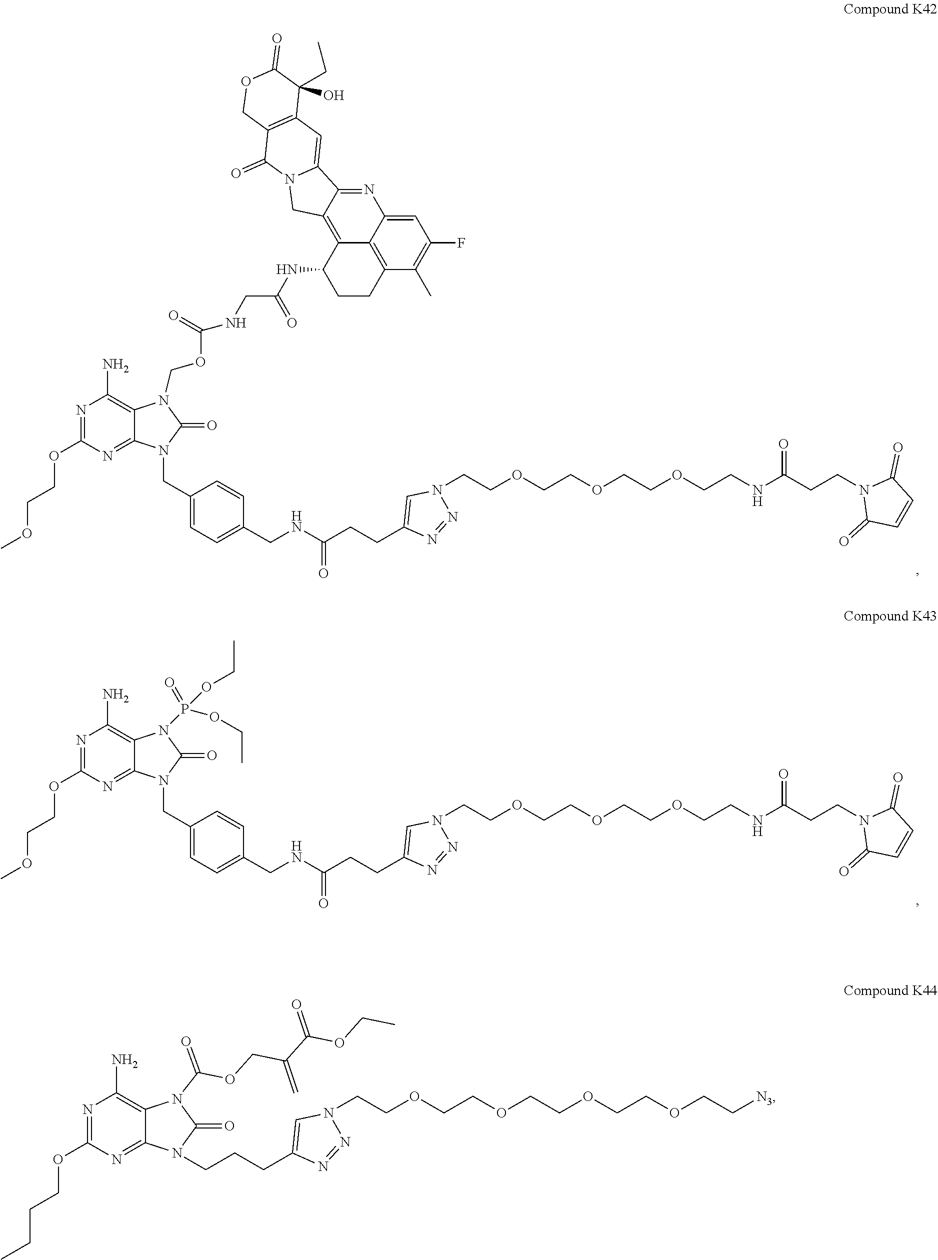 [SMILES: CC[C@@]1(O)C(=O)OCc2c1cc1n(c2=O)Cc2c-1nc1cc(F)c(C)c3c1c2[C@@H](NC(=O)CNC(=O)OCn1c(=O)n(Cc2ccc(CNC(=O)CCc4cn(CCOCCOCCOCCNC(=O)CCN5C(=O)C=CC5=O)nn4)cc2)c2nc(OCCOC)nc(N)c21)CC3 ; CCOP(=O)(OCC)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21 ; C=C(COC(=O)n1c(=O)n(CCCc2cn(CCOCCOCCOCCOCCN=[N+]=[N-])nn2)c2nc(OCCCC)nc(N)c21)C(=O)OCC]

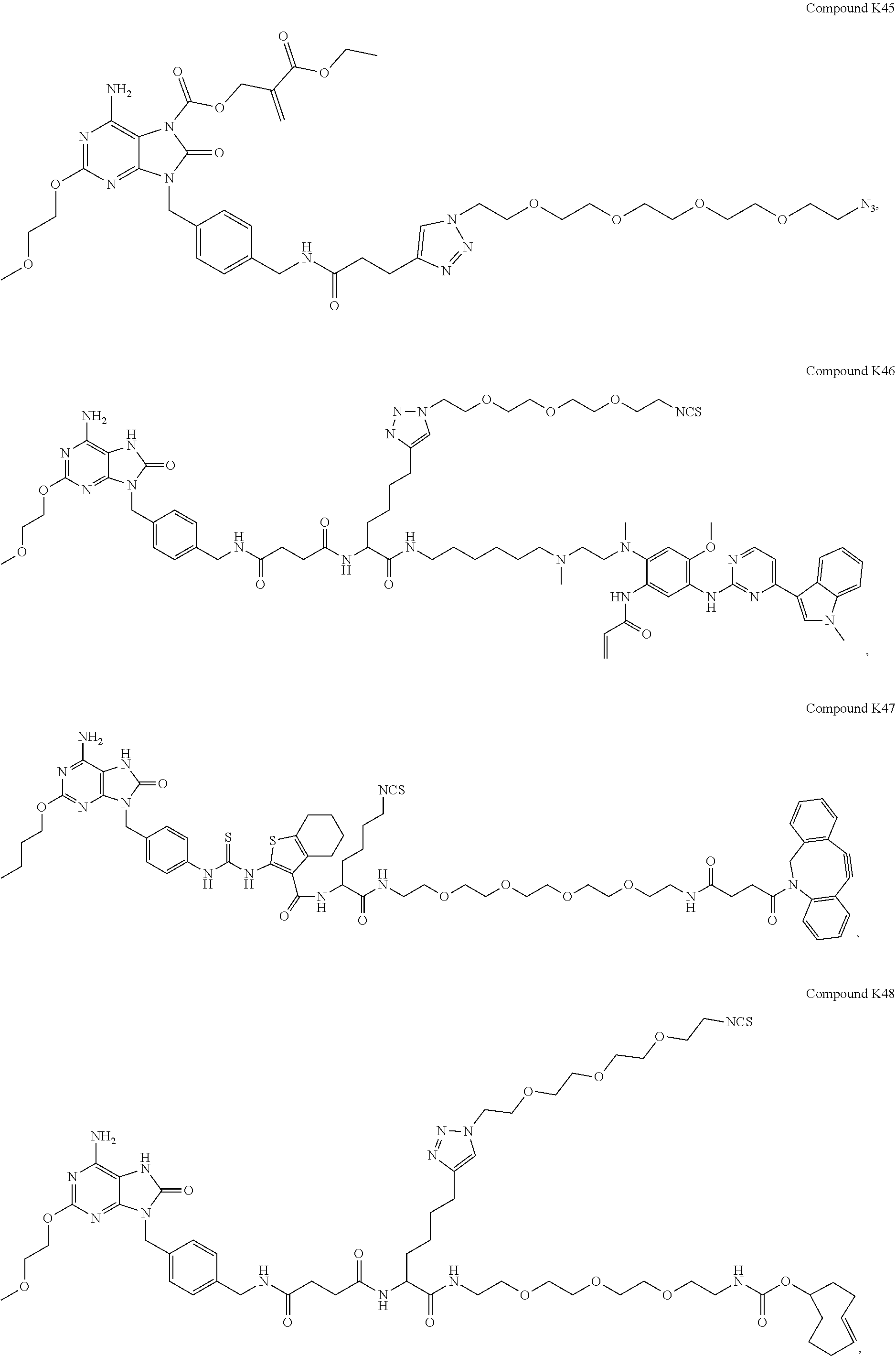

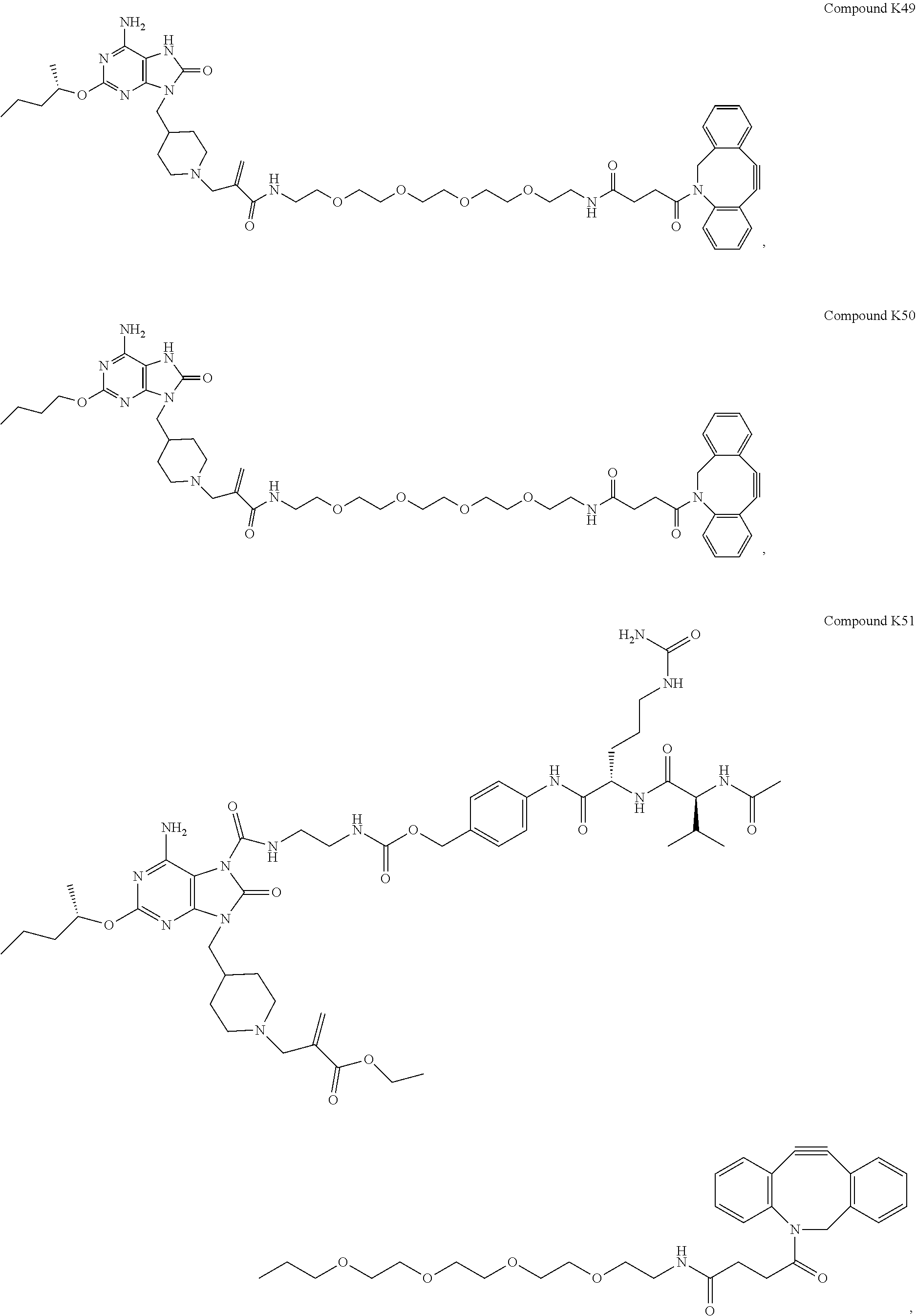

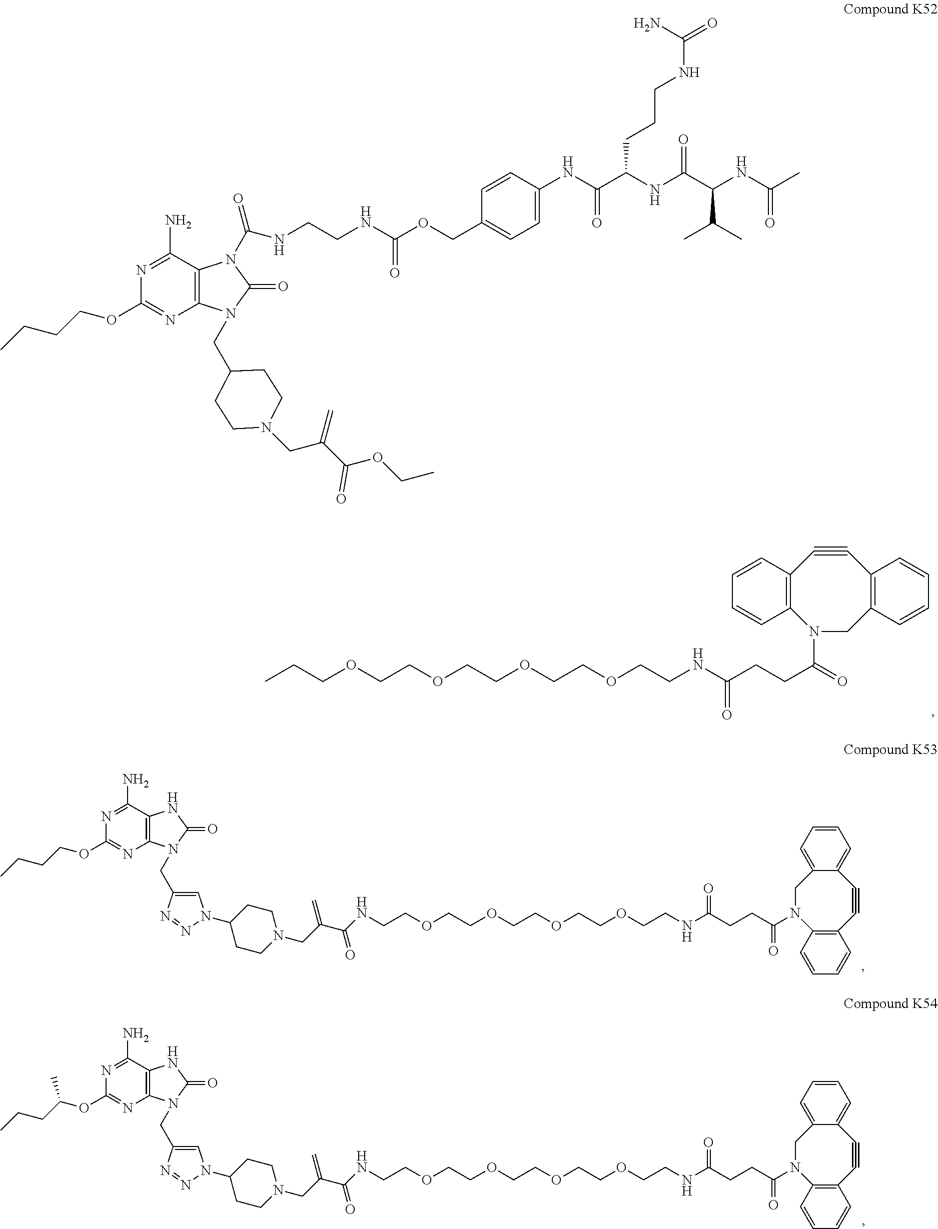

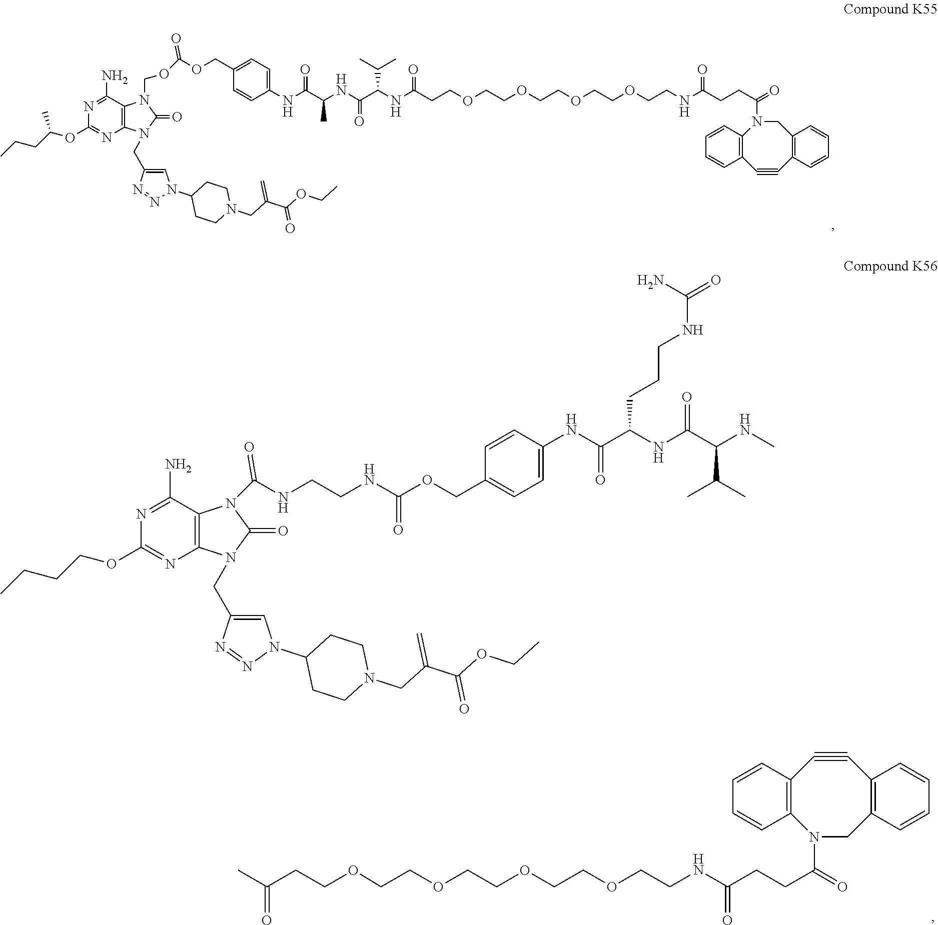

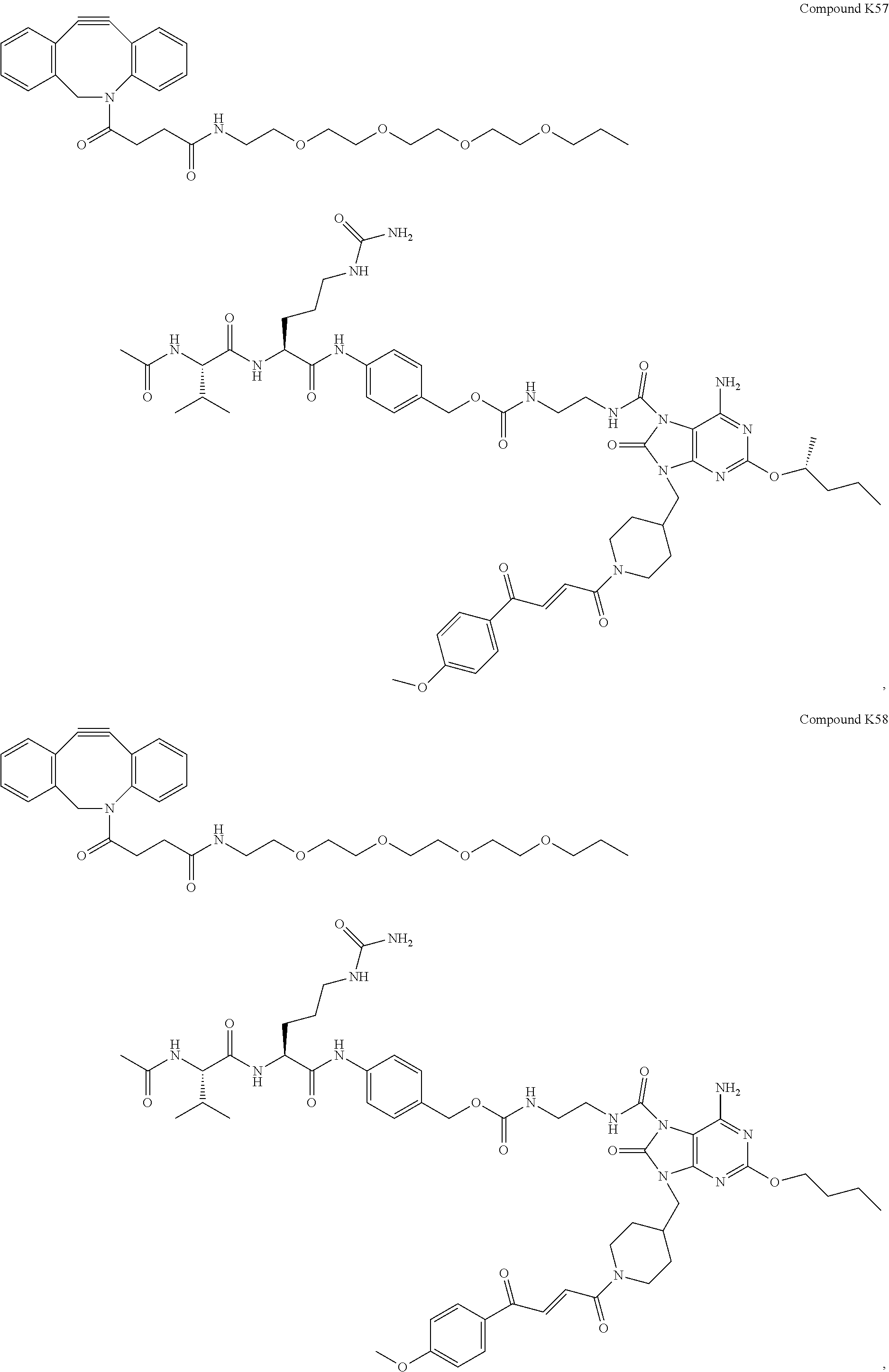

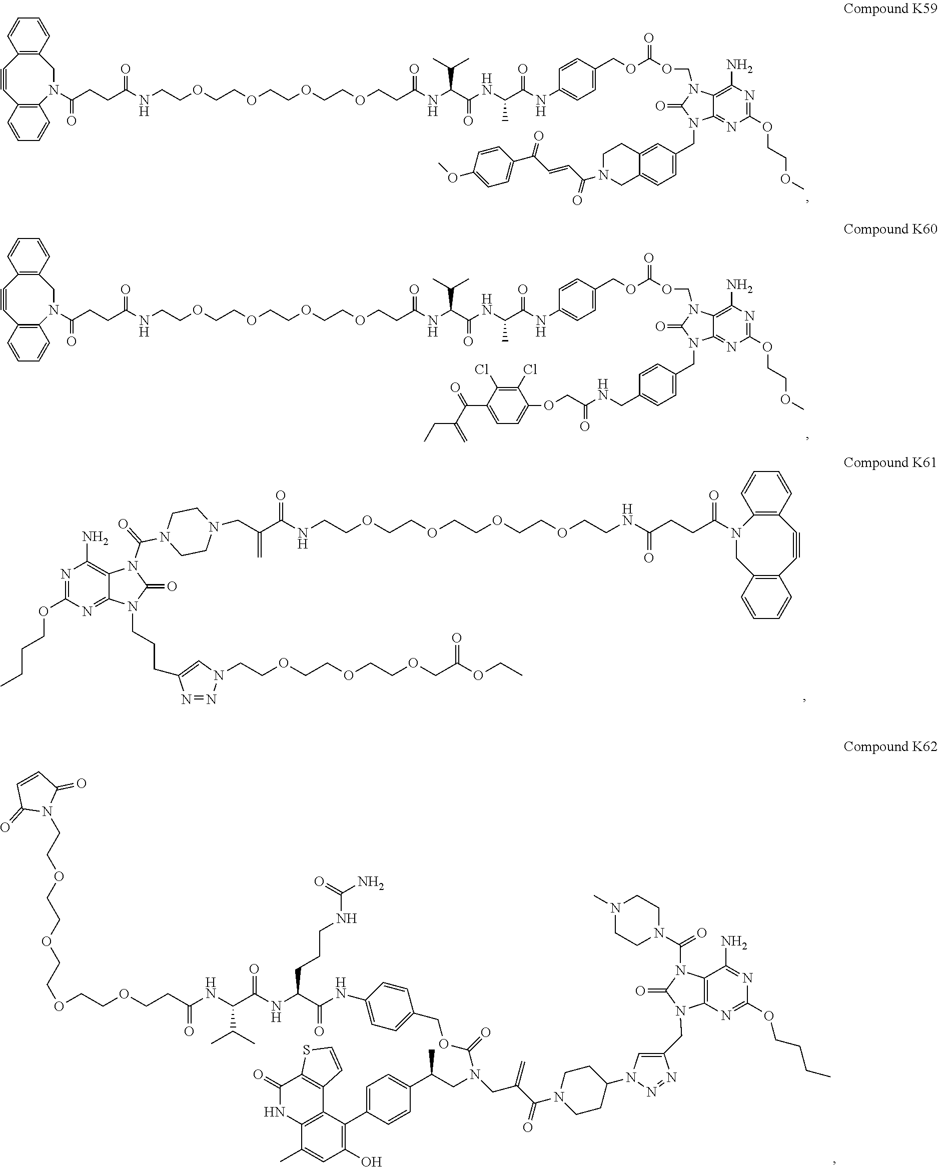

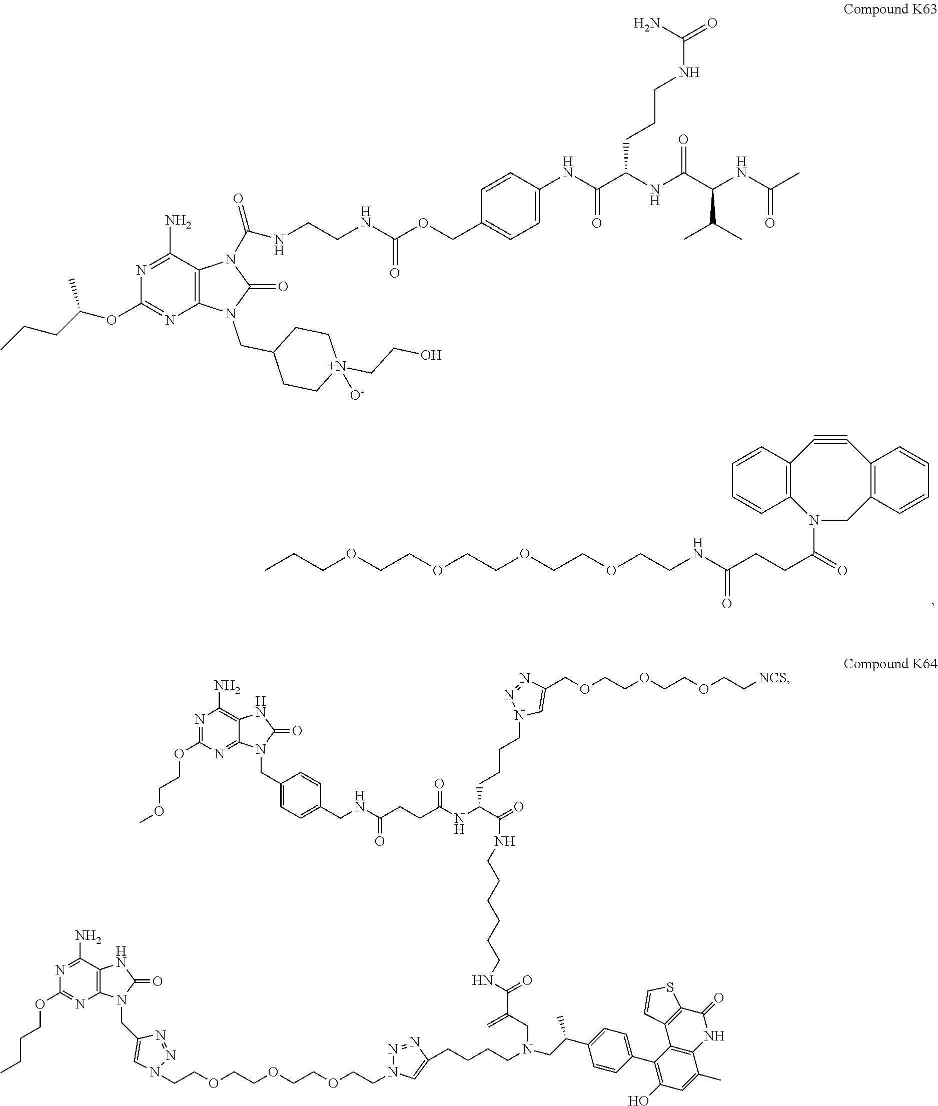

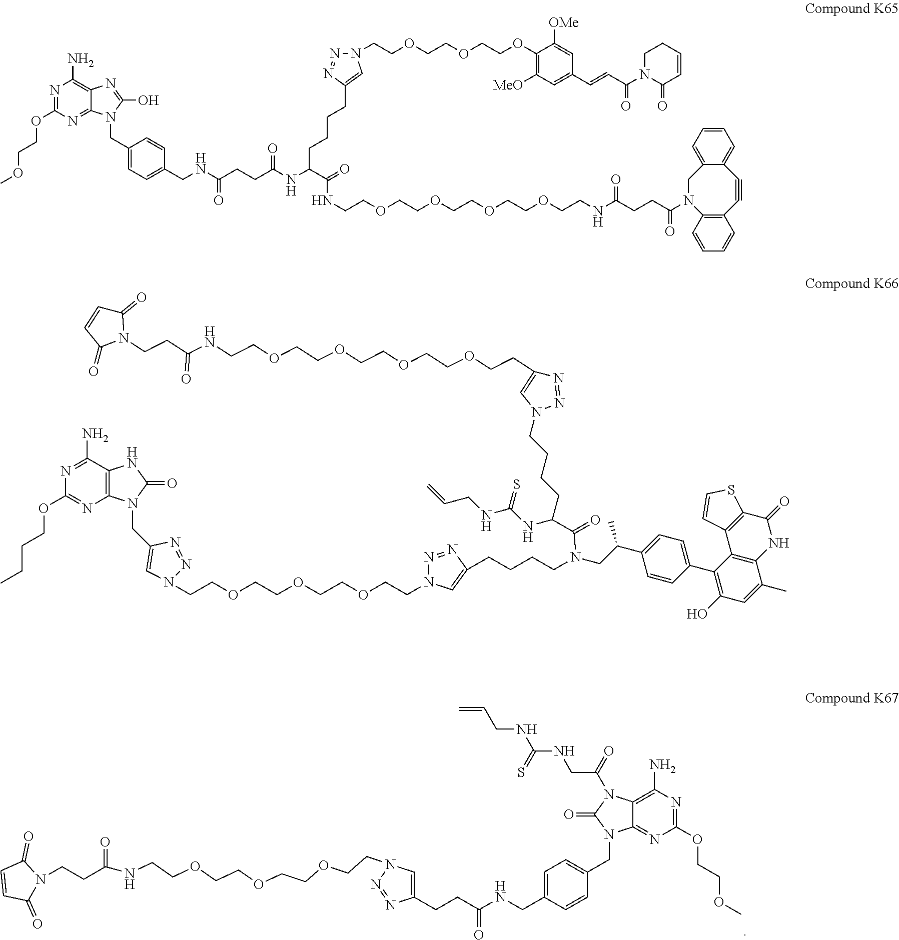 [SMILES: COCCOc1nc(N)c2nc(O)n(Cc3ccc(CNC(=O)CCC(=O)NC(CCCCc4cn(CCOCCOCCOc5c(OC)cc(/C=C/C(=O)N6CCC=CC6=O)cc5OC)nn4)C(=O)NCCOCCOCCOCCOCCNC(=O)CCC(=O)N4Cc5ccccc5C#Cc5ccccc54)cc3)c2n1 ; C=CCNC(=S)NC(CCCCn1cc(CCOCCOCCOCCOCCNC(=O)CCN2C(=O)C=CC2=O)nn1)C(=O)N(CCCCc1cn(CCOCCOCCOCCn2cc(Cn3c(=O)[nH]c4c(N)nc(OCCCC)nc43)nn2)nn1)C[C@H](C)c1ccc(-c2c(O)cc(C)c3[nH]c(=O)c4sccc4c23)cc1 ; C=CCNC(=S)NCC(=O)n1c(=O)n(Cc2ccc(CNC(=O)CCc3cn(CCOCCOCCOCCNC(=O)CCN4C(=O)C=CC4=O)nn3)cc2)c2nc(OCCOC)nc(N)c21]

2. The antibody-conjugated immune-activating small molecule compound according to claim 1, characterized in that the PD-L1 antibody is Atezolizumab, and the linking functional group is maleimide group, forming the general formula I: 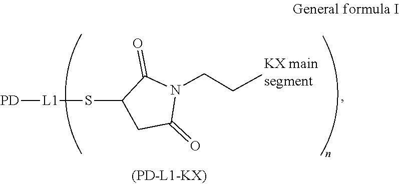
KX is an immune-activating small molecule compound, n is the DAR value, and the DAR value is 3.5-4.5.

3. The antibody-conjugated immune-activating small molecule compound according to claim 1, characterized in that the conjugation group is an azide group, forming the general formula II: 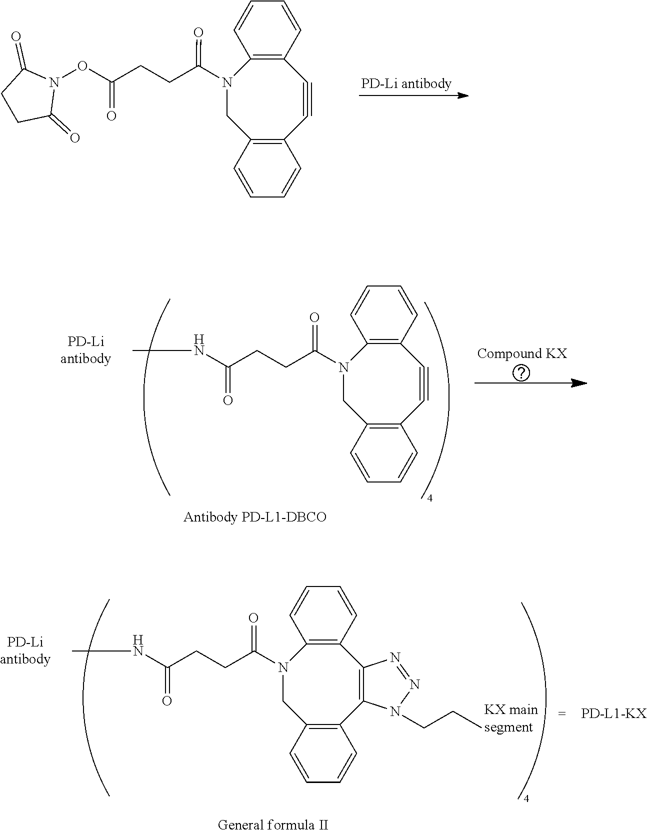
the general formula II represents PD-L1-K11, PD-L1-K21, PD-L1-K23, PD-L1-K44, and PD-L1-K45.

4. The antibody-conjugated immune-activating small molecule compound according to claim 1, characterized in that the conjugation group is DBCO group, forming the general formula III: 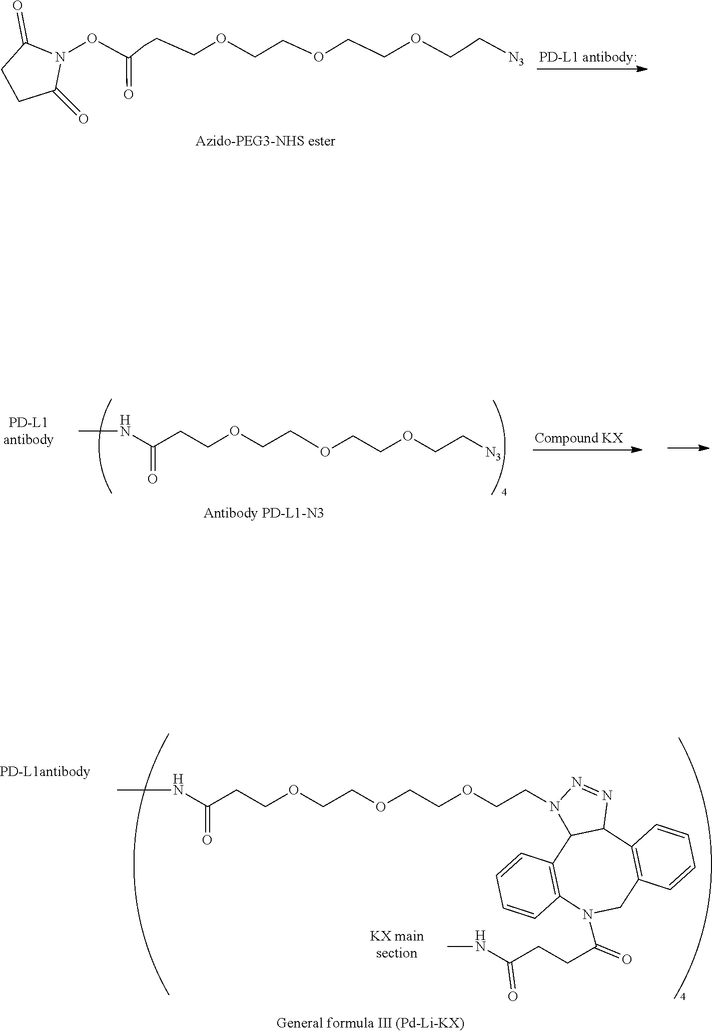
wherein the general formula III represents the following conjugated antibodies: PD-L1-K28, PD-L1-K28-1, PD-L1-K31, PD-L1-K35, PD-L1-K36, PD-L1-K37, PD-L1-K38, PD-L1-K39, PD-L1-K49, PD-L1-K50, PD-L1-K51, PD-L1-K52, PD-L1-K53, PD-L1-K54, PD-L1-K55, PD-L1-K56, PD-L1-K57, PD-L1-K58, PD-L1-K59, PD-L1-K60, PD-L1-K61, PD-L1-K63, PD-L1-K65.

5. The antibody-conjugated immune-activating small molecule compound according to claim 1, characterized in that the conjugation group is carboxyl, forming the general formula IV: 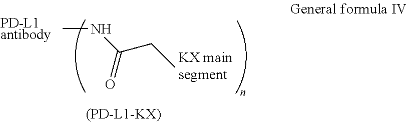
wherein the general formula IV represents the following conjugated antibodies: PD-L1-K4, PD-L1-K5, PD-L1-K9, PD-L1-K12.

6. The antibody-conjugated immune-activating small molecule compound according to claim 1, characterized in that the conjugation group is isothiocyano, forming the general formula V: 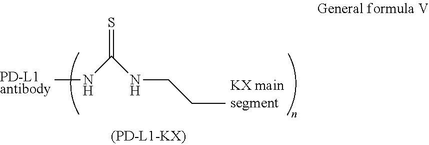
wherein the general formula V represents the following conjugated antibodies: PD-L1-K40, PD-L1-K46, PD-L1-K47, PD-L1-K48, PD-L1-K64.

7. Use of the antibody-conjugated immune-activating small molecule compound according to claim 1 in the manufacture of an immunomodulatory drug.

8. Use of the antibody-conjugated immune-activating small molecule compound according to claim 1 in the manufacture of a conjugated antibody.

9. Use of the antibody-conjugated immune-activating small molecule compound according to claim 1 in the manufacture of an antitumor drug.
